(19) United States
(12) Patent Application Publication
(10) Pub. No.: US 2023/0285377 A1
(43) Pub. Date: Sep. 14, 2023

(54) KCNT1 INHIBITORS AND METHODS OF USE

(71) Applicant: Praxis Precision Medicines, Inc., Boston, MA (US)

(72) Inventors: Gabriel MARTINEZ BOTELLA, Wayland, MA (US); Andrew Mark GRIFFIN, L'lle Bizard (CA)

(21) Appl. No.: 17/906,893

(22) PCT Filed: Mar. 23, 2021
(86) PCT No.: PCT/US2021/023653

§ 371 Date: Sep. 21, 2022

Related U.S. Application Data
(60) Provisional application No. 62/993,359, filed on Mar. 23, 2020.

(51) Int. Cl. A61K 31/454; A61K 31/4545; A61K 31/4709; A61K 31/538

(52) U.S. Cl. CPC A61K 31/454; A61K 31/538; A61K 31/4545; A61K 31/4709

(57) ABSTRACT

The present invention is directed to, in part, compounds and compositions useful for preventing and/or treating a neurological disease or disorder, a disease or condition relating to excessive neuronal excitability, and/or a gain-of-function mutation in a gene (e.g., KCNT1). Methods of treating a neurological disease or disorder, a disease or condition relating to excessive neuronal excitability, and/or a gain-of-function mutation in a gene such as KCNT1 are also provided herein.

### CROSS-REFERENCE TO RELATED APPLICATIONS

This application claims priority to and the benefit of U.S. Provisional Pat. Application Number 62/993,359 filed Mar. 23, 2020, the content of each of which is incorporated herein by reference in their entirety.

### BACKGROUND

KCNT1 encodes sodium-activated potassium channels known as Slack (Sequence like a calcium-activated K⁺ channel). These channels are found in neurons throughout the brain and can mediate a sodium-activated potassium current I_{KNa}. This delayed outward current can regulate neuronal excitability and the rate of adaption in response to maintained stimulation. Abnormal Slack activity have been associated with development of early onset epilepsies and intellectual impairment. Accordingly, pharmaceutical compounds that selectively regulate sodium-activated potassium channels, e.g., abnormal KCNT1, abnormal I_{KNa}, are useful in treating a neurological disease or disorder or a disease or condition related to excessive neuronal excitability and/or KCNT1 gain-of-function mutations.

### SUMMARY OF THE INVENTION

Described herein are compounds and compositions useful for preventing and/or treating a disease, disorder, or condition, e.g., a neurological disease or disorder, a disease, disorder, or condition associated with excessive neuronal excitability and/or a gain-of-function mutation in a gene, for example, KCNT1.

Thus, in one aspect, provided herein is a pharmaceutical composition comprising a compound having the Formula A:

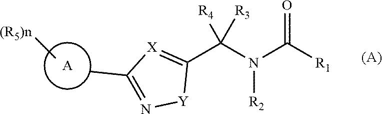

X is CR₇ or N and Y is S; or
X is CR₇ and Y is O;
ring A is selected from the group consisting of phenyl, 6-membered heteroaryl, and 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₋₆alkoxy, C₁₋₆haloalkoxy, and C₃₋scycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 6-membered heterocyclyl or 6-membered heteroaryl, R₁ is not thiophene;
provided that when R₃ is hydrogen and ring A is 6-membered heteroaryl or 5-membered heterocyclyl, R₁ is not phenyl; or a pharmaceutically acceptable salt thereof,
and a pharmaceutically acceptable carrier.

In another aspect, provided herein is a pharmaceutical composition comprising a compound having the Formula A-1:

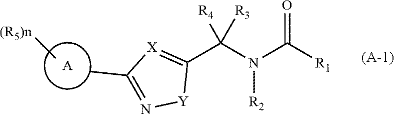

X is CR₇ or N and Y is S; or
X is CR₇ and Y is O;
ring A is 6-membered heteroaryl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₋₆alkoxy, C₁₋₆haloalkoxy, and C₃₋scycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 6-membered heteroaryl, R₁ is not thiophene or phenyl; or a pharmaceutically acceptable salt thereof,
and a pharmaceutically acceptable carrier.

In another aspect, provided herein is a pharmaceutical composition comprising a compound having the Formula A-2:

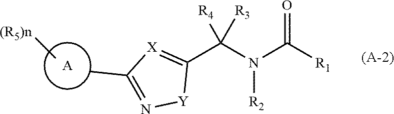

X is CR₇ or N and Y is S; or
X is CR₇ and Y is O;
ring A is 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₋₆alkoxy, C₁₋₆haloalkoxy, and C₃₋scycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 5-6-membered heterocyclyl, R₁ is not thiophene or phenyl; or a pharmaceutically acceptable salt thereof,
and a pharmaceutically acceptable carrier.

In one aspect, provided herein is a compound having the Formula I:

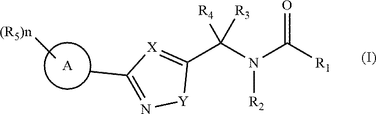

or a pharmaceutically acceptable salt thereof, wherein:
X is CR₇ or N and Y is S; or
X is CR₇ and Y is O;
ring A is selected from the group consisting of phenyl, 6-membered heteroaryl, and 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋scycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

In an aspect, provided herein is a compound having the Formula I-A:

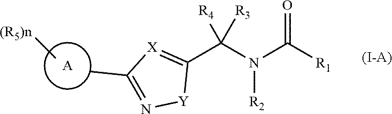

or a pharmaceutically acceptable salt thereof, wherein:
X is CR₇ or N and Y is S; or
X is CR₇ and Y is O;
ring A is 6-membered heteroaryl or 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-10 membered carbocyclyl, and 5-10 membered heterocyclyl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋scycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

In an aspect, provided herein is a compound having the Formula I-B:

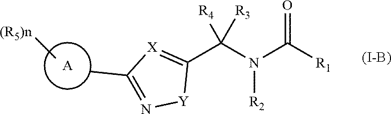

or a pharmaceutically acceptable salt thereof, wherein:
X is CR₇ or N and Y is S; or
X is CR₇ and Y is O;
ring A is phenyl or 6-membered heteroaryl;
R₁ is phenyl or 5-6 membered heteroaryl, wherein the phenyl or 5-6 membered heteroaryl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋scycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆haloalkyl, C₁₋₆alkoxy, C₁₋₆haloalkoxy, —S(O)₂R₈, —S(O)₂—N(R₉)₂, and C₃-scycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

In one aspect, the present disclosure provides a method of treating neurological disease or disorder, wherein the method comprises administering to a subject in need thereof a compound disclosed herein (e.g., compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B) or a pharmaceutical composition disclosed herein (e.g., a pharmaceutical composition comprising a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B), or a pharmaceutically acceptable salt thereof, and a pharmaceutically acceptable excipient).

In another aspect, the present disclosure provides a method of treating a disease or condition associated with excessive neuronal excitability, wherein the method comprises administering to a subject in need thereof a compound disclosed herein (e.g., compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B) or a pharmaceutical composition disclosed herein (e.g., a pharmaceutical composition comprising a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3) (I-IB4), (I-IC), (I-IC2), (I-IC3), (I-IC4), (II), (II-A), or (II-B), or a pharmaceutically acceptable salt thereof, and a pharmaceutically acceptable excipient).

In another aspect, the present disclosure provides a method of treating a disease or condition associated with a gain-of-function mutation of a gene (e.g. KCNT1), wherein the method comprises administering to a subject in need thereof a compound disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B) or a pharmaceutical composition disclosed herein (e.g., a pharmaceutical composition comprising a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3) (I-IC4), (II), (II-A), or (II-B), or a pharmaceutically acceptable salt thereof, and a pharmaceutically acceptable excipient).

In some embodiments, the neurological disease or disorder, the disease or condition associated with excessive neuronal excitability, or the disease or condition associated with a gain-of-function mutation of a gene (e.g., KCNT1) is epilepsy, an epilepsy syndrome, or an encephalopathy.

In some embodiments, the neurological disease or disorder, the disease or condition associated with excessive neuronal excitability, or the disease or condition associated with a gain-of-function mutation of a gene (e.g., KCNT1) is a genetic or pediatric epilepsy or a genetic or pediatric epilepsy syndrome.

In some embodiments, the neurological disease or disorder, the disease or condition associated with excessive neuronal excitability, or the disease or condition associated with a gain-of-function mutation of a gene (e.g., KCNT1) is a cardiac dysfunction.

In some embodiments, the neurological disease or disorder, the disease or condition associated with excessive neuronal excitability, or the disease or condition associated with a gain-of-function mutation of a gene (e.g., KCNT1) is selected from epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, cerebellar ataxia).

In some embodiments, the neurological disease or disorder, the disease or condition associated with excessive neuronal excitability, or the disease or condition associated with a gain-of-function mutation of a gene (e.g., KCNT1) is selected from the group consisting of cardiac arrhythmia, sudden unexpected death in epilepsy, Brugada syndrome, and myocardial infarction.

In some embodiments, the neurological disease or disorder, the disease or condition associated with excessive neuronal excitability, or the disease or condition associated with a gain-of-function mutation of a gene (e.g., KCNT1) is selected from pain and related conditions (e.g. neuropathic pain, acute/chronic pain, migraine, etc).

In some embodiments, the neurological disease or disorder, the disease or condition associated with excessive neuronal excitability, or the disease or condition associated with a gain-of-function mutation of a gene (e.g., KCNT1) is a muscle disorder (e.g. myotonia, neuromyotonia, cramp muscle spasms, spasticity).

In some embodiments, the neurological disease or disorder, the disease or condition associated with excessive neuronal excitability, or the disease or condition associated with a gain-of-function mutation of a gene (e.g., KCNT1) is selected from itch and pruritis, ataxia and cerebellar ataxias.

In some embodiments, the neurological disease or disorder, the disease or condition associated with excessive neuronal excitability, or the disease or condition associated with a gain-of-function mutation of a gene (e.g., KCNT1) is selected from psychiatric disorders (e.g. major depression, anxiety, bipolar disorder, schizophrenia).

In some embodiments, the neurological disease or disorder or the disease or condition associated with excessive neuronal excitability and/or a gain-of-function mutation in a gene (e.g., KCNT1) is selected from the group consisting of learning disorders, Fragile X, neuronal plasticity, and autism spectrum disorders.

In some embodiments, the neurological disease or disorder, the disease or condition associated with excessive neuronal excitability, or the disease or condition associated with a gain-of-function mutation of a gene (e.g., KCNT1) is selected from the group consisting of epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.

Other objects and advantages will become apparent to those skilled in the art from a consideration of the ensuing Detailed Description, Examples, and Claims.

### DETAILED DESCRIPTION OF THE INVENTION

As generally described herein, the present invention provides compounds and compositions useful for preventing and/or treating a disease, disorder, or condition described herein, e.g., a disease, disorder, or condition associated with excessive neuronal excitability, and/or a disease, disorder, or condition associated with gain-of-function mutations in KCNT1. Exemplary diseases, disorders, or conditions include epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, and Lennox Gastaut syndrome, seizures, leukodystrophy, leukoencephalopathy, Intellectual disability, Multifocal Epilepsy, Generalized tonic clonic seizures, Drug resistant epilepsy, Temporal lobe epilepsy, cerebellar ataxia, Asymmetric Tonic Seizures) and cardiac dysfunctions (e.g., cardiac arrhythmia, Brugada syndrome, sudden unexpected death in epilepsy, myocardial infarction), pain and related conditions (e.g. neuropathic pain, acute/chronic pain, migraine, etc), muscle disorders (e.g. myotonia, neuromyotonia, cramp muscle spasms, spasticity), itch and pruritis, ataxia and cerebellar ataxias, and psychiatric disorders (e.g. major depression, anxiety, bipolar disorder, schizophrenia).

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March’s Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

The following terms are intended to have the meanings presented therewith below and are useful in understanding the description and intended scope of the present invention. When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

The term “heteroalkyl” as used herein refers to an “alkyl” group in which at least one carbon atom has been replaced with an O or S atom. The heteroalkyl may be, for example, an -O-C₁-C₁₀alkyl group, an -C₁-C₆alkylene-O-C₁-C₆alkyl group, or a C₁-C₆ alkylene-OH group. In certain embodiments, the “heteroalkyl” may be 2-8 membered heteroalkyl, indicating that the heteroalkyl contains from 2 to 8 atoms selected from the group consisting of carbon, oxygen, nitrogen, and sulfur. In yet other embodiments, the heteroalkyl may be a 2-6 membered, 4-8 membered, or a 5-8 membered heteroalkyl group (which may contain for example 1 or 2 heteroatoms selected from the group oxygen and nitrogen). In certain embodiments, the heteroalkyl is an “alkyl” group in which 1-3 carbon atoms have been replaced with oxygen atoms. One type of heteroalkyl group is an “alkoxy” group.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂-6 alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

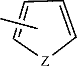

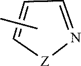

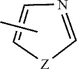

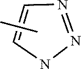

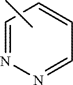

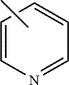

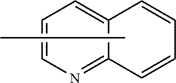

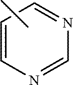

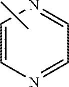

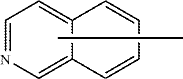

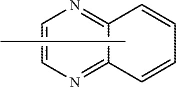

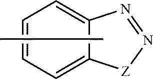

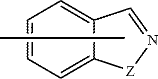

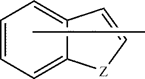

wherein each Z is selected from carbonyl, N, NR⁶⁵, O, and S; and R⁶⁵ is independently hydrogen, C₁-C₈ alkyl, C₃-C₁₀ carbocyclyl, 4-10 membered heterocyclyl, C₆-C₁₀ aryl, and 5-10 membered heteroaryl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃),cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. Cycloalkyl groups can be fused to other cycloalkyl, aryl, or heterocyclyl groups. In certain embodiments, the cycloalkyl group is not substituted, i.e., it is unsubstituted.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl; carbocyclyl, e.g., heterocyclyl; aryl, e.g,. heteroaryl; and the like having from 1 to 5, and particularly from 1 to 3 heteroatoms.

As used herein, “cyano” refers to —CN.

As used herein, “halo” or “halogen” refers to fluoro (F), chloro (Cl), bromo (Br) and iodo (I). In certain embodiments, the halo group is either fluoro or chloro.

As used herein, “haloalkyl” refers to an alkyl group substituted with one or more halogen atoms.

As used herein, “nitro” refers to —NO₂.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, -N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, “prophylactic treatment” contemplates an action that occurs before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, a “disease or condition associated with a gain-of-function mutation in KCNT1” refers to a disease or condition that is associated with, is partially or completely caused by, or has one or more symptoms that are partially or completely caused by, a mutation in KCNT1 that results in a gain-of-function phenotype, i.e. an increase in activity of the potassium channel encoded by KCNT1 resulting in an increase in whole cell current.

As used herein, a “gain-of-function mutation” is a mutation in KCNT1 that results in an increase in activity of the potassium channel encoded by KCNT1.Activity can be assessed by, for example, ion flux assay or electrophysiology (e.g. using the whole cell patch clamp technique). Typically, a gain-of-function mutation results in an increase of at least or about 20%, 30%, 40%, 50%, 60%, 70%, 80%, 90%, 100%, 125%, 150%, 175%, 200%, 225%, 250%, 275%, 300%, 325%, 350%, 375%, 400% or more compared to the activity of a potassium channel encoded by a wild-type KCNT1.

### Compounds and Compositions

In one aspect, provided herein is a compound having the Formula A:

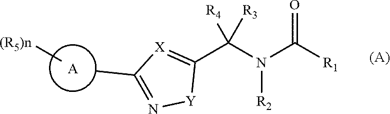

X is CR₇ or N and Y is S; or
X is CR₇ and Y is O;
ring A is selected from the group consisting of phenyl, 6-membered heteroaryl, and 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 6-membered heterocyclyl or 6-membered heteroaryl, R₁ is not thiophene;
provided that when R₃ is hydrogen and ring A is 6-membered heteroaryl or 5-membered heterocyclyl, R₁ is not phenyl; or a pharmaceutically acceptable salt thereof.

In another aspect, provided herein is a compound having the Formula A-1:

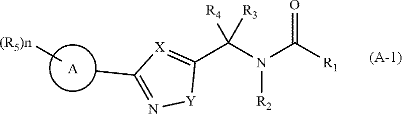

X is CR₇ or N and Y is S; or
X is CR₇ and Y is O;
ring A is 6-membered heteroaryl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 6-membered heteroaryl, R₁ is not thiophene or phenyl; or a pharmaceutically acceptable salt thereof.

In some embodiments of Formula A or A-1, ring A is pyridyl.

In some embodiments of Formula A or A-1, the compound is a compound of Formula A-1A or Formula A-1B:

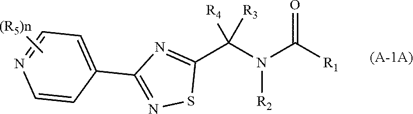

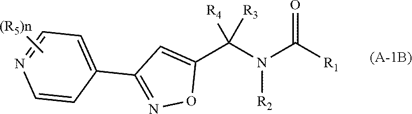

or a pharmaceutically acceptable salt thereof.

In another aspect, provided herein is a compound having the Formula A-2:

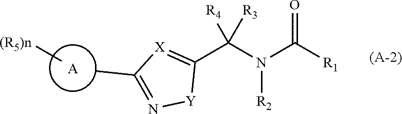

X is CR₇ or N and Y is S; or
X is CR₇ and Y is O;
ring A is 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 5-6-membered heterocyclyl, R₁ is not thiophene or phenyl; or a pharmaceutically acceptable salt thereof.

In some embodiments of Formula A or A-2, the compound is a compound of Formula A-2A:

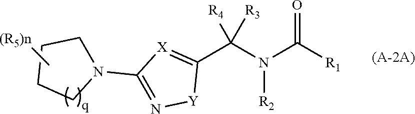

wherein q is 1 or 2; or a pharmaceutically acceptable salt thereof.

In some embodiments of Formula A, A-1, or A-2, X is N and Y is S. In other embodiments of Formula A, A-1, or A-2, X is CH and Y is O.

In some embodiments of Formula A, A-1, or A-2, R₃ is C₁₋₆alkyl. For example, R₃ is methyl.

In some embodiments of Formula A, A-1, or A-2, R₃ is hydrogen.

In some embodiments of Formula A, A-1, or A-2, R₂ is hydrogen.

In some embodiments of Formula A, A-1, or A-2, R₅ is C₁₋₆alkyl, C₁₋₆alkylene-O-C₁_ ₆alkyl, C₁₋₆haloalkyl, C₁₋₆alkoxy, or C₃₋₈cycloalkyl. For example, R₅ is cyclopropyl, -CF₃, methyl, —OCH₃, or —CH₂OCH₃.

In some embodiments of Formula A, A-1, or A-2, R₁ is 5-6 membered heteroaryl optionally substituted with one or more R₆. In some embodiments, the heteroaryl is pyrazolyl.

In some embodiments of Formula A, A-1, or A-2, R₁ is phenyl optionally substituted with one or more R₆.

In some embodiments of Formula A, A-1, or A-2, R₁ is -CH₂-phenyl optionally substituted with one or more R₆. In some embodiments, the 10-membered heterocyclyl is a bicyclic heterocyclyl.

In some embodiments of Formula A, A-1, or A-2, R₁ is selected from the group consisting of:

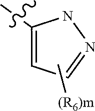

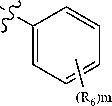

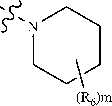

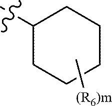

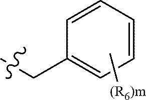

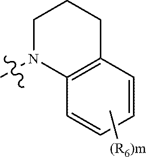

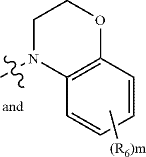

, wherein m is 0, 1, or 2. In some embodiments, m is 0. In some embodiments, m is 1. In some embodiments m is 2.

In some embodiments of Formula A, A-1, or A-2, R₆ is halogen, C₁₋₆alkyl, or C₁₋₆haloalkyl.

In another aspect, provided herein is a compound having the Formula I:

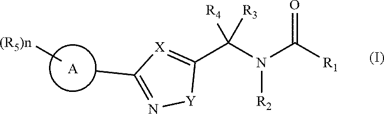

or a pharmaceutically acceptable salt thereof, wherein:
X is CR₇ or N and Y is S; or
X is CR₇ and Y is O;
ring A is selected from the group consisting of phenyl, 6-membered heteroaryl, and 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

In another aspect, provided herein is a Formula I-A:

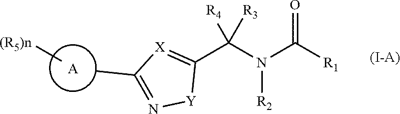

or a pharmaceutically acceptable salt thereof, wherein:
X is CR₇ or N and Y is S; or
X is CR₇ and Y is O;
ring A is 6-membered heteroaryl or 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-10 membered carbocyclyl, and 5-10 membered heterocyclyl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

In another aspect, provided herein is a compound having the Formula I-B:

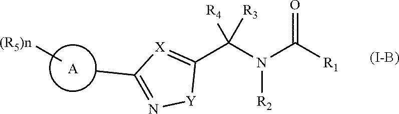

or a pharmaceutically acceptable salt thereof, wherein:
X is CR₇ or N and Y is S; or
X is CR₇ and Y is O;
ring A is phenyl or 6-membered heteroaryl;
R₁ is phenyl or 5-6 membered heteroaryl, wherein the phenyl or 5-6 membered heteroaryl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆haloalkyl, C₁₋₆alkoxy, C₁₋₆haloalkoxy, —S(O)₂R₈, —S(O)₂—N(R₉)₂, and C₃-₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

In some embodiments of Formula I, I-A, or I-B, ring A is 6-membered heteroaryl. In some embodiments of Formula I, I-A, or I-B, ring A is pyridyl.

In some embodiments of Formula I, I-A, or I-B, X is N and Y is S.

In some embodiments of Formula I, I-A, or I-B, X is CH and Y is O.

In some embodiments of Formula I, I-A, or I-B, R₃ is C₁₋₆alkyl. For example, R₃ is methyl.

In some embodiments of Formula I, I-A, or I-B, R₂ is hydrogen.

In some embodiments of Formula I or I-A, R₅ is C₁₋₆alkyl, C₁₋₆alkylene-O-C₁₋₆alkyl, C₁₋₆haloalkyl, C₁₋₆alkoxy, or C₃₋₈cycloalkyl. For example, R₅ is cyclopropyl, —CF₃, methyl, —OCH₃, or —CH₂OCH₃,

In some embodiments of Formula I, I-A, or I-B, R₅ is C₃₋₈cycloalkyl or C₁₋₆haloalkyl. In some embodiments of Formula I, I-A, or I-B, R₅ is cyclopropyl or —CF₃.

In some embodiments of Formula I, I-A, or I-B, n is 0 or 1. In some embodiments of Formula I, I-A, or I-B, n is 1. In some embodiments of Formula I, I-A, or I-B, n is 0.

In some embodiments of Formula I, I-A, or I-B, R₁ is 5-6 membered heteroaryl optionally substituted with one or more R₆. In some embodiments, the heteroaryl is pyrazolyl.

In some embodiments of Formula I, I-A, or I-B, R₁ is phenyl optionally substituted with one or more R₆.

In some embodiments of Formula I or I-A, R₁ is -CH2-phenyl optionally substituted with one or more R₆.

In some embodiments of Formula I or I-A, R₁ is 10-membered heterocyclyl optionally substituted with one or more R₆. In some embodiments, the 10-membered heterocyclyl is a bicyclic heterocyclyl.

In some embodiments of Formula I, I-A, or I-B, R₆ is halogen, C₁₋₆alkyl, or C₁₋₆haloalkyl.

In some embodiments of Formula I, I-A, or I-B, R₆ is C₁₋₆alkyl or C₁₋₆haloalkyl.

In some embodiments of Formula I, I-A, or I-B, the compound is a compound of Formula I-IA or Formula I-IB:

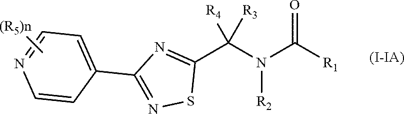

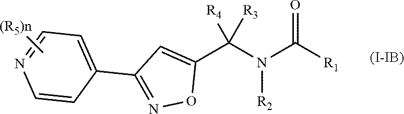

or a pharmaceutically acceptable salt thereof.

In some embodiments of Formula I, I-A, or I-B, the compound is a compound of Formula I-IA2 or Formula I-IB2:

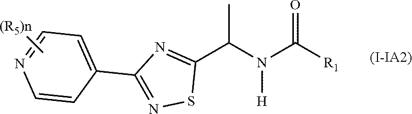

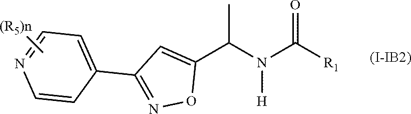

or a pharmaceutically acceptable salt thereof.

In some embodiments of Formula I, I-A, or I-B, the compound is a compound of Formula I-IA3, Formula I-IA4, Formula I-IB3, or Formula I-IB4:

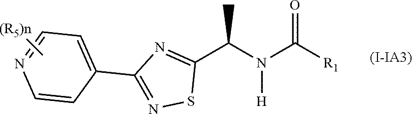

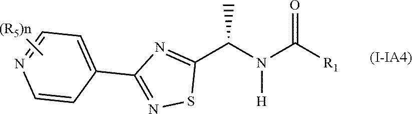

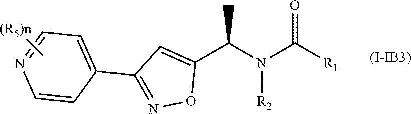

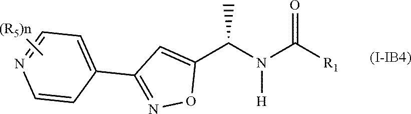

or a pharmaceutically acceptable salt thereof.

In some embodiments of Formula I or I-A, the compound is a compound of Formula I-IC:

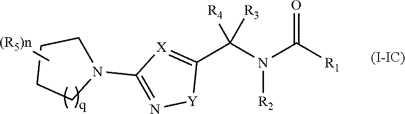

wherein q is 1 or 2; or a pharmaceutically acceptable salt thereof.

In some embodiments of Formula I or I-A, the compound is a compound of Formula I-IC2:

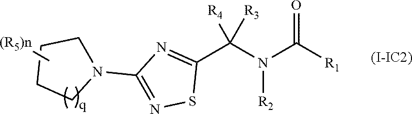

wherein q is 1 or 2; or a pharmaceutically acceptable salt thereof.

In some embodiments of Formula I or I-A, the compound is a compound of Formula I-IC3 or Formula I-IC4:

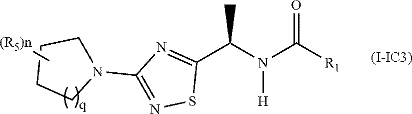

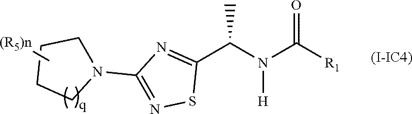

or a pharmaceutically acceptable salt thereof.

In some embodiments of Formula I, I-A, or I-B, R₁ is selected from the group consisting of:

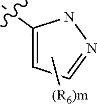

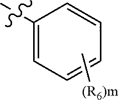

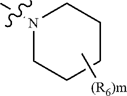

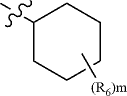

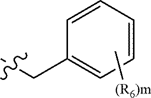

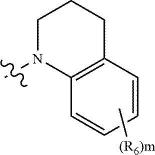

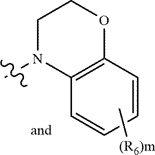

, wherein m is 0, 1, or 2. In some embodiments, m is 0. In some embodiments, m is 1. In some embodiments m is 2.

In some embodiments of Formula I, I-A, or I-B, R₁ is pyrazolyl or phenyl optionally substituted with one or more R₆.

In one aspect, the present invention features a compound of Formula (II):

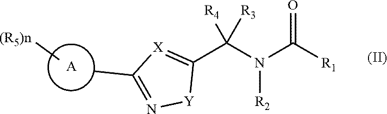

or a pharmaceutically acceptable salt thereof, wherein:
X is CR₇ or N and Y is S; or
X is CR₇ and Y is O;
ring A is phenyl or 6-membered heteroaryl;
R₁ is phenyl or 5-6 membered heteroaryl, wherein the phenyl or 5-6 membered heteroaryl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋₈cycloalkylene or 3-7 membered heterocycloalkylene;
R₅ and R₆ are each independently selected from the group consisting of halogen, C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

In some embodiments, ring A is 6-membered heteroaryl (e.g., pyridyl).

Ins some embodiments, X is N and Y is S. In some embodiments, X is CH and Y is O.

In some embodiments of Formula II, the compound is a compound of Formula II-A or Formula 11-B:

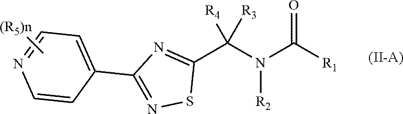

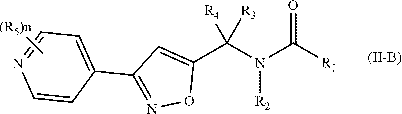

or a pharmaceutically acceptable salt thereof.

In some embodiments of Formula II, R₃ is C₁₋₆alkyl (e.g., methyl).

In some embodiments of Formula II, R₂ is hydrogen.

In some embodiments of Formula II, n is 0 or 1. In some embodiments of Formula II, n is 1.

In some embodiments of Formula II, R₅ is C₃₋₈cycloalkyl (e.g., cyclopropyl) or C₁₋₆haloalkyl (e.g., CF₃).

In some embodiments of Formula II, R₁ is 5-6 membered heteroaryl (e.g., pyrazolyl) optionally substituted with one or more R₆. In some embodiments of Formula II, R₁ is phenyl optionally substituted with one or more R₆. In some embodiments of Formula II, R₆ is C₁₋₆alkyl or C₁₋₆haloalkyl.

In some embodiments, the compound is selected from the group consisting of:

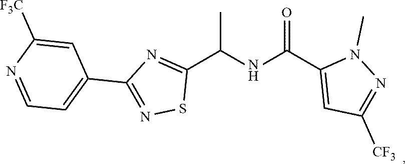

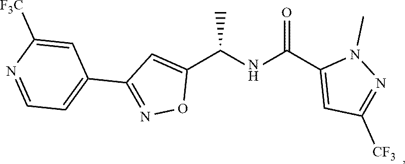

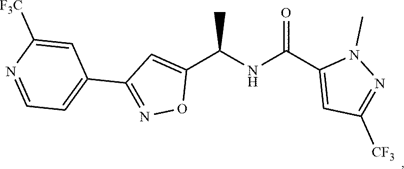

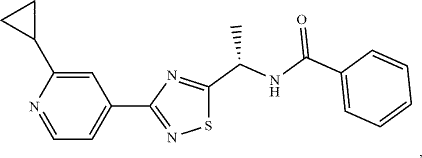

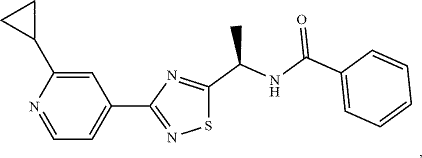

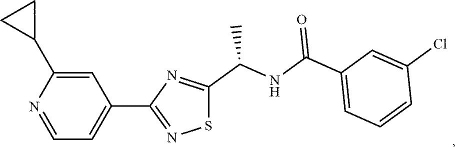

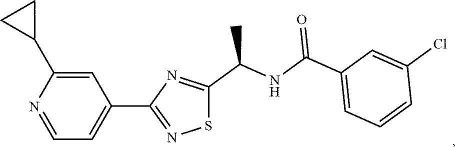

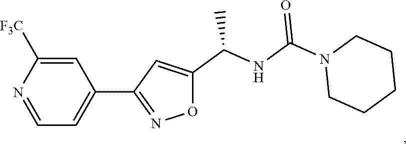

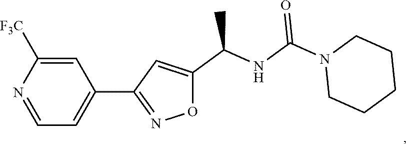

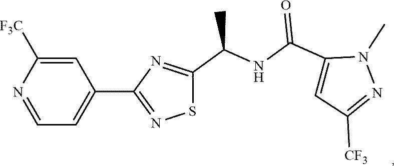

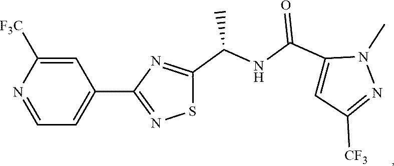

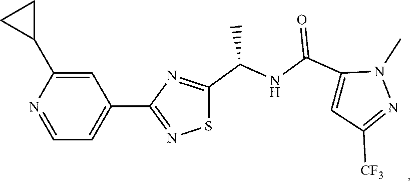

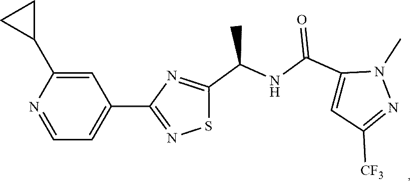

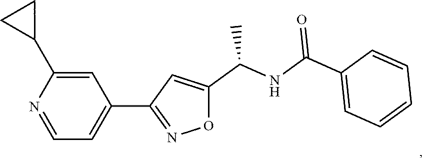

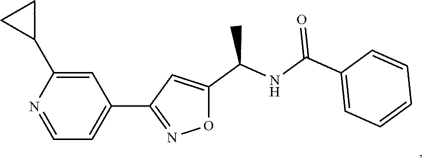

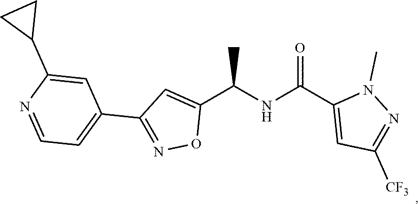

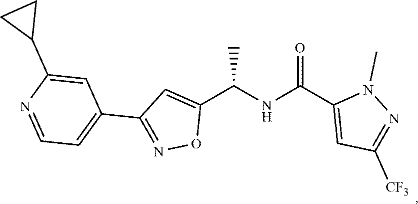

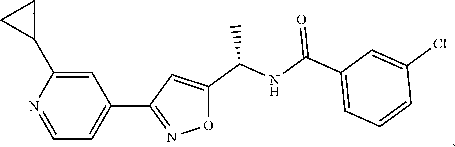

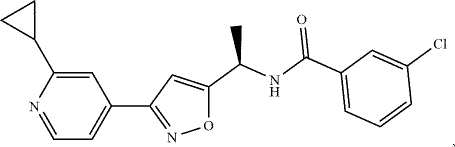

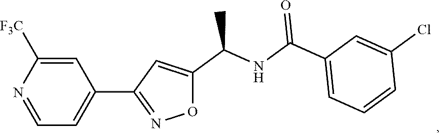

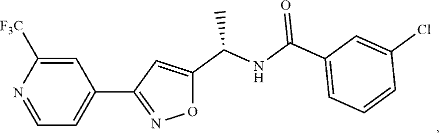

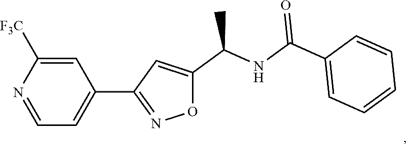

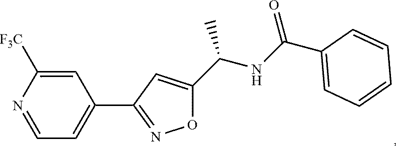

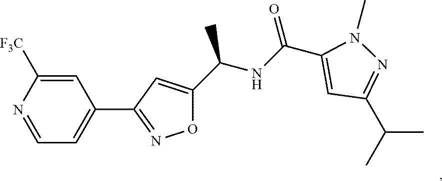

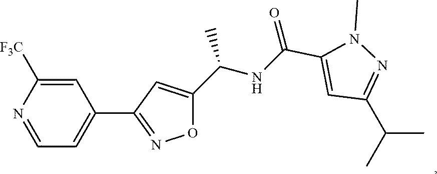

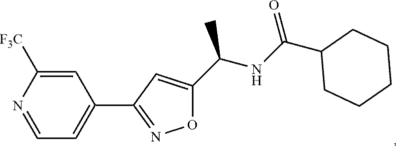

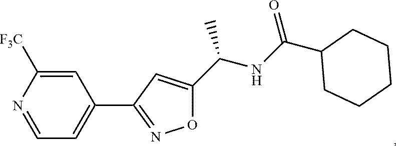

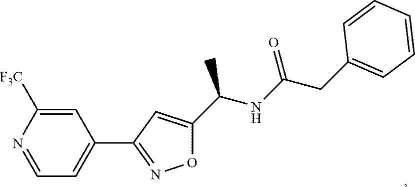

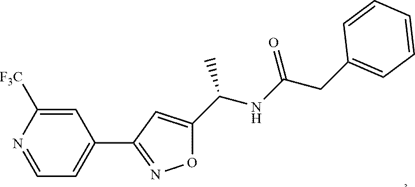

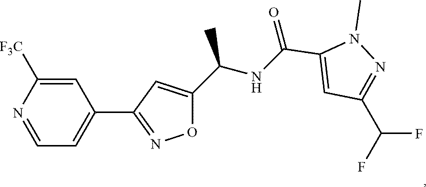

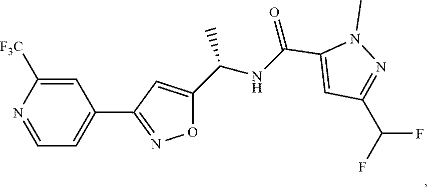

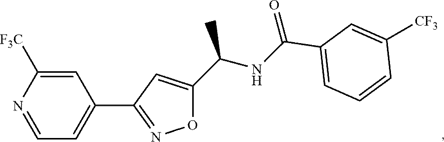

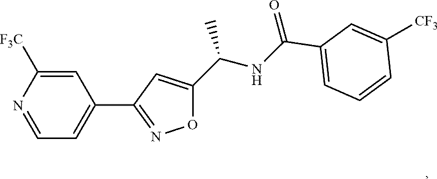

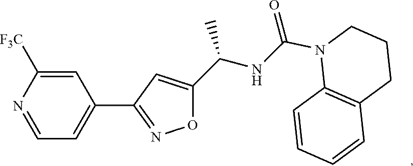

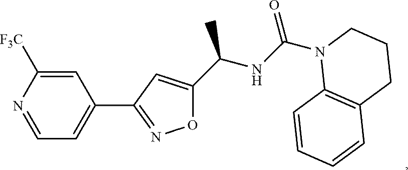

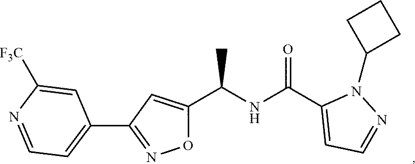

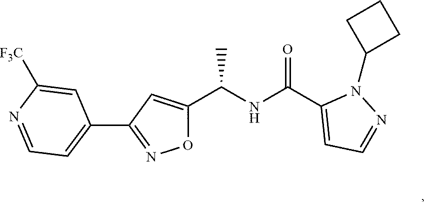

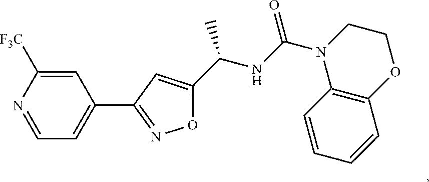

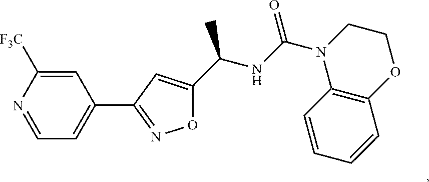

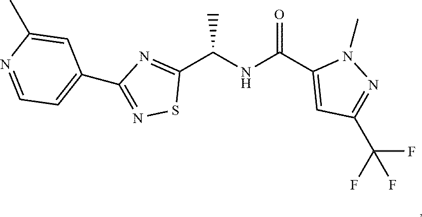

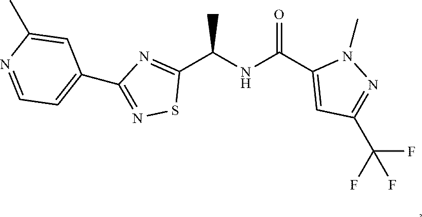

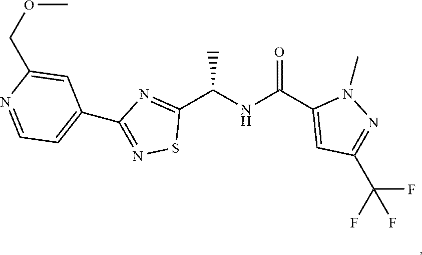

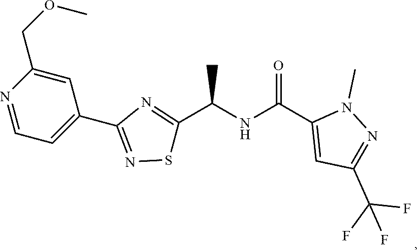

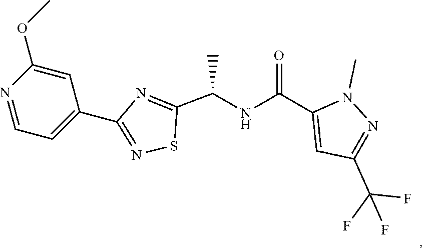

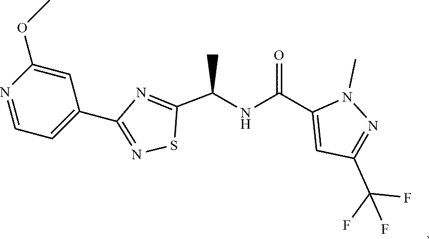

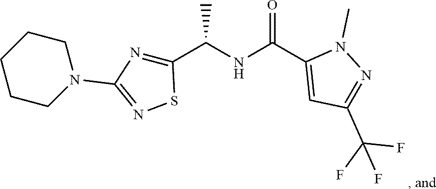

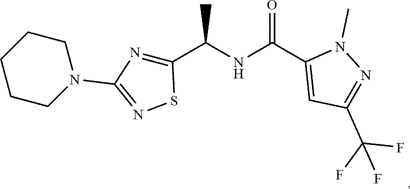

or a pharmaceutically acceptable salt thereof.

In another aspect, provided herein is a pharmaceutical composition comprising a compound disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B), or a pharmaceutically acceptable salt thereof) and a pharmaceutically acceptable excipient.

### General Synthetic Schemes

Exemplary methods for preparing compounds described herein are illustrated in the following synthetic schemes. These schemes are given for the purpose of illustrating the invention, and should not be regarded in any manner as limiting the scope or the spirit of the invention.

### Scheme 1

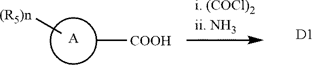

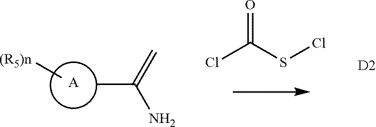

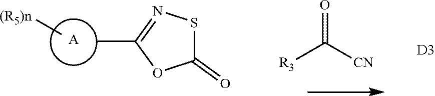

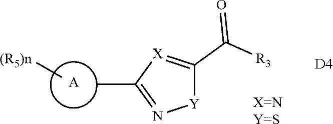

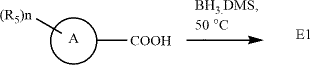

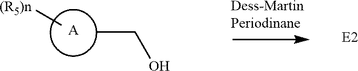

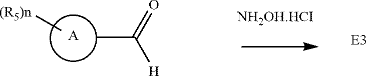

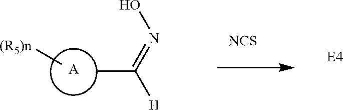

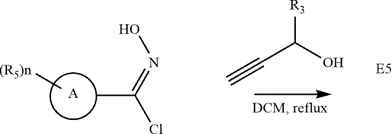

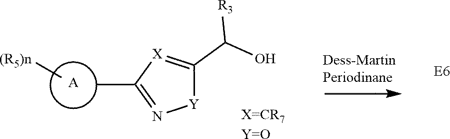

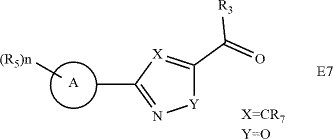

The synthetic route illustrated in Scheme 1 depicts an exemplary procedure for preparing intermediates D4 and E7. In the first step, compound D1 is reacted with (COCl)₂ and ammonia to form amide D2. Then, amide D2 is reacted with chlorocarbonylsulfenyl chloride to form D3, which is reacted with R₃-containing cyanide to form D4. To form intermediate E7, carboxylic acid E1 is reacted with borane to form E2, which is then reacted with Dess-Martin Periodinane to form E3. Then, E3 is reacted with hydroxylamine to form E4, which is reacted with N-chlorosuccinimide to form E5. E5 is then reacted with R₃-containing alcohol to form E6, which is reacted with Dess-Martin Periodinane to form intermediate E7.

### Scheme 2

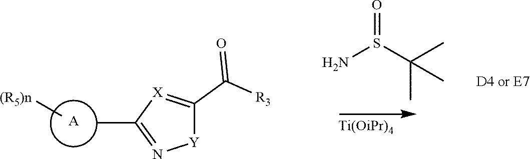

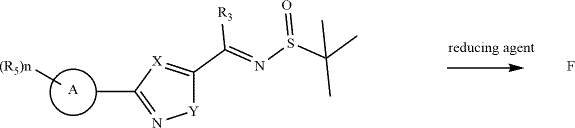

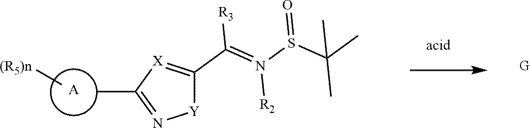

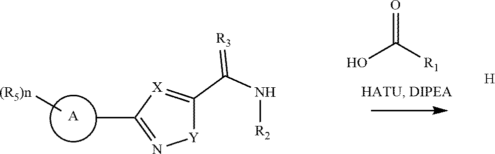

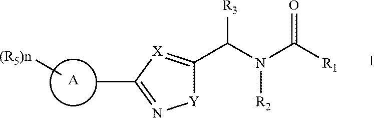

The synthetic route illustrated in Scheme 2 represents an exemplary procedure for preparing a compound of formula I from intermediates D4 or E7 as described in Scheme 1. Intermediate D4 or E7 is reacted with a sulfinamide to form F, which is subsequently reduced to form G. Then, G is reacted with an acid to form H, which is reacted with R₁-containing carboxylic acid to form a compound of formula I.

### Scheme 3

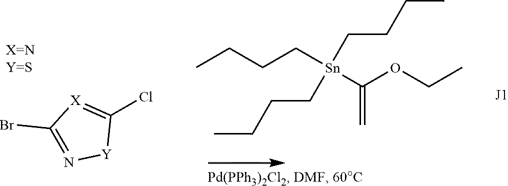

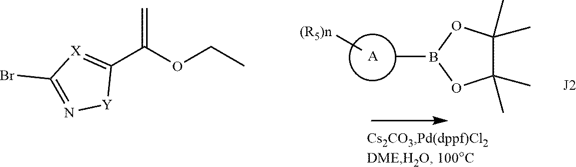

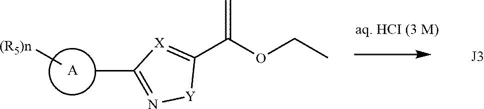

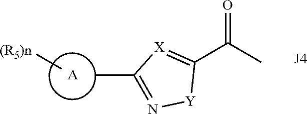

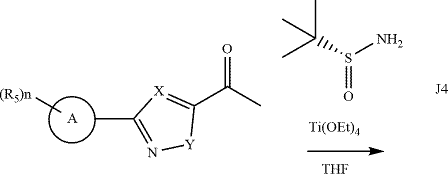

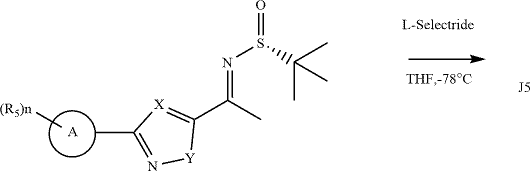

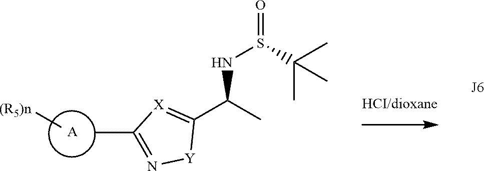

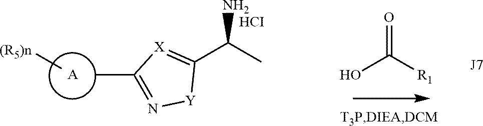

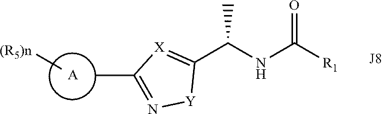

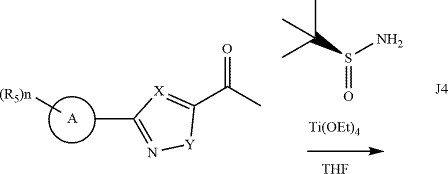

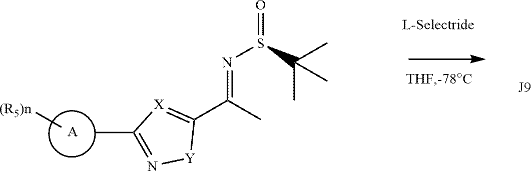

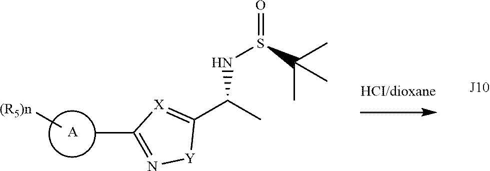

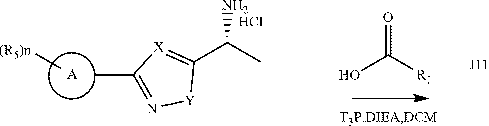

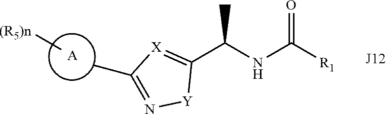

The synthetic route illustrated in Scheme 3 depicts an exemplary procedure for preparing J8 and J12 which are compounds of Formula I. In the first step, compound J1 is reacted with 1-ethoxyvinyltri-n-butyltin to form J2. Then, J2 is reacted with A-containing dioxaborolane to form J3, which is reacted an acid to form J4. J4 is then reacted with either (R)-2-methylpropane-2-sulfinamide or (S)-2-methylpropane-2-sulfinamide to form J5 or J9, which is then reacted with L-selectride to form J6 or J10. Then J6 or J10 is independently reacted with an acid to form amine J7 or J11, which is then reacted with R₁-containing carboxylic acid to form J8 or J12.

### Scheme 4

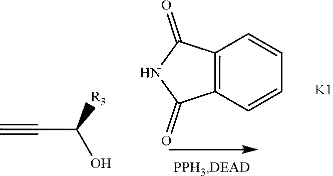

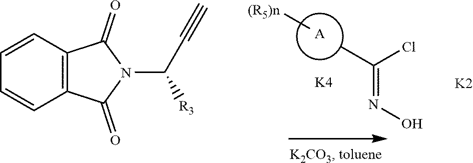

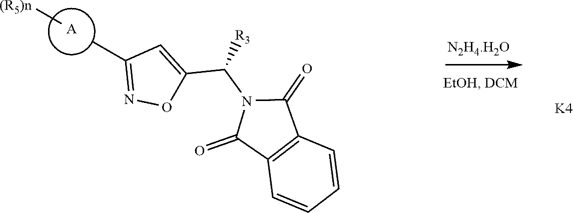

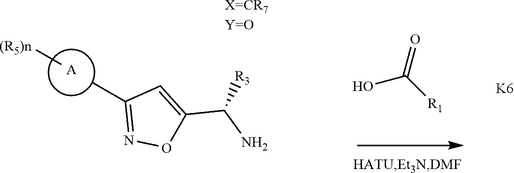

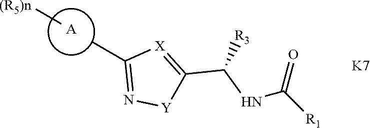

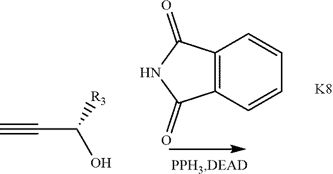

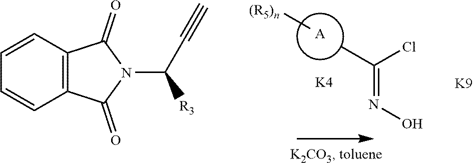

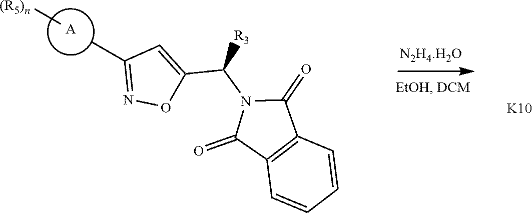

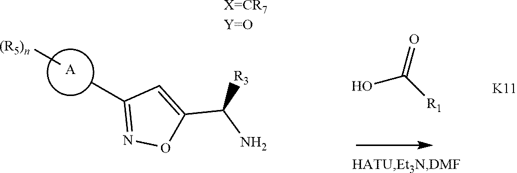

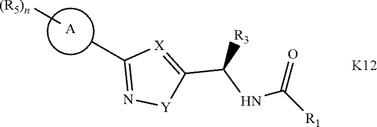

The synthetic route illustrated in Scheme 4 depicts an exemplary procedure for preparing K7 and K12 which are compounds of Formula I. In the first step, compound K1 or K8 is reacted with phthalimide to form K2 or K9, respectively. Then, K2 or K9 is reacted with A-containing carboximidoyl chloride to form K4 or K10, which is subsequently reacted with hydrazine to form K6 or K11. Then K6 or K11 is reacted with R₁-containing carboxylic acid to form K7 or K12.

### Methods of Treatment

The compounds and compositions described above and herein can be used to treat a neurological disease or disorder or a disease or condition associated with excessive neuronal excitability and/or a gain-of-function mutation in a gene (e.g., KCNT1). Exemplary diseases, disorders, or conditions include epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, developmental and epileptic encephalopathy (DEE), early infantile epileptic encephalopathy (EIEE), generalized epilepsy, focal epilepsy, multifocal epilepsy, temporal lobe epilepsy, Ohtahara syndrome, early myoclonic encephalopathy and Lennox Gastaut syndrome, drug resistant epilepsy, seizures (e.g., frontal lobe seizures, generalized tonic clonic seizures, asymmetric tonic seizures, focal seizures), leukodystrophy, hypomyelinating leukodystrophy, leukoencephalopathy, and sudden unexpected death in epilepsy, cardiac dysfunctions (e.g., cardiac arrhythmia, Brugada syndrome, myocardial infarction), pulmonary vasculopathy / hemorrhage, pain and related conditions (e.g. neuropathic pain, acute/chronic pain, migraine, etc), muscle disorders (e.g. myotonia, neuromyotonia, cramp muscle spasms, spasticity), itch and pruritis, movement disorders (e.g., ataxia and cerebellar ataxias), psychiatric disorders (e.g. major depression, anxiety, bipolar disorder, schizophrenia, attention-deficit hyperactivity disorder), neurodevelopmental disorder, learning disorders, intellectual disability, Fragile X, neuronal plasticity, and autism spectrum disorders.

In some embodiments, the neurological disease or disorder or the disease or condition associated with excessive neuronal excitability and/or a gain-of-function mutation in a gene (e.g., KCNT1) is selected from EIMFS, ADNFLE and West syndrome. In some embodiments, the neurological disease or disorder or the disease or condition associated with excessive neuronal excitability and/or a gain-of-function mutation in a gene (e.g., KCNT1) is selected from infantile spasms, epileptic encephalopathy, focal epilepsy, Ohtahara syndrome, developmental and epileptic encephalopathy and Lennox Gastaut syndrome. In some embodiments, the neurological disease or disorder or the disease or condition associated with excessive neuronal excitability and/or a gain-of-function mutation in a gene (e.g., KCNT1) is seizure. In some embodiments, the neurological disease or disorder or the disease or condition associated with excessive neuronal excitability and/or a gain-of-function mutation in a gene (e.g., KCNT1) is selected from cardiac arrhythmia, Brugada syndrome, and myocardial infarction.

In some embodiments, the neurological disease or disorder or the disease or condition associated with excessive neuronal excitability and/or a gain-of-function mutation in a gene (e.g., KCNT1) is selected from the group consisting of the learning disorders, Fragile X, intellectual function, neuronal plasticity, psychiatric disorders, and autism spectrum disorders.

Accordingly, the compounds and compositions thereof can be administered to a subject with a neurological disease or disorder or a disease or condition associated with excessive neuronal excitability and/or a gain-of-function mutation in a gene such as KCNT1 (e.g., EIMFS, ADNFLE, West syndrome, infantile spasms, epileptic encephalopathy, focal epilepsy, Ohtahara syndrome, developmental and epileptic encephalopathy, and Lennox Gastaut syndrome, seizures, cardiac arrhythmia, Brugada syndrome, and myocardial infarction).

EIMFS is a rare and debilitating genetic condition characterized by an early onset (before 6 months of age) of almost continuous heterogeneous focal seizures, where seizures appear to migrate from one brain region and hemisphere to another. Patients with EIMFS are generally intellectually impaired, non-verbal and non-ambulatory. While several genes have been implicated to date, the gene that is most commonly associated with EIMFS is KCNT1. Several de novo mutations in KCNT1 have been identified in patients with EIMFS, including V271F, G288S, R428Q, R474Q, R474H, R474C, I760M, A934T, P924L, G243S, H257D, A259D, R262Q, Q270E, L274I, F346L, C377S, R398Q, P409S, A477T, F502V, M516V, Q550del, K629E, K629N, I760F, E893K, M896K, R933G, R950Q, K1154Q (Barcia et al. (2012) Nat Genet. 44: 1255-1260; Ishii et al. (2013) Gene 531:467-471; McTague et al. (2013) Brain. 136: 1578-1591; Epi4K Consortium & Epilepsy Phenome/Genome Project. (2013) Nature 501:217-221; Lim et al. (2016) Neurogenetics; Ohba et al. (2015) Epilepsia 56:el21-el28; Zhou et al. (2018) Genes Brain Behav. e12456; Moller et al. (2015) Epilepsia. e114-20; Numis et al. (2018) Epilepsia. 1889-1898; Madaan et al. Brain Dev. 40(3):229-232; McTague et al. (2018) Neurology. 90(1):e55-e66; Kawasaki et al. (2017) J Pediatr. 191:270-274; Kim et al. (2014) Cell Rep. 9(5):1661-1672; Ohba et al. (2015) Epilepsia. 56(9):e121-8; Rizzo et al. (2016) Mol Cell Neurosci. 72:54-63; Zhang et al. (2017) Clin Genet. 91(5):717-724; Mikati et al. (2015) Ann Neurol. 78(6):995-9; Baumer et al. (2017) Neurology. 89(21):2212; Dilena et al. (2018) Neurotherapeutics. 15(4):1112-1126). These mutations are gain-of-function, missense mutations that are dominant (i.e. present on only one allele) and result in change in function of the encoded potassium channel that causes a marked increase in whole cell current when tested in Xenopus oocyte or mammalian expression systems (see e.g. Milligan et al. (2015) Ann Neurol. 75(4): 581-590; Barcia et al. (2012) Nat Genet. 44(11): 1255-1259; and Mikati et al. (2015) Ann Neurol. 78(6): 995-999).

ADNFLE has a later onset than EIMFS, generally in mid-childhood, and is generally a less severe condition. It is characterized by nocturnal frontal lobe seizures and can result in psychiatric, behavioural and cognitive disabilities in patients with the condition. While ADNFLE is associated with genes encoding several neuronal nicotinic acetylcholine receptor subunits, mutations in the KCNT1 gene have been implicated in more severe cases of the disease (Heron et al. (2012) Nat Genet. 44: 1188-1190). Functional studies of the mutated KCNT1 genes associated with ADNFLE indicated that the underlying mutations (M896I, R398Q, Y796H and R928C) were dominant, gain-of-function mutations (Milligan et al. (2015) Ann Neurol. 75(4): 581-590; Mikati et al. (2015) Ann Neurol. 78(6): 995-999).

West syndrome is a severe form of epilepsy composed of a triad of infantile spasms, an interictal electroencephalogram (EEG) pattern termed hypsarrhythmia, and mental retardation, although a diagnosis can be made one of these elements is missing. Mutations in KCNT1, including G652V and R474H, have been associated with West syndrome (Fukuoka et al. (2017) Brain Dev 39:80-83 and Ohba et al. (2015) Epilepsia 56:el21-el28). Treatment targeting the KCNT1 channel suggests that these mutations are gain-of-function mutations (Fukuoka et al. (2017) Brain Dev 39:80-83).

In one aspect, the present invention features a method of treating treat a disease or condition associated with excessive neuronal excitability and/or a gain-of-function mutation in a gene such as KCNT1 (for example, epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy (DEE), and Lennox Gastaut syndrome, seizures, leukodystrophy, leukoencephalopathy, intellectual disability, Multifocal Epilepsy, Generalized tonic clonic seizures, Drug resistant epilepsy, Temporal lobe epilepsy, cerebellar ataxia, Asymmetric Tonic Seizures) and cardiac dysfunctions (e.g., cardiac arrhythmia, Brugada syndrome, sudden unexpected death in epilepsy, myocardial infarction), pain and related conditions (e.g. neuropathic pain, acute/chronic pain, migraine, etc), muscle disorders (e.g. myotonia, neuromyotonia, cramp muscle spasms, spasticity), itch and pruritis, ataxia and cerebellar ataxias, psychiatric disorders (e.g. major depression, anxiety, bipolar disorder, schizophrenia), learning disorders, Fragile X, neuronal plasticity, and autism spectrum disorders) comprising administering to a subject in need thereof a compound disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC) (I-IC2), (I-IC3), (I-IC4), (II), (II-A), or (II-B)) or a pharmaceutically acceptable salt thereof) or a pharmaceutical composition disclosed herein (e.g., a pharmaceutical composition comprising a compound disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B)) or a pharmaceutically acceptable salt thereof), and a pharmaceutically acceptable excipient).

In some examples, the subject presenting with a disease or condition that may be associated with a gain-of-function mutation in KCNT1 is genotyped to confirm the presence of a known gain-of-function mutation in KCNT1 prior to administration of the compounds and compositions thereof. For example, whole exome sequencing can be performed on the subject. Gain-of-function mutations associated with EIMFS may include, but are not limited to, V271F, G288S, R428Q, R474Q, R474H, R474C, I760M, A934T, P924L, G243S, H257D, A259D, R262Q, Q270E, L274I, F346L, C377S, R398Q, P409S, A477T, F502V, M516V, Q550del, K629E, K629N, I760F, E893K, M896K, R933G, R950Q, and K1154Q. Gain-of-function mutations associated with ADNFLE may include, but are not limited to, M896I, R398Q, Y796H, R928C, and G288S. Gain-of-function mutations associated with West syndrome may include, but are not limited to, G652V and R474H. Gain-of-function mutations associated with temporal lobe epilepsy may include, but are not limited to, R133H and R565H. Gain-of-function mutations associated with Lennox-Gastaut may include, but are not limited to, R209C. Gain-of-function mutations associated with seizures may include, but are not limited to, A259D, G288S, R474C, R474H. Gain-of-function mutations associated with leukodystrophy may include, but are not limited to, G288S and Q906H. Gain-of-function mutations associated with Multifocal Epilepsy may include, but are not limited to, V340M. Gain-of-function mutations associated with EOE may include, but are not limited to, F346L and A934T. Gain-of-function mutations associated with Early-onset epileptic encephalopathies (EOEE) may include, but are not limited to, R428Q. Gain-of-function mutations associated with developmental and epileptic encephalopathies may include, but are not limited to, F346L, R474H, and A934T. Gain-of-function mutations associated with epileptic encephalopathies may include, but are not limited to, L437F, Y796H, P924L, R961H. Gain-of-function mutations associated with Early Infantile Epileptic Encephalopathy (EIEE) may include, but are not limited to, M896K. Gain-of-function mutations associated with drug resistent epilepsy and generalized tonic-clonic seizure may include, but are not limited to, F346L. Gain-of-function mutations associated with migrating partial seizures of infancy may include, but are not limited to, R428Q. Gain-of-function mutations associated with Leukoencephalopathy may include, but are not limited to, F932I. Gain-of-function mutations associated with NFLE may include, but are not limited to, A934T and R950Q. Gain-of-function mutations associated with Ohtahara syndrome may include, but are not limited to, A966T. Gain-of-function mutations associated with infantile spasms may include, but are not limited to, P924L. Gain-of-function mutations associated with Brugada Syndrome may include, but are not limited to, R1106Q. Gain-of-function mutations associated with Brugada Syndrome may include, but are not limited to, R474H.

In other examples, the subject is first genotyped to identify the presence of a mutation in KCNT1 and this mutation is then confirmed to be a gain-of-function mutation using standard in vitro assays, such as those described in Milligan et al. (2015) Ann Neurol. 75(4): 581-590. Typically, the presence of a gain-of-function mutation is confirmed when the expression of the mutated KCNT1 allele results an increase in whole cell current compared to the whole cell current resulting from expression of wild-type KCNT1 as assessed using whole-cell electrophysiology (such as described in Milligan et al. (2015) Ann Neurol. 75(4): 581-590; Barcia et al. (2012) Nat Genet. 44(11): 1255-1259; Mikati et al. (2015) Ann Neurol. 78(6): 995-999; or Rizzo et al. Mol Cell Neurosci. (2016) 72:54-63). This increase of whole cell current can be, for example, an increase of at least or about 50%, 100%, 150%, 200%, 250%, 300%, 350%, 400% or more. The subject can then be confirmed to have a disease or condition associated with a gain-of-function mutation in KCNT1.

In particular examples, the subject is confirmed as having a KCNT1 allele containing a gain-of-function mutation (e.g. V271F, G288S, R398Q, R428Q, R474Q, R474H, R474C, G652V, I760M, Y796H, M896I, P924L, R928C or A934T).

The compounds disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B)) or a pharmaceutically acceptable salt thereof) or the pharmaceutical composition disclosed herein (e.g., a pharmaceutical composition comprising a compound disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B)) or a pharmaceutically acceptable salt thereof), and a pharmaceutically acceptable excipient) can also be used therapeutically for conditions associated with excessive neuronal excitability where the excessive neuronal excitability is not necessarily the result of a gain-of-function mutation in KCNT1. Even in instances where the disease is not the result of increased KCNT1 expression and/or activity, inhibition of KCNT1 expression and/or activity can nonetheless result in a reduction in neuronal excitability, thereby providing a therapeutic effect. Thus, the compounds disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B)) or a pharmaceutically acceptable salt thereof) or the pharmaceutical composition disclosed herein (e.g., a pharmaceutical composition comprising a compound disclosed herein (e.g., a compound of Formula (A), (A-1), (A-1A), (A-1B), (A-2), (A-2A), (I), (I-A), (I-IA), (I-IA2), (I-IA3), (I-IA4), (I-B), (I-IB), (I-IB2), (I-IB3), (I-IB4), (I-IC), (I-IC2), (I-IC3), (I-IC4), (II), (II-A), or (II-B)) or a pharmaceutically acceptable salt thereof), and a pharmaceutically acceptable excipient) can be used to treat a subject with conditions associated with excessive neuronal excitability, for example, epilepsy and other encephalopathies (e.g., epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, and Lennox Gastaut syndrome, seizures) or cardiac dysfunctions (e.g., cardiac arrhythmia, Brugada syndrome, myocardial infarction), regardless of whether or not the disease or disorder is associated with a gain-of-function mutation in KCNT1.

### Pharmaceutical Compositions and Routes of Administration

Compounds provided in accordance with the present invention are usually administered in the form of pharmaceutical compositions. This invention therefore provides pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington’s Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient’s symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprising a disclosed compound, or pharmaceutically acceptable salt thereof, and a pharmaceutically acceptable carrier.

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

List of Abbreviations

| | |
|---|---|
| THF | tetrahydrofuran |
| TFA | trifluoroacetic acid |
| DMF | N,N-dimethylformamide |
| MeOH | methanol |
| EtOH | ethanol |
| DCM | dichloromethane |
| MeCN or ACN | acetonitrile |
| EtOAc | ethyl acetate |
| DIPEA N,N, | -diisopropylethyl amine |
| HATU | o-(7-azabenzotriazol-1-yl)-N,N,N′,N′-tetramethyluronium hexafluorophosphate |
| Ti(OEt)₄ | titanium(IV) ethoxide |
| Ti(OiPr)₄ | titanium(IV) isopropoxide |
| T₃P | propanephosphonic acid anhydride |
| L-selectride | lithium tri-s-butylborohydride |
| K-Selectride | potassium tri-sec-butylborohydride |
| DIEA | N, N-diisopropylethylamine |
| Pd(dppf)Cl₂ | [1,1′-bis(diphenylphosphino)ferrocene]dichloropalladium(II) |
| Pd(PPh₃)₂Cl₂ | dichlorobis(triphenylphosphine)palladium(II) |
| DMSO | dimethyl sulfoxide |
| DMS | dimethylsulfide |
| EGTA | ethylene glycol-bis(β-aminoethyl ether)-N,N,N′,N′-tetraacetic acid |
| NMDG | N-methyl-D-glucamine |
| HEPES | 4-(2-hydroxyethyl)piperazine-1-ethanesulfonic acid |
| IC₅₀ | half maximal inhibitory concentration |
| TLC | thin layer chromatography |
| LCMS | liquid chromatography-mass spectrometry |
| HPLC | high-performance liquid chromatagraphy |
| SFC | supercritical fluid chromatography |
| MS | mass spectrometry |
| NMR | nuclear magnetic resonance |

DIEA N, N-diisopropylethylamine Pd(dppf)Cl₂ [1,1′-bis(diphenylphosphino)ferrocene]dichloropalladium(II) Pd(PPh₃)₂Cl₂ dichlorobis(triphenylphosphine)palladium(II) DMSO dimethyl sulfoxide DMS dimethylsulfide e

### Example 1. Synthesis of 1-methyl-3-(trifluoromethyl)-N-(1-(3-(2-(trifluoromethyl)pyridin-4-yl)-1,2,4-thiadiazol-5-yl)ethyl)-1H-pyrazole-5-carboxamide (1)

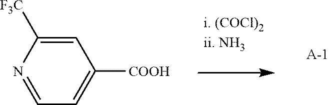

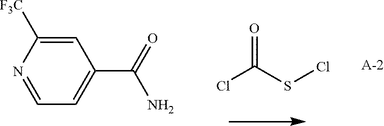 [SMILES: NC(=O)c1ccnc(C(F)(F)F)c1 ; O=C(Cl)SCl]

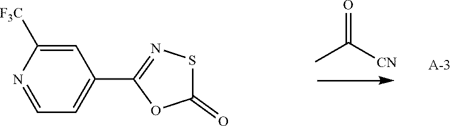

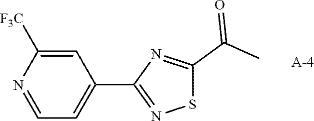 [SMILES: CC(=O)c1nc(-c2ccnc(C(F)(F)F)c2)ns1]

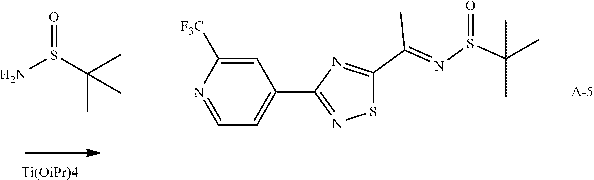

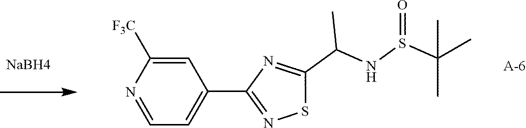 [SMILES: "NaBH4" ; CC(NS(=O)C(C)(C)C)c1nc(-c2ccnc(C(F)(F)F)c2)ns1]

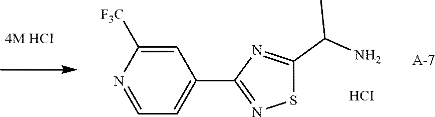

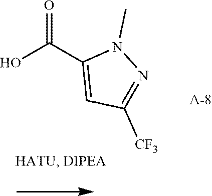

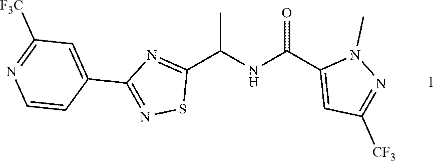 [SMILES: CC(NC(=O)c1cc(C(F)(F)F)nn1C)c1nc(-c2ccnc(C(F)(F)F)c2)ns1]

### Synthesis of 2-(trifluoromethyl)pyridine-4-carboxamide (A-2)

To stirred solution of A-1 (10 g, 52.33 mmol) in DCM (10 mL) at 0° C. was added DMF (1 mL) and oxalyl chloride (4.71 mL, 54.94 mmol) and the reaction mixture was stirred at RT for 2 h. The reaction mixture was concentrated to give a residue which was dissolved in MeCN (100 mL) and charged with aq. ammonia solution (150 mL, 52.33 mmol). The mixture was quenched using water (100 mL) and diluted with EtOAc (200 mL × 2). The organic layer was separated, dried over Na₂SO₄, filtered and concentrated under reduced pressure to give a residue, which was purified by column chromatography using 100-200 silica and 30-80% EtOAc/hexane as an eluent to give A-2 (7 g, 33.13 mmol, 63% yield).

### Synthesis of 5-[2-(trifluoromethyl)-4-pyridyl]-1,3,4-oxathiazol-2-one (A-3)

A solution of A-2 (1.5 g, 7.89 mmol) and chlorocarbonylsulfenyl chloride (1.2 g, 9.47 mmol) in toluene (20 mL) was stirred for 16 h at 120° C. The reaction was quenched with water (100 mL), diluted with EtOAc (100 mL × 2), and the organic layer was separated. The organic layer was dried over Na₂SO₄, filtered and concentrated to give a residue which was purified by column chromatography using 100-200 silica and 5-50% EtOAc/Hexane as an eluent to give A-3 (1.5 g, 5.43 mmol, 69% yield).

### Synthesis of 1-[3-[2-(trifluoromethyl)-4-pyridyl]-1,2,4-thiadiazol-5-yl]ethanone (A-4)

A mixture of A-3 (1 g, 4.03 mmol) and acetyl cyanide (278.27 mg, 4.03 mmol) in 1,2-dichlorobenzene (10 mL) was stirred at 24 h at 160° C. The reaction mixture was quenched with water (100 mL), diluted with EtOAc (100 mL × 2), and the organic layer was separated, dried over Na₂SO₄, filtered, and concentrated under reduced pressure to give a residue, which was purified by column chromatography using 100-200 silica and 10-50% EtOAc/Hexane as an eluent to give A-4 (0.4 g, 1.39 mmol, 34 % yield).

### Synthesis of (E)-2-methyl-N-[1-[3-[2-(trifluoromethyl)-4-pyridyl]-1,2,4-thiadiazol-5-yl]ethylidene]propane-2-sulfinamide (A-5)

To stirred the solution of A-4 (100 mg, 0.37 mmol) and 2-methylpropane-2-sulfinamide (66.54 mg, 0.55 mmol) in toluene (10 mL) was added titanium(IV) ethoxide (0.12 mL, 0.55 mmol) and the mixture was stirred at 80° C. for 16 h. The reaction mixture was quenched using water and diluted with ethyl acetate. The organic layer was separated, dried with sodium sulfate, and concentrated to give a residue which was purified by column chromatography using 100-200 silica and 10-30% EtOAc/hexane as an eluent to give A-5 (100 mg, 0.13 mmol, 36% yield) as a liquid.

### Synthesis of 2-methyl-N-[1-[3-[2-(trifluoromethyl)-4-pyridyl]-1,2,4-thiadiazol-5-yl]ethyl]propane-2-sulfinamide (A-6)

To stirred the solution of A-5 (100 mg, 0.27 mmol) in methanol (10 mL) at 0° C. was added sodium borohydride (15.07 mg, 0.4 mmol) and the mixture was stirred at RT for 1 h. The reaction mixture was diluted with ethyl acetate and the organic layer was washed with water. The organic layer was dried with sodium sulphate and concentrated under reduced pressure to give A-6 (80 mg, 0.10 mmol, 40% yield).

### Synthesis of 1-[3-[2-(trifluoromethyl)-4-pyridyl]-1,2,4-thiadiazol-5-yl]ethanamine hydrochloride (A-7)

To a stirred solution of A-6 (80 mg, 0.21 mmol) in 1,4 dioxane (5 mL) at 0° C. was added 4 M HCl in 1,4 dioxane (5 mL, 0.21 mmol) and the mixture was stirred at RT for 2 h. The reaction mixture was concentrated under reduced pressure to give a residue which was washed using diethyl ether to give A-7 (65 mg, 0.15 mmol, 69% yield).

### Synthesis of 1-methyl-3-(trifluoromethyl)-N-(1-(3-(2-(trifluoromethyl)pyridin-4-yl)-1,2,4-thiadiazol-5-yl)ethyl)-1H-pyrazole-5-carboxamide (1)

To a stirred solution of A-7 (70 mg, 0.18 mmol) and A-8 (41.98 mg, 0.22 mmol) in DCM (10 mL) was added HATU (102.79 mg, 0.27 mmol) and DIPEA (0.06 mL, 0.36 mmol) at RT. The reaction mixture was stirred at RT for 2 h then was quenched with water (100 mL) and diluted with DCM (100 mL × 2). The organic layer was dried over anhydrous sodium sulphate, filtered and concentrated under reduced pressure to give a residue. The residue was purified by column chromatography using 100-200 silica and 30-80% EtOAc/hexane as eluent to give 1 (10 mg, 0.022 mmol, 12% yield). HPLC: Rt 9.346 min, 97.6%; Column: X-Select CSH C18 (4.6 × 150) mm, 3.5 µm; Mobile phase: A: 0.1% Formic acid in water: ACN (95:05), B: ACN; Flow Rate: 1.0 mL/min. LCMS : 450.9 (M+H), Rt 2.32 min; Column: X-select CSH C18 (3*50) mm, 2.5 µm. ¹H NMR (400 MHz, DMSO-d6) δ_{H} = 9.55 (d, 1H), 8.97 (d, 1H), 8.44 (s, 1H), 8.40 (d, 1H), 7.46 (s, 1H), 5.62-5.58 (m, 1H), 4.13 (s, 3H), 1.71 (d, 3H).

Examples 2 and 3. Synthesis of (S)-1-methyl-3-(trifluoromethyl)-N-(1-(3-(2-(trifluoromethyl)pyridin-4-yl)isoxazol-5-yl)ethyl)-1H-pyrazole-5-carboxamide (2) and (R)-1-methyl-3-(trifluoromethyl)-N-(1-(3-(2-(trifluoromethyl)pyridin-4-yl)isoxazol-5-yl)ethyl)-1H-pyrazole-5-carboxamide (3). Note that stereochemistry is randomly assigned.

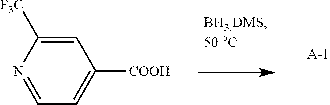

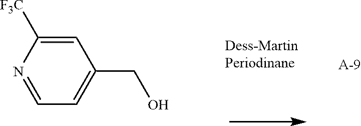

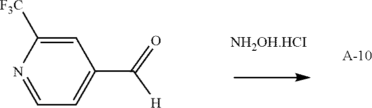

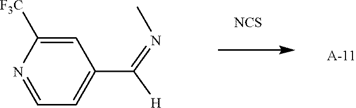

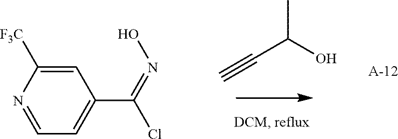

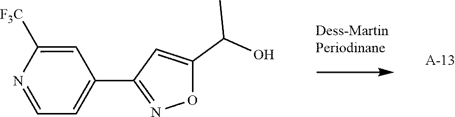

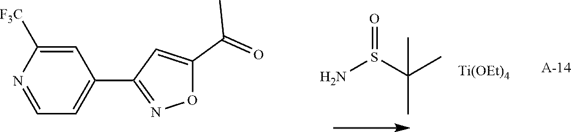

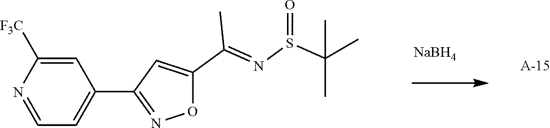 [SMILES: C/C(=N\S(=O)C(C)(C)C)c1cc(-c2ccnc(C(F)(F)F)c2)no1 ; "NaBH4"]

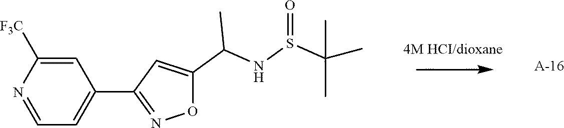

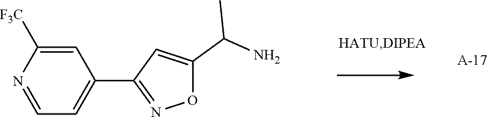

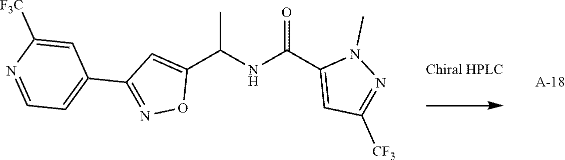

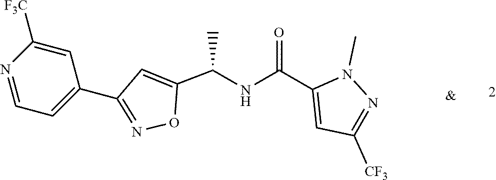

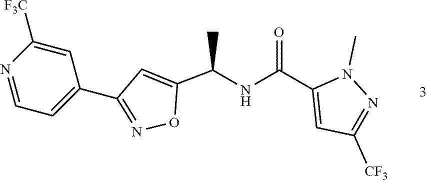 [SMILES: C[C@@H](NC(=O)c1cc(C(F)(F)F)nn1C)c1cc(-c2ccnc(C(F)(F)F)c2)no1]

### Synthesis of (2-(trifluoromethyl)pyridin-4-yl)methanol (A-9)

To a stirred solution of A-1 (7 g, 36.63 mmol) in THF (30 mL) was added borane DMS (2 M in THF) (36.6 mL, 73.26 mmol) at 0° C. and the mixture was stirred at RT for 3 h. The reaction mixture was then heated to 50° C. for 12 h and then cooled to RT. The reaction mixture was slowly quenched using MeOH (30 mL) at 0° C. and stirred at RT 30 min. The mixture was concentrated under reduced pressure and the residue was cooled to 0° C. The residue was rendered alkaline with 1 N sodium hydroxide (30 mL) and diluted with EtOAc (100 mL) and the phases were separated. The organic layer was dried over anhydrous sodium sulphate, filtered and concentrated under reduced pressure to give A-9 (2.8 g, 11.2 mmol, 31 % yield) as an oil.

### Synthesis of 2-(trifluoromethyl)pyridine-4-carbaldehyde (A-10)

To a stirred solution of A-9 (2.8 g, 15.81 mmol) in DCM (20 mL) was added desmartin periodinane (13.41 g, 31.62 mmol) at 0° C. and stirred at RT for 16 h. The reaction mixture was diluted with DCM (20 mL), saturated sodium thiosulphate (30 mL) and saturated sodium bicarbonate (30 mL) and the layers were separated. The organic layer was washed with water (2 × 30 mL) then saturated brine solution (30 mL). The organic layer was then separated and dried over MgSO₄ and concentrated under reduced pressure to give A-10 (2.5 g, 7.56 mmol, 48% yield) as an oil.

### Synthesis of (4Z)-2-(trifluoromethyl)pyridine-4-carbaldehyde Oxime (A-11)

To a stirred solution of A-10 (2.5 g, 14.28 mmol) in ethanol (10 mL) and water (20 mL) was added Na₂CO₃ (1.82 g, 17.13 mmol), hydroxyl amine hydrochloride (1.19 g, 17.13 mmol) and the mixture was stirred at RT for 12 h. The reaction mixture was concentrated and the residue was diluted with EtOAc (20 mL) and water (10 mL) and separated. The organic layer was washed with water (2 × 10 mL), saturated brine solution (10 mL), separated then dried over MgSO₄ and concentrated under reduced pressure. The residue was then purified by flash column chromatography using 30% EtOAc in hexane as an eluent to give A-11 (1.9 g, 9.36 mmol, 65% yield) as a solid.

### Synthesis of (4E)-N-hydroxy-2-(trifluoromethyl)pyridine-4-carboximidoyl Chloride (A-12)

To a solution of A-11 (1.9 g, 9.99 mmol) in DMF (5 mL) was added N-chloro succenamide (2.67 g, 19.99 mmol) and the mixture was stirred at RT for 6 h. The reaction mixture was diluted with EtOAc (50 mL) and water (20 mL) and the phases were separated. The organic layer was washed with water (2 × 20 mL), then saturated brine solution (20 mL), and the organic layer was separated and dried over MgSO₄ then concentrated. The residue was purified by flash column chromatography, eluting with 30% EtOAc in hexane. The desired fractions were concentrated under reduced pressured to give A-12 (1.3 g, 4.39 mmol, 44% yield) as a solid.

### Synthesis of 1-[3-[2-(trifluoromethyl)-4-pyridyl]isoxazol-5-yl]ethanol (A-13)

To a stirred solution of A-12 (0.4 g, 1.78 mmol) in toluene (10 mL) was added but-3-yn-2-ol (0.25 g, 3.56 mmol) and triethyl amine (0.18 g, 1.78 mmol) at 0° C. and stirred at RT for 1 h then heated at 60° C. for 3 h. The reaction mixture was concentrated under reduced pressure and the residue was diluted with EtOAc (20 mL) and water (10 mL), separated, and the organic layer was washed with water (2 × 10 mL) then saturated brine solution (10 mL). The organic layer was separated and dried over MgSO₄ then concentrated under reduced pressure. The residue was purified by flash column chromatography eluting 80% EtOAc in hexane. The desired fractions were concentrated under reduced pressure to give A-13 (0.45 g, 1.69 mmol, 95% yield) as an oil.

### Synthesis of 1-[3-[2-(trifluoromethyl)-4-pyridyl]isoxazol-5-yl]ethanone (A-14)

To stirred solution of A-13 (0.45 g, 1.74 mmol) in DCM (10 mL) was added desmartin periodinane (1.48 g, 3.49 mmol) and the reaction mixture was stirred at RT for 12 h. The reaction mixture was diluted with DCM (30 mL) and saturated sodium thiosulphate 10 (mL) and washed with saturated bicarbonate (10 mL). The organic layer was then separated, dried over MgSO₄ and evaporated to dryness to give a residue which was purified by flash column chromatography using 80% EtOAc in hexane as an eluent to give A-14 (0.2 g, 0.73 mmol, 42% yield) as a solid.

### Synthesis of (NE)-2-methyl-N-[1-[3-[2-(trifluoromethyl)-4-pyridyl]isoxazol-5-yl]ethylidene]propane-2-sulfinamide (A-15)

To a stirred solution of A-14 (0.15 g, 0.59 mmol) in toluene (10 mL) was added 2-methyl-2-propane sulfinamide (0.11 g, 0.88 mmol) and titanium(IV) ethoxide (0.2 g, 0.88 mmol) at RT. The reaction mixture was heated to 80° C. for 12 h. The reaction mixture was diluted with water and EtOAc (30 mL) and separated. The organic layer was dried over MgSO₄ and evaporated to dryness. The residue was then purified by flash column chromatography using 80% EtOAc in hexane as an eluent to give A-15 (0.14 g, 0.32 mmol, 54% yield) as an oil.

### Synthesis of 2-methyl-N-[1-[3-[2-(trifluoromethyl)-4-pyridyl]isoxazol-5-yl]ethyl]propane-2-sulfinamide (A-16)

To stirred the solution of A-15 (0.46 g, 1.28 mmol) in methanol (5 mL) at 0° C. was added sodium borohydride (0.048 g, 1.28 mmol) and the reaction mixture was stirred at RT for 1 h. The reaction was quenched with water, diluted with ethyl acetate and the organic layer was separated. The organic layer was dried over MgSO₄ and concentrated under reduced pressure to give a residue which was purified by flash column chromatography using 80% EtOAc in hexane as an eluent to give A-16 (450 mg, 1.24 mmol, 97% yield).

### Synthesis of 1-[3-[2-(trifluoromethyl)-4-pyridyl]isoxazol-5-yl]ethanamine hydrochloride (A-17)

To a stirred solution of A-16 (430 mg, 1.19 mmol) in 1,4 dioxane (2 mL) at 0° C. was added 4 M HCl in 1,4 dioxane (8.6 mL, 61.6 mmol) and stirred at RT for 2 h. The reaction mixture was evaporated to give A-17 (310 mg, 1.05 mmol, 89% yield).

### Synthesis of (S)-1-methyl-3-(trifluoromethyl)-N-(1-(3-(2-(trifluoromethyl)pyridin-4-yl)isoxazol-5-yl)ethyl)-1H-pyrazole-5-carboxamide (2) and (R)-1-methyl-3-(trifluoromethyl)-N-(1-(3-(2-(trifluoromethyl)pyridin-4-yl)isoxazol-5-yl)ethyl)-1H-pyrazole-5-carboxamide (3). Note that stereochemistry is randomly assigned

To stirred solution of A-17 (0.07 g, 0.24 mmol) in DCM (10 mL) was added 2-methyl-5-(trifluoromethyl)pyrazole-3-carboxylic acid (0.05 g, 0.24 mmol), HATU (90.63 mg, 0.24 mmol), and DIPEA (0.08 mL, 0.48 mmol) at 0° C. and the mixture was stirred at RT for 6 h. The reaction mixture was diluted with DCM (20 mL) and water (10 mL), and the organic layer was separated. The organic layer was washed with water (2 × 10 mL), saturated brine solution (10 mL), separated and dried over MgSO₄ and concentrated to dryness to give a residue, which was then purified by flash column chromatography eluting 80% EtOAc in hexane. The desired fractions were concentrated to dryness to give A-18 as an oil which was purified by chiral prep HPLC to give 2 (10 mg, 0.023 mmol, 9% yield) and 3 (8 mg, 0.018 mmol, 8% yield). Note: absolute stereochemistry was randomly assigned. The separation was done using prep HPLC condition SFC using following conditions. DIACEL CHIRALPAK-IG (250 mm × 4.6 mm, 5 um), - Mobile Phase: A) n-Hexane+0.1% Iso-propyl-amine B) EtOH: MeOH (50:50), Isocratic:20% B; Wavelength: 293 nm, Flow: 1.0 mL/min.

2: HPLC: Rt 9.172 min, 99.7%; Column: X-Select CSH C18 (4.6 × 150) mm, 3.5 µm; Mobile phase: A: 0.1% Formic acid in water: ACN (95:05), B: ACN; Flow Rate: 1.0 mL/min. LCMS : 434.25 (M+H), Rt 2.018 min, Column: X-select CSH (3*50) mm, 2.5 µm. ¹H NMR (400 MHz, DMSO-d6) δ_{H} = 9.28 (d, 1H), 8.93 (d, 1H), 8.33 (s, 1H), 8.21 (d, 1H), 7.45 (s, 1H), 7.37 (s, 1H), 5.40 (quin, 1H), 4.15 (s, 3H), 1.60 (d, 3H). Chiral method: Rt 5.392 min, 100%: DIACEL CHIRALPAK-IG (250 mm ×4.6 mm,5 u), - Mobile Phase: A) n-Hexane+0.1% Iso-propyl-amine B) EtOH: MeOH (50:50), Isocratic:20% B; Wavelength: 293 nm, Flow: 1.0 mL/min.

3: HPLC: Rt 9.146 min, 99.8%; Column: X-Select CSH C18 (4.6 × 150) mm, 3.5 µm; Mobile phase: A: 0.1% Formic acid in water: ACN (95:05), B: ACN; Flow Rate: 1.0 mL/min. LCMS : 433.95 (M+H), Rt 2.012 min, Column: X-select CSH (3*50) mm, 2.5 µm. ¹H NMR (400 MHz, DMSO-d6) δ_{H} = 9.29 (d, 1H), 8.93 (d, 1H), 8.33 (s, 1H), 8.21 (d, 1H), 7.45 (s, 1H), 7.38 (s, 1H), 5.40 (quin, 1H), 4.15 (s, 3H), 1.61 (d, 3H). Chiral method: Rt 4.989 min, 98%: DIACEL CHIRALPAK-IG (250 mm ×4.6 mm,5 u), - Mobile Phase: A) n-Hexane+0.1% Iso-propyl-amine B) EtOH: MeOH (50:50), Isocratic:20% B; Wavelength: 254 nm, Flow: 1.0 mL/min.

Example 2-1. Synthesis of (S)-1-methyl-3-(trifluoromethyl)-N-(1-(3-(2-(trifluoromethyl)pyridin-4-yl)isoxazol-5-yl)ethyl)-1H-pyrazole-5-carboxamide (2-1)

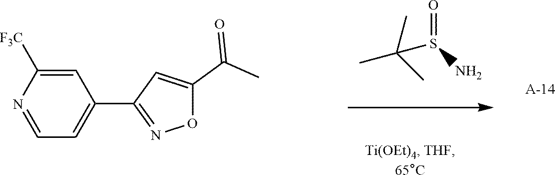

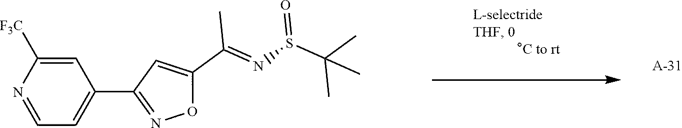

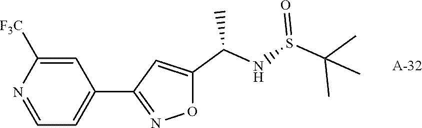 [SMILES: C[C@H](N[S@](=O)C(C)(C)C)c1cc(-c2ccnc(C(F)(F)F)c2)no1]

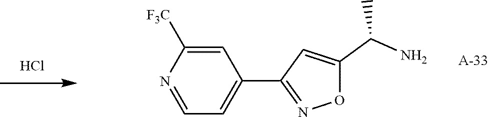 [SMILES: Cl ; C[C@H](N)c1cc(-c2ccnc(C(F)(F)F)c2)no1]

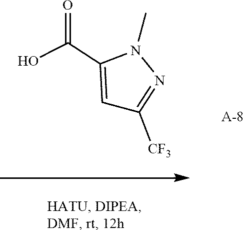

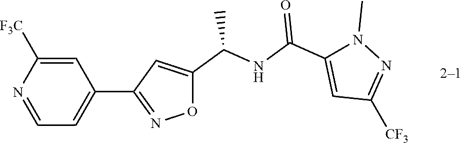 [SMILES: C[C@H](NC(=O)c1cc(C(F)(F)F)nn1C)c1cc(-c2ccnc(C(F)(F)F)c2)no1]

### Synthesis of (R,Z)-2-methyl-N-(1-(3-(2-(trifluoromethyl)pyridin-4-yl)isoxazol-5-yl)ethylidene)propane-2-sulfinamide (A-31)

To a stirred solution of A-14 (1.2 g, 4.68 mmol) and (R)-2-methylpropane-2-sulfinamide (850.18 mg, 7.01 mmol) in THF (20 mL) was added titaniumethoxide (2.97 mL, 14.05 mmol) and the mixture was stirred at 65° C. for 6 h. The reaction mixture was quenched using water and diluted with ethyl acetate. The organic layer was separated, dried over anhydrous Na₂SO₄ and concentrated under reduced pressure to afford A-31 (1.4 g, 1.17 mmol, 25% yield).

### Synthesis of (R)-2-methyl-N-((S)-1-(3-(2-(trifluoromethyl)pyridin-4-yl)isoxazol-5-yl)ethyl)propane-2-sulfinamide (A-32)

To a stirred solution of A-31 (700 mg, 1.95 mmol) in THF (10 mL) was added L-selectride (221.76 mg, 5.84 mmol) at 0° C. and the reaction mixture was stirred at room temperature for 3 h. The reaction mixture was concentrated under reduced pressure, treated with water and extracted with DCM (20 mL). The combined organic layers were dried over anhydrous Na₂SO₄, filtered and evaporated to get a residue which was purified by column chromatography using 100-200 silica and 50-60% EtOAc/hexane as an eluent to afford A-32 (250 mg, 0.64 mmol, 32% yield) as a liquid.

### Synthesis of (S)-1-(3-(2-(trifluoromethyl)pyridin-4-yl)isoxazol-5-yl)ethan-1-amine (A-33)

To a stirred the solution of A-32 (250 mg, 0.69 mmol) in 1,4-dioxane (1 mL) was added 4 M HCl in dioxane (0.5 mL, 0.69 mmol) at 0° C. and stirred at room temperature for 2 h. The reaction mixture was concentrated under reduced pressure and triturated using diethyl ether to afford A-33 (150 mg,0.566 mmol, 81% yield) as a solid.

### Synthesis of (S)-1-methyl-3-(trifluoromethyl)-N-(1-(3-(2-(trifluoromethyl)pyridin-4-yl)isoxazol-5-yl)ethyl)-1H-pyrazole-5-carboxamide (2-1)

To a stirred solution of A-33 (180 mg, 0.7000 mmol) in DCM (10 mL) was added 2-methyl-5-(trifluoromethyl)pyrazole-3-carboxylic acid (203.76 mg, 1.05 mmol), HATU (399.14 mg, 1.05 mmol), and DIPEA (0.37 mL, 2.1 mmol), and the reaction mixture was stirred at room temperature for 2 h. The reaction mixture was diluted with DCM (20 mL), water (10 mL), and organic layer was separated. The organic layer was washed with water (2 × 10 mL), saturated brine solution (10 mL), separated and dried over MgSO₄ and concentrated under reduced pressure. The residue was then purified by flash column chromatography eluting 30-50% EtOAc in hexane followed by preparative HPLC to afford 2-1 (95 mg, 0.218 mmol, 31% yield). HPLC: Rt 8.484 min, 99.58%; Column: XSELECT CSH C18 (150 × 4.6 mm, 3.5 µ); Mobile Phase-A: 0.1% TFA in Water; Mobile Phase-B:Acetonitrile; LCMS : 434.1 (M+H), Rt 2.381 min, Column:X-Bridge BEH C-18(3.0×50 mm,2.5 µm); Mobile Phase: A: 0.025% FA in Water, B: ACN; Flow rate: 1.2ml/min; Chiral HPLC: Rt 4.869 min, 98.80%; Column: CHIRAL PAK IG (250*4.6 mm*5 µm); Mobile Phase A: 0.1%IP Amine in n-HEXANE; Mobile Phase B:ETOH:MEOH(1:1); AB : 80:20; Flow: 1.0 mL/min. ¹H NMR (400 MHz, DMSO-d6) δ_{H} = 9.27 (d, 1H), 8.93 (d, 1H), 8.33 (s, 1H), 8.23 - 8.19 (m, 1H), 7.45 (s, 1H), 7.39 - 7.36 (m, 1H), 5.40 (quin, 1H), 4.15 (s, 3H), 1.61 (d, 3H).

Example 3-1. Synthesis of (R)-1-methyl-3-(trifluoromethyl)-N-(1-(3-(2-(trifluoromethyl)pyridin-4-yl)isoxazol-5-yl)ethyl)-1H-pyrazole-5-carboxamide (3-1):

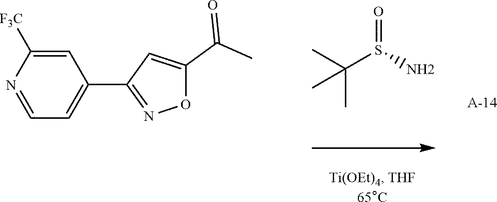

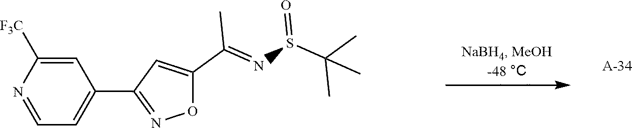

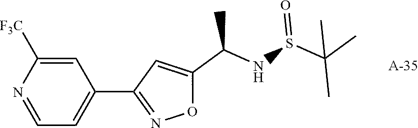 [SMILES: C[C@@H](N[S@@](=O)C(C)(C)C)c1cc(-c2ccnc(C(F)(F)F)c2)no1]

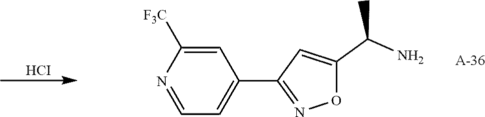 [SMILES: "HCI" ; C[C@@H](N)c1cc(-c2ccnc(C(F)(F)F)c2)no1]

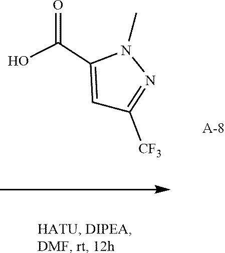

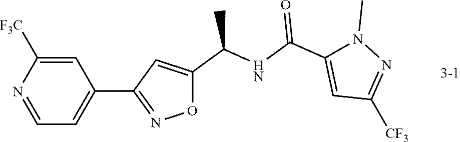 [SMILES: C[C@@H](NC(=O)c1cc(C(F)(F)F)nn1C)c1cc(-c2ccnc(C(F)(F)F)c2)no1]

### Synthesis of (S,E)-2-methyl-N-(1-(3-(2-(trifluoromethyl)pyridin-4-yl)isoxazol-5-yl)ethylidene)propane-2-sulfinamide (A-34)

To a stirred solution of A-14 (600 mg, 2.34 mmol) and (S)-2-methylpropane-2-sulfinamide (425.09 mg, 3.51 mmol) in toluene (20 mL) was added titanium ethoxide (1.48 mL, 7.03 mmol) and the mixture was stirred at 90° C. for 6 h. The reaction mixture was quenched using water and diluted with ethyl acetate. The organic layer was separated, dried over anhydrous Na₂SO₄ and concentrated under reduced pressure to afford A-34 (500 mg, 0.64 mmol, 27% yield).

### Synthesis of (S)-2-methyl-N-((R)-1-(3-(2-(trifluoromethyl)pyridin-4-yl)isoxazol-5-yl)ethyl)propane-2-sulfinamide (A-35)

To a stirred solution of A-34 (500 mg, 1.39 mmol) in methanol (10 mL) was added sodium borohydride (105.6 mg, 2.78 mmol) at -40° C. and the reaction mixture was stirred at the same temperature for 1 h. The reaction mixture was quenched using water (25 mL) and diluted with EtOAc (2 × 50 mL). The organic layer was separated, dried over anhydrous Na₂SO₄, filtered and evaporated to get a residue which was purified by column chromatography using 100-200 silica and 30-80% EtOAc/hexane as an eluent to afford A-35 (270 mg, 0.7322 mmol, 52% yield).

### Synthesis of (R)-1-(3-(2-(trifluoromethyl)pyridin-4-yl)isoxazol-5-yl)ethan-1-amine (A-36)

To a stirred solution of A-35 (270 mg, 0.7500 mmol) in 1,4-dioxane (1 mL) was added 4 M HCl in dioxane (0.5 mL, 0.7500 mmol) at 0° C. and the mixture was stirred at room temperature for 2 h. The reaction mixture was concentrated under reduced pressure. The residue was washed with diethyl ether to afford A-36 (180 mg, 0.6578 mmol, 88% yield).

### Synthesis of (R)-1-methyl-3-(trifluoromethyl)-N-(1-(3-(2-(trifluoromethyl)pyridin-4-yl)isoxazol-5-yl)ethyl)-1H-pyrazole-5-carboxamide (3-1)

To a stirred solution of A-36 (180 \.mg, 0.7000 mmol) in DCM (10 mL) was added 2-methyl-5-(trifluoromethyl)pyrazole-3-carboxylic acid (203.76 mg, 1.05 mmol), HATU (399.14 mg, 1.05 mmol), and DIPEA (0.37 mL, 2.1 mmol) at 0° C., and the mixture was stirred at room temperature for 6 h. The reaction mixture was diluted with DCM (20 mL), water (10 mL), and organic layer was separated. The organic layer was washed with water (2 Synthesis of (R)-1-methyl-3-(trifluoromethyl)-N-(1-(3-(2-(trifluoromethyl)pyridin-4-yl)isoxazol-5-yl)ethyl)-1H-pyrazole-5-carboxamide (3-1): 10 mL), saturated brine solution (10 mL), separated and dried over MgSO₄ before concentration to dryness. The residue was then purified by flash column chromatography eluting 30-50% EtOAc in hexane followed by preparative HPLC to afford 3-1 (70 mg, 0.1596 mmol, 23% yield). HPLC: Rt 7.85 min, 98.78%; Column: X SELECT CSH C18 (150×4.6 mm,3.5 u); Mobile Phase A; 0.05% TFA IN WATER;ACN(95:05); Mobile Phase B : 0.05%; FA IN WATER:ACN(05:95); Flow :1.0 mL/min; LCMS : 434.1 (M+H), Rt 2.342 min, Column: X-Bridge BEH C-18(3.0×50 mm,2.5 µm); Mobile Phase: A: 0.025% FA in Water, B: ACN; Flow rate: 1.2 ml/min

Chiral method: Rt 4.919 min, 100% COLUMNE: Chiral pak-IG (250*4.6 mm) 5 µm; MOBILE PHASE A: 0.1%IP Amine n-Hexane MOBILE PHASE B: ETOH : MEOH (50:50); PROGRAM- AB 80:20; FLOW RATE: 1.0 ML/MIN. ¹H NMR (400 MHz, DMSO-d6) δ_{H} = 9.27 (d, 1H), 8.93 (d, 1H), 8.33 (s, 1H), 8.21 (d, 1H), 7.45 (s, 1H), 7.37 (d, 1H), 5.40 (quin, 1H), 4.15 (s, 3H), 1.61 (d, 3H).

Examples 2-2 and 3-2. Synthesis of 2-methyl-N-[(1S)-1-[3-[2-(trifluoromethyl)-4-pyridyl]isoxazol-5-yl]ethyl]-5-(trifluoromethyl)pyrazole-3-carboxamide & 2-methyl-N-[(1R)-1-[3-[2-(trifluoromethyl)-4-pyridyl]isoxazol-5-yl]ethyl]-5-(trifluoromethyl)pyrazole-3-carboxamide. Note that stereochemistry is randomly assigned.

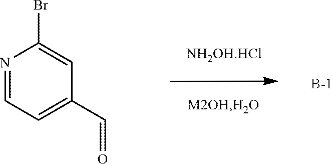

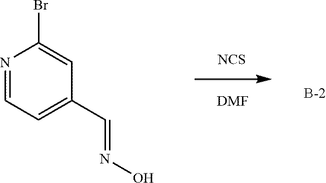

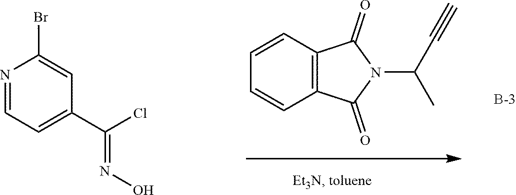

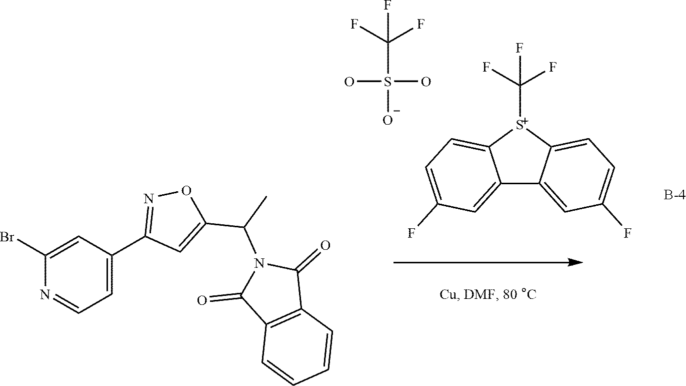

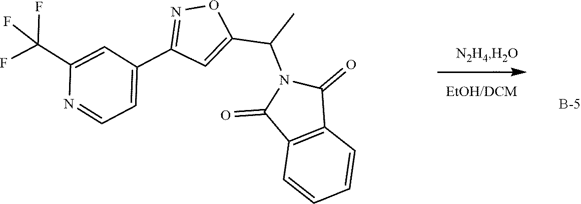

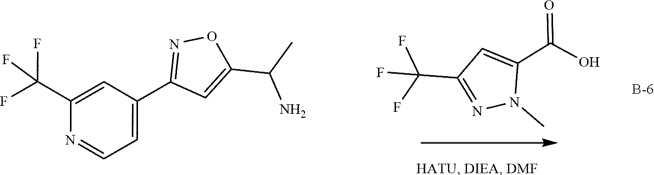

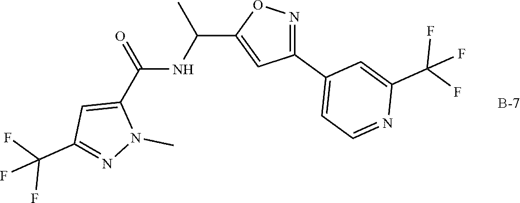 [SMILES: CC(NC(=O)c1cc(C(F)(F)F)nn1C)c1cc(-c2ccnc(C(F)(F)F)c2)no1]

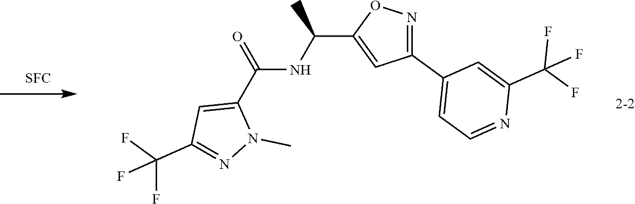 [SMILES: "SFC" ; C[C@H](NC(=O)c1cc(C(F)(F)F)nn1C)c1cc(-c2ccnc(C(F)(F)F)c2)no1]

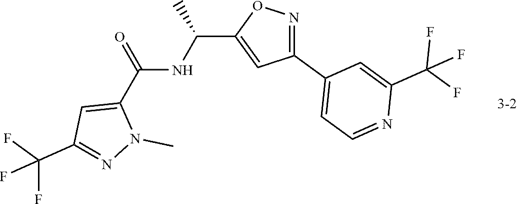 [SMILES: C[C@@H](NC(=O)c1cc(C(F)(F)F)nn1C)c1cc(-c2ccnc(C(F)(F)F)c2)no1]

### (4E)bromopyridine-4-carbaldehyde Oxime (B-2)

To a mixture of 2-bromopyridine-4-carbaldehyde (20.0 g, 107 mmol) in water (120 mL) and MeOH (120 mL) was added NH₂OH.HC1 (33.2 g, 161 mmol). The mixture was stirred at 60° C. for 12 hours under N₂. After cooling to 30° C., the mixture was filtered, washed with water (50 mL) and concentrated to give the product (22.0 g, 76.6 mmol, 71% yield) as a solid. ¹H NMR (DMSO-d6, 400 MHz) δ_{H} = 12.14-11.93 (m, 1H), 8.43-8.32 (m, 1H), 8.20-8.13 (m, 1H), 7.80-7.73 (m, 1H), 7.66-7.57 (m, 1H).

### (4Z)bromo-N-hydroxy-pyridine-4-carboximidoyl Chloride (B-3)

To a mixture of (4E)-2-bromopyridine-4-carbaldehyde oxime (22.0 g, 76.6 mmol) in DMF (60 mL) was added NCS (12.3 g, 91.9 mmol) at 0° C. The mixture was stirred at 20° C. for 3 days. The mixture was poured into water (100 mL) and stirred for 20 mins. The aqueous phase was extracted with EtOAc (3 × 50 mL). The combined organic phase was washed with saturated brine (2 × 50 mL), dried over anhydrous Na₂SO₄, filtered and concentrated. The mixture was triturated by PE (50 mL) to afford the product (15.0 g, 63.7 mmol, 83% yield) as a solid. LCMS Rₜ = 0.849 min in 1.5 min chromatography, 5-95AB, MS ESI calcd. for C₆H₅BrClN₂O [M+H]⁺234.9, found 236.7

### 2-[3-(2-Bromo-4-pyridyl)isoxazol-5-yl]ethyl]isoindoline-1,3-dione (B-4)

To a mixture of 2-(1-methylprop-2-ynyl)isoindoline-1,3-dione (2.28 g, 11.5 mmol) in toluene (50.0 mL) was added Et₃N (3.53 mL, 25.5 mmol) and (4Z)-2-bromo-N-hydroxy-pyridine-4-carboximidoyl chloride (3.0 g, 12.7 mmol). The mixture was stirred at 120° C. for 16 hours. The mixture was poured into water (100 mL) and stirred for 20 min. The aqueous phase was extracted with EtOAc (3 × 100 mL). The combined organic phase was washed with saturated brine (2 × 100 mL), dried over anhydrous Na₂SO₄, filtered and concentrated. The residue was purified by silica gel chromatography (PE/EtOAc = 5/1 to 3/1) to afford the product (1.30 g, 3.26 mmol, 26% yield) as an oil. ¹H NMR (CDCl₃, 400 MHz) δ_{H} = 8.47 (d, 1H), 7.92-7.84 (m, 3H), 7.80-7.74 (m, 2H), 7.68-7.64 (m, 1H), 6.66 (s, 1H), 5.79-5.67 (m, 1H), 1.94 (d, 3H).

### 2-[3-[2-(Trifluoromethyl)-4-pyridyl]isoxazol-5-yl]ethyl]isoindoline-1,3-dione (B-5)

To a mixture of Cu (479 mg, 7.5 mmol) and 2,8-difluoro-5-(trifluoromethyl)-5H-dibenzo[b,d]thiophen-5-ium trifluoromethanesulfonate (2.20 g, 5.0 mmol) was added 2-[1-[3-(2-bromo-4-pyridyl)isoxazol-5-yl]ethyl]isoindoline-1,3-dione (1.0 g, 2.5 mmol) in DMF (15 mL) at N₂. The mixture was stirred 0° C. for 1 h and then stirred at 80° C. for 3 hours. The mixture was poured into water (50 mL) extracted with EtOAc (3 × 50 mL). The combined organic phase was washed with brine (3 × 30 mL), dried over anhydrous Na₂SO₄, filtered and concentrated. The mixture was purified by silica gel chromatography (PE/EtOAc = 5/1 to 3/1) to afford the product (720 mg, 1.90 mmol, 74% yield) as a solid. ¹H NMR (CDCl₃, 400 MHz) δ_{H} = 8.84 (d, 1H), 8.08-7.99 (m, 1H), 7.93-7.84 (m, 3H), 7.82-7.68 (m, 2H), 6.74 (d, 1H), 5.80-5.67 (m, 1H), 1.96 (d, 3H).

### 1- [2-(Trifluoromethyl)-4-pyridyl] Isoxazol-5-yl] Ethanamine- [4,3-a] Pyrazine (B-6)

To a solution of 2-[1-[3-[2-(trifluoromethyl)-4-pyridyl]isoxazol-5-yl]ethyl]isoindoline-1,3-dione (300 mg, 0.77 mmol) in DCM (10 mL) and EtOH (2.0 mL) was added N₂H₄.H₂O (0.23 mL, 4.70 mmol) dropwise at 25° C. After stirring at 25° C. for 16 hours, the mixture was filtered and the filter cake was washed with DCM (3 × 10 mL). The filtrate was concentrated to afford the product (200 mg, 0.78 mmol, 100% yield) as a solid which was used directly for the next step.

### 2-methyl-5-(trifluoromethyl)-N-[1-[3-[2-(trifluoromethyl)-4-pyridyl] Isoxazol-5-yl]ethyl]pyrazole-3-carboxamide (B-7)

To a solution of 2-methyl-5-(trifluoromethyl)pyrazole-3-carboxylic acid (125 mg, 0.64 mmol), DIEA (0.30 mL, 1.8 mmol), HATU (443 mg, 1.2 mmol) in DMF (2.0 mL) was added 1-[3-[2-(trifluoromethyl)-4-pyridyl]isoxazol-5-yl]ethanamine (150 mg, 0.58 mmol) at 20° C. After stirring for 1 hour, the mixture was poured into water (15 mL) and extracted with EtOAc (2 × 20 mL). The combined organic phase was washed with brine (2 × 20 mL), dried over anhydrous Na₂SO₄, filtered and concentrated. The residue was purified by silica gel chromatography (PE/EtOAc = 5/1 to 3/1) to afford the product (150 mg, 0.35 mmol, 59% yield) as a solid. ¹H NMR (CDCl₃, 400 MHz) δ_{H} = 8.86 (d, 1H), 8.11-8.03 (m, 1H), 7.88 (d, 1H), 6.89-6.81 (m, 1H), 6.68-6.61 (m, 1H), 6.42-6.31 (m, 1H), 5.59-5.45 (m, 1H), 4.23 (s, 3H), 1.75 (d, 3H).

### 2-methyl-N-[(1S)-1-[3-[2-(trifluoromethyl)-4-pyridyl]isoxazol-5-yl]ethyl]-5-(trifluoromethyl)pyrazole-3-carboxamide & 2-methyl-N-[(1R)-1-[3-[2-(trifluoromethyl)-4-pyridyl]isoxazol-5-yl]ethyl]-5-(trifluoromethyl)pyrazole-3-carboxamide

The mixture of 2-methyl-5-(trifluoromethyl)-N-[1-[3-[2-(trifluoromethyl)-4-pyridyl]isoxazol-5-yl]ethyl]pyrazole-3-carboxamide (200 mg, 0.46 mmol) was purified by SFC (Column DAICEL CHIRALCEL OJ-H (250 mm * 30 mm, 5 µm), Condition: 0.1%NH₃H₂O-EtOH, Begin B: 15%, End B: 15%, FlowRate (mL/min): 60) to give 2-methyl-N-[(1S)-1-[3-[2-(trifluoromethyl)-4-pyridyl]isoxazol-5-yl]ethyl]-5-(trifluoromethyl)pyrazole-3-carboxamide (60.4 mg, 0.14 mmol, 30% yield, peak 1) as a solid and 2-methyl-N-[(1R)-1-[3-[2-(trifluoromethyl)-4-pyridyl]isoxazol-5-yl]ethyl]-5-(trifluoromethyl)pyrazole-3-carboxamide (69.1 mg, 0.16 mmol, 34% yield) as a solid.

2-2: ¹H NMR (CDCl₃, 400 MHz) δ_{H} = 8.85 (d, 1H), 8.05 (s, 1H), 7.88 (d, 1H), 6.86 (s, 1H), 6.64 (s, 1H), 6.41 (d, 1H), 5.59-5.50 (m, 1H), 4.22 (s, 3H), 1.74 (d, 3H). ¹⁹F NMR (376.5 MHz, CDCl₃) δ_{F} = -62.214, -68.145. LCMS Rₜ = 1.251 min in 2.0 min chromatography, 10-80AB, MS ESI calcd. for C₁₇H₁₄F₆N₅O₂ [M+H]⁺ 434.1, found 434.1.

3-2: ¹H NMR (CDCl₃, 400 MHz) δ_{H} = 8.85 (d, 1H), 8.06 (s, 1H), 7.90-7.85 (m, 1H), 6.86 (s, 1H), 6.64 (s, 1H), 6.41 (d, 1H), 5.59-5.49 (m, 1H), 4.28-4.16 (m, 3H), 1.74 (d, 3H).¹⁹F NMR (376.5 MHz, CDCl₃) δ_{F} = -62.214, -68.145. LCMS Rₜ = 1.229 min in 2.0 min chromatography, 10-80AB, MS ESI calcd. for C₁₇H₁₄F₆N₅O₂ [M+H]⁺ 434.2, found 434.2. Example 2-3. Synthesis of 2-methyl-5-(trifluoromethyl)-N-[(1S)-1-[3-[2-(trifluoromethyl)-4-pyridyl]isoxazol-5-yl]ethyl]pyrazole-3-carboxamide (2-3)

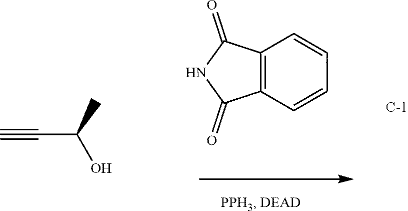

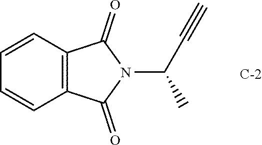

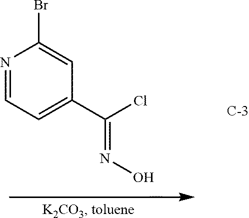

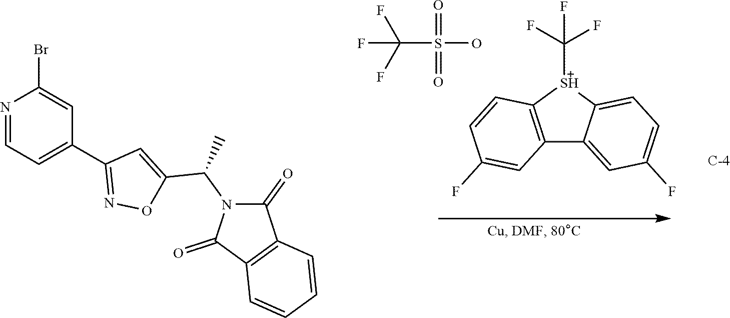

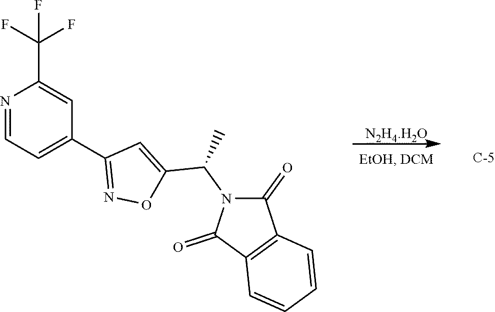

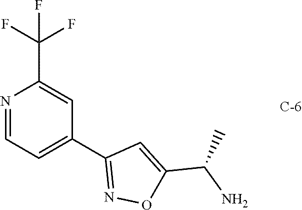 [SMILES: C[C@H](N)c1cc(-c2ccnc(C(F)(F)F)c2)no1]

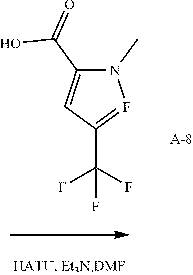

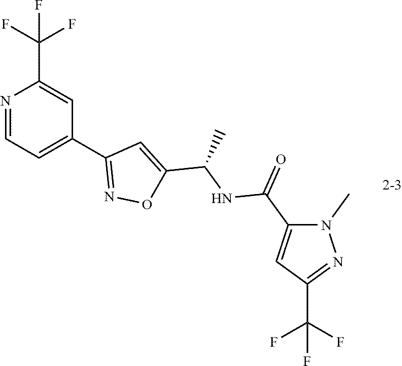 [SMILES: C[C@H](NC(=O)c1cc(C(F)(F)F)nn1C)c1cc(-c2ccnc(C(F)(F)F)c2)no1]

### 2-[(1S)methylprop-2-ynyl]isoindoline-1,3-dione (C-2)

To a mixture of (2R)-but-3-yn-2-ol (2.0 g, 29 mmol), phthalimide (4.2 g, 29 mmol), and PPh₃ (11 g, 43 mmol) in THF (25 mL) was added DEAD (6.8 mL, 43 mmol) at 25° C. After stirring at 25° C. for 16 hours, the mixture was poured into water (100 mL) and extracted with EtOAc (2 MHz 50 mL). The combined organic layer was washed with brine (2 MHz 50 mL), dried over anhydrous Na₂SO₄, filtered and concentrated. The residue was purified by flash column (0~20% of EtOAc in PE) to give the product as a solid.¹H NMR (CDCl₃, 400 MHz) δ_{H} = 7.96-7.81 (m, 2H), 7.78-7.65 (m, 2H), 5.28-5.13 (m, 1H), 2.34 (d, 1H), 1.71 (d, 3H).

### 2-[(1S)[3-(2-bromo-4-pyridyl)isoxazol-5-yl]ethyl]isoindoline-1,3-dione (C-4)

To a mixture of 2-[(1S)-1-methylprop-2-ynyl]isoindoline-1,3-dione (1.1 g, 5.7 mmol) in toluene (13 mL) was added K₂CO₃ (2.6 g, 19 mmol) and (4Z)-2-bromo-N-hydroxypyridine-4-carboximidoyl chloride (1.5 g, 6.4 mmol). After stirring at 120° C. for 12 hours, the mixture was poured into water (50 mL) and stirred for 20 mins. The aqueous phase was extracted with EtOAc (3 × 30 mL). The combined organic phase was washed with saturated brine (2 × 100 mL), dried over anhydrous Na₂SO₄, filtered and concentrated. The residue was purified by silica gel chromatography (PE/EtOAc = 5/1 to 3/1) to afford the product (1.1 g, 2.8 mmol, 43% yield) as a solid. ¹H NMR (CDC1₃ 400 MHz) δ_{H} = 8.47 (d, 1H), 7.92-7.84 (m, 3H), 7.78-7.74 (m, 2H), 7.68-7.61 (m, 1H), 6.66 (d, 1H), 5.77-5.69 (m, 1H), 1.94 (d, 3H).

### 2- [(1 S)-1- [3- [2-(trifluoromethyl)-4-pyridyl] Isoxazol-5-yl] Ethyl] Isoindoline-1,3 Dione (C-5):

To a mixture of Cu (287.3 mg, 4.52 mmol) and 2,8-difluoro-5-(trifluoromethyl)-5H-dibenzo[b,d]thiophen-5-ium trifluoromethanesulfonate (1.33 g, 3.01 mmol) in DMF (15mL) was added 2-[(1S)-1-[3-(2-bromo-4-pyridyl)isoxazol-5-yl]ethyl]isoindoline-1,3-dione (600 mg, 1.51 mmol) under N₂ and stirred at 0° C. for 1 h. After stirring at 80° C. for 3 hours, the mixture was poured into water (30 mL) and extracted with EtOAc (3 × 10 mL). The combined organic phase was washed with saturated brine (3 × 30 mL), dried over anhydrous Na₂SO₄, filtered and concentrated. The mixture was purified by silica gel chromatography (PE/EtOAc = 5/1 to 3/1) to afford the product (520 mg, 1.34 mmol, 89% yield) as an oil. The product (100 mg, 0.26 mmol) was purified by SFC (Column DAICEL CHIRALPAK AD (250 mm^{∗}30 mm, 10 um) Condition Neu-ETOH Begin B 40 End B 40 Gradient Time (min) 100% B)) to give the product (17.0 mg, 0.0437 mmol, 24% yield) as a solid.¹H NMR (CDC1₃, 400 MHz) δ_{H} = 8.83 (d, 1H), 8.06 (s, 1H), 7.92 - 7.85 (m, 3H), 7.80 - 7.73 (m, 2H), 6.73 (s, 1H), 5.85 - 5.67 (m, 1H), 1.96 (d, 3H). ¹⁹F NMR (376.5 MHz, CDC1₃) δ_{F} = -68.155. LCMS Rₜ = 1.029 min in 1.5 min chromatography, 5-95AB, MS ESI calcd. for C₁₉H₁₃F₃N₃O₃ [M+H]⁺ 387.8, found 387.8.

### (1S)[3-[2-(trifluoromethyl)-4-pyridyl]isoxazol-5-yl]ethanamine (C-6)

To a solution of 2-[(1S)-1-[3-[2-(trifluoromethyl)-4-pyridyl]isoxazol-5-yl]ethyl]isoindoline-1,3-dione (250 mg, 0.65 mmol) in DCM (10 mL) and EtOH (2 mL) was added N₂H₄.H₂O (0.19 mL, 3.87 mmol) dropwise at 25° C. After stirring at 25° C. for 16 hrs, the mixture was filtered and the filter cake was washed with DCM (3 × 10 mL). The filtrate was concentrated to afford the product (160 mg, 0.311 mmol, 48% yield) as a solid.

### 2-methyl-5-(trifluoromethyl)-N-[(1S)-1-[3-[2-(trifluoromethyl)-4-pyridyl]isoxazol-5-yl]ethyl]pyrazole-3-carboxamide(2-3)

To a solution of 2-methyl-5-(trifluoromethyl)pyrazole-3-carboxylic acid (132.8 mg, 0.68 mmol) HATU (473 mg, 1.24 mmol) in DMF (10 mL) was added Et₃N (0.26 mL, 1.87 mmol) and (1S)-1-[3-[2-(trifluoromethyl)-4-pyridyl]isoxazol-5-yl]ethanamine (160 mg, 0.62 mmol). After stirring at 20° C. for 12 hours, the reaction mixture was diluted with water (30 mL) and extracted with EtOAc (3 × 20 mL), the organic layer was washed with water (3 × 30 mL) and brine (3 × 30 mL), dried over Na₂SO₄, filtered and concentrated. The residue was purified was purified by flash chromatography on silica gel (EtOAc in PE= 0% to 40%) to afford the product (200 mg, 0.323 mmol, 52% yield) as an oil. The product was purified by SFC (Column DAICEL CHIRALPAK AD (250 mm^{∗}30 mm, 10 um) Condition 0.1% NH₃H₂O ETOH Begin B 25 End B 25 Gradient Time (min) 100% B) to give the product (72.2 mg, 0.166 mmol, 36% yield) as a solid. ¹H NMR (CDC1₃, 400 MHz) δ_{H} = 8.88 (d, 1H), 8.08 (s, 1H), 7.90 (d, 1H), 6.87 (s, 1H), 6.66 (s, 1H), 6.40 - 6.30 (m, 1H), 5.65 - 5.48 (m, 1H), 4.25 (s, 3H), 1.77 (d, 3H). ¹⁹F NMR (376.5 MHz, CDC1₃) δ_{F} = -62.232, -68.164.LCMS Rₜ = 1.022 min in 1.5 min chromatography, 5-95AB, MS ESI calcd. for C₁₇H₁₄F₆N₅O₂ [M+H]⁺434.0, found 434.0.

Example 3-3. Synthesis of 2-methyl-N-[(1R)-1-[3-[2-(trifluoromethyl)-4-pyridyl]isoxazol-5-yl]ethyl]-5-(trifluoromethyl)pyrazole-3-carboxamide (3-3):

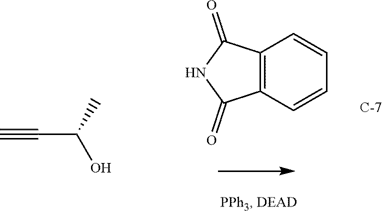

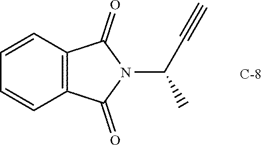

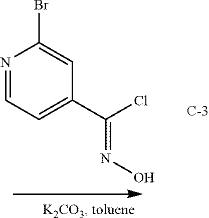

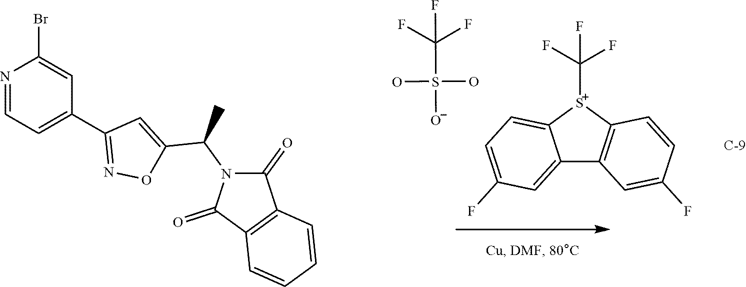

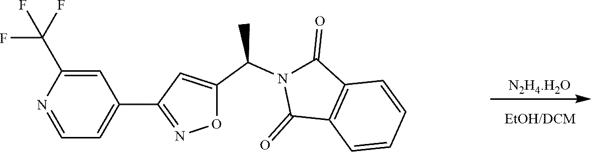

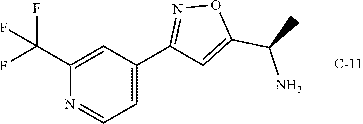 [SMILES: C[C@@H](N)c1cc(-c2ccnc(C(F)(F)F)c2)no1]

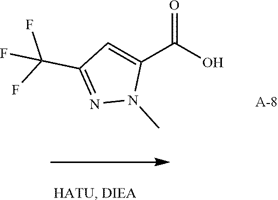

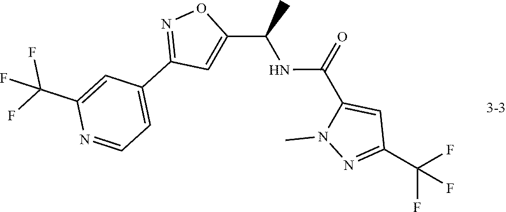 [SMILES: C[C@@H](NC(=O)c1cc(C(F)(F)F)nn1C)c1cc(-c2ccnc(C(F)(F)F)c2)no1]

### 2-Methylprop-2-ynyl)isoindoline-1,3-dione (C-8)

To a mixture of but-3-yn-2-ol (25 g, 357 mmol), phthalimide (53 g, 357 mmol), triphenylphosphine (140 g, 535 mmol) in THF (500 mL) was added DEAD (85 mL, 535 mmol) at 20° C. After stirring at 20° C. for 16 hours, the mixture was poured into water (600 mL) and extracted with EtOAc (2 × 300 mL). The combined organic layer was washed with brine (2 × 300 mL), dried over anhydrous Na₂SO₄, filtered and concentrated. The residue was triturated from V_{PE}/V_{DCM}= 6/1 (total 800 mL) at 25° C. The mother liquid concentrated to give product which was purified by flash column (0~20% of EtOAc in PE) to give the product (27 g, 133 mmol, 37% yield) as a solid. ¹H NMR (CDC1₃, 400 MHz) δ_{H} = 7.92-7.83 (m, 2H), 7.78-7.70 (m, 2H), 5.35-5.08 (m, 1H), 2.35 (d, 1H), 1.72 (d, 3H).

### 2-[(1R)[3-(2-bromo-4-pyridyl)isoxazol-5-yl]ethyl]isoindoline-1,3-dione (C-9)

The mixture of 2-[(1R)-1-methylprop-2-ynyl]isoindoline-1,3-dione (1.1 g, 5.7 mmol) in toluene (13 mL) was added K₂CO₃ (2.6 g, 19 mmol) and (4Z)-2-bromo-N-hydroxypyridine-4-carboximidoyl chloride (1.5 g, 6.4 mmol). After stirring at 120° C. for 3 hours, the mixture was poured into water (100 mL) and extracted with EtOAc (3 × 100 mL). The combined organic phase was washed with brine (2 × 100 mL), dried over anhydrous Na₂SO₄, filtered and concentrated. The residue was purified by silica gel chromatography (PE/EtOAc= 5/1 to 3/1) to afford the product (1.1 g, 2.8 mmol, 43% yield) as an oil. The product (50 mg, 0.13 mmol) was purified by prep-TLC (PE/EtOAc= 3/1) to give the product (30 mg, 0.070 mmol, 55% yield) as a solid. ¹H NMR (CDC₁₃ 400 MHz) δ_{H} = 8.50-8.40 (m, 1H), 7.92-7.84 (m, 3H), 7.80-7.74 (m, 2H), 7.68-7.60 (m, 1H), 6.69-6.63 (m, 1H), 5.77-5.69 (m, 1H), 1.94 (d, 3H).

### 2-[(1R)[3-[2-(trifluoromethyl)-4-pyridyl]isoxazol-5-yl]ethyl]isoindoline-1,3-dione (C-10):

To a mixture of Cu (239 mg, 3.8 mmol) and 2,8-difluoro-5-(trifluoromethyl)-5H-dibenzo[b,d]thiophen-5-ium trifluoromethanesulfonate (1.1 g, 2.5 mmol) was added 2-[(1R)-1-[3-(2-bromo-4-pyridyl)isoxazol-5-yl]ethyl]isoindoline-1,3-dione (500 mg, 1.3 mmol) in DMF (15 mL) at N₂. The mixture was stirred 0° C. for 1 h then heated to 80° C. and stirred for 3 hours. The mixture was extracted with EtOAc (3 × 50 mL). The combined organic phase was washed with saturated brine (3 × 30 mL), dried over anhydrous Na₂SO₄, filtered and concentrated. The mixture was purified by silica gel chromatography (PE/EtOAc = 5/1 to 3/1) to afford the product (350 mg, 0.90 mmol, 7% yield) as a solid. The product (100 mg, 0.26 mmol) was purified by perp-TLC (DCM/acetone= 50/1) to afford the product (41 mg, 0.11 mmol, 41% yield) as a solid. ¹H NMR (CDC1₃, 400 MHz) δ_{H} = 8.84 (d, 1H), 8.06 (s, 1H), 7.94-7.84 (m, 3H), 7.81-7.71 (m, 2H), 6.73 (d, 1H), 5.81-5.69 (m, 1H), 1.96 (d, 3H). LCMS Rₜ = 1.224 min in 2.0 min chromatography, 10-80AB, MS ESI calcd. for C₁₉H₁₃F₃N₃O₃ [M+H]⁺ 388.1, found 388.1.

### (1R)[3-[2-(trifluoromethyl)-4-pyridyl]isoxazol-5-yl]ethanamine (C-11)

To a solution of 2-[(1R)-1-[3-[2-(trifluoromethyl)-4-pyridyl]isoxazol-5-yl]ethyl]isoindoline-1,3-dione (150 mg, 0.39 mmol) in DCM (10 mL) and Ethanol (2.0 mL) was added N₂H₄.H₂O (0.12 mL, 2.3 mmol) dropwise at 25° C. The mixture was stirred at 25° C. for 16 hours. The mixture was filtered and the filter cake was washed with DCM (10 × 3 mL). The filtrate was concentrated and purified by silica gel chromatography (DCM/MeOH = 100/1 to 10/1) to afford the product (60 mg, 0.23 mmol, 60% yield) as an oil. ¹H NMR (CDCl₃, 400 MHz) δ_{H} = 8.84 (d, 1H), 8.11-8.02 (m, 1H), 7.88 (d, 1H), 6.62-6.55 (m, 1H), 4.42-4.28 (m, 1H), 1.60-1.58 (m, 5H).

### 2-methyl-N-[(1R)-1-[3-[2-(trifluoromethyl)-4-pyridyl]isoxazol-5-yl]ethyl]-5-(trifluoromethyl)pyrazole-3-carboxamide (3-3)

To a solution of 2-methyl-5-(trifluoromethyl)pyrazole-3-carboxylic acid (50 mg, 0.26 mmol), DIPEA (0.12 mL, 0.70 mmol), HATU (177 mg, 0.47 mmol) in DMF (5.0 mL) was added (1R)-1-[3-[2-(trifluoromethyl)-4-pyridyl]isoxazol-5-yl]ethanamine (60 mg, 0.23 mmol), the mixture was stirred at 20° C. for 1 hours. The residue was poured into water (15 mL) and stirred for 20 min. The aqueous phase was extracted with EtOAc (2 × 20 mL). The combined organic phase was washed with saturated brine (2 × 20 mL), dried over anhydrous Na₂SO₄, filtered and concentrated. The residue was purified by prep-TLC (DCM/actone= 50/1) to afford the product (61.57 mg, 0.14 mmol, 60% yield) as a solid. ¹H NMR (CDC1₃, 400 MHz) δ_{H} = 8.86 (d, 1H), 8.06 (s, 1H), 7.91-7.83 (m, 1H), 6.85 (s, 1H), 6.64 (s, 1H), 6.33 (d, 1H), 5.60-5.46 (m, 1H), 4.23 (s, 3H), 1.75 (d, 3H). LCMS Rₜ = 1.212 min in 2.0 min chromatography, 10-80 AB, MS ESI calcd. for C₁₇H₁₄F₆N₅O₂ [M+H]⁺ 434.3, found 434.3.

Examples 4 and 5. Synthesis of (S)-N-(1-(3-(2-cyclopropylpyridin-4-yl)-1,2,4-thiadiazol-5-yl)ethyl)benzamide (4) and (R)-N-(1-(3-(2-cyclopropylpyridin-4-yl)-1,2,4-thiadiazol-5-yl)ethyl)benzamide (5). Note that stereochemistry is randomly assigned.

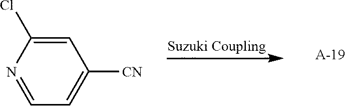

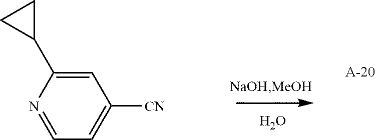

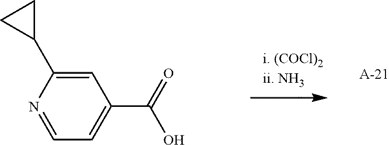

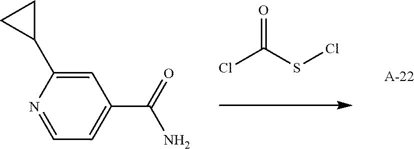 [SMILES: NC(=O)c1ccnc(C2CC2)c1 ; O=C(Cl)SCl]

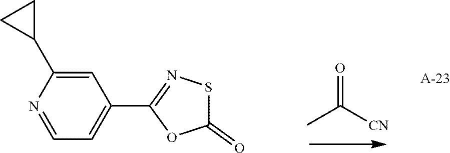

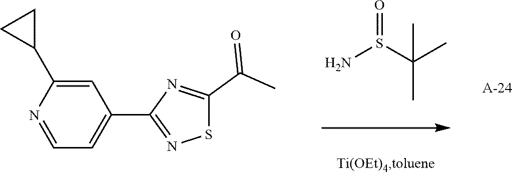

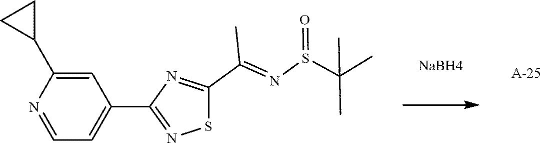

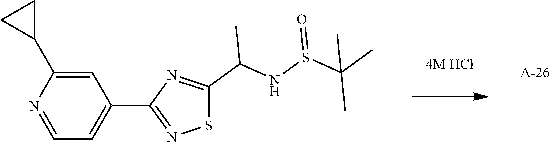

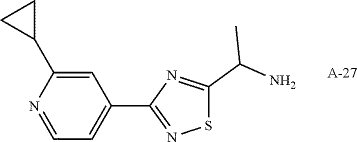 [SMILES: CC(N)c1nc(-c2ccnc(C3CC3)c2)ns1]

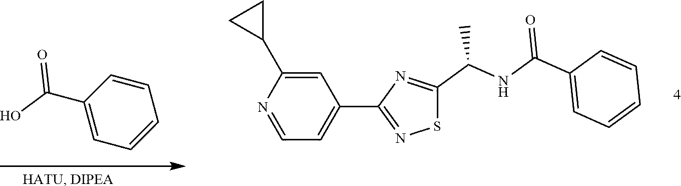

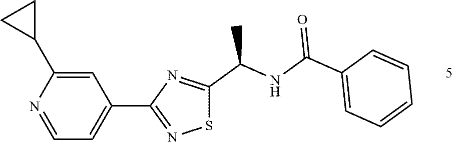 [SMILES: C[C@@H](NC(=O)c1ccccc1)c1nc(-c2ccnc(C3CC3)c2)ns1]

### Synthesis of 2-cyclopropylisonicotinonitrile (A-20)

To a stirred solution of A-19 (10 g, 72.18 mmol) in 1,4-Dioxane (100 mL), was added K₃PO₄ (38.31 g, 180.44 mmol) and cyclopropylboronic acid (12.4 g, 144.35 mmol)at room temperature. Reaction mixture was purged with Argon for 20 min. To this solution, silver oxide (3.35 g, 14.44 mmol) and Pd(dppf)Cl₂ (5.28 g, 7.22 mmol) were added and the reaction mixture was stirred at 100° C. for 3 h. The reaction mixture was cooled to room temperature and filtered through a pad of celite and washed with ethyl acetate (50 mL). The organic layer was washed with water (3 × 25 mL), separated, dried over anhydrous sodium sulfate and concentrated under reduced pressure. The obtained residue was purified by column chromatography using 100-200 silica and 5-10% EtOAc/hexane as an eluent to give A-20 (5.3 g, 31.17 mmol, 43% yield) as a solid.

### Synthesis of 2-cyclopropylisonicotinic Acid (A-21)

To a stirred solution of A-20 (2 g, 13.87 mmol) in methanol/water (15 mL/10 mL), NaOH (1.66 g, 41.62 mmol) was added and the reaction mixture was stirred for 5 h. The volatile solvent was removed under reduced pressure. The residue was diluted with water and extracted with EtOAc. The aqueous layer was acidified with 1 N HCl. The precipitated solid was collected by filtration and dried under reduced pressure to give A-21 (1.7 g).

### Synthesis of 2-cyclopropylisonicotinamide (A-22)

To a stirred solution of A-21 (1.5 g, 9.19 mmol) in DCM (20 mL) at 0° C. was added DMF (2.5 mL) and oxalyl chloride (2.33 g, 18.39 mmol) in dropwise manner and resultant reaction mixture was stirred at room temperature for 2 h. The reaction mixture was evaporated under inert nitrogen atmosphere to get the residue which was dissolved in MeCN (20 mL) and charged with aq. ammonia solution (20 mL). The reaction mixture was quenched using water (25 mL) and diluted with EtOAc (2 × 50 mL). The organic layer was separated, dried over anhydrous Na₂SO₄, filtered and evaporated to get a residue which was purified by column chromatography using 100-200 silica and 30-80% EtOAc/hexane as an eluent to give A-22 (1.2 g, 6.51 mmol, 70% yield) as a solid.

### Synthesis of 5-(2-cyclopropylpyridin-4-yl)-1,3,4-oxathiazol-2-one (A-23)

To a stirred solution of A-22 (1.2 g, 6.51 mmol) in toluene (10 mL), chloromethanethioate (0.852 g, 6.51 mmol) was added at room temperature and the reaction mixture was stirred at 120° C. for 16 h. The reaction was quenched by adding water (50 mL), diluted with EtOAc (2 × 100 mL) and organic layer was separated. The organic layer dried over anhydrous Na₂SO₄, filtered and evaporated to get a residue which was purified by column chromatography using 100-200 silica and 5-50% EtOAc/hexane as an eluent to give A-23 (0.5 g, 2.01 mmol, 30% yield) as a solid.

### Synthesis of 1-(3-(2-cyclopropylpyridin-4-yl)-1,2,4-thiadiazol-5-yl)ethan-1-one (A-24)

A mixture of A-23 (441.99 mg, 2.01 mmol) and acetyl cyanide (831.52 mg, 12.04 mmol) in 1,2-dichlorobenzene (10 mL) was stirred at 160° C. for 24 h. The reaction mixture was quenched using water (10 mL), diluted with EtOAc (20 mL) and organic layer was separated, dried over anhydrous Na₂SO₄, filtered and evaporated under reduced pressure to give a residue which was purified by column chromatography using 100-200 silica and 10-50% EtOAc/hexane as an eluent to give A-24 (300 mg, 0.734 mmol, 36% yield) as a solid.

### Synthesis of (E)-N-(1-(3-(2-cyclopropylpyridin-4-yl)-1,2,4-thiadiazol-5-yl)ethylidene)-2-methylpropane-2-sulfinamide (A-25)

To a stirred solution of A-24 (180.73 mg, 0.74 mmol) and 2-methylpropane-2-sulfinamide (89.3 mg, 0.74 mmol) in toluene (10 mL) was added titanium ethoxide (0.16 mL, 0.74 mmol) and stirred at 80° C. for 16 h. The reaction mixture was quenched using water and diluted with ethyl acetate. The organic layer was separated, dried by over anhydrous sodium sulfate, evaporated under reduced pressure to get a residue which was purified by column chromatography using 100-200 silica and 10-30% EtOAC/hexane as an eluent to give A-25 (250 mg, 0.487 mmol, 66% yield) as a liquid.

### Synthesis of N-(1-(3-(2-cyclopropylpyridin-4-yl)-1,2,4-thiadiazol-5-yl)ethyl)-2-methylpropane-2-sulfinamide (A-26)

To a stirred solution of A-25 (250 mg, 0.72 mmol) in methanol (10 mL) at 0° C. was added sodium borohydride (54.28 mg, 1.43 mmol) and the mixture was stirred at room temperature for 1 h. The reaction mixture was diluted with water and extracted with ethyl acetate. The organic layer was dried over anhydrous sodium sulphate and evaporated under reduced pressure to give A-26 (235 mg) as a solid.

### Synthesis of 1-(3-(2-cyclopropylpyridin-4-yl)-1,2,4-thiadiazol-5-yl)ethan-1-amine (A-27)

To a stirred solution of A-26 (235 \. mg, 0.47 mmol) in 1,4-dioxane (2 mL) at 0° C. was added 4 M HCl in 1,4-dioxane (10 mL, 0.47 mmol) and the mixture was stirred at room temperature for 2 h. The reaction mixture was evaporated to get a residue which was purified by washing with diethyl ether to get A-27 (125 mg).

### Step-9: Synthesis of (S)-N-(1-(3-(2-cyclopropylpyridin-4-yl)-1,2,4-thiadiazol-5-yl)ethyl)benzamide (4) and (R)-N-(1-(3-(2-cyclopropylpyridin-4-yl)-1,2,4-thiadiazol-5-yl)ethyl)benzamide (5)

To a stirred solution of A-27 (282 mg, 1.02 mmol) and benzoic acid (149.51 mg, 1.22 mmol) in DCM (10 mL) was added HATU (581.9 mg, 1.53 mmol) and DIPEA (0.18 mL, 1.02 mmol) at room temperature. The reaction mixture was stirred at room temperature for 2 h. The reaction mixture was quenched with water (10 mL) and diluted with DCM (2 × 100 mL). The organic layer was dried over anhydrous Na₂SO₄, filtered and evaporated to get a residue. The residue compound was purified by column chromatography using 100-200 silica and 30-80% EtOAc/hexane as an eluent to get racemic mixture which was then purified by SFC column chromatography followed by chiral HPLC to afford 4 (39.22 mg, 0.1109 mmol, 11% yield) and 5 (20.89 mg, 0.0593 mmol, 6% yield). Note: absolute stereochemistry was randomly assigned.

4: HPLC: Rt 7.411 min, Column : X-Select CSH C18 (4.6^{∗}150) mm 5 u; Mobile Phase: A -0.1% Formic acid in water : Acetonitrile (95:05); B - Acetonitrile; Flow Rate: 1.0 \. mL/minute; LCMS : 351.1 (M+H), Rt 1.639 min, X-Select CSH C18 (3.0^{∗}50) mm 2.5 u; Mobile Phase: A: 0.05% Formic acid in water : ACN (95:5); B: 0.05% Formic acid in ACN; Flow Rate: 1.2 \. mL/minute; Chiral HPLC: Rt 7.89 min, 99.55%; Column : PHENOMENEX CELLULOSE-3, 250 mm ^{∗}4.6 mm, 5 u; Mobile Phase: A: n-HEXANE+0.1%TFA; B: ETHANOL:MEOH(50:50); Flow rate: 1.0 mL/min; Isocratic: 20%B. ¹H NMR (400 MHz, DMSO-d6) δ_{H} = 9.43 - 9.36 (m, 1H), 8.58 (d, 1H), 8.03 (s, 1H), 7.94 (d, 2H), 7.83 (dd, 1H), 7.64 - 7.57 (m, 1H), 7.56 - 7.49 (m, 2H), 5.66 - 5.55 (m, 1H), 2.31 - 2.23 (m, 1H), 1.75 (d, 3H), 1.04 - 0.95 (m, 4H).

5: HPLC: Rt 7.412 min, Column : X-Select CSH C18 (4.6^{∗}150) mm 5u; Mobile Phase: A -0.1% Formic acid in water : Acetonitrile (95:05); B - Acetonitrile; Flow Rate: 1.0 \. mL/minute; LCMS : 351.1 (M+H), Rt 1.629 min, X-Select CSH C18 (3.0^{∗}50) mm 2.5 u; Mobile Phase: A: 0.05% Formic acid in water : ACN (95:5); B: 0.05% Formic acid in ACN; Flow Rate: 1.2. mL/minute; Chiral HPLC: Rt 6.690 min, 100%; Column : PHENOMENEX CELLULOSE-3, 250 mm ^{∗}4.6 mm, 5 u; Mobile Phase: A: n-HEXANE+0.1%TFA; B: ETHANOL:MEOH(50:50); Flow rate: 1.0 mL/min; Isocratic: 20%B. ¹H NMR (400 MHz, DMSO-d6) δ_{H} = 9.43 - 9.35 (m, 1H), 8.58 (d, 1H), 8.03 (s, 1H), 7.94 (d, 2H), 7.83 (dd, 1H), 7.64 - 7.57 (m, 1H), 7.56 - 7.49 (m, 2H), 5.66 - 5.56 (m, 1H), 2.32 - 2.22 (m, 1H), 1.75 (d, 3H), 1.05 - 0.94 (m, 4H).

Examples 6 and 7. Synthesis of (S)-3-chloro-N-(1-(3-(2-cyclopropylpyridin-4-yl)-1,2,4-thiadiazol-5-yl)ethyl)benzamide (6) and (R)-3-chloro-N-(1-(3-(2-cyclopropylpyridin-4-yl)-1,2,4-thiadiazol-5-yl)ethyl)benzamide (7). Note the stereochemistry is randomly assigned.

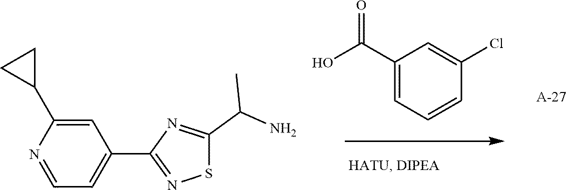

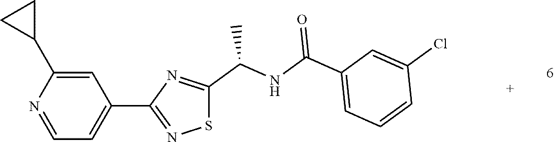

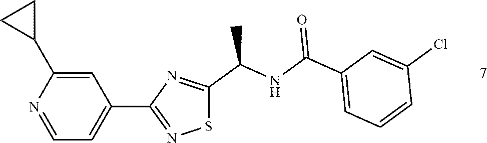 [SMILES: C[C@@H](NC(=O)c1cccc(Cl)c1)c1nc(-c2ccnc(C3CC3)c2)ns1]

To a stirred solution of A-27 (190 mg, 0.6900 mmol) and 3-chlorobenzoic acid (100.74 mg, 0.6400 mmol) in DCM (15 mL) was added HATU (392.06 mg, 1.03 mmol) and DIPEA (0.12 mL, 0.6900 mmol) at room temperature. The reaction mixture was stirred at room temperature for 2 h. The reaction mixture was quenched with water (10 mL) and diluted with DCM (2 × 100 mL). The organic layer was dried over anhydrous Na₂SO₄, filtered and evaporated to give a residue. The residue was purified by column chromatography using 100-200 silica and 30-80% EtOAc/hexane as an eluent to get racemic mixture which was then purified by SFC column chromatography followed by chiral HPLC to afford 6 (52.46 mg, 0.1348 mmol, 20% yield) and 7 (54.49 mg, 0.1412 mmol, 21% yield). Note the stereochemistry is randomly assigned.

6: HPLC: Rt 6.325 min, 98.93%; Column: XSELECT CSH C18 (150 × 4.6 mm, 3.5 u); Mobile Phase-A: 0.05% TFA in Water:ACN( 95:5); Mobile Phase-B:Mobile phase A:Acetonitrile(5:95); Flow : 1.0 mL/min; LCMS : 385.1 (M+H), Rt 2.354 min, Column:X-Bridge BEH C-18(3.0×50 mm,2.5 µm); Mobile Phase: A: 0.025% FA in Water, B: ACN; Flow rate: 1.2 ml/min(Gradient); Chiral HPLC: Rt 9.649 min, 99.33% Column :CHIRAL PAK IG (250^{∗}4.6 mm^{∗}5 µm); Mobile Phase A: 0.1%DEA in n-HEXANE; Mobile Phase B:DCM:MEOH(50:50); AB : 75:25; Flow :: 1.0 mL/min. ¹H NMR (400 MHz, DMSO-d6) δ_{H} = 9.49 (d, 1H), 8.58 (d, 1H), 8.05 - 7.97 (m, 2H), 7.93 - 7.87 (m, 1H), 7.83 (dd, 1H), 7.72 -7.64 (m, 1H), 7.61 - 7.53 (m, 1H), 5.60 (quin, 1H), 2.31 - 2.22 (m, 1H), 1.74 (d, 3H), 1.04 -0.93 (m, 4H).

7: HPLC: Rt 6.322 min, 99.76%; Column: XSELECT CSH C18 (150 × 4.6 mm, 3.5 u); Mobile Phase-A: 0.05% TFA in Water:ACN( 95:5); Mobile Phase-B:Mobile phase A:Acetonitrile(5:95); Flow : 1.0 mL/min; LCMS : 385.1 (M+H), Rt 2.338 min, Column:X-Bridge BEH C-18(3.0×50 mm,2.5 µm); Mobile Phase: A: 0.025% FA in Water, B: ACN; Flow rate: 1.2 ml/min(Gradient); Chiral HPLC: Rt 20.168 min, 99.31% Column :CHIRAL PAK IG (250^{∗}4.6 mm^{∗}5 µm); Mobile Phase A: 0.1%DEA in n-HEXANE; Mobile Phase B:DCM:MEOH(50:50); AB : 75:25; Flow: 1.0mL/min.¹H NMR (400 MHz, DMSO-d6) δ_{H} = 9.49 (d, 1H), 8.58 (d, 1H), 8.05 - 7.97 (m, 2H), 7.93 - 7.87 (m, 1H), 7.83 (dd, 1H), 7.71 -7.65 (m, 1H), 7.60 - 7.53 (m, 1H), 5.60 (quin, 1H), 2.31 - 2.22 (m, 1H), 1.74 (d, 3H), 1.05 -0.92 (m, 4H).

Examples 8 and 9. Synthesis of (S)-N-(1-(3-(2-(trifluoromethyl)pyridin-4-yl)isoxazol-5-yl)ethyl)piperidine-1-carboxamide (8) and (R)-N-(1-(3-(2-(trifluoromethyl)pyridin-4-yl)isoxazol-5-yl)ethyl)piperidine-1-carboxamide (9). Note the stereochemistry is randomly assigned.

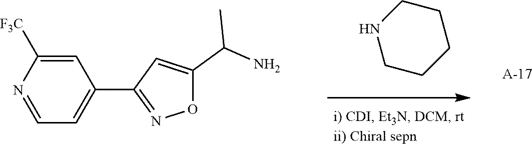

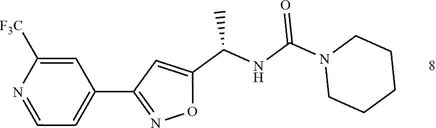 [SMILES: C[C@H](NC(=O)N1CCCCC1)c1cc(-c2ccnc(C(F)(F)F)c2)no1]

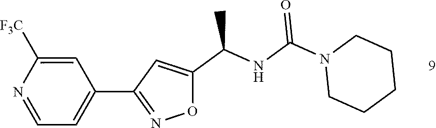 [SMILES: C[C@@H](NC(=O)N1CCCCC1)c1cc(-c2ccnc(C(F)(F)F)c2)no1]

To a stirred solution of A-17 (300 mg, 1.17 mmol) and piperidine (0.23 mL, 2.33 mmol) in DCM(10 mL) was added CDI (378.25 mg, 2.33 mmol) and TEA (0.49 mL, 3.5 mmol) at room temperature. The reaction mixture was allowed to stir at room temperature for 12 h. The reaction mixture was quenched with water (10 mL) and extracted with DCM (2× 50 mL). The combined extracts were dried over anhydrous Na₂SO₄, filtered and concentrated under reduced pressure. The residue was purified by Combi-Flash column chromatography (100-200 silica gel) by using 30-50% EtOAc/Hexane as eluent followed by preparative chiral HPLC to afford 8 (90 mg, 0.2365 mmol, 20% yield) and 9 (70 mg, 0.1897 mmol, 16% yield). Note the stereochemistry is randomly assigned.

8: HPLC: Rt: 8.242 min, 96.79%; Column: XSELECT CSH C18 (150 × 4.6 mm, 3.5 i); Mobile Phase-A: 0.1%FA in Water; Mobile Phase-B:Acetonitrile; Flow: 1.2 mL/min. LCMS : 369.1 (M+H), Rt 2.050 min, Column: X-Bridge BEH C-18 (3.0×50 mm,2.5 µm); Mobile Phase: A: 0.025% FA in Water, B: ACN; Flow rate: 1.2 ml/min CHIRAL HPLC: Rt: 5.535 min, 99.9%;

COLUMN: Chiral pak-IG (250^{∗}4.6 mm) 5 µm; MOBILE PHASE A: 0.1%DEA in n-Hexane MOBILE PHASE B: ETOH: MEOH (50:50); PROGRAM- AB 70:30; FLOW RATE: 1.0 ¹H NMR (400 MHz, DMSO-d₆) δ_{H}= 8.92 (d, 1H), 8.32 (s, 1H), 8.20 (d, 1H), 7.16 (s, 1H), 6.96 (d, 1H), 5.11 (quin, 1H), 3.37- 3.32 (m, 2H), 3.30 - 3.23 (m, 2H), 1.60 - 1.38 (m, 9H).

9: HPLC: Rt: 8.223 min, 99.83%; Column: XSELECT CSH C18 (150 × 4.6 mm, 3.5 µ); Mobile Phase-A: 0.1%FA in Water; Mobile Phase-B:Acetonitrile; Flow: 1.2 mL/min. LCMS : 369.1 (M+H), Rt 2.051 min, Column: X-Bridge BEH C-18 (3.0 × 50 mm, 2.5 µm); Mobile Phase: A: 0.025% FA in Water, B: ACN; Flow rate: 1.2 ml/min (Gradient); CHIRAL HPLC: Rt 7.686 min, 99.53%; COLUMN: Chiral pak-IG (250^{∗}4.6 mm) 5 µm; MOBILE PHASE A: 0.1%DEA in n-Hexane; MOBILE PHASE B: ETOH: MEOH (50:50);

PROGRAM- AB 70:30; FLOW RATE : 1.0 ML/MIN ¹H NMR (400 MHz, DMSO-d₆) δ_{H} = 8.92 (d, 1H), 8.32 (s, 1H), 8.20 (d, 1H), 7.17 (s, 1H), 6.96 (d, 1H), 5.11 (quin, 1H), 3.36 (br s, 2H), 3.30 - 3.22 (m, 2H), 1.59 - 1.39 (m, 9H).

Examples 10 and 11. Synthesis of (R)-1-methyl-3-(trifluoromethyl)-N-(1-(3-(2-(trifluoromethyl)pyridin-4-yl)-1,2,4-thiadiazol-5-yl)ethyl)-1H-pyrazole-5-carboxamide (10) and (S)-1-methyl-3-(trifluoromethyl)-N-(1-(3-(2-(trifluoromethyl)pyridin-4-yl)-1,2,4-thiadiazol-5-yl)ethyl)-1H-pyrazole-5-carboxamide (11). Note the stereochemistry is randomly assigned.

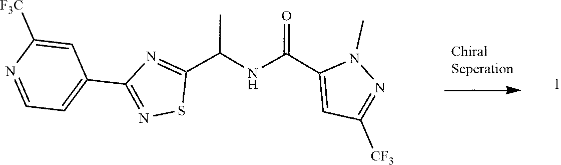

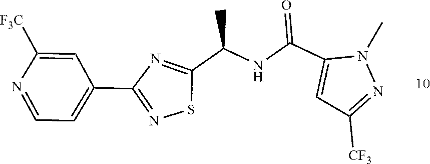 [SMILES: C[C@@H](NC(=O)c1cc(C(F)(F)F)nn1C)c1nc(-c2ccnc(C(F)(F)F)c2)ns1]

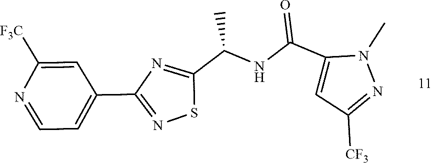 [SMILES: C[C@H](NC(=O)c1cc(C(F)(F)F)nn1C)c1nc(-c2ccnc(C(F)(F)F)c2)ns1]

1 was purified by chiral HPLC to get 10 (10 mg, 0.022 mmol, 8 %yield) and 11 (10 mg, 0.022 mmol, 8% yield).

10: HPLC: Rt 9.349 min, 99.77%; Column: X-Select CSH C18 (4.6 × 150) mm, 3.5 µm;Mobile phase: A: 0.1% Formic acid in water: ACN (95:05), B: ACN; Flow Rate: 1.0 mL/min. LCMS : 450.9 (M+H), Rt 2.117 min, Column: X-select CSH C18 (3^{∗}50) mm, 2.5 µm, ¹H NMR (400 MHz, DMSO-d6) δ 9.55 (d, 1H), 8.99 (d, 1H), 8.46 (s, 1H), 8.42 (d, 1H), 7.48 (s, 1H), 5.66-5.58 (m, 1H), 4.15 (s, 3H), 1.71 (d, 3H). Chiral method: Rt 4.458 min, 99.93%; column: PHENOMENEX CELLULOSE-3 (250 mm ×4.6 mm,5 u)- Mobile Phase: A) n-Hexane+0.1% TFA B) EtOH:MeOH (50:50), Isocratic:20%B; Wavelength: 240 nm, Flow: 1.0 mL/min.

11: HPLC: Rt 9.352 min, 99.87%; Column: X-Select CSH C18 (4.6 × 150) mm, 3.5 µm; Mobile phase: A: 0.1% Formic acid in water: ACN (95:05), B: ACN; Flow Rate: 1.0 mL/min; LCMS : 449.2 (M-H), Rt 2.182 min, Column: X-select CSH C18 (3^{∗}50) mm, 2.5 µm; ¹H NMR (400 MHz, DMSO-d6) δ 9.55 (d, 1H), 8.99 (d, 1H), 8.46 (s, 1H), 8.42 (d, 1H), 7.48 (s, 1H), 5.66-5.58 (m, 1H), 4.15 (s, 3H), 1.71 (d, 3H). Chiral method: Rt 6.579 min, 99.87%; column: PHENOMENEX CELLULOSE-3 (250 mm ×4.6 mm,5 u)- Mobile Phase: A) n-Hexane+0.1% TFA B) EtOH:MeOH (50:50), Isocratic:20%B; Wavelength: 240 nm, Flow: 1.0 mL/min.

Example 11-1. Synthesis of 2-methyl-N-[(1S)-1-[3-[2-(trifluoromethyl)-4-pyridyl]-1,2,4-thiadiazol-5-yl]ethyl]-5-(trifluoromethyl)pyrazole-3-carboxamide (11-1)

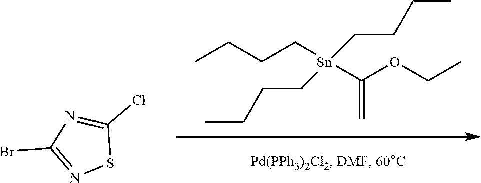

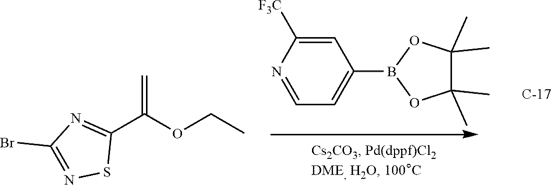

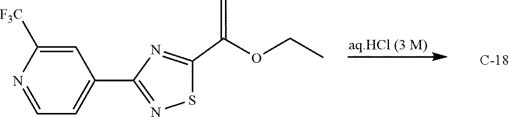

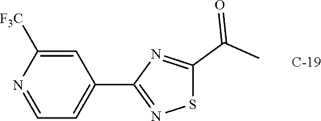 [SMILES: CC(=O)c1nc(-c2ccnc(C(F)(F)F)c2)ns1]

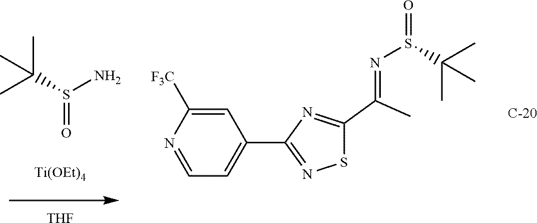

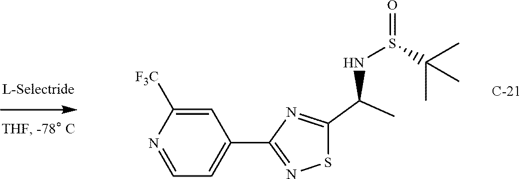

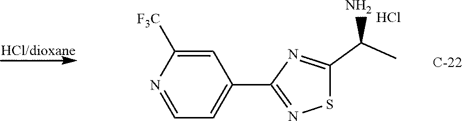

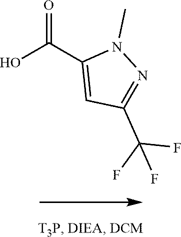

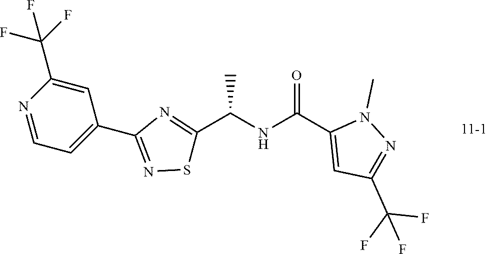 [SMILES: C[C@H](NC(=O)c1cc(C(F)(F)F)nn1C)c1nc(-c2ccnc(C(F)(F)F)c2)ns1]

### 3-Bromo(1-ethoxyvinyl)-1,2,4-thiadiazole (C-17)

To a mixture of 3-bromo-5-chloro-1,2,4-thiadiazole (10.0 g, 50.1 mmol) and 1-ethoxyvinyltri-n-butyltin (20.5 mL, 60.2 mmol) in DMF (150 mL) was added Pd(PPh₃)₂Cl₂ (3.52 g, 5.01 mmol) under N₂ and the reaction mixture was heated at 60° C. for 4 h. The reaction mixture was quenched with aq. KF (10.0 g in 300 mL water) and stirred for 30 mins and filtered. The filtrate was extracted with EtOAc (2 × 300 mL). The organic layer was dried over Na₂SO₄, filtered and concentrated under reduced pressure. The residue was purified by column chromatography on silica gel (PE/EtOAc = 20/1) to give the product (7.0 g, 29.8 mmol, 59% yield) as a solid. ¹H NMR (CDCl₃, 400 MHz) δ_{H} = 5.53 (d, 1H), 4.58 (d, 1H), 4.02 (q, 2H), 1.43 (t, 3H).

### 5-Ethoxyvinyl)-3-[2-(trifluoromethyl)-4-pyridyl]-1,2,4-thiadiazole (C-18)

To a solution of 3-bromo-5-(1-ethoxyvinyl)-1,2,4-thiadiazole (2.0 g, 8.51 mmol) in DME (20.0 mL, 8.51 mmol) and water (4.0 mL) was added C_{S2}CO₃ (8.31 g, 25.5 mmol), 4-(4,4,5,5-tetramethyl-1,3,2-dioxaborolan-2-yl)-2-(trifluoromethyl)pyridine (3.02 g, 11.1 mmol) and Pd(dppf)Cl₂ (622 mg, 0.85 mmol) under N₂. After stirring at 100° C. for 1.5 hours, the reaction mixture was cooled to 25° C., filtered and concentrated under reduced pressure. The residue was purified by chromatography on silica gel with petroleum/ethyl acetate= 10/1 to give the product (1.80 g, 5.97 mmol, 70% yield) as an oil. ¹H NMR (CDC1₃, 400 MHz) δ_{H} = 8.87 (d, 1H), 8.57 (s, 1H), 8.37 (d, 1H), 5.63 (d, 1H), 4.62 (d, 1H), 4.13-3.95 (m, 2H), 1.47 (t, 3H).

### 1- [2-(Trifluoromethyl)-4-pyridyl] -1,2,4-thiadiazol-5-yl] Ethenone (C-19)

To a solution of 5-(1-ethoxyvinyl)-3-[2-(trifluoromethyl)-4-pyridyl]-1,2,4-thiadiazole (1.80 g, 5.97 mmol) in acetone (20.0 mL) was added 3 M HCl (1.09 g, 29.9 mmol) at 25° C. After stirring at 25° C. for 16 hr, the reaction mixture was quenched with sat. NaHCO₃ (50.0 mL) and extracted with EtOAc (2 × 50.0 mL). The combined organic layer was washed with brine (50.0 mL) and dried over Na₂SO₄, filtered and concentrated under reduced pressure to give the product (1.60 g, 5.86 mmol, 98% yield) as a solid which was used directly for next step. ¹H NMR (CDC1₃, 400 MHz) δ_{H} = 8.92 (d, 1H), 8.58 (s, 1H), 8.39 (d, 1H), 2.85 (s, 3H).

### (R,E)methyl-N-[1-[3-[2-(trifluoromethyl)-4-pyridyl]-1,2,4-thiadiazol-5-yl]ethylidene]propanesulfinamide (C-20)

To a solution of 1-[3-[2-(trifluoromethyl)-4-pyridyl]-1,2,4-thiadiazol-5-yl]ethanone (300 mg, 1.10 mmol) in THF (10.0 mL) was added (R)-2-methylpropane-2-sulfinamide (200 mg, 1.65 mmol) and Ti(OEt)₄ (751 mg, 3.29 mmol) at 25° C. under N₂. The mixture was heated to 65° C. and stirred for 16 hr. The reaction mixture was quenched with saturated aq. NaHCO₃ (20.0 mL) and filtered. The filtrate was extracted with EtOAc (2 × 20.0 mL). The combined organic layer was dried over Na₂SO₄, filtered and concentrated under reduced pressure. The product was purified by column chromatographyon SiO₂ (PE/EtOAc= 10/1) to give the product (120 mg, 0.32 mmol, 29% yield) as an oil. ¹H NMR (CDC1₃, 400 MHz) δ_{H} = 8.91 (d, 1H), 8.56 (s, 1H), 8.37 (d, 1H), 2.98 (s, 3H), 1.37 (s, 9H).

### (R)methyl-N-[(1S)-1-[3-[2-(trifluoromethyl)-4-pyridyl]-1,2,4-thiadiazol-5-yl]ethyl]propanesulfinamide (C-21)

To a solution of (R,E)-2-methyl-N-[1-[3-[2-(trifluoromethyl)-4-pyridyl]-1,2,4-thiadiazol-5-yl]ethylidene]propane-2-sulfinamide (100 mg, 0.27 mmol) in THF (2.0 mL) was added L-Selectride (0.53 mL, 0.53 mmol) under N₂ at -78° C. The reaction mixture was stirred at -78° C. for 30 mins. NH₄C1 (10.0 mL) was added at -78° C. to the mixture. The mixture was extracted with EtOAc (2 × 20.0 mL). The combined organic layer was washed with brine (20.0 mL), dried over Na₂SO₄, filtered and concentrated under reduced pressure to give the product (110 mg, 0.29 mmol) as an oil which was used directly for next step. LCMS Rₜ = 0.727 min in 1.0 min chromatography, 5-95AB, MS ESI calcd. for C₁₄H₁₈F₃N₄OS₂ [M+H]⁺379.0, found 379.0.

### (1S)[3-[2-(trifluoromethyl)-4-pyridyl]-1,2,4-thiadiazol-5-yl]ethanamine hydrochloride (C-22)

To a solution of (R)-2-methyl-N-[(1S)-1-[3-[2-(trifluoromethyl)-4-pyridyl]-1,2,4-thiadiazol-5-yl]ethyl]propane-2-sulfinamide (150 mg, 0.40 mmol) in 1,4-Dioxane (1.0 mL) was added 4 M HCl/dioxane (2.0 mL, 1.98 mmol) at 25° C. After stirring at 25° C. for 2 hr, the reaction mixture was concentrated under reduced pressure to give the product (100 mg, 0.32 mmol, 81% yield) as a solid. LCMS Rₜ = 0.744 min in 1.5 min chromatography, 5-95AB, MS ESI calcd. for C₁₀H₁₀F₃N₄S [M+H]⁺274.8, found 274.8.

### 2-methyl-N-[(1S)-1-[3-[2-(trifluoromethyl)-4-pyridyl]-1,2,4-thiadiazol-5-yl]ethyl]-5-(trifluoromethyl)pyrazole-3-carboxamide (11-1)

To a solution of 2-methyl-5-(trifluoromethyl)pyrazole-3-carboxylic acid (62.5 mg, 0.32 mmol) in DCM (3.0 mL) was added DIPEA (0.45 mL, 2.57 mmol), T₃P (734 mg, 0.97 mmol). After stirring at 25° C. for 20 mins, (1S)-1-[3-[2-(trifluoromethyl)-4-pyridyl]-1,2,4-thiadiazol-5-yl]ethanamine hydrochloride (100 mg, 0.32 mmol) was added and the reaction mixture was stirred at 25° C. for 16 hr. The reaction mixture was quenched with water (20.0 mL) and extracted with DCM (2 × 20.0 mL). The combined organic layer was washed with brine (20.0 mL) and dried over Na₂SO₄, filtered and concentrated under reduced pressure to give the product which was purified by prep-HPLC (Column: Phenomenex Gemini-NX 80 ^{∗} 40 mm ^{∗} 3 µm, Condition: water (0.05%NH₃H₂O)-ACN, Begin B: 48, End B: 78, Gradient Time (min): 8, 100%B Hold Time (min): 2, FlowRate (mL/min): 30, Injections: 5) to give the product (90.0 mg, 0.20 mmol, 62% yield) as as a solid. The product (90.0 mg, 0.20 mmol) was purified by SFC (Column: DAICEL CHIRALCEL OJ (250 mm ^{∗} 30 mm, 10 µm), Condition: 0.1% NH₃H₂O-EtOH, Begin B: 15%, End B: 15%, FlowRate (mL/min): 60, Injections: 30) to give the product (54.3 mg, 0.12 mmol, 60% yield) as a solid. ¹H NMR (CDC1₃, 400 MHz) δ_{H} = 8.88 (d, 1H), 8.53 (s, 1H), 8.34 (d, 1H), 6.90 (s, 1H), 6.60 (d, 1H), 5.76-5.66 (m, 1H), 4.24 (s, 3H), 1.86 (d, 3H). ¹⁹F NMR (376.5 MHz, CDC1₃) δ_{F} -62.206, -68.055. LCMS Rt = 2.496 min in 3.0 min chromatography, 30-90AB, MS ESI calcd. for C ₆H₃F₆N₆OS [M+H]⁺451.2, found 451.2. 99.72%ee.

Example 10-1. Synthesis of 2-methyl-N-[(1R)-1-[3-[2-(trifluoromethyl)-4-pyridyl]-1,2,4-thiadiazol-5-yl]ethyl]-5-(trifluoromethyl)pyrazole-3-carboxamide (10-1)

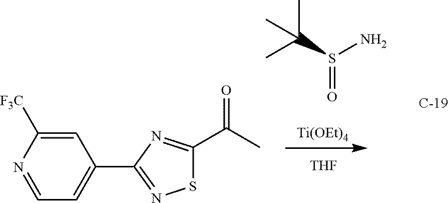

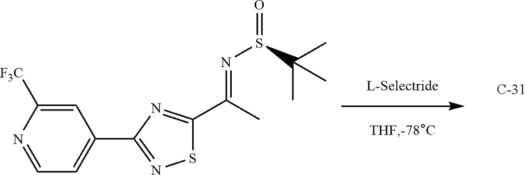

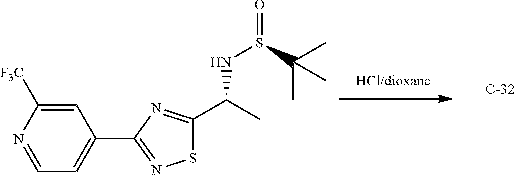

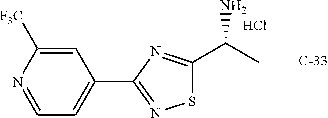 [SMILES: C[C@@H](N)c1nc(-c2ccnc(C(F)(F)F)c2)ns1 ; Cl]

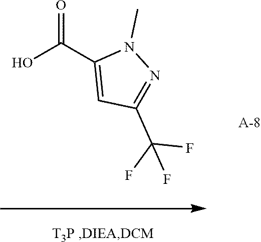

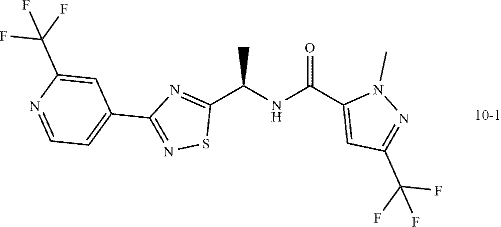 [SMILES: C[C@@H](NC(=O)c1cc(C(F)(F)F)nn1C)c1nc(-c2ccnc(C(F)(F)F)c2)ns1]

### (S,E)methyl-N-[1-[3-[2-(trifluoromethyl)-4-pyridyl]-1,2,4-thiadiazol-5-yl]ethylidene]propanesulfinamide (C-31)

To a solution of 1-[3-[2-(trifluoromethyl)-4-pyridyl]-1,2,4-thiadiazol-5-yl]ethanone (300 mg, 1.10 mmol) in THF (10.0 mL) was added (S)-2-methylpropane-2-sulfinamide (200 mg, 1.65 mmol) and Ti(OEt)₄ (751 mg, 3.29 mmol) at 25° C. under N₂. The mixture was heated to 65° C. and stirred for 16 hr. The reaction mixture was quenched with saturated aq. NaHCO₃ (20.0 mL) and filtered. The filtrate was extracted with EtOAc (2 × 20.0 mL). The combined organic layer was dried over Na₂SO₄, filtered and concentrated under reduced pressure. The product was purified by column chromatographyon SiO₂ (PE/EtOAc= 10/1) to give the product (110 mg, 0.29 mmol, 27% yield) as an oil.

### (S)methyl- N - [(1R)-1- [3-[2-(trifluoromethyl)-4-pyridyl]-1,2,4-thiadiazol-5-yl]ethyl]propanesulfinamide (C-32)

To a solution of (S,E)-2-methyl-N-[1-[3-[2-(trifluoromethyl)-4-pyridyl]-1,2,4-thiadiazol-5-yl]ethylidene]propane-2-sulfinamide (100 mg, 0.27 mmol) in THF (2.0 mL) was added L-Selectride (0.53 mL, 0.53 mmol) under N₂ at -78° C. After stirring at -78° C. for 30 mins, sat. NH₄Cl (10.0 mL) was added at -78° C. to the mixture. The mixture was extracted with EtOAc (2 x 20.0 mL). The combined organic layer was washed with brine (20.0 mL), dried over Na₂SO₄, filtered and concentrated under reduced pressure to give the product (100 mg, 0.26 mmol, 99% yield) as an oil which was used directly for next step. LCMS Rₜ = 0.728 min in 1.0 min chromatography, 5-95AB, MS ESI calcd. for C₁₄H₁₈F₃N₄OS₂ [M+H]⁺379.0, found 379.0.

### (1R)[3-[2-(trifluoromethyl)-4-pyridyl]-1,2,4-thiadiazol-5-yl]ethanamine hydrochloride (C-33)

To a solution of (S)-2-methyl-N-[(1R)-1-[3-[2-(trifluoromethyl)-4-pyridyl]-1,2,4-thiadiazol-5-yl]ethyl]propane-2-sulfinamide (130 mg, 0.34 mmol) in 1,4-Dioxane (1.0 mL) was added HCl/dioxane (3.0 mL, 4 M) at 25° C. After stirring at 25° C. for 2 hr, the reaction mixture was concentrated under reduced pressure to give the product (90.0 mg, 0.29 mmol, 84% yield) as as a solid which was used directly for next step. LCMS Rₜ = 0.754 min in 1.5 min chromatography, 5-95AB, MS ESI calcd. for C₁₀H₁₀F₃N₄S [M+H]⁺274.8, found 274.8.

### 2-methyl-N-[(1R)-1-[3-[2-(trifluoromethyl)-4-pyridyl]-1,2,4-thiadiazol-5-yl]ethyl]-5-(trifluoromethyl)pyrazole-3-carboxamide (10-1)

To a solution of 2-methyl-5-(trifluoromethyl)pyrazole-3-carboxylic acid (56.2 mg, 0.29 mmol) in DCM (3.0 mL) was added T₃P (661 mg, 0.87 mmol), DIEA (0.40 mL, 2.32 mmol). After stirring at 25° C. for 20 mins, (1R)-1-[3-[2-(trifluoromethyl)-4-pyridyl]-1,2,4-thiadiazol-5-yl]ethanamine hydrochloride (90.0 mg, 0.29 mmol) was added and the reaction mixture was stirred at 25° C. for 16 hr. The reaction mixture was quenched with water (20.0 mL) and extracted with DCM (2 × 20.0 mL). The combined organic layer was washed with brine (20.0 mL) and dried over Na₂SO₄, filtered and concentrated under reduced pressure to give the product which was purified by prep-HPLC (Column: Phenomenex Gemini-NX 80 ^{∗} 40 mm ^{∗} 3 µm, Condition: water (0.05% NH₃H₂O)-ACN, Begin B: 47, End B: 77, Gradient Time (min): 8, 100%B Hold Time (min): 2, FlowRate (mL/min): 30, Injections: 4) to give the product (70.0 mg, 0.16 mmol, 54% yield) as as a solid. The product (70.0 mg, 0.16 mmol) was purified by SFC (Column: DAICEL CHIRALCEL OJ (250 mm ^{∗} 30 mm, 10 µm), Condition: 0.1%NH₃H₂O EtOH, Begin B: 15%, End B: 15%, FlowRate (mL/min): 60, Injections: 20) to give the product (22.9 mg, 0.05 mmol, 33% yield) as a solid. ¹H NMR (CDC1₃, 400 MHz) δ_{H} = 8.88 (d, 1H), 8.53 (s, 1H), 8.33 (d,1H), 6.90 (s, 1H), 6.63 (d, 1H), 5.77-5.64 (m, 1H), 4.24 (s, 3H), 1.85 (d, 3H). ¹⁹F NMR (376.5 MHz, CDC1₃) δ_{F} -62.206, 68.046. LCMS Rt = 2.451 min in 3.0 min chromatography, 30-90AB, MS ESI calcd. for Ci₆Hᵢ₃F₆N₆OS [M+H]⁺451.1, found 451.1. 100%ee.

Examples 12 and 13. Synthesis of (S)-N-(1-(3-(2-cyclopropylpyridin-4-yl)-1,2,4-thiadiazol-5-yl)ethyl)-1-methyl-3-(trifluoromethyl)-1H-pyrazole-5-carboxamide (12) and (R)-N-(1-(3-(2-cyclopropylpyridin-4-yl)-1,2,4-thiadiazol-5-yl)ethyl)-1-methyl-3-(trifluoromethyl)-1H-pyrazole-5-carboxamide (13). Note the stereochemistry is randomly assigned.

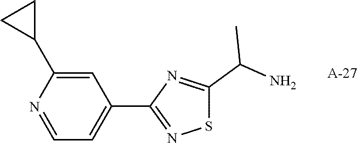 [SMILES: CC(N)c1nc(-c2ccnc(C3CC3)c2)ns1]

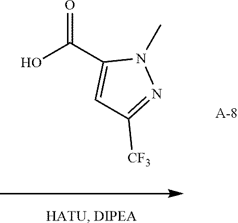

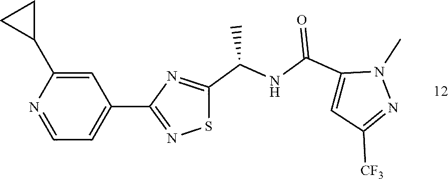 [SMILES: C[C@H](NC(=O)c1cc(C(F)(F)F)nn1C)c1nc(-c2ccnc(C3CC3)c2)ns1]

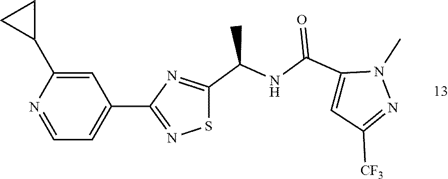 [SMILES: C[C@@H](NC(=O)c1cc(C(F)(F)F)nn1C)c1nc(-c2ccnc(C3CC3)c2)ns1]

To a stirred solution of A-27 (125 mg, 0.31 mmol) and 2-methyl-5-(trifluoromethyl)pyrazole-3-carboxylic acid (66.07 mg, 0.34 mmol) in DCM (10 mL) was added HATU (117.65 mg, 0.31 mmol) and DIPEA (0.11 mL, 0.62 mmol) at room temperature. The reaction mixture was stirred at room temperature for 2 h. The reaction mixture was quenched with water (10 mL) and diluted with DCM (2 × 100 mL). The organic layer was dried over anhydrous Na₂SO₄, filtered and evaporated to get a residue. The residue was purified by column chromatography using 100-200 silica and 30-80% EtOAc/hexane as an eluent to get racemic mixture which was then purified by SFC column chromatography to give 12 (10 mg, 0.0234 mmol, 8% yield) and 13 (10 mg, 0.0234 mmol, 8% yield).

12: HPLC: Rt 8.686 min, 99.87%; Column: X-Select CSH C18 (4.6 × 150) mm, 3.5 µm; Mobile phase: A: 0.1% Formic acid in water: ACN (95:05), B: ACN; Flow Rate: 1.0 mL/min; LCMS : 422.9 (M+H), Rt 1.89 min, Column: X-select CSH C18 (3^{∗}50) mm, 2.5 µm; ¹H NMR (400 MHz, DMSO-d6) δ 9.46 (d, 1H), 8.61 (d, 1H), 7.85 (s, 1H), 7.66-7.64 (m, 1H), 7.45 (s, 1H), 5.50-5.45 (m, 1H), 4.13 (s, 3H), 2.30-2.26 (m, 1H), 1.68 (d, 3H), 1.03-0.97 (m, 4H).Chiral method: Rt 4.755 min, 100%; column: PHENOMENEX CELLULOSE-3 (250 mm ×4.6 mm,5 u)- Mobile Phase: A) n-Hexane+0.1% TFA B) EtOH:MeOH (50:50), Isocratic:20%B; Wavelength: 240 nm, Flow: 1.0 mL/min.

13: HPLC: Rt 8.348 min, 97.85%; Column: X-Select CSH C18 (4.6 × 150) mm, 3.5 µm; Mobile phase: A: 0.1% Formic acid in water: ACN (95:05), B: ACN; Flow Rate: 1.0 mL/min; LCMS : 422.9 (M+H), Rt 1.894 min, Column: X-select CSH C18 (3^{∗}50) mm, 2.5 µm; ¹H NMR (400 MHz, DMSO-d6) δ 9.46 (d, 1H), 8.61 (d, 1H), 7.85 (s, 1H), 7.66-7.64 (m, 1H), 7.44 (s, 1H), 5.50-5.46 (m, 1H), 4.13 (s, 3H), 2.30-2.26 (m, 1H), 1.68 (d, 3H), 1.03-0.97 (m, 4H).Chiral method: Rt 8.044 min, 100%; column: PHENOMENEX CELLULOSE-3 (250 mm ×4.6 mm,5 u)- Mobile Phase: A) n-Hexane+0.1% TFA B) EtOH:MeOH (50:50), Isocratic:20%B; Wavelength: 240 nm, Flow: 1.0 mL/min.

Examples 12-1 and 13-1. Synthesis of 2-methyl-N-[(1R)-1-[3-(2-cyclopropyl-4-pyridyl)-1,2,4-thiadiazol-5-yl] ethyl] -5-(trifluoromethyl)pyrazole-3-carboxamide & 2-methyl-N-[(1 S)-1- [3-(2-cyclopropyl-4-pyridyl)-1,2,4-thiadiazol-5-yl]ethyl]-5-(trifluoromethyl)pyrazole-3-carboxamide

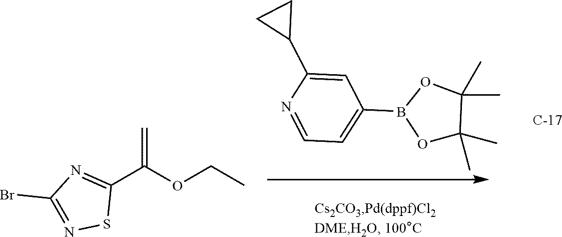

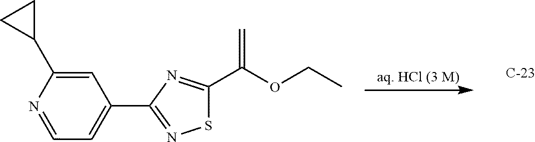

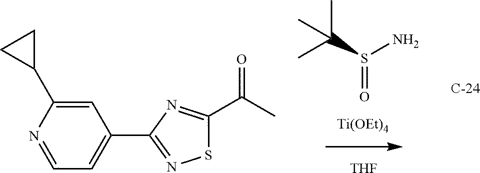

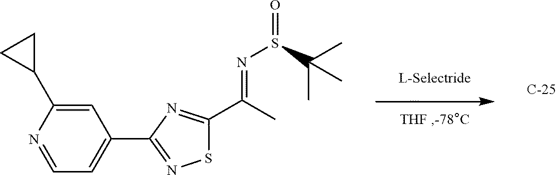

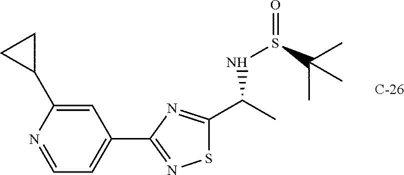 [SMILES: C[C@@H](N[S@@](=O)C(C)(C)C)c1nc(-c2ccnc(C3CC3)c2)ns1]

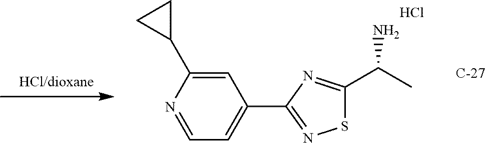

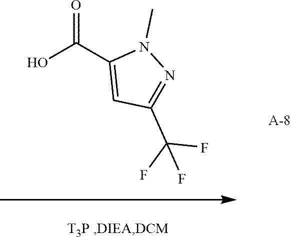

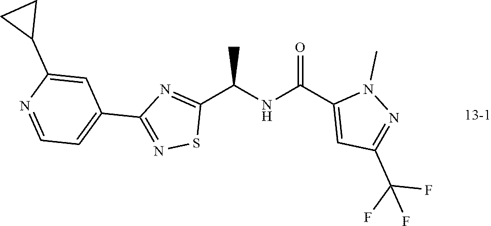 [SMILES: C[C@@H](NC(=O)c1cc(C(F)(F)F)nn1C)c1nc(-c2ccnc(C3CC3)c2)ns1]

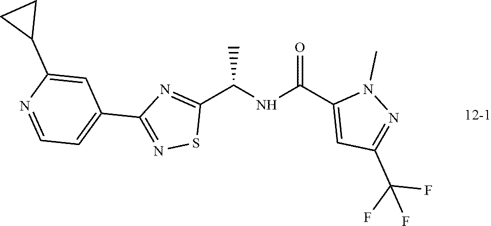 [SMILES: C[C@H](NC(=O)c1cc(C(F)(F)F)nn1C)c1nc(-c2ccnc(C3CC3)c2)ns1]

### 3-Cyclopropyl-4-pyridyl)-5-(1-ethoxyvinyl)-1,2,4-thiadiazole (C-23)

To a solution of 3-bromo-5-(1-ethoxyvinyl)-1,2,4-thiadiazole (2.0 g, 8.51 mmol) in DME (10.0 mL, 8.51 mmol) and water (2.0 mL) was added 2-cyclopropyl-4-(4,4,5,5-tetramethyl-1,3,2-dioxaborolan-2-yl)pyridine (2.29 g, 9.36 mmol), C_{S2}CO₃ (5.54 g, 17.0 mmol) and Pd(dppf)Cl₂ (0.62 g, 0.85 mmol) under N₂. The reaction mixture was stirred at 100° C. for 1.5 hrs. After cooling to 25° C., the reaction mixture was filtered and the filtrate was concentrated under reduced pressure. The residue was purified by chromatography on silica gel with petroleum/ethyl acetate= 20/1) to give the product (1.60 g, 5.85 mmol, 69% yield) as an oil. LCMS Rt = 0.676 min in 1.0 min chromatography, 5-95AB, MS ESI calcd. for C₁₄H₁₆N₃OS [M+H]⁺274.0, found 274.0.

### 1-(2-Cyclopropyl-4-pyridyl)-1,2,4-thiadiazol-5-yl]ethanone (C-24)

To a solution of 3-(2-cyclopropyl-4-pyridyl)-5-(1-ethoxyvinyl)-1,2,4-thiadiazole (1.6 g, 5.85 mmol) in acetone (20.0 mL) was added 3 M HCl (1.07 g, 29.3 mmol) at 25° C. After stirring at 25° C. for 16 hr, the reaction mixture was quenched with sat. NaHCO₃ (30.0 mL) and extracted with EtOAc (2 × 30.0 mL). The combined organic layer was washed with brine (30.0 mL) and dried over Na₂SO₄, filtered and concentrated under reduced pressure to give the product (1.10 g, 4.48 mmol, 77% yield) as a solid which was used directly for next step. ¹H NMR DMSO-d₆ 400 MHz δ_{H} = 8.72 (d, 1H), 8.18 (s, 1H), 8.13 (d, 1H), 2.79 (s, 3H), 1.37-1.34 (m, 1H), 1.26-1.11 (m, 4H).

### (S,E)-N-[1-[3-(2-cyclopropyl-4-pyridyl)-1,2,4-thiadiazol-5-yl]ethylidene]-2-methylpropane-2-sulfinamide (C-25)

To a solution of 1-[3-(2-cyclopropyl-4-pyridyl)-1,2,4-thiadiazol-5-yl]ethanone (500 mg, 2.04 mmol) in THF (10.0 mL) was added (S)-2-methylpropane-2-sulfinamide (371 mg, 3.06 mmol) and Ti(OEt)₄ (1.39 g, 6.11 mmol) at 25° C. After stirring at 50° C. for 16 hr, the reaction mixture was cooled to 25° C. and quenched with sat. NaHCO₃ (10.0 mL) and filtered. The filtrate was extracted with EtOAc (2 × 20.0 mL). The combined organic layer was dried over Na₂SO₄, filtered and concentrated under reduced pressure. The product was purified by chromatography column (EtOAc in PE, 5%~10%) to give the product (280 mg, 0.80 mmol, 39% yield) as a solid. ¹H NMR (CDC1₃, 400 MHz) δ_{H} = 8.60 (d, 1H), 8.00 (s, 1H), 7.91-7.88 (m, 1H), 2.97 (s, 3H), 2.23-2.11 (m, 1H), 1.37 (s, 9H), 1.15-1.00 (m, 4H).

### (S)methyl-N-[(1R)-1-[3-(2-cyclopropyl-4-pyridyl)-1,2,4-thiadiazol-5-yl]ethyl]propanesulfinamide (C-26)

To a solution of (S,E)-N-[1-[3-(2-cyclopropyl-4-pyridyl)-1,2,4-thiadiazol-5-yl]ethylidene]-2-methyl-propane-2-sulfinamide (280 mg, 0.80 mmol) in THF (5.0 mL) was added L-Selectride (1.61 mL, 1.61 mmol) at -78° C. under N₂. After stirring at -78° C. for 1 h, the reaction mixture was quenched with sat. NH₄Cl (20.0 mL) and extracted with EtOAc (2 × 20.0 mL). The combined organic layer was dried over Na₂SO₄, filtered and concentrated under reduced pressure to give the product (200 mg, 0.57 mmol, 71% yield) as an oil which was used directly for next step. LCMS Rₜ = 0.805 min in 1.5 min chromatography, 5-95AB, MS ESI calcd. for C₁₆H₂₃N₄OS₂ [M+H]⁺350.9, found 350.9.

### (1R)[3-(2-cyclopropyl-4-pyridyl)-1,2,4-thiadiazol-5-yl]ethanamine (C-27)

To a solution of (S)-2-methyl-N-[(1R)-1-[3-(2-cyclopropyl-4-pyridyl)-1,2,4-thiadiazol-5-yl]ethyl]propane-2-sulfinamide (200 mg, 0.57 mmol) in 1,4-dioxane (3.0 mL) was added 4 M HCl/dioxane (0.43 mL, 1.71 mmol) at 25° C. After stirring at 25° C. for 2 hrs, the reaction mixture was quenched with sat. NaHCO₃ (20.0 mL) and extracted with EtOAc (2 × 20.0 mL). The combined organic layer was dried over Na₂SO₄, filtered and concentrated under reduced pressure to give the product (140 mg, 0.57 mmol, 99% yield) as an oil which was used directly for next step. LCMS Rₜ = 0.437 min in 1.5 min chromatography, 5-95AB, MS ESI calcd. for C₁₂H₁₅N₄S [M+H]⁺246.8, found 246.8.

### 2-methyl-N-[(1R)-1-[3-(2-cyclopropyl-4-pyridyl)-1,2,4-thiadiazol-5-yl]ethyl]-5-(trifluoromethyl)pyrazole-3-carboxamide & 2-methyl-N-[(1S)-1-[3-(2-cyclopropyl-4-pyridyl)-1,2,4-thiadiazol-5-yl]ethyl]-5-(trifluoromethyl)pyrazole-3-carboxamide

To a solution of 2-methyl-5-(trifluoromethyl)pyrazole-3-carboxylic acid (100 mg, 0.52 mmol) in DCM (2.0 mL) was added DIEA (0.47 mL, 2.71 mmol), T₃P (617 mg, 0.81 mmol). After stirring at 25° C. for 10 mins, (1R)-1-[3-(2-cyclopropyl-4-pyridyl)-1,2,4-thiadiazol-5-yl]ethanamine hydrochloride (140 mg, 0.50 mmol) in DCM (2.0 mL) was added and the reaction mixture was stirred at 25° C. for 16 hr. The reaction mixture was quenched with water (20.0 mL) and extracted with DCM (2 × 20.0 mL). The combined organic layer was washed with brine (20.0 mL) and dried over Na₂SO₄, filtered and concentrated under reduced pressure to give product which was purified by prep-HPLC (Column: Phenomenex Gemini-NX 80 ^{∗} 40 mm ^{∗} 3 µm, Condition: water (0.05% NH₃H₂O)-ACN, Begin B: 44, End B: 74, Gradient Time (min): 8, 100%B Hold Time (min): 2.8, FlowRate (mL/min): 30, Injections: 8) to give the product (90.0 mg, 0.21 mmol, 41% yield) as an oil which was purified by SFC (Column: (s,s) WHELK-O1 (250 mm ^{∗} 30 mm, 5 µm), Condition: 0.1%NH₃H₂O-EtOH, Begin B: 35%, End B: 35%, FlowRate (mL/min): 80, Injections: 50) to give 2-methyl-N-[(1R)-1-[3-(2-cyclopropyl-4-pyridyl)-1,2,4-thiadiazol-5-yl ] ethyl]-5-(trifluoromethyl)pyrazole-3-carboxamide (61.82 mg, 0.14 mmol, 68% yield) as a solid and 2-methyl-N-[(1S)-1-[3-(2-cyclopropyl-4-pyridyl)-1,2,4-thiadiazol-5-yl]ethyl]-5-(trifluoromethyl)pyrazole-3-carboxamide (10.76 mg, 0.03 mmol, 12% yield) as a solid. 13-1: ¹H NMR (CDC1₃, 400 MHz)δ_{H} = 8.58 (d, 1H), 7.96 (s, 1H), 7.87-7.84 (m, 1H), 6.89 (s, 1H), 6.69 (d, 1H), 5.76-5.65 (m, 1H), 4.24 (s, 3H), 2.22-2.08 (m, 1H), 1.83 (d, 3H), 1.14-1.01 (m, 4H).¹⁹F NMR (376.5 MHz, CDC1₃) δ_{F} -62.212.LCMS Rₜ= 2.131 min in 3.0 min chromatography, 10-80CD, MS ESI calcd. for C₁₈H₁₈F₃N₆OS [M+H]⁺423.0, found 423.0. 100%ee.

12-1: ¹H NMR (CDC1₃, 400 MHz) δ_{H} = 8.58 (d, 1H), 7.96 (s, 1H), 7.87-7.83 (m, 1H), 6.90 (s, 1H), 6.70 (d, 1H), 5.76-5.65 (m, 1H), 4.24 (s, 3H), 2.22-2.08 (m, 1H), 1.83 (d, 3H), 1.13-1.00 (m, 4H). ¹⁹F NMR (376.5 MHz, CDCl₃) δ_{F} -62.210. LCMS Rₜ = 2.120 min in 3.0 min chromatography, 10-80CD, MS ESI calcd. for C₁₈H₁₈F₃N₆OS [M+H]⁺423.0, found 423.0. 99.5%ee.

Examples 12-2 and 13-2. Synthesis of 2-methyl-N-[(1R)-1-[3-(2-cyclopropyl-4-pyridyl)-1,2,4-thiadiazol-5-yl]ethyl]-5-(trifluoromethyl)pyrazole-3-carboxamide & 2-methyl-N-[(1S)-1- [3-(2-cyclopropyl-4-pyridyl)-1,2,4-thiadiazol-5-yl] ethyl]-5-(trifluoromethyl)pyrazole-3-carboxamide

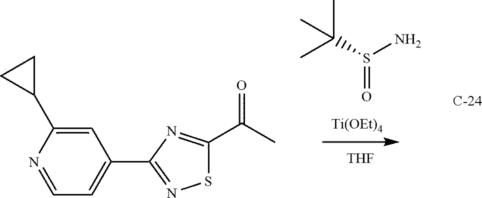

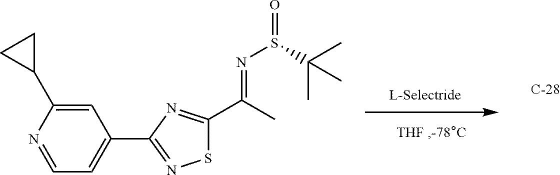

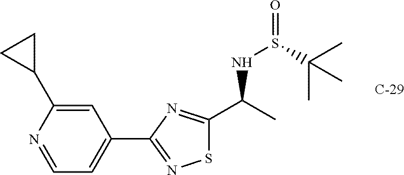 [SMILES: C[C@H](N[S@](=O)C(C)(C)C)c1nc(-c2ccnc(C3CC3)c2)ns1]

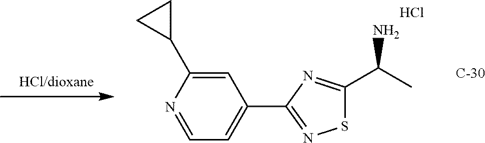

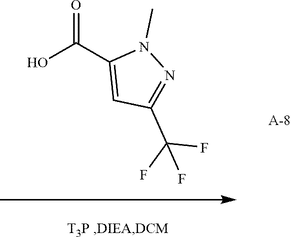

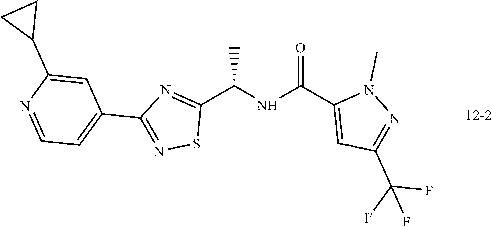 [SMILES: C[C@H](NC(=O)c1cc(C(F)(F)F)nn1C)c1nc(-c2ccnc(C3CC3)c2)ns1]

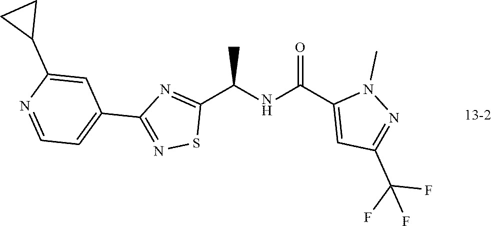 [SMILES: C[C@@H](NC(=O)c1cc(C(F)(F)F)nn1C)c1nc(-c2ccnc(C3CC3)c2)ns1]

### (R,E)-N-[1-[3-(2-cyclopropyl-4-pyridyl)-1,2,4-thiadiazol-5-yl]ethylidene]-2-methylpropane-2-sulfinamide (C-28)

To a solution of 1-[3-(2-cyclopropyl-4-pyridyl)-1,2,4-thiadiazol-5-yl]ethanone (500 mg, 2.04 mmol) in THF (10.0 mL) was added and Ti(OEt)₄ (1.39 g, 6.11 mmol) at 25° C. After stirring at 50° C. for 16 hr, the reaction mixture was cooled to 25° C. and quenched with sat. NaHCO₃ (40.0 mL) and filtered. The filtrate was extracted with EtOAc (2 × 40.0 mL). The combined organic layer was dried over Na₂SO₄, filtered and concentrated under reduced pressure. The product was purified by chromatography column (EtOAc in PE, 5%~10%) to give the product (300 mg, 0.86 mmol, 42% yield) as a solid. ¹H NMR (CDCl₃, 400 MHz) δ_{H} = 8.60 (d, 1H), 8.00 (s, 1H), 7.90 (d, 1H), 2.97 (s, 3H), 1.36 (s, 9H), 1.15-1.02 (m, 1H), 0.92-0.75 (m, 4H).

### (R)methyl-N-[(1S)-1-[3-(2-cyclopropyl-4-pyridyl)-1,2,4-thiadiazol-5-yl]ethyl] Propanesulfinamide (C-29)

To a solution of (R,E)-N-[1-[3-(2-cyclopropyl-4-pyridyl)-1,2,4-thiadiazol-5-yl]ethylidene]-2-methyl-propane-2-sulfinamide (300 mg, 0.86 mmol) in THF (5.0 mL) was added L-Selectride (1.72 mL, 1.72 mmol) at -78° C. under N₂. After stirring at -78° C. for 1 h, the reaction mixture was quenched with sat. NH₄Cl (20.0 mL) and extracted with EtOAc (2 × 20.0 mL). The combined organic layer was dried over Na₂SO₄, filtered and concentrated under reduced pressure to give the product (350 mg, 1.00 mmol) as an oil which was used directly for next step. LCMS Rₜ = 0.791 min in 1.5 min chromatography, 5-95AB, MS ESI calcd. for C₁₆H₂₃N₄OS₂ [M+H]⁺351.2, found 351.2.

### (1S)[3-(2-cyclopropyl-4-pyridyl)-1,2,4-thiadiazol-5-yl]ethanamine Hydrochloride (C-30)

To a solution of (R)-2-methyl-N-[(1S)-1-[3-(2-cyclopropyl-4-pyridyl)-1,2,4-thiadiazol-5-yl]ethyl]propane-2-sulfinamide (330 mg, 0.94 mmol) in 1,4-Dioxane (3.0 mL) was added 4 M HCl/dioxane (0.71 mL, 2.82 mmol) at 25° C. After stirring at 25° C. for 2 hr, the reaction mixture was quenched with sat. NaHCO₃ (20.0 mL) and extracted with EtOAc (2 × 30.0 mL). The combined organic layer was dried over Na₂SO₄, filtered and concentrated under reduced pressure to give the product (200 mg, 0.71 mmol, 75% yield) as an oil which was used directly for next step. ¹H NMR (DMSO-d₆400 MHz) δ_{H}= 8.56 (d, 1H), 8.00 (s, 1H), 7.84-7.80 (m, 1H), 7.28 (s, 1H), 6.53 (s, 1H), 4.43 (q, 1H), 1.49 (d, 2H), 1.04-0.94 (m, 3H), 0.89-0.79 (m, 3H).

### 2-methyl-N-[(1R)-1-[3-(2-cyclopropyl-4-pyridyl)-1,2,4-thiadiazol-5-yl]ethyl]-5-(trifluoromethyl)pyrazole-3-carboxamide & 2-methyl-N-[(1S)-1-[3-(2-cyclopropyl-4-pyridyl)-1,2,4-thiadiazol-5-yl]ethyl]-5-(trifluoromethyl)pyrazole-3-carboxamide

To a solution of 2-methyl-5-(trifluoromethyl)pyrazole-3-carboxylic acid (82.4 mg, 0.42 mmol) in DCM (2.0 mL) was added DIEA (0.62 mL, 3.54 mmol), T₃P (807 mg, 1.06 mmol). After stirring at 25° C. for 10 mins, (1S)-1-[3-(2-cyclopropyl-4-pyridyl)-1,2,4-thiadiazol-5-yl]ethanamine hydrochloride (100 mg, 0.35 mmol) in DCM (2.0 mL) was added and the reaction mixture was stirred at 25° C. for 16 hr. The reaction mixture was quenched with water (20.0 mL) and extracted with DCM (2 × 20.0 mL). The combined organic layer was washed with brine (20.0 mL) and dried over Na₂SO₄, filtered and concentrated under reduced pressure to give the product which was purified by prep-HPLC (Column: Phenomenex Gemini-NX 80 ^{∗} 40 mm ^{∗} 3 µm, Condition: water (0.05% NH₃H₂O)-ACN, Begin B: 43, End B: 73, Gradient Time(min): 8, 100%B Hold Time (min): 2, FlowRate (mL/min): 30, Injections: 5) to give 2the product (80.0 mg, 0.19 mmol, 54% yield) as an oil which was used for SFC separation. The product (80.0 mg, 0.19 mmol) was purified by SFC (Column: DAICEL CHIRALCEL OD-H (250 mm ^{∗} 30 mm, 5 µm), Condition: 0.1% NH₃H₂O-EtOH, Begin B: 35%, End B: 35%, FlowRate (mL/min): 80, Injections: 45) to give 2-methyl-N-[(1R)-1-[3-(2-cyclopropyl-4-pyridyl)-1,2,4-thiadiazol-5-yl]ethyl]-5-(trifluoromethyl)pyrazole-3-carboxamide (3.29 mg, 0.01 mmol, 4% yield) as a solid and 2-methyl-N-[(1S)-1-[3-(2-cyclopropyl-4-pyridyl)-1,2,4-thiadiazol-5-yl]ethyl]-5-(trifluoromethyl)pyrazole-3-carboxamide (34.82 mg, 0.08 mmol, 44% yield) as a solid.

2-2: ¹H NMR (CDCl₃, 400 MHz) δ_{H} = 8.58 (d, 1H), 7.96 (s, 1H), 7.87-7.84 (m, 1H), 6.89 (s, 1H), 6.69 (d, 1H), 5.76-5.65 (m, 1H), 4.24 (s, 3H), 2.22-2.08 (m, 1H), 1.83 (d, 3H), 1.14-1.01 (m, 4H). ¹⁹F NMR (376.5 MHz, CDC1₃) δ_{F} -62.186. LCMS Rₜ = 2.296 min in 3.0 min chromatography, 10-80AB, MS ESI calcd. for C₁₈H₁₈F₃N₆OS [M+H]⁺423.4, found 423.4. 100%ee.

13-2: ¹H NMR (CDC1₃, 400 MHz) δ_{H} = 8.58 (d, 1H), 7.96 (s, 1H), 7.87-7.83 (m, 1H), 6.90 (s, 1H), 6.70 (d, 1H), 5.76-5.65 (m, 1H), 4.24 (s, 3H), 2.22-2.08 (m, 1H), 1.83 (d, 3H), 1.13-1.00 (m, 4H). ¹⁹F NMR (376.5 MHz, CDCl₃) δ_{F} -62.177. LCMS Rₜ = 2.265 min in 3.0 min chromatography, 10-80AB, MS ESI calcd. for C₁₈H₁₈F₃N₆OS [M+H]⁺423.2, found 423.2. 100%ee.

Examples 14 and 15. Synthesis of (S)-N-(1-(3-(2-cyclopropylpyridin-4-yl)isoxazol-5-yl)ethyl)benzamide (14) and Synthesis of (R)-N-(1-(3-(2-cyclopropylpyridin-4-yl)isoxazol-5-yl) ethyl) benzamide (15). Note the stereochemistry is randomly assigned.

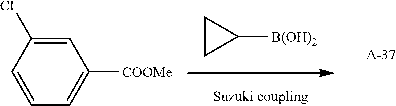

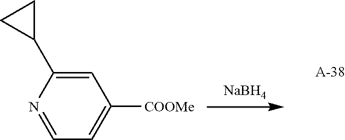 [SMILES: COC(=O)c1ccnc(C2CC2)c1 ; "NaBH4"]

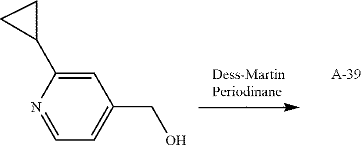

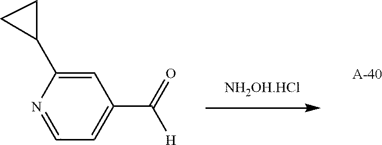

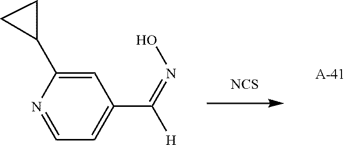 [SMILES: O/N=C\c1ccnc(C2CC2)c1 ; "NCS"]

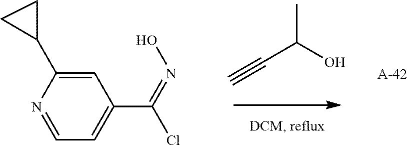

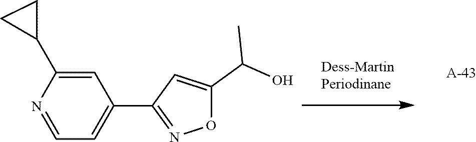

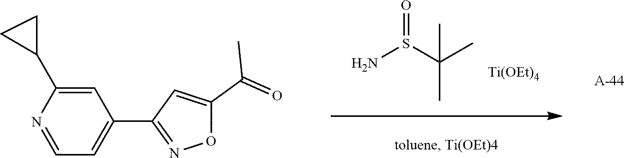

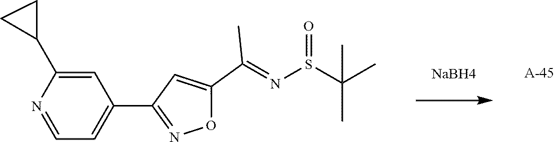 [SMILES: C/C(=N\S(=O)C(C)(C)C)c1cc(-c2ccnc(C3CC3)c2)no1 ; "NaBH4"]

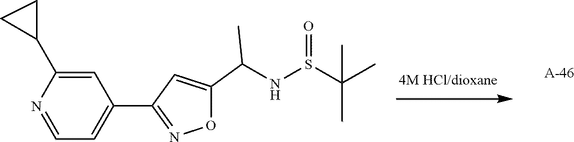

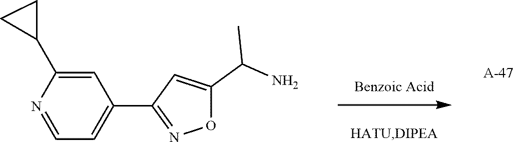

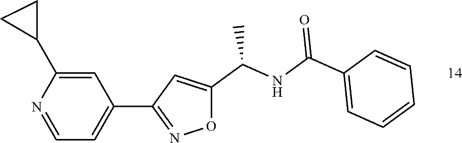 [SMILES: C[C@H](NC(=O)c1ccccc1)c1cc(-c2ccnc(C3CC3)c2)no1]

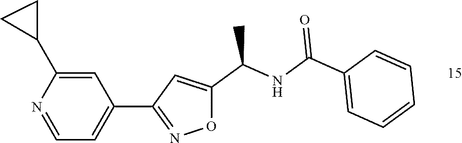 [SMILES: C[C@@H](NC(=O)c1ccccc1)c1cc(-c2ccnc(C3CC3)c2)no1]

### Synthesis of Methyl 2-cyclopropylisonicotinate (A-38)

To a stirred solution of A-37 (4. g, 23.31 mmol) in 1,4-Dioxane (50 mL) was added Cyclopropyl Boronic Acid (2.38 g, 27.98 mmol), K₃PO₄ (9.9 g, 46.63 mmol) and Ag₂O (2.7 g, 11.66 mmol). To this solution Pd(dppf)Cl₂ (1.71 g, 2.33 mmol) was added and the mixture was stirred at 100° C. for 12 h. The reaction mixture was cooled to RT and filtered. The filtrate was concentrated under reduced pressure. The residue was purified by 100-200 silica gel column chromatography using 20-30% EtOAc/hexane as an eluent to afford A-38 (2.6 g, 14.12 mmol, 61%) as an oil.

### Synthesis of (2-cyclopropylpyridin-4-yl)methanol (A-39)

To a stirred solution of A-38 (2.5 g, 14.11 mmol) in Methanol (10 mL) was added NaBH4 (1.07 g, 28.22 mmol) at 0° C. and the mixture was stirred at RT for 6 h. The reaction mixture was quenched with ice cold water and extracted with DCM. Organic layer was separated, dried over anhydrous sodium sulfate and concentrated under reduced pressure to afford the A-39 (2 g, 12.8 mmol, 91%) as a liquid.

### Synthesis of 2-cyclopropylisonicotinaldehyde (A-40)

To a stirred solution of A-40 (2 g, 13.41 mmol) in DCM (20 mL) was added Dess martin periodinane (5.68 g, 13.41 mmol) at 0° C. and the reaction mixture was stirred at RT for 16 h. The reaction mixture was diluted with DCM (50 mL), saturated sodium thiosulphate (20 mL) and saturated sodium bicarbonate (20 mL). The organic layer was separated, washed with water (2 × 30 mL) and saturated brine solution (30 mL). The organic layer was separated and dried over anhydrous MgSO₄ and concentrated under reduced pressure. The residue was purified by 100-200 silica gel column chromatography using 20-30% EtOAc/hexane as an eluent to afford A-40 (1.6 g, 8.83 mmol, 66%) as an oil.

### Synthesis of (Z)-2-cyclopropylisonicotinaldehyde Oxime (A-41)

To a stirred solution of A-40 (1.6 g, 10.87 mmol) in ethanol (5 mL) and water (25 mL) was added Hydroxyl amine hydrochloride (0.91 g, 13.05 mmol) and stirred at RT for 12 h. The reaction mixture was concentrated under reduced pressure. The residue was diluted with water (20 mL) and extracted with EtOAc (50 mL). The organic layer was washed with water (2 × 20 mL) and saturated brine solution (20 mL). The organic layer was separated and dried over anhydrous MgSO₄ and concentrated under reduced pressure. The residue was purified by 100-200 silica gel column chromatography using 20-30% EtOAc/hexane as an eluent to afford A-41 (1.6 g, 6.35 mmol, 58%) as a solid.

### Synthesis of (E)-2-cyclopropyl-N-hydroxyisonicotinimidoyl Chloride (A-42)

To a stirred solution of A-41 (1.6 g, 9.86 mmol) in DMF (20 mL) was added N-Chlorosuccinimide (2.63 g, 19.73 mmol) and stirred at RT for 6 h. The reaction mixture was diluted with EtOAc (50 mL) and water (20 mL). The organic layer was washed with water (2 × 20 mL) and saturated brine solution (20 mL). The organic layer was separated and dried over anhydrous MgSO₄ and concentrated under reduced pressure. The residue was purified by 100-200 silica gel column chromatography using 20-30% EtOAc/hexane as an eluent to A-42 (1.2 g, 4.91 mmol, 50%) as a solid.

### Synthesis of 1-(3-(2-cyclopropylpyridin-4-yl)isoxazol-5-yl)ethan-1-ol (A-43)

To a stirred solution of A-42 (1.2 g, 6.1 mmol) in THF (15 mL) were added but-3-yn-2-ol (0.86 g, 12.21 mmol) and triethyl amine (0.62 g, 6.1 mmol) and stirred at 60° C. for 3 h. The reaction mixture was concentrated under reduced pressure. The residue was diluted with water (20 mL) and extracted with EtOAc (50 mL). The organic layer was washed with water (2 × 20 mL) and saturated brine solution (20 mL). The organic layer was separated and dried over anhydrous MgSO₄ and concentrated under reduced pressure. The residue was purified by 100-200 silica gel column chromatography using 20-30% EtOAc/hexane as an eluent to afford A-43 (0.8 g, 3.47 mmol, 57%) as an oil.

### Synthesis of 1-(3-(2-cyclopropylpyridin-4-yl)isoxazol-5-yl)ethan-1-one (A-44)

To a stirred solution of A-43 (0.8 g, 3.47 mmol) in DCM (20 mL) was added Dess martin Periodinane (2.95 g, 6.95 mmol). The reaction mixture was stirred at RT for 12 h. After completion reaction mass was diluted with DCM (30 mL) and saturated sodium thiosulphate 10 (mL) and saturated bicarbonate (10 mL). The organic layer was separated and dried over anhydrous MgSO₄ and concentrated under reduced pressure. The residue was purified by 100-200 silica gel column chromatography using 70-80% EtOAc/hexane as an eluent to afford A-44 (0.62 g, 2.394 mmol, 69%) as a solid.

### Synthesis of (E)-N-(1-(3-(2-cyclopropylpyridin-4-yl)isoxazol-5-yl)ethylidene)-2-methylpropane-2-sulfinamide (A-45)

To a stirred solution of A-44 (0.62 g, 2.72 mmol) in Toluene (10 mL) was added Ti(OEt)₄ (0.93 g, 4.07 mmol) and stirred at 100° C. for 12 h. 2) After completion reaction mass was diluted with EtOAc (30 mL) and water (10 mL) and filtered through a pad of celite. The organic layer was separated, dried over anhydrous MgSO₄ and concentrated under reduced pressure. The residue was purified by 100-200 silica gel column chromatography using 70-80%EtOAc/hexane as an eluent to afford A-45 (0.7 g, 1.3 mmol, 46.31%) as an oil.

### Synthesis of N-(1-(3-(2-cyclopropylpyridin-4-yl)isoxazol-5-yl)ethyl)-2-methylpropane-2-sulfinamide (A-46)

To a stirred the solution of A-45 (700 mg, 2.11 mmol) in methanol (10 mL) at 0° C. Sodium borohydride (159.8 mg, 4.22 mmol) was added. The reaction mixture was stirred at RT for 1 h. The reaction mixture was diluted with water and extracted with ethyl acetate (2 × 20 mL). The organic layer was separated and dried over anhydrous MgSO₄ and concentrated under reduced pressure to afford A-46 (600 mg, 1.44 mmol, 68%).

### Synthesis of 1-(3-(2-cyclopropylpyridin-4-yl)isoxazol-5-yl)ethan-1-amine (A-47)

To a stirred the solution of A-46 (700 mg, 2.1 mmol) in 1,4 dioxane (3 mL) at 0° C. was added 4M HCl 1,4 dioxane (10 mL, 2.1 mmol). The reaction mixture was stirred at RT for 2 h. After completion, the reaction mixture was concentrated under reduced pressure. The residue was purified by trituration with diethyl ether to afford A-47 (500 mg, 1.83 mmol, 87%).

### Synthesis of (S)-N-(1-(3-(2-cyclopropylpyridin-4-yl)isoxazol-5-yl)ethyl)benzamide (14) and Synthesis of (R)-N-(1-(3-(2-cyclopropylpyridin-4-yl)isoxazol-5-yl)ethyl)benzamide (15): Note that stereochemistry is randomly assigned

To a stirred solution of A-47 (200 mg, 0.73 mmol) and Benzoic Acid (106.98 mg, 0.88 mmol) in DCM (10 mL) were added HATU (416.37 mg, 1.1 mmol) and DIPEA (0.25 mL, 1.46 mmol) at RT. The reaction mixture was stirred at RT for 2 h. After completion, the reaction mixture was quenched with water (10 mL) and extracted with DCM (2 × 50 mL). Organic layer was separated, dried over anhydrous Na₂SO₄, filtered and concentrated under reduced pressure. The residue was purified by 100-200 silica gel column chromatography using 80% EtOAc/hexane as an eluent to afford racemic mixture which was then purified by SFC column chromatography to give 14 (15 mg, 0.045 mmol, 6%) and 15 (10 mg, 0.03 mmol, 4%).

14: HPLC: Rt 6.55 min, 99.64%; Column: X-Select CSH C18 (4.6 × 150) mm, 3.5 µm; Mobile phase: A: 0.1% Formic acid in water: ACN (95:05), B: ACN; Flow Rate: 1.0 mL/min; LCMS : 333.9 (M+H), Rt 1.612 min, Column: X-select CSH C18 (3^{∗}50) mm, 2.5 µm ; ¹H NMR (400 MHz, DMSO-d6) δ 9.06 (d, 1H), 8.56 (d, 1H), 7.92 (d, 2H), 7.80 (s, 1H), 7.68 (d, 1H), 7.58-7.54 (m, 1H), 7.52-7.45 (m, 2H), 7.15 (s, 1H), 5.44 (p, 1H), 2.25-2.20 (m, 1H), 1.61 (d, 3H), 1.10- 0.97 (m, 4H).Chiral method: Rt 5.034 min, 100%; column: PHENOMENEX CELLULOSE-3 (250×4.6 mm,5 u), Mobile Phase: A) n-Hexane+0.1% TFA, B) EtOH: MeOH (50:50), Isocratic: 35% B; Wavelength: 287 nm, Flow: 1.0 mL/min.

15: HPLC: Rt 6.86 min, 98.74%; Column: X-Select CSH C18 (4.6 × 150) mm, 3.5 µmMobile phase: A: 0.1% Formic acid in water: ACN (95:05), B: ACN; Flow Rate: 1.0 mL/min; LCMS : 334 (M+H), Rt 1.612 min, Column: X-select CSH C18 (3^{∗}50) mm, 2.5 µm. ¹H NMR (400 MHz, DMSO-d6) δ 9.06 (d, 1H), 8.52 (d, 1H), 7.96 (d, 2H), 7.77 (s, 1H), 7.60-7.46 (m, 4H), 7.11 (s, 1H), 5.46-5.40 (m 1H), 2.20-2.18 (m, 1H), 1.60 (d, 3H), 1.00-0.96 (m, 4H). Chiral method: Rt 5.523 min, 100%; column: PHENOMENEX CELLULOSE-3 (250×4.6 mm,5 u), Mobile Phase: A) n-Hexane+0.1% TFA, B) EtOH: MeOH (50:50), Isocratic: 35% B; Wavelength: 287 nm, Flow: 1.0 mL/min.

Examples 16 and 17. Synthesis of (R)-N-(1-(3-(2-cyclopropylpyridin-4-yl)isoxazol-5-yl)ethyl)-1-methyl-3-(trifluoromethyl)-1H pyrazole-5-carboxamide(16) and (S)-N-(1-(3-(2-cyclopropylpyridin-4-yl)isoxazol-5-yl)ethyl)-1-methyl-3-(trifluoromethyl)-1H-pyrazole-5-carboxamide (17). Note the stereochemistry is randomly assigned.

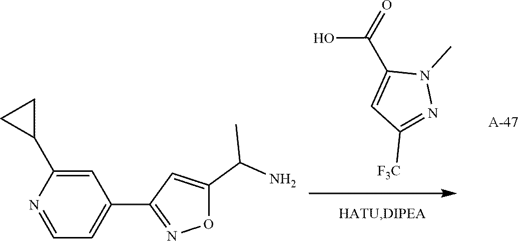

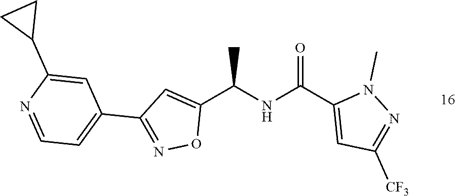 [SMILES: C[C@@H](NC(=O)c1cc(C(F)(F)F)nn1C)c1cc(-c2ccnc(C3CC3)c2)no1]

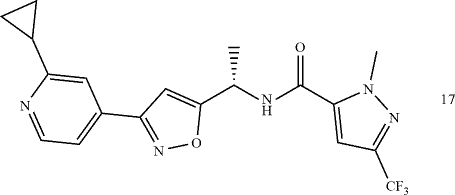 [SMILES: C[C@H](NC(=O)c1cc(C(F)(F)F)nn1C)c1cc(-c2ccnc(C3CC3)c2)no1]

To a stirred solution of A-47 (200 mg, 0.73 mmol) and 2-methyl-5-(trifluoromethyl)pyrazole-3-carboxylic acid (170.05 mg, 0.88 mmol) in DCM (10 mL) was added HATU (322.7 mg, 0.85 mmol) and DIPEA (0.25 mL, 1.41 mmol) at RT. The reaction mixture was stirred at RT for 2 hr. The reaction mixture was quenched with water (10 mL) and extracted with DCM (2 × 50 mL). Organic layer was separated, dried over anhydrous Na₂SO₄, filtered and concentrated under reduced pressure. The residue was purified by 100-200 silica gel column chromatography using 30-80% EtOAc/hexane as an eluent to afford racemic mixture which was then purified by SFC column chromatography to give 16 (10 mg, 0.0245 mmol, 3%) and 17 (10 mg, 0.0245 mmol, 3%).

16: HPLC: Rt 7.804 min, 99.35%; Column: X-Select CSH C18 (4.6 × 150) mm, 3.5 µm; Mobile phase: A: 0.1% Formic acid in water: ACN (95:05), B: ACN; Flow Rate: 1.0 mL/min; LCMS : 406.45 (M+H), Rt 1.921 min, Column: X-select CSH C18 (3^{∗}50) mm, 2.5 µm; ¹H NMR (400 MHz, DMSO-d6) δ 8.94 (d, 1H), 8.59 (d, 1H), 7.86-7.80 (m, 2H), 7.63 (d, 1H), 6.67 (s, 1H), 5.48-5.40 (m, 1H), 3.93 (s, 3H), 2.35-2.25 (m, 1H), 1.67 (d, 3H), 1.05-0.95 (m, 4H).Chiral method: Rt : 10.283 min,100%; column: YMC CHIRAL ART CELLULOSE-SC (250 × 4.6 mm, 5 u), Mobile Phase: A) n-Hexane+0.1% Iso-propyl amine, B) DCM: MeOH (50:50), Isocratic: 20% B; Wavelength: 287 nm, Flow: 1.0 mL/min.

17: HPLC: Rt 7.804 min, 99.35%; Column: X-Select CSH C18 (4.6 × 150) mm, 3.5 µm; Mobile phase: A: 0.1% Formic acid in water: ACN (95:05), B: ACN; Flow Rate: 1.0 mL/min; LCMS : 406.45 (M+H), Rt 1.921 min, Column: X-select CSH C18 (3^{∗}50) mm, 2.5 µm; ¹H NMR (400 MHz, DMSO-d6) δ 8.94 (d, 1H), 8.59 (d, 1H), 7.86-7.80 (m, 2H), 7.63 (d, 1H), 6.67 (s, 1H), 5.48-5.40 (m, 1H), 3.93 (s, 3H), 2.30-2.25 (m, 1H), 1.67 (d, 3H), 1.05-0.95 (m, 4H).Chiral method: Rt: 12.792 min, 97.84%; column: YMC CHIRAL ART CELLULOSE-SC (250 × 4.6 mm, 5u), Mobile Phase: A) n-Hexane+0.1% Iso-propyl amine, B) DCM: MeOH (50:50), Isocratic: 20% B; Wavelength: 287 nm, Flow: 1.0 mL/min.

Examples 16-1 and 17-1. Synthesis of 2-methyl-N-[(1R)-1-[3-(2-cyclopropyl-4-pyridyl)isoxazol-5-yl]ethyl]-5-(trifluoromethyl)pyrazole-3-carboxamide & N-[(1S)-1-[3-(2-cyclopropyl-4-pyridyl)isoxazol-5-yl]ethyl]-2-methyl-5-(trifluoromethyl)pyrazole-3-carboxamide. Note that stereochemistry is randomly assigned.

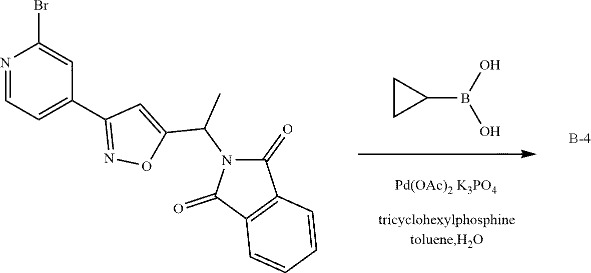

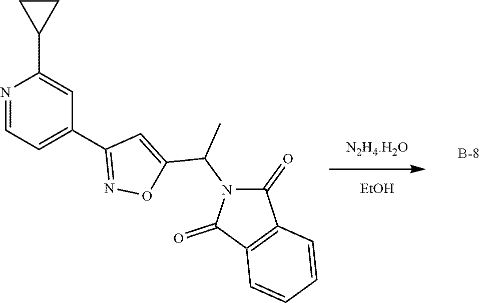

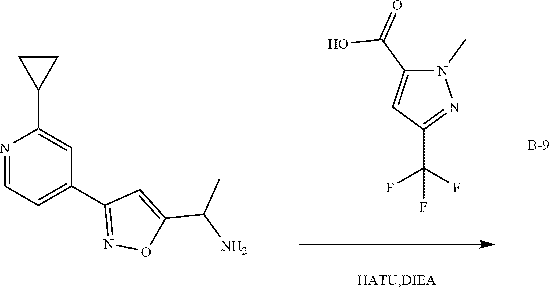

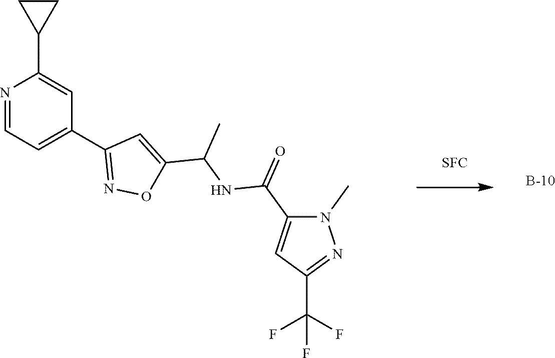 [SMILES: CC(NC(=O)c1cc(C(F)(F)F)nn1C)c1cc(-c2ccnc(C3CC3)c2)no1 ; "SFC"]

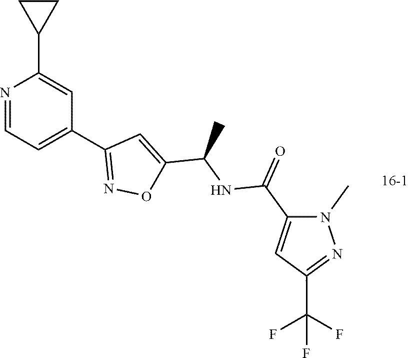 [SMILES: C[C@@H](NC(=O)c1cc(C(F)(F)F)nn1C)c1cc(-c2ccnc(C3CC3)c2)no1]

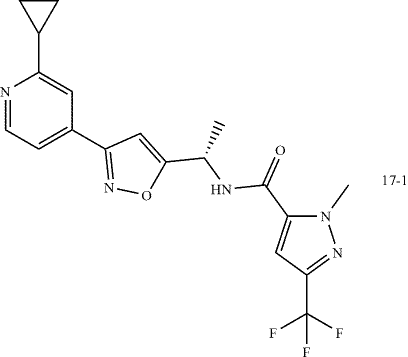 [SMILES: C[C@H](NC(=O)c1cc(C(F)(F)F)nn1C)c1cc(-c2ccnc(C3CC3)c2)no1]

### 2-[3-(2-Cyclopropyl-4-pyridyl)isoxazol-5-yl]ethyl]isoindoline-1,3-dione (B-8)

To a mixture of 2-[1-[3-(2-bromo-4-pyridyl)isoxazol-5-yl]ethyl]isoindoline-1,3-dione (1 g, 2.51 mmol), cyclopropylboronic acid (431.4 mg, 5.02 mmol), K₃PO₄ (1.07 g, 5.02 mmol), Pd(OAc)₂ (28.2 mg, 0.13 mmol) in water (5 mL) and toluene (25 mL) was added PCy₃ (70.4 mg, 0.25 mmol). The mixture was stirred at 120° C. for 16 hours under N₂. The mixture was poured into water (30 mL) and stirred for 20 min. The aqueous phase was extracted with EtOAc (3 × 20 mL). The combined organic phase was washed with saturated brine (2 × 20 mL), dried over anhydrous Na₂SO₄, filtered and concentrated. The residue was purified by silica gel chromatography (PE/EtOAc = 5/1 to 3/1) to afford the product (240 mg, 0.47 mmol, 19% yield) as an oil. LCMS Rₜ = 0.846 min in 1.5 min chromatography, 5-95AB, MS ESI calcd. For C₂₁H₁₈N₃O₃ [M+H]⁺360.1, found 360.0

### 1-(2-Cyclopropyl-4-pyridyl)isoxazol-5-yl]ethanamine (B-9)

To a solution of 2-[1-[3-(2-cyclopropyl-4-pyridyl)isoxazol-5-yl]ethyl]isoindoline-1,3-dione (240 mg, 0.67 mmol) in DCM (10 mL) and ethanol (2 mL) was added NH₂NH₂.H₂O (0.2 mL, 4.01 mmol) dropwise at 25° C. The mixture was stirred at 25° C. for 16 hours. The mixture was filtered and the filter cake was washed with DCM (10 × 3 mL). The filtrate was concentrated to afford the product (150 mg, 0.654 mmol, 98% yield) as a solid. LCMS Rt = 0.21 min in 1.5 min chromatography, 5-95AB, MS ESI calcd. for C₁₃H₁₆N₃O [M+H]⁺230.1, found 229.9

### N-[3-(2-cyclopropyl-4-pyridyl)isoxazol-5-yl]ethyl]-2-methyl-5-(trifluoromethyl)pyrazole-3-carboxamide (B-10)

To a solution of 2-methyl-5-(trifluoromethyl)pyrazole-3-carboxylic acid (139.7 mg, 0.72 mmol), HATU (497.5 mg, 1.31 mmol) in DMF (5 mL) was added Et₃N (0.27 mL, 1.96 mmol) and 1-[3-(2-cyclopropyl-4-pyridyl)isoxazol-5-yl]ethanamine (150 mg, 0.65 mmol). The mixture was stirred at 20° C. for 12 hours, diluted with water (30 mL) and extracted with EtOAc (3 × 20 mL). The organic layers were washed with brine (3 × 30 mL), dried over Na₂SO₄, filtered and the filtrate was concentrated to afford the product, which was purified by flash chromatography on silica gel (MeOH in DCM = 0% to 4%) to afford the product (300 mg) as an oil. ¹H NMR (CDC1₃, 400 MHz) δ_{H} = 8.53 (d, 1H), 7.52-7.49 (m, 1H), 7.40-7.35 (m, 1H), 6.86 (s, 1H), 6.56-6.53 (m, 1H), 6.47 (d, 1H), 5.59-5.49 (m, 1H), 4.23 (s, 3H), 2.14-2.04 (m, 1H), 1.72 (d, 3H), 1.12-0.94 (m, 4H).

### 2-methyl-N-[(1R)-1-[3-(2-cyclopropyl-4-pyridyl)isoxazol-5-yl]ethyl]-5-(trifluoromethyl)pyrazole-3-carboxamide & N-[(1S)-1-[3-(2-cyclopropyl-4-pyridyl)isoxazol-5-yl]ethyl]-2-methyl-5-(trifluoromethyl)pyrazole-3-carboxamide

The mixture of N-[1-[3-(2-cyclopropyl-4-pyridyl)isoxazol-5-yl]ethyl]-2-methyl-5-(trifluoromethyl)pyrazole-3-carboxamide (300 mg, 0.740 mmol) was purified by SFC (Column DAICEL CHIRALCEL OJ-H(250 mm^{∗}30 mm, 5 um), Condition 0.1%NH₃H₂O ETOH, Begin B 30, End B 30, FlowRate(ml/min) 60) to give Peak 1 (90 mg) as a solid and Peak 2 (87.6 mg, 0.213 mmol, 29% yield) as a solid.

The mixture of Peak 1 (90 mg) was purified by prep-TLC (DCM: MeOH=10:1) to give 2-methyl-N-[(1R)-1-[3-(2-cyclopropyl-4-pyridyl)isoxazol-5-yl]ethyl]-5-(trifluoromethyl)pyrazole-3-carboxamide (54.1 mg, 0.134 mmol, 60% yield) as a solid.

16-1: ¹H NMR (CDCl₃, 400 MHz) δ_{H} = 8.51 (d, 1H), 7.50 (s, 1H), 7.41-7.36 (m, 1H), 6.90 (s, 1H), 6.62 (d, 1H), 6.56 (s, 1H), 5.60-5.45 (m, 1H), 4.22 (s, 3H), 2.15-2.10 (m, 1H), 1.72 (d, 3H), 1.12-0.96 (m, 4H). LCMS Rₜ = 1.01 min in 2.0 min chromatography, 10-80AB, MS ESI calcd. for C₁₉H₁₉F₃N₅O₂ [M+H]⁺406.1, found 406.1

17-1: ¹H NMR (CDCl₃, 400 MHz) δ_{H} = 8.53 (d, 1H), 7.51 (s, 1H), 7.40-7.36 (m, 1H), 6.85 (s, 1H), 6.56 (s, 1H), 6.37 (d, 1H), 5.60-5.47 (m, 1H), 4.23 (s, 3H), 2.13-2.01 (m, 1H), 1.72 (d, 3H), 1.13-0.99 (m, 4H). LCMS Rₜ = 1.00 min in 2.0 min chromatography, 10-80AB, MS ESI calcd. for C₁₉H₁₉F₃N₅O₂ [M+H]⁺406.1, found 406.1.

Example 16-2. Synthesis of 2-methyl-N-[(1R)-1-[3-(2-cyclopropyl-4-pyridyl)isoxazol-5-yl]ethyl]-5-(trifluoromethyl)pyrazole-3-carboxamide (16-2)

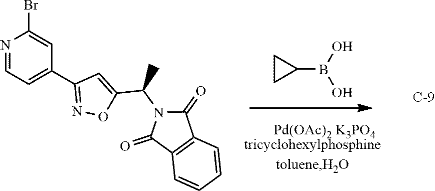

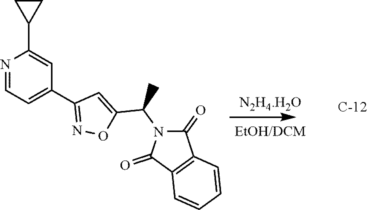

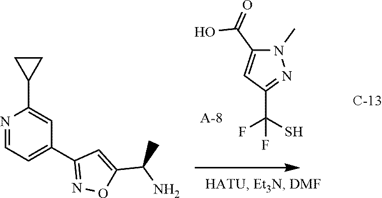

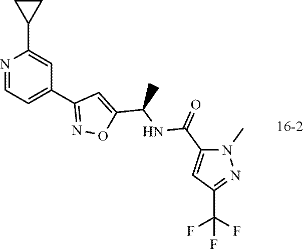 [SMILES: C[C@@H](NC(=O)c1cc(C(F)(F)F)nn1C)c1cc(-c2ccnc(C3CC3)c2)no1]

### 2-[(1R)[3-(2-cyclopropyl-4-pyridyl)isoxazol-5-yl]ethyl]isoindoline-1,3-dione (C-12)

To a mixture of 2-[-(1R)-1-[3-(2-bromo-4-pyridyl)isoxazol-5-yl]ethyl]isoindoline-1,3-dione (500 mg, 1.3 mmol), cyclopropylboronic acid (216 mg, 2.5 mmol), K₃PO₄ (533 mg, 2.5 mmol), PCy₃ (35 mg, 0.13 mmol) in H₂O (5.0 mL) and toluene (25 mL) was added Pd(OAc)₂ (14 mg, 0.060 mmol) under N₂. After stirring at 110° C. for 16 hour, the mixture was poured into water (30 mL) and extracted with EtOAc (3 × 20 mL). The combined organic phase was washed with saturated brine (2 × 20 mL), dried over anhydrous Na₂SO₄, filtered and concentrated. The residue was purified by silica gel chromatography (PE/EtOAc = 5/1 to 3/1) to afford the product (270 mg, 0.53 mmol, 42% yield) as an oil. The mixture (70 mg, 0.19 mmol) was purified by prep-HPLC (Column: Phenomenex Gemini-NX 80 ^{∗} 30 mm ^{∗} 3 µm, Condition: water (10 mM NH₄HCO₃)-CAN; Begin B: 40, End B: 70, Gradient Time(min): 9) and prep-TLC (DCM/acetone= 50/1) to afford the product (19.65 mg, 0.050 mmol, 28% yield) a solid. ¹H NMR (CDCl₃, 400 MHz) δ_{H} = 8.52 (d, 1H), 7.92-7.84 (m, 2H), 7.80-7.72 (m, 2H), 7.51 (s, 1H), 7.41-7.37 (m, 1H), 6.67-6.63 (m, 1H), 5.77-5.69 (m, 1H), 2.14-2.02 (m, 1H), 1.95 (d, 3H), 1.13-0.96 (m, 4H). LCMS Rₜ = 0.995 min in 2.0 min chromatography, 10-80AB, MS ESI calcd. for C₂₁H₁₈N₃O₃ [M+H]⁺ 360.1, found 360.1.

### (1R)[3-(2-cyclopropyl-4-pyridyl)isoxazol-5-yl]ethanamine (C-13)

To a solution of 2-[(1R)-1-[3-(2-cyclopropyl-4-pyridyl)isoxazol-5-yl]ethyl]isoindoline-1,3-dione (100 mg, 0.28 mmol) in DCM (10 mL) and EtOH (2.0 mL) was added N₂H₄.H₂O (0.080 mL, 1.7 mmol) dropwise at 25° C. After stirring at 25° C. for 16 hours, the mixture was filtered and the filter cake was washed with DCM (3 × 10 mL). The filtrate was concentrated to afford the product (60 mg, 0.26 mmol, 94% yield) as a solid. LCMS Rₜ = 0.203 min in 1.5 min chromatography, 5-95AB, MS ESI calcd. for C₁₃H₁₆N₃O [M+H]⁺229.9, found 229.9

### 2-methyl-N-[(1R)-1-[3-(2-cyclopropyl-4-pyridyl)isoxazol-5-yl]ethyl]-5-(trifluoromethyl)pyrazole-3-carboxamide

To a solution of 2-methyl-5-(trifluoromethyl)pyrazole-3-carboxylic acid (47 mg, 0.24 mmol), HATU (166 mg, 0.44 mmol) in DMF (5.0 mL) was added Et₃ N (0.090 mL, 0.65 mmol) and (1R)-1-[3-(2-cyclopropyl-4-pyridyl)isoxazol-5-yl]ethanamine (50 mg, 0.22 mmol) at 20° C. After stirring for 1 hour, water (10 mL) was added and the solution was extracted with EtOAc (3 × 10 mL), The organic layer was washed with brine (3 × 10 mL), dried over Na₂SO₄, filtered and concentrated to give the product which was purified by prep-HPLC (Column: Phenomenex Gemini-NX 80 ^{∗} 30 mm ^{∗} 3 µm, Condition: water (10 mM NH₄HCO₃)-ACN, Begin: B 40, End B: 70, Gradient Time (min): 9) and purified by prep-TLC (DCM/acetone= 50/1) to afford the product (40.9 mg, 0.10 mmol, 58% yield) as a solid. ¹H NMR (CDCl₃, 400 MHz) δ_{H} = 8.54 (d, 1H), 7.52 (s, 1H), 7.44-7.37 (m, 1H), 6.88-6.82 (m, 1H), 6.58-6.52 (m, 1H), 6.41-6.33 (m, 1H), 5.58-5.47 (m, 1H), 4.23 (s, 3H), 2.20-2.06 (m, 1H), 1.73 (d, 3H), 1.13-0.98 (m, 4H). ¹⁹F NMR (376.5 MHz, CDCl3) δ_{F} -62.214. LCMS Rt = 0.980 min in 2.0 min chromatography, 10-80AB, MS ESI calcd. for C₁₉H₁₉F₃N₅O₂ [M+H]⁺406.2, found 406.2.

Example 17-2. Synthesis of 2-methyl-N-[(1S)-1-[3-(2-cyclopropyl-4-pyridyl)isoxazol-5-yl]ethyl]-5-(trifluoromethyl)pyrazole-3-carboxamide (17-2)

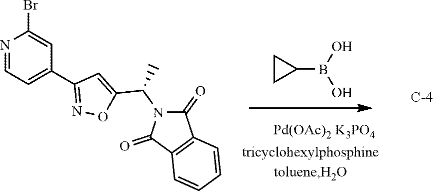

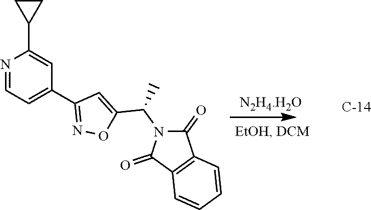

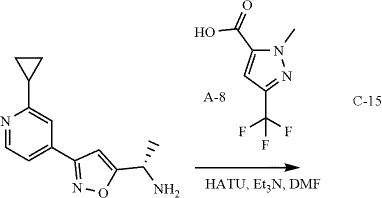

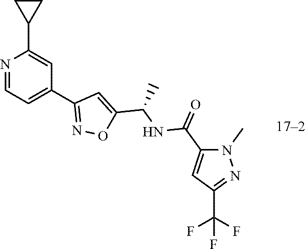 [SMILES: C[C@H](NC(=O)c1cc(C(F)(F)F)nn1C)c1cc(-c2ccnc(C3CC3)c2)no1]

### 2-[(1S)[3-(2-cyclopropyl-4-pyridyl)isoxazol-5-yl]ethyl]isoindoline-1,3-dione (C-14)

To a mixture of 2-[(1S)-1-[3-(2-bromo-4-pyridyl)isoxazol-5-yl]ethyl]isoindoline-1,3-dione (500 mg, 1.3 mmol), cyclopropylboronic acid (216 mg, 2.5 mmol), K₃PO₄ (533 mg, 2.5 mmol), Pd(OAc)₂ (14 mg, 0.060 mmol) in H₂O (2.0 mL) and toluene (10 mL)was added tricyclohexylphosphine (35 mg, 0.13 mmol). After stirring at 110° C. for 16 hours under N₂, the mixture was poured into water (30 mL) and stirred for 20 mins. The aqueous phase was extracted with EtOAc (3 × 20 mL). The combined organic phase was washed with saturated brine (2 × 80 mL), dried over anhydrous Na₂SO₄, filtered and concentrated. The residue was purified by silica gel chromatography (PE/EtOAc = 5/1 to 3/1) to afford the product (390 mg, 0.75 mmol, 61% yield) as an oil. The product (100 mg, 0.28 mmol) was purified by HPLC (Column Phenomenex Gemini-NX 80 ^{∗} 30 mm ^{∗} 3 µm; Condition: water (10 mM NH₄HCO₃)-CAN; Begin B: 42; End B: 72; Gradient Time (min): 9; 100% B Hold Time (min): 1.5; FlowRate (mL/min): 30) to afford the product (14.5 mg, 0.040 mmol, 36% yield) as a solid. ¹H NMR (CDCl₃, 400 MHz) δ_{H} = 8.52 (d, 1H), 7.89-7.85 (m, 2H), 7.78-7.74 (m, 2H), 7.51 (s, 1H), 7.40 (d, 1H), 6.66 (d,1H), 5.80-5.64 (m, 1H), 2.19-2.05 (m, 1H), 1.95 (d, 3H), 1.13-0.97 (m, 4H). LCMS Rₜ = 0.871 min in 1.5 min chromatography, 5-95AB, MS ESI calcd. for C₂₁H₁₈N₃O₃ [M+H]⁺ 360.0, found 360.0.

### (1S)[3-(2-cyclopropyl-4-pyridyl)isoxazol-5-yl]ethanamine (C-15)

To a solution of 2-[(1S)-1-[3-(2-cyclopropyl-4-pyridyl)isoxazol-5-yl]ethyl]isoindoline-1,3-dione (140 mg, 0.39 mmol) in DCM (15 mL) and EtOH (3.0 mL) was added N₂H_{4.}H₂O (0.12 mL, 2.3 mmol) dropwise at 25° C. After stirring at 25° C. for 16 hrs, the mixture was filtered and the filter cake was washed with DCM (3 × 10 mL). The filtrate was concentrated to afford the product (100 mg, 0.30 mmol, 78% yield) as a solid which was used directly for the next step.

### 2-methyl-N-[(1S)-1-[3-(2-cyclopropyl-4-pyridyl)isoxazol-5-yl]ethyl]-5-(trifluoromethyl)pyrazole-3-carboxamide

To a solution of 2-methyl-5-(trifluoromethyl)pyrazole-3-carboxylic acid (93 mg, 0.48 mmol), HATU (332 mg, 0.87 mmol) in DMF (10 mL) was added Et₃N (0.18 mL, 1.3 mmol) and (1S)-1-[3-(2-cyclopropyl-4-pyridyl)isoxazol-5-yl]ethanamine (100 mg, 0.44 mmol). After stirring at 20° C. for 12 hours, the reaction mixture was diluted with water (30 mL) and extracted with EtOAc (3 × 20 mL), The organic layer was washed with water (3 × 30 mL) and brine (3 × 30 mL), dried over Na₂SO₄, filtered and the filtrate was concentrated to give the product which was purified by prep-HPLC (Column Phenomenex Gemini-NX 80 ^{∗} 30 mm ^{∗} 3 µm Condition: water (10 mM NH₄HCO₃)- CAN; Begin B: 42; End B: 72; Gradient Time (min): 9; 100% B Hold Time (min): 1.5; FlowRate (mL/min): 30) and SFC (Column: DAICEL CHIRALPAK AD (250 mm ^{∗} 30 mm, 10 um); Condition: 0.1% NH₃H₂O IPA; Begin B: 15%; End B: 15%; FlowRate (mL/min): 50) to afford the product (27.1 mg, 0.067 mmol, 46% yield) as a solid. ¹H NMR (CDCl₃, 400 MHz) δ_{H} = 8.54 (d, 1H), 7.51 (s, 1H), 7.39 (d, 1H), 6.85 (s, 1H), 6.56 (s, 1H), 6.41-6.25 (m, 1H), 5.64-5.45 (m, 1H), 4.23 (s, 3H), 2.18-2.02 (m, 1H), 1.73 (d, 3H), 1.14-0.98 (m, 4H). ¹⁹F NMR (376.5 MHz, CDC13) δ_{F} = -62.223. LCMS Rₜ = 0.870 min in 1.5 min chromatography, 5-95AB, MS ESI calcd. for C₁₉H₁₉F₃N₅O₂ [M+H]⁺405.9, found 405.9.

Examples 18 and 19. Synthesis of (S)-3-chloro-N-(1-(3-(2-cyclopropylpyridin-4-yl)isoxazol-5-yl)ethyl)benzamide (18) and (R)-3-chloro-N-(1-(3-(2-cyclopropylpyridin-4-yl)isoxazol-5-yl)ethyl)benzamide (19). Note the stereochemistry is randomly assigned.

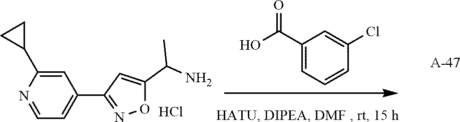

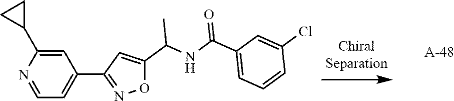

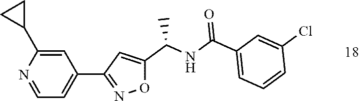 [SMILES: C[C@H](NC(=O)c1cccc(Cl)c1)c1cc(-c2ccnc(C3CC3)c2)no1]

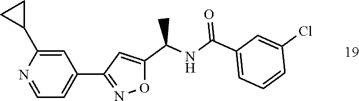 [SMILES: C[C@@H](NC(=O)c1cccc(Cl)c1)c1cc(-c2ccnc(C3CC3)c2)no1]

To a stirred solution of 3-chlorobenzoic acid (0.204 g, 1.310 mmol) in DMF (2 mL) was added DIPEA (0.76 mL, 4.360 mmol) and HATU (0.663 g, 1.740 mmol) and stirred for 5 min. To the resulting solution was added a solution of A-47 (0.400 g, 1.744 mmol) in DMF (1 mL) at room temperature and stirred for 15 h. The reaction mixture was quenched with water (20 mL) and extracted with ethyl acetate (4 × 10 mL). The combined organic layer was washed with water (20 mL), separated and dried over anhydrous Na₂SO₄, filtered and concentrated under reduced pressure to give crude A-48. Chiral separation of A-48 was performed by preparative chiral HPLC to afford 18 (0.044 g, 0.119 mmol, 14% yield) and 19 (0.048 g, 0.125 mmol, 14% yield) as an oil.

18: LCMS : 367.95 (M+H), Rₜ= 1.883 min, Column : Kinetex EVO C18 (50^{∗}3) mm; 2.6 u; Mobile Phase: A: 5 mM Ammonium Bicarbonate in water; B: Acetonitrile; HPLC: Rₜ = 5.400 min, 99.42%; Column; X SELECT CSH C18 (150×4.6 mm,3.5 um); Mobile Phase A 5 mM AMMONIUM BICARBONATE; Mobile Phase B : ACETONITRILE; CHIRAL HPLC: Rₜ = 8.100 min, 96.42%; Column: CHIRAL PAK IC (250 × 4.6 mm, 5 µm), Mobile Phase: A) 0.1% DEA in n-Hexane, B) EtOH (50:50), A:B: 75:25; Flow: 1.00 mL/min. ¹H NMR (400 MHz, DMSO-d₆) δ ppm 9.17 (d, 1H), 8.51 (d, 1H), 7.97 (s, 1H), 7.88 (d, 1H), 7.76 (s, 1H), 7.64 (d, 1H), 7.52-7.58 (m, 2H), 7.12 (s, 1H), 5.40-5.44 (m, 1H), 2.10-2.25 (m, 1H), 1.60 (d, 3H), 0.90-1.01 (m, 4H).

19: LCMS : 367.95 (M+H), Rₜ= 1.882 min, Column : Kinetex EVO C18 (50*3) mm; 2.6 u; Mobile Phase: A: 5 mM Ammonium Bicarbonate in water; B: Acetonitrile; HPLC: Rₜ = 7.300 min, 96.20%Column; X SELECT CSH C18 (150×4.6 mm,3.5 um); Mobile Phase A 5 mM AMMONIUM BICARBONATE; Mobile Phase B : ACETONITRILE; CHIRAL HPLC: Rₜ = 6.409 min, 97.83%; Column: CHIRAL PAK IC (250 × 4.6 mm, 5 µm), Mobile Phase: A) 0.1% DEA in n-Hexane, B) EtOH (50:50), A:B :: 75:25; Flow: 1.00 mL/min. ¹H NMR (400 MHz, DMSO-d₆) δ ppm 9.18 (d, 1H), 8.51 (d, 1H), 7.98 (s, 1H), 7.88 (d, 1H), 7.76 (s, 1H), 7.64 (d, 1H), 7.52-7.58 (m, 2H), 7.12 (s, 1H), 5.40-5.44 (m, 1H), 2.10-2.25 (m, 1H), 1.60 (d, 3H), 0.90-1.02 (m, 4H).

Examples 20 and 21. Synthesis of (R)-3-chloro-N-(1-(3-(2-(trifluoromethyl)pyridin-4-yl)isoxazol-5-yl)ethyl)benzamide (20) and (S)-3-chloro-N-(1-(3-(2-(trifluoromethyl)pyridin-4-yl)isoxazol-5-yl)ethyl)benzamide (21). Note the stereochemistry is randomly assigned.

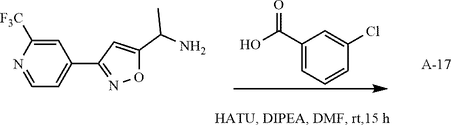

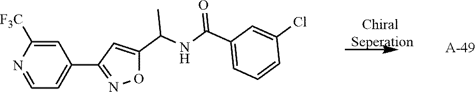

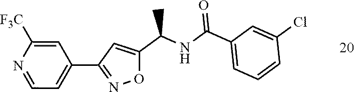 [SMILES: C[C@@H](NC(=O)c1cccc(Cl)c1)c1cc(-c2ccnc(C(F)(F)F)c2)no1]

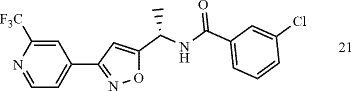 [SMILES: C[C@H](NC(=O)c1cccc(Cl)c1)c1cc(-c2ccnc(C(F)(F)F)c2)no1]

To a stirred solution of A-17 (0.200 g, 0.777 mmol) and 3-chlorobenzoic acid (0.243 g, 1.555 mmol) in DMF (5 mL) was added HATU (0.591 g, 1.555 mmol) followed by DIPEA (0.677 mL, 3.887 mmol) at room temperature and stirred for 15 h. The reaction mixture was diluted with water (10 mL) and extracted with ethyl acetate (2 × 25 mL). The combined organic layer was washed with water (20 mL), dried over anhydrous Na₂SO₄, filtered and concentrated under reduced pressure resulting in the residue (220 mg) as a liquid. The residue was purified by Combiflash column chromatography eluting with 0-40% ethyl acetate in n-hexane to afford A-49 (0.145 g) as a solid. Chiral separation of A-49 was performed by preparative chiral HPLC to afford 20 (0.034 g, 0.086 mmol, 11% yield) and 21 (0.036 g, 0.088 mmol, 11% yield) as solids.

20: LCMS : 393.90 (M-H), Rₜ = 2.118 min, Column: Kinetex EVO C18 (50^{∗}3) mm 2.6 µ; Mobile Phase: A: 2.5 mM Ammonium Bicarbonate in water, B: Acetonitrile; HPLC: Rₜ = 6.030 min, 99.20%; Column: X SELECT CSH C18 (150 × 4.6 mm, 3.5 um); Mobile Phase A 5 mM AMMONIUM ACETATE; Mobile Phase B: ACETONITRILE; Flow: 1.0 mL/min. CHIRAL HPLC: Rₜ = 7.878 min, 99.25%Column: Chiralpak IG (250 ×4.6 mm, 5 µm); Mobile Phase:A-0.1% DEA in n-Hexane

Mobile Phase: DCM: MEOH (50:50); A:B: 80:20; Flow Rate : 1.0 mL/min. ¹H NMR (400 MHz, DMSO-d₆) δ ppm 9.21 (d, 1H), 8.93 (d, 1H), 8.34 (s, 1H), 8.22 (d, 1H), 7.99 (t, 1H), 7.89 (d, 1H), 7.62-7.67 (m, 1H), 7.52-7.57 (m, 1H), 7.35 (s, 1H), 5.40-5.47 (m, 1H), 1.62 (d, 3H).

21: LCMS : 393.95 (M+H), Rₜ= 2.119 min, Column: Kinetex EVO C18 (50^{∗}3) mm 2.6 µ; Mobile Phase: A: 2.5 mM Ammonium Bicarbonate in water, B: Acetonitrile; HPLC: Rₜ = 12.29 min, 96.04%; Column: X SELECT CSH C18 (150 × 4.6 mm, 3.5 um); Mobile Phase A 0.05% TFA in water: Acetonitrile (95:05); Mobile Phase B: 0.05% TFA in water: Acetonitrile (5:95); Flow: 1.0 mL/min..CHIRAL HPLC: Rₜ = 14.46 min, 99.57%; Column: Chiralpak IG (250 ×4.6 mm, 5 µm); Mobile Phase:A-0.1% DEA in n-Hexane; Mobile Phase: DCM: MEOH (50:50); A:B: 80:20; Flow Rate : 1.0 mL/min; ¹H NMR (400 MHz, DMSO-d₆) δ ppm 9.20 (d, 1H), 8.92 (d, 1H), 8.33 (s, 1H), 8.21 (d, 1H), 7.98 (t, 1H), 7.88 (d, 1H), 7.64 (dd, 1H), 7.51-7.57 (m, 1H), 7.35 (s, 1H), 5.40-5.47 (m, 1H), 1.61 (d, 3H).

Examples 22 and 23. Synthesis of (R)-N-(1-(3-(2-(trifluoromethyl)pyridin-4-yl)isoxazol-5-yl)ethyl)benzamide (22) and (S)-N-(1-(3-(2-(trifluoromethyl)pyridin-4-yl)isoxazol-5-yl)ethyl)benzamide (23). Note the stereochemistry is randomly assigned.

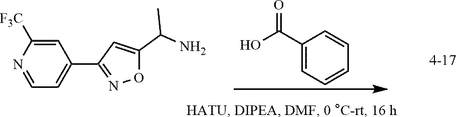

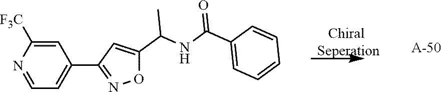

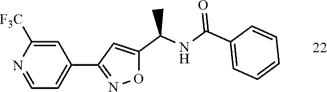 [SMILES: C[C@@H](NC(=O)c1ccccc1)c1cc(-c2ccnc(C(F)(F)F)c2)no1]

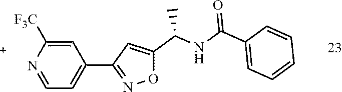 [SMILES: C[C@H](NC(=O)c1ccccc1)c1cc(-c2ccnc(C(F)(F)F)c2)no1]

To a stirred solution of benzoic acid (0.142 g, 1.166 mmol) in DMF (2 mL) at 0° C. was added DIPEA (0.677 mL, 3.884 mmol) followed by HATU (0.591 g, 1.554 mmol) and stirred for 5 min. To the resulting solution was added a solution of A-17 (0.200 g, 0.777 mmol) in DMF (2 mL). The reaction mixture was allowed to attain room temperature and stirred for 16 h. The reaction mixture was diluted with water (20 mL) and extracted with ethyl acetate (4 × 10 mL). The combined organic layer was washed with water (20 mL), dried over anhydrous Na₂SO₄, filtered and concentrated under reduced pressure resulting in the residue A-50. The residue A-50 was subjected to Chiral HPLC purification to afford 22 (0.025 g, 0.069 mmol, 9% yield) and 23 (0.026 g, 0.072 mmol, 9% yield) as a solid.

22: LCMS : 360.05 (M+H), Rₜ = 2.022 min, Column: Kinetex EVO C18 (50^{∗}3) mm 2.6 µ; Mobile Phase: A: 2.5 mM Ammonium Bicarbonate in water, B: Acetonitrile; HPLC: Rₜ = 6.920 min, 99.47%

Column; X SELECT CSH C18 (150 × 4.6 mm,3.5 um); Mobile Phase A 5 mM AMMONIUM BICARBONATE; Mobile Phase B : ACETONITRILE; CHIRAL HPLC: Rₜ = 9.089 min, 100%; Column: Chiralpak IG (250 ×4.6 mm, 5 µm); Mobile Phase:A-0.1% DEA in n-Hexane; Mobile Phase: DCM: MEOH (50:50); A:B: 80:20; Flow Rate : 1.0 mL/min. ¹H NMR (400 MHz, DMSO-d₆) δ 9.07 (d, 1H), 8.92 (d, 1H), 8.34 (s, 1H), 8.22 (dd, 1H), 7.90-7.96 (m, 2H), 7.47-7.60 (m, 3H), 7.33 (d, 1H), 5.42-5.49 (m, 1H), 1.62 (d, 3H).

23: LCMS : 362.10 (M+H), Rₜ= 2.165 min, Column: X-Bridge BEH C-18 (3.0^{∗}50 mm, 2.5 µm); Mobile Phase: A: 0.02 \.5% Formic acid in water, B: Acetonitrile; HPLC: Rt = 5.580 min, 95.35%; Column: X SELECT CSH C18 (150 ×4.6 mm, 3.5 um); Mobile Phase A 5 mM AMMONIUM ACETATE; Mobile Phase B: ACETONITRILE; CHIRAL HPLC: Rₜ = 12.12 min, 97.07%; Column: Chiralpak IG (250 ×4.6 mm, 5 µm); Mobile Phase: A-0.1% DEA in n-Hexane; Mobile Phase: DCM: MEOH (50:50); A:B: 80:20; Flow Rate: 1.0 mL/min. ¹H NMR (400 MHz, DMSO-d₆) δ 9.07 (d, 1H), 8.92 (d, 1H), 8.34 (s, 1H), 8.21 (d, 1H), 7.89-7.95 (m, 2H), 7.47-7.59 (m, 3H), 7.33 (d, 1H), 5.41-5.49 (m, 1H), 1.62 (d, 3H).

Examples 24 and 25. Synthesis of (R)-3-isopropyl-1-methyl-N(1-(3-(2-(trifluoromethyl)pyridin-4-yl)isoxazol-5-yl)ethyl)-1H-pyrazole-5-carboxamide (24) and (N)-3-isopropyl-1-methyl-N-(1-(3-(2-(trifluoromethyl)pyridin-4-yl)isoxazol-5-yl)ethyl)-1H pyrazole-5-carboxamide (25). Note the stereochemistry is randomly assigned.

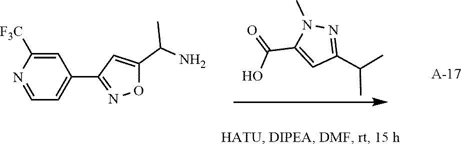

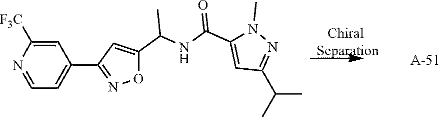

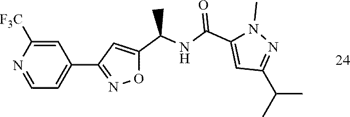 [SMILES: CC(C)c1cc(C(=O)N[C@H](C)c2cc(-c3ccnc(C(F)(F)F)c3)no2)n(C)n1]

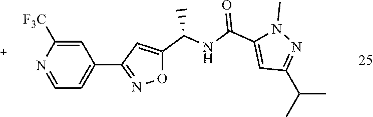 [SMILES: CC(C)c1cc(C(=O)N[C@@H](C)c2cc(-c3ccnc(C(F)(F)F)c3)no2)n(C)n1]

To a stirred solution of A-17 (0.300 g, 1.166 mmol) and 3-isopropyl-1-methyl-1H-pyrazole-5-carboxylic acid (0.226 g, 1.341 mmol) in DMF (5 mL) was added HATU (0.886 g, 2.332 mmol) followed by DIPEA (1.01 mL, 5.830 mmol) at room temperature and stirred for 15 h. The reaction mixture was diluted with water (10 mL) and extracted with ethyl acetate (2 × 25 mL). The combined organic layer was washed with water (20 mL), dried over anhydrous Na₂SO₄, filtered and concentrated under reduced pressure resulting in the residue A-51 (198 mg) as a liquid. The residue was purified by Combiflash column chromatography eluting with 0-40% ethyl acetate in n-hexane to afford A-51 (0.200 g) as a solid. Chiral separation of A-51 was done by preparative chiral HPLC to afford 24 (0.060 g, 0.147 mmol, 13% yield) and 25 (0.086 g, 0.211 mmol, 18% yield) as solids.

24: LCMS : 407.95 (M+H), Rₜ = 2.722 min, Column: Kinetex EVO C18 (50^{∗}3) mm 2.6 µ; Mobile Phase: A: 2.5 mM Ammonium Bicarbonate in water, B: Acetonitrile; HPLC: Rₜ = 4.959 min, 98.71%

Column; X SELECT CSH C18 (150 × 4.6 mm, 3.5 um); Mobile Phase A 5 mM AMMONIUM BICARBONATE; Mobile Phase B : ACETONITRILE; CHIRAL HPLC: Rₜ = 7.233 min, 95.77%; Column: Chiralpak IG (250 ×4.6 mm, 5 µm); Mobile Phase: A-0.1% DEA in n-Hexane; Mobile Phase B: EtOH; A:B: 80:20; Flow Rate : 1.0 mL/min. ¹H NMR (400 MHz, DMSO-d₆) δ ppm 8.97 (d, 1H), 8.93 (d, 1H), 8.34 (s, 1H), 8.20-8.23 (m, 1H), 7.34 (d, 1H), 6.82 (s, 1H), 5.35-5.42 (m, 1H), 3.99 (s, 3H), 2.84-2.91 (m, 1H), 1.59 (d, 3H), 1.20 (d, 6H). 25: LCMS : 408.20 (M+H), Rₜ = 2.232 min, Column: X-Bridge BEH C-18 (3.0^{∗}50 mm, 2.5 µm); Mobile Phase: A: 0.02 \.5% Formic acid in water, B: Acetonitrile; HPLC: Rₜ = 7.240 min, 94.88%

Column; X SELECT CSH C18 (150 × 4.6 mm, 3.5 um); Mobile Phase A 5 mM AMMONIUM BICARBONATE; Mobile Phase B: ACETONITRILE; CHIRAL HPLC: Rₜ = 6.330 min, 99.04%; Column: Chiralpak IG (250 ×4.6 mm, 5 µm); Mobile Phase: A-0.1% DEA in n-Hexane; Mobile Phase B: EtOH; A:B: 80:20; Flow Rate : 1.0 mL/min. ¹H NMR (400 MHz, DMSO-d₆) δ ppm 8.97 (d, 1H), 8.93 (d, 1H), 8.34 (s, 1H), 8.21 (d, 1H), 7.34 (d, 1H), 6.82 (s, 1H), 5.35-5.42 (m, 1H), 3.99 (s, 3H), 2.84-2.91 (m, 1H), 1.59 (d,, 3H), 1.20 (d, 6H).

Examples 26 and 27. Synthesis of (R)-N-(1-(3-(2-(trifluoromethyl)pyridin-4-yl)isoxazol-5-yl)ethyl)cyclohexanecarboxamide (26) and (S)-N-(1-(3-(2-(trifluoromethyl)pyridin-4-yl)isoxazol-5-yl)ethyl)cyclohexanecarboxamide (27). Note the stereochemistry is randomly assigned.

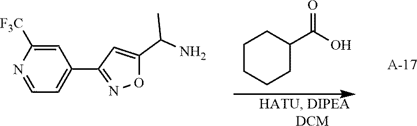

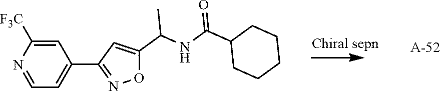

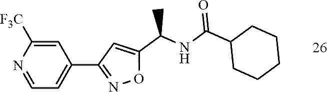 [SMILES: C[C@@H](NC(=O)C1CCCCC1)c1cc(-c2ccnc(C(F)(F)F)c2)no1]

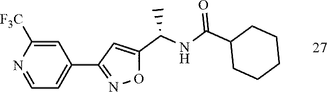 [SMILES: C[C@H](NC(=O)C1CCCCC1)c1cc(-c2ccnc(C(F)(F)F)c2)no1]

To a stirred reaction mixture of A-17 (0.200 g, 0.780 mmol) and cyclohexylcarboxylic acid (249.21 mg, 1.56 mmol) in DMF (5.00 mL) was added HATU (591.31 mg, 1.56 mmol) followed by N,N-Diisopropylethylamine (0.68 mL, 3.89 mmol) at room temperature and stirred at RT for 15 h. The reaction mixture was quenched by adding water (10.0 mL) and then the reaction mixture was extarcted with EtOAc (2×25 mL), the combined extracts were dried over anhydrous Na₂SO₄, filtered, concentrated under reduced pressure to obtain the residue A-52 (198 mg) as a liquid. The residue was purified by Combi-Flash column chromatography (100-200 silica gel) by eluting 0-40% EtOAc in hexanes followed by reverse phase preparative chiral HPLC to obtain 26 (31 mg, 0.084 mmol, 11%) and 27 (32 mg, 0.087 mmol, 11%) both as solids.

26: HPLC: Rt: 10.64 min, 99.51%; Column; X SELECT CSH C18 (150×4.6 mm,3.5 um); Mobile Phase A ;5 mM AMMONIUM BICARBONATE; Mobile Phase B : ACETONITRILE; LCMS : 366.05 (M-H), Rt 2.184 min, Column: Kinetex EVO C18 (50^{∗}3) mm 2.6 u; Mobile Phase: A: 2.5 mM Ammonium Bicarbonate in water, B: Acetonitrile Inj Volume: 2 µL; Flow Rate: 1.2 mL/minute; CHIRAL HPLC: Rt: 7.479 min, 100%; COLUMN: CHIRAL PAK IA (150^{∗}4.6 mm, 3 µm); MOBILE PHASE A: 0.1%DEA in n-Hexane; MOBILE PHASE B: IPA.¹H NMR (400 MHz, DMSO-d₆) δ 8.96 - 8.88 (m, 1H), 8.37 (d, 1H), 8.33 - 8.29 (m, 1H), 8.23 - 8.16 (m, 1H), 7.22 - 7.15 (m, 1H), 5.23 - 5.11 (m, 1H), 2.22 - 2.10 (m, 1H), 1.72 (br d, 4H), 1.66 - 1.56 (m, 1H), 1.52 - 1.42 (m, 3H), 1.42 - 1.28 (m, 2H), 1.25 - 1.08 (m, 3H).

27: HPLC: Rt: 10.63 min, 99.85%; Column: X SELECT CSH C18 (150×4.6 mm,3.5 um); Mobile Phase A ;5 mM AMMONIUM BICARBONATE; Mobile Phase B : ACETONITRILE; LCMS : 368.05 (M+H), Rt 2.155 min, Column: Kinetex EVO C18 (50^{∗}3) mm 2.6 u; Mobile Phase: A: 2.5 mM Ammonium Bicarbonate in water; B: Acetonitrile; Inj Volume: 2 µL, Flow Rate: 1.2 mL/minute; CHIRAL HPLC: Rt 12.717 min, 99.85%; COLUMN: CHIRAL PAK IA (150^{∗}4.6 mm, 3 µm); MOBILE PHASE A: 0.1%DEA in n-Hexane; MOBILE PHASE B: IPA.¹H NMR (400 MHz, DMSO-d₆) δ 8.95 - 8.90 (m, 1H), 8.37 (d, 1H), 8.33 - 8.29 (m, 1H), 8.20 (dd, 1H), 7.20 - 7.15 (m, 1H), 5.22 - 5.12 (m, 1H), 2.22 - 2.11 (m, 1H), 1.72 (br d, 4H), 1.66 - 1.57 (m, 1H), 1.46 (d, 3H), 1.42 - 1.28 (m, 2H), 1.28 - 1.11 (m, 3H).

Examples 28 and 29. Synthesis of (R)-2-phenyl-N-(1-(3-(2-(trifluoromethyl)pyridin-4-yl)isoxazol-5-yl)ethyl)acetamide (28) and (S)-2-phenyl-N-(1-(3-(2-(trifluoromethyl)pyridin-4-yl)isoxazol-5-yl)ethyl)acetamide (29). Note the stereochemistry is randomly assigned.

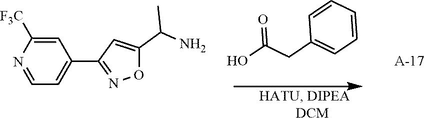

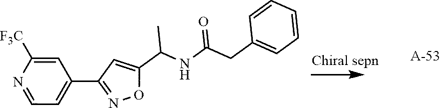

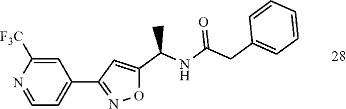 [SMILES: C[C@@H](NC(=O)Cc1ccccc1)c1cc(-c2ccnc(C(F)(F)F)c2)no1]

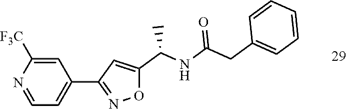 [SMILES: C[C@H](NC(=O)Cc1ccccc1)c1cc(-c2ccnc(C(F)(F)F)c2)no1]

To a solution of phenylacetic acid (127.04 mg, 0.930 mmol) in DMF (3 mL) were added N,N-Diisopropylethylamine (0.68 mL, 3.89 mmol), HATU (591.31 mg, 1.56 mmol) and A-17 (dissolved in 1 mL DMF, 200 mg, 0.78 mmol) at 0° C. and stirred at room temperature for 12 h. The reaction mixture was quenched by adding water (10.0 mL) and then the reaction mixture was extracted with EtOAc (2×25 mL), the combined extracts were dried over anhydrous Na2SO4, filtered, concentrated under reduced pressure. The residue was purified by Combi-Flash column chromatography (100-200 silica gel) followed by reverse phase preparative chiral HPLC obtain to 28 (38 mg, 0.101 mmol, 13%) and 29 (40 mg, 0.103 mmol, 13%) both as solids.

28: HPLC: Rt: 10.02 min, 99.68%; Column; X SELECT CSH C18 (150×4.6 mm,3.5 um); Mobile Phase A ;5 mM AMMONIUM BICARBONATE; Mobile Phase B : ACETONITRILE; LCMS : 374.05 (M-H), Rt 2.325 min, Column : Kinetex EVO C18 (50^{∗}3) mm 2.6 u; Mobile Phase: A: 2.5 mM Ammonium Bicarbonate in water; B: Acetonitrile; Inj Volume: 2 µL, Flow Rate: 1.2 mL/minute; CHIRAL HPLC: Rt: 11.139 min, 99.77%; COLUMN: CHIRAL PAK IC (150^{∗}4.6 mm, 3 µm); MOBILE PHASE A: 0.1%DEA in n-Hexane; MOBILE PHASE B: DCM:MEOH(50:50).¹H NMR (400 MHz, DMSO-d₆) δ 8.97 -8.88 (m, 1H), 8.86 - 8.76 (m, 1H), 8.33 - 8.25 (m, 1H), 8.21 - 8.13 (m, 1H), 7.37 - 7.16 (m, 6H), 5.24 - 5.11 (m, 1H), 3.54 - 3.43 (m, 2H), 1.49 (d, 3H).

29: HPLC: Rt: 7.17 min, 97.32%; Column: X SELECT CSH C18 (150×4.6 mm,3.5 um); Mobile Phase A ;0.05% FORMIC ACID IN WATER; Mobile Phase B : ACETONITRILE; LCMS : 374.05 (M-H), Rt 2.109 min, Column: Kinetex EVO C18 (50^{∗}3) mm 2.6 u; Mobile Phase: A: 2.5 mM Ammonium Bicarbonate in water; B: Acetonitrile; Inj Volume: 2 µL, Flow Rate: 1.2 mL/minute; CHIRAL HPLC: Rt 13.073 min, 100%; COLUMN: CHIRAL PAK IC (150^{∗}4.6 mm, 3 µm); MOBILE PHASE A: 0.1%DEA in n-Hexane ; MOBILE PHASE B: DCM:MEOH(50:50).¹H NMR (400 MHz, DMSO-d₆) δ 8.97 - 8.91 (m, 1H), 8.85 - 8.77 (m, 1H), 8.32 - 8.24 (m, 1H), 8.20 - 8.12 (m, 1H), 7.35 - 7.15 (m, 6H), 5.24 - 5.11 (m, 1H), 3.56 -3.41 (m, 2H), 1.49 (d, 3H).

Examples 30 and 31. Synthesis of (R)-3-(difluoromethyl)-1-methyl-N-(1-(3-(2-(trifluoromethyl)pyridin-4-yl)isoxazol-5-yl)ethyl)-1H-pyrazole-5-carboxamide (30) and (S)-3-(difluoromethyl)-1-methyl-N-(1-(3-(2-(trifluoromethyl)pyridin-4-yl)isoxazol-5-yl)ethyl)-1H-pyrazole-5-carboxamide (31). Note the stereochemistry is randomly assigned.

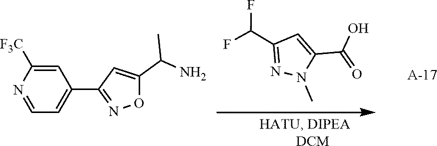

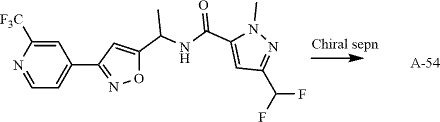

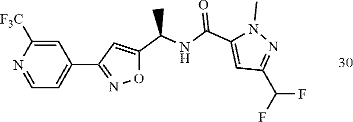 [SMILES: C[C@@H](NC(=O)c1cc(C(F)F)nn1C)c1cc(-c2ccnc(C(F)(F)F)c2)no1]

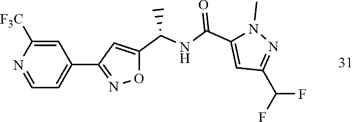 [SMILES: C[C@H](NC(=O)c1cc(C(F)F)nn1C)c1cc(-c2ccnc(C(F)(F)F)c2)no1]

To a stirred reaction mixture of A-17 (0.200 g, 0.780 mmol) and 3-(difluoromethyl)-1-methyl-1H-pyrazole-5-carboxylic acid (150.94 mg, 0.86 mmol) in DMF (5.00 mL) was added HATU (443 mg, 3.5 mmol) followed by N,N-Diisopropylethylamine (0.68 mL, 3.89 mmol) at room temperature and stirred at RT for 15 h. The reaction mixture was was quenched by adding water (10.0 mL) and then the reaction mixture was extracted with EtOAc (2×25 mL), the combined extracts were dried over anhydrous Na2SO4, filtered, concentrated under reduced pressure to obtain the residue (198 mg) as a liquid. The residue was purified by Combi-Flash column chromatography (100-200 silica gel) by eluting 0-40% EtOAc in hexanes followed by reverse phase preparative chiral HPLC to afford 30 (28 mg, 0.0663 mmol, 9%) and 31 (30 mg, 0.0711 mmol, 9%) both as solids.

30: HPLC: Rt: 7.05 min, 98.38%; Column; X SELECT CSH C18 (150×4.6 mm,3.5 um); Mobile Phase A ;5 mM AMMONIUM BICARBONATE; Mobile Phase B : ACETONITRILE; LCMS : 413.95 (M-H), Rt : 1.976 min, Column: Kinetex EVO C18 (50*3) mm 2.6 u; Mobile Phase: A: 2.5 mM Ammonium Bicarbonate in water; B: Acetonitrile; Inj Volume: 2 µL, Flow Rate: 1.2 mL/minute; CHIRAL HPLC: Rt: 8.837 min, 97.21%; COLUMN: CHIRAL PAK -IA(150×4.6 mm 3 µm); MOBILE PHASE A: 0.1% DEA n-Hexane; MOBILE PHASE B: IPA.¹H NMR (400 MHz, DMSO-d₆) δ 9.25 - 9.17 (m, 1H), 8.97 - 8.89 (m, 1H), 8.37 - 8.30 (m, 1H), 8.21 (d, 1H), 7.40 - 7.31 (m, 1H), 7.27 (s, 1H), 7.21 - 6.88 (m, 1H), 5.46 - 5.34 (m, 1H), 4.11 (s, 3H), 1.61 (d, 3H).

31: HPLC: Rt: 7.05 min, 98.37%; Column; X SELECT CSH C18 (150×4.6 mm,3.5 um); Mobile Phase A ;5 mM AMMONIUM BICARBONATE; Mobile Phase B : ACETONITRILE; LCMS : 413.95 (M-H), Rt 1.958 min, Column : Kinetex EVO C18 (50*3) mm 2.6 u; Mobile Phase: A: 2.5 mM Ammonium Bicarbonate in water; B: Acetonitrile; Inj Volume: 2 µL, Flow Rate: 1.2 mL/minute; CHIRAL HPLC: Rt 12.893 min, 100%; COLUMN: CHIRAL PAK -IA(150×4.6 mm 3 µm); MOBILE PHASE A: 0.1% DEA n-Hexane MOBILE PHASE B: IPA.¹H NMR (400 MHz, DMSO-d₆) δ 9.28 - 9.17 (m, 1H), 8.99 - 8.90 (m, 1H), 8.35 (s, 1H), 8.23 (br d, 1H), 7.38 (s, 1H), 7.29 (s, 1H), 7.23 - 6.90 (m, 1H), 5.47 - 5.35 (m, 1H), 4.13 (s, 3H), 1.62 (d, 3H).

Examples 32 and 33. Synthesis of (R)-3-(trifluoromethyl)-N-(1-(3-(2-(trifluoromethyl)pyridin-4-yl)isoxazol-5-yl)ethyl)benzamide (32) and (S)-3-(trifluoromethyl)-N-(1-(3-(2-(trifluoromethyl)pyridin-4-yl)isoxazol-5-yl)ethyl)benzamide (33). Note the stereochemistry is randomly assigned.

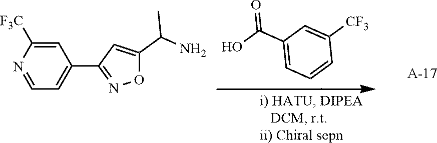

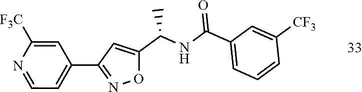 [SMILES: C[C@H](NC(=O)c1cccc(C(F)(F)F)c1)c1cc(-c2ccnc(C(F)(F)F)c2)no1]

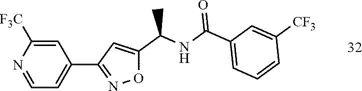 [SMILES: C[C@@H](NC(=O)c1cccc(C(F)(F)F)c1)c1cc(-c2ccnc(C(F)(F)F)c2)no1]

To a stirred reaction mixture of A-17 (300 \.mg, 1.17 mmol) and 3-(trifluoromethyl)benzoic acid (226.4 mg, 1.19 mmol) in DMF (5.00 mL) was added HATU (495 mg, 1.3 mmol) followed by N,N-Diisopropylethylamine (0.7 mL, 5.83 mmol) at room temperature and stirred at RT for 15 h. The reaction mixture was quenched by adding water (10.0 mL) and then the reaction mixture was extarcted with EtOAc (2×25 mL), the combined extracts were dried over anhydrous Na2SO4, filtered, concentrated under reduced pressure to obtain the residue (198 mg) as a colorless viscous liquid. The residue was purified by Combi-Flash column chromatography (100-200 silica gel) by eluting 0-40% EtOAc in hexanes followed by reverse phase preparative chiral HPLC obtain 32 (51 mg, 0.1186 mmol, 10%) and 33 (25 mg, 0.0578 mmol, 5%).

32. HPLC: Rt: 5.795 min, 99.83%; Column: XSELECT CSH C18 (150 × 4.6 mm, 3.5 µ); Mobile Phase-A: 0.05%TFA: Acetonitrile (95:05); Mobile Phase-B: Acetonitrile :0.05%TFA(95:05); LCMS : 428.25 (M-H), Rt 2.110 min, Column : X-SELECT CSH C18 (50*3) mm 2.5 u; Mobile Phase: A: 2.5 mM Ammonium Bicarbonate in water; B: Acetonitrile; CHIRAL HPLC: Rt: 9.192 min, 99.08%; COLUMN: Chiral pak- IG (250×4.6 mm 5 µm); MOBILE PHASE A: 0.1%DEA in n-Hexane; MOBILE PHASE B: ETOH.¹H NMR (400 MHz, DMSO-d₆) δ 9.34 (d, 1H), 8.93 (d,1H), 8.34 (s, 1H), 8.28 (s, 1H), 8.26 - 8.17 (m, 2H), 7.95 (br d, 1H), 7.76 (t, J=8 Hz, 1H), 7.38 (s, 1H), 5.53 - 5.40 (m, 1H), 1.64 (d, 3H).

33. HPLC: Rt: 5.707 min, 99.37%; Column: XSELECT CSH C18 (150 × 4.6 mm, 3.5 µ); Mobile Phase-A: 0.05%TFA: Acetonitrile (95:05); Mobile Phase-B: Acetonitrile :0.05%TFA(95:05); LCMS : 428.20 (M-H), Rt 2.097 min, Column : X-SELECT CSH C18 (50*3) mm 2.5 u; Mobile Phase: A: 2.5 mM Ammonium Bicarbonate in water; B: Acetonitrile; Inj Volume: 2 µL, Flow Rate: 1.2 mL/minute; CHIRAL HPLC: Rt: 5.364 min, 99.74%; COLUMN: Chial pak- IG (250x4.6 mm 5 µm); MOBILE PHASE A: 0.1%DEA in n-Hexane. ¹H NMR (400 MHz, DMSO-d₆) δ 9.34 (d, 1H), 8.93 (d, 1H), 8.38 - 8.17 (m, 4H), 7.95 (d, 1H), 7.81 - 7.72 (m, 1H), 7.37 (s, 1H), 5.52 - 5.42 (m, 1H), 1.64 (d, 3H).

Examples 34 and 35. Synthesis of (S)-N-(1-(3-(2-(trifluoromethyl)pyridin-4-yl)isoxazol-5-yl)ethyl)-3,4-dihydroquinoline-1(2H)-carboxamide (34) and (R)-N-(1-(3-(2-(trifluoromethyl)pyridin-4-yl)isoxazol-5-yl)ethyl)-3,4-dihydroquinoline-1(2H)-carboxamide (35). Note the stereochemistry is randomly assigned.

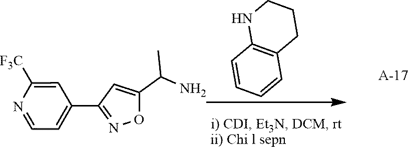

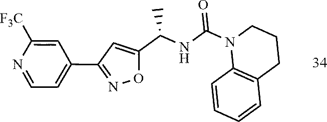 [SMILES: C[C@H](NC(=O)N1CCCc2ccccc21)c1cc(-c2ccnc(C(F)(F)F)c2)no1]

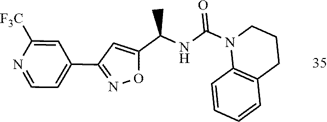 [SMILES: C[C@@H](NC(=O)N1CCCc2ccccc21)c1cc(-c2ccnc(C(F)(F)F)c2)no1]

To a stirred solution of A-17 (300 mg, 1.17 mmol) and 1,2,3,4-tetrahydroquinoline (310.7 mg, 2.33 mmol) in DCM (10 mL) were added CDI (378.25 mg, 2.33 mmol) and TEA (0.49 mL, 3.5 mmol) at room temperature. The reaction mixture was allowed to stir for 12 h at room temperature. The reaction mixture was quenched with water (10 mL) and extracted with DCM (2× 50 mL). The combined extracts were dried over anhydrous Na₂SO₄, filtered, concentrated under reduced pressure. The residue was purified by Combi-Flash column chromatography (100-200 silica gel) followed by preparative chiral HPLC obtain 34 (55 mg,0.1311 mmol, 11% yield) and 35 (60 mg, 0.1435 mmol, 12% yield)

34: HPLC: Rt: 7.925 min, 99.23%; Column: XSELECT CSH C18 (150 × 4.6 mm, 3.5 µ); Mobile Phase-A: 0.05% TFA : Acetonitrile (95:05); Mobile Phase-B: Acetonitrile :0.05% TFA (95:05); LCMS : 417.2 (M+H), Rt 2.359 min, Column:X-Bridge BEH C-18(3.0×50 mm,2.5 µm); Mobile Phase: A: 0.025% FA in Water, B: ACN; CHIRAL HPLC: Rt: 4.904 min, 100%; COLUMN: Chial pak- IA (150×4.6 mm,3 µm) Date Acquired May 01, 2021 13:08:58 IST; MOBILE PHASE A: 0.1%DEA in n-Hexane; MOBILE PHASE B: DCM:MEOH; FLOW RATE : 0.70 mL/min.¹H NMR (400 MHz, DMSO-d₆) δ 8.93 (d, 1H), 8.33 (s, 1H), 8.24 - 8.19 (m, 1H), 7.49 (d, 1H), 7.32 (d, 1H), 7.26 (s, 1H), 7.13 - 7.05 (m, 2H), 6.96 - 6.89 (m, 1H), 5.23 - 5.13 (m, 1H), 3.71 - 3.56 (m, 2H), 2.74 - 2.65 (m, 2H), 1.86 (quin, 2H), 1.56 (d, 3H).

35: HPLC: Rt: 7.926 min, 99.62%; Column: XSELECT CSH C18 (150 × 4.6 mm, 3.5 µ); Mobile Phase-A: 0.05% TFA: Acetonitrile (95:05); Mobile Phase-B: Acetonitrile: 0.05% TFA (95:05); LCMS : 417.1 (M+H), Rt 2.279 min, Column:Xselect CSH C18(4.6×150 mm,3.5 µm); Mobile Phase: A:0.025%mM aq Formic Acid, B:ACN; CHIRAL HPLC: Rt 7.094 min, 98.78%; Method File Name : CHIRAL-A.1 cm; COLUMN::CHIRAL PAK IA(150 mm× 4.6 mm,3 µm); Mobile Phase A :0.1% DEA in n-HEXANE; Mobile Phase B:DCM:MEOH(1:1); A:B::80:20; Flow:0.70 mL/min.¹H NMR (400 MHz, DMSO-d₆) δ 8.93 (d, 1H), 8.33 (s, 1H), 8.24 - 8.19 (m, 1H), 7.49 (d, 1H), 7.31 (d, 1H), 7.26 (s, 1H), 7.13 - 7.05 (m, 2H), 6.96 - 6.90 (m, 1H), 5.23 - 5.13 (m, 1H), 3.71 - 3.57 (m, 2H), 2.74 - 2.65 (m, 2H), 1.86 (quin, 2H), 1.56 (d, 3H).

Examples 36 and 37. Synthesis of (R)-1-cyclobutyl-N-(1-(3-(2-(trifluoromethyl)pyridin-4-yl)isoxazol-5-yl)ethyl)-1H-pyrazole-5-carboxamide (36) and (S)-1-cyclobutyl-N-(1-(3-(2-(trifluoromethyl)pyridin-4-yl)isoxazol-5-yl)ethyl)-1H-pyrazole-5-carboxamide (37). Note the stereochemistry is randomly assigned.

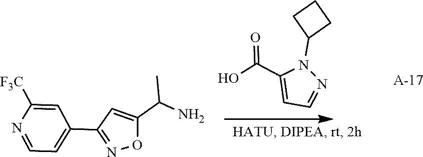

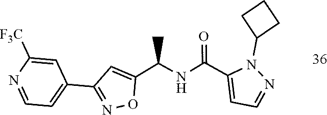 [SMILES: C[C@@H](NC(=O)c1ccnn1C1CCC1)c1cc(-c2ccnc(C(F)(F)F)c2)no1]

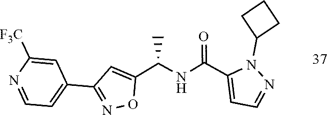 [SMILES: C[C@H](NC(=O)c1ccnn1C1CCC1)c1cc(-c2ccnc(C(F)(F)F)c2)no1]

To a stirred solution of 2-cyclobutylpyrazole-3-carboxylic acid (226.4 mg, 1.36 mmol) and A-17 (300 mg, 1.17 mmol) in DMF (5 mL) were added HATU (495 mg, 1.3 mmol) followed by N,N-diisopropylethylamine (0.7 mL, 4.32 mmol) at 0° C. and stirred at room temperature for 15 h. The reaction mixture was quenched by adding water (10 mL) and then the reaction mixture was extracted with EtOAc (2×25 mL), the combined extracts were dried over anhydrous Na₂SO₄, filtered, concentrated under reduced pressure. The residue was purified by Combi-Flash column chromatography (100-200 silica gel) using 0-40% EtOAc in hexanes as eluent followed by reverse phase preparative chiral HPLC obtain 36 (16 mg,0.0383 mmol, 3% yield) and 37 (12 mg, 0.0291 mmol, 2%) both as solids.

36: HPLC: Rt: 10.84 min, 97.10%; Column; X SELECT CSH C18 (150×4.6 mm,3.5 um); Mobile Phase A ;5 mM AMMONIUM ACETATE; Mobile Phase B : ACETONITRILE; LCMS : 404.20 (M-H), Rt 2.005 min, Column : X-SELECT CSH C18 (50*3) mm 2.5 u; Mobile Phase: A: 2.5 mM Ammonium Bicarbonate in water; B: Acetonitrile; CHIRAL HPLC: Rt: 20.326 min, 100%; COLUMN: Chial pak-IG(250×4.6 mm 3 µm); MOBILE PHASE A: 0.1%DEA in n-Hexane; MOBILE PHASE B: IPA; ¹H NMR (400 MHz, DMSO-d₆) δ 9.03 (d, 1H), 8.93 (d, 1H), 8.34 (s, 1H), 8.21 (d, 1H), 7.57 (s, 1H), 7.34 (s, 1H), 6.94 (s, 1H), 5.65 (quin, 1H), 5.45 - 5.33 (m, 1H), 2.38 - 2.25 (m, 4H), 1.84 - 1.69 (m, 2H), 1.60 (d, 3H).

37: HPLC: Rt: 10.84 min, 98.44%; Column; X SELECT CSH C18 (150×4.6 mm,3.5 um); Mobile Phase A ;5 mM AMMONIUM ACETATE; Mobile Phase B : ACETONITRILE;LCMS : 404.30 (M-H), Rt 2.002 min, Column : X-SELECT CSH C18 (50*3) mm 2.5 u; Mobile Phase: A: 2.5 mM Ammonium Bicarbonate in water; B: Acetonitrile; CHIRAL HPLC: Rt 14.486 min, 100%; COLUMN: Chial pak-IG(250×4.6 mm 3 µm); MOBILE PHASE A: 0.1%DEA in n-Hexane; MOBILE PHASE B: IPA; ¹H NMR (400 MHz, DMSO-d₆) δ 9.34 (d, 1H), 8.93 (d, 1H), 8.34 (s, 1H), 8.21 (d, 1H), 7.57 (s, 1H), 7.34 (s, 1H), 6.94 (s, 1H), 5.64 (quin, 1H), 5.45 - 5.33 (m, 1H), 2.38 - 2.24 (m, 4H), 1.85 -1.67 (m, 2H), 1.59 (d, 3H).

Examples 38 and 39. Synthesis of (S)-N-(1-(3-(2-(trifluoromethyl)pyridin-4-yl)isoxazol-5-yl)ethyl)-2,3-dihydro-4H-benzo[b] [1,4]oxazine-4-carboxamide (38) and (R)-N-(1-(3-(2-(trifluoromethyl)pyridin-4-yl)isoxazol-5-yl)ethyl)-2,3-dihydro-4H-benzo[b] [1,4]oxazine-4-carboxamide (39). Note the stereochemistry is randomly assigned.

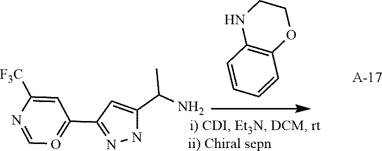

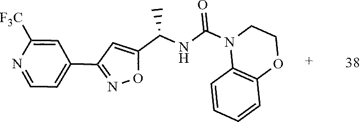

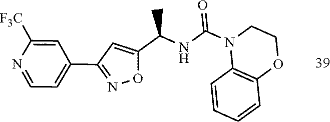 [SMILES: C[C@@H](NC(=O)N1CCOc2ccccc21)c1cc(-c2ccnc(C(F)(F)F)c2)no1]

To a stirred solution of A-17 (250 mg, 0.9700 mmol) and 3,4-dihydro-2H-1,4-benzoxazine (258.91 mg, 1.92 mmol) in DCM (10 mL) was added CDI (315.21 mg, 1.94 mmol) and TEA (0.41 mL, 2.92 mmol) at room temperature. The reaction mixture was allowed to stir at room temperature for 12 h. The reaction mixture was quenched with water (10 mL) and extracted with DCM (2× 50 mL). The combined extracts were dried over anhydrous Na₂SO₄, filtered, concentrated under reduced pressure. The residue was purified by Combi-Flash column chromatography (100-200 silica gel) by using 30-50% EtOAc/Hexane as eluent followed by preparative chiral HPLC to afford 38 (70 mg,0.1663 mmol, 17% yield) and 39 (55 mg, 0.1313 mmol, 13% yield).

38: HPLC: Rt: 7.37 min, 99.41%; Column:ATLANTIS T3 (150 × 4.6 mm, 3.5 µ); Mobile Phase A : 0.05% TFA IN WATER;ACN(95;05); Mobile Phase B : 0.05% TFA IN WATER;ACN(05;95); LCMS : 419.1 (M+H), Rt 2.153 min, Column:X-Bridge BEH C-18(3.0×50 mm,2.5 µm); Mobile Phase: A: 0.025% FA in Water, B: ACN;CHIRAL HPLC: Rt: 6.046 min, 100%; COLUMN: Chiral pak-IG (250×4.6 mm,5 µm); MOBILE PHASE A: 0.1%DEA in n-Hexane; ¹H NMR (400 MHz, DMSO-d₆) δ 8.93 (d, 1H), 8.33 (s, 1H), 8.21 (d, 1H), 7.50 (d, 1H), 7.57 (d, 1H), 7.27 (d, 1H), 6.96 - 6.89 (m, 1H), 6.88 - 6.80 (m, 2H), 5.22 - 5.12 (m, 1H), 4.26 - 4.17 (m, 2H), 3.86 - 3.69 (m, 2H), 1.57 (d, 3H).

39: HPLC: Rt: 7.17 min, 97.32%; Column: X SELECT CSH C18 (150×4.6 mm,3.5 um); Mobile Phase A ;0.05% FORMIC ACID IN WATER; Mobile Phase B : ACETONITRILE; LCMS : 374.05 (M-H), Rt 2.109 min, Column: Kinetex EVO C18 (50^{∗}3) mm 2.6 u; Mobile Phase: A: 2.5 mM Ammonium Bicarbonate in water; B: Acetonitrile; Inj Volume: 2 µL, Flow Rate: 1.2 mL/minute; CHIRAL HPLC: Rt 13.073 min, 100%; COLUMN: CHIRAL PAK IC (150*4.6 mm, 3 µm); MOBILE PHASE A: 0.1%DEA in n-Hexane ; MOBILE PHASE B: DCM:MEOH(50:50).¹H NMR (400 MHz, DMSO-d₆) δ 8.93 (d, 1H), 8.33 (s, 1H), 8.21 (d, 1H), 7.49 (d, 1H), 7.31 (d, 1H), 7.26 (d, 1H), 7.12 - 7.05 (m, 1H), 6.96 - 6.89 (m, 2H), 5.18 (quin, 1H), 4.25 - 4.20 (m, 2H), 3.83 - 3.72 (m, 2H), 1.56 (d, 3H).

Example 40. 2-methyl-N-[(1S)-1-[3-(2-methyl-4-pyridyl)-1,2,4-thiadiazol-5-yl]ethyl]-5-(trifluoromethyl)pyrazole-3-carboxamide (40)

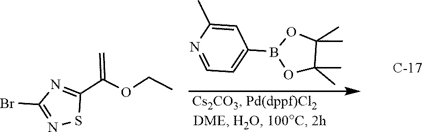

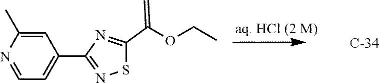

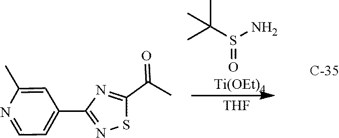

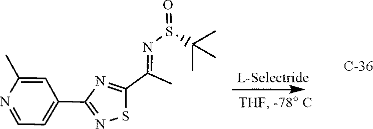

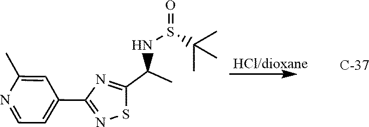

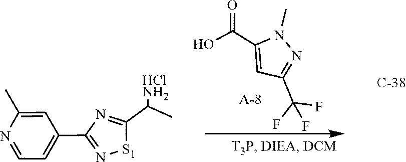

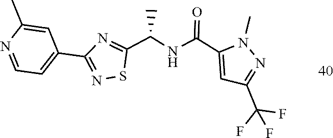 [SMILES: Cc1cc(-c2nsc([C@H](C)NC(=O)c3cc(C(F)(F)F)nn3C)n2)ccn1]

### 5-Ethoxyvinyl)-3-(2-methyl-4-pyridyl)-1,2,4-thiadiazole (C-34)

A mixture of 3-bromo-5-(1-ethoxyvinyl)-1,2,4-thiadiazole (1.5 g, 6.38 mmol) in DME (30.0 mL) was added (2-methyl-4-pyridyl)boronic acid (1.05 g, 7.66 mmol), Cs₂CO₃ (6.24 g, 19.1 mmol), water (6.0 mL) and Pd(dppf)Cl₂ (0.47 g, 0.64 mmol. After stirring at 100° C. for 3 hours, the mixture was filtered and concentrated, and the residue was purified by chromatography on silica gel (0~30% of EtOAc in PE) to give the product (1.20 g, 4.61 mmol, 72% yield) as a solid. ¹H NMR (400 MHz, CDC13) δ_{H} = 8.63 (d, 1H), 8.04 (s, 1H), 7.97 (d, 1H), 5.60 (d, 1H), 4.57 (d, 1H), 4.08 - 3.99 (m, 2H), 2.66 (s, 3H), 1.49 - 1.41 (m, 3H).

### 1-(2-Methyl-4-pyridyl)-1,2,4-thiadiazol-5-yl]ethenone (C-35)

To a mixture of 5-(1-ethoxyvinyl)-3-(2-methyl-4-pyridyl)-1,2,4-thiadiazole (1.20 g, 4.85 mmol) in acetone (15.0 mL) was added HCl (8.0 mL, 2 M, 4.85 mmol). After stirring at 50° C. for 16 h, the mixture was diluted with water (15.0 mL) and extracted with EtOAc (3 × 10.0 mL). The combined organic phase was washed with brine (30.0 mL), dried over anhydrous Na₂SO₄, filtered and concentrated to afford the product (1.10 g, 4.52 mmol, 93% yield) as an oil. ¹H NMR (400 MHz, CDC13) δ_{H} = 8.68 (d, 1H), 8.06 (s, 1H), 7.99 (d, 1H), 2.83 (s, 3H), 2.69 (s, 3H).

### (R,E)methyl-N-[1-[3-(2-methyl-4-pyridyl)-1,2,4-thiadiazol-5-yl]ethylidene]propanesulfinamide (C-36)

To a solution of 1-[3-(2-methyl-4-pyridyl)-1,2,4-thiadiazol-5-yl]ethanone (300 mg, 1.37 mmol) in THF (5.0 mL) and (R)-2-methylpropane-2-sulfinamide (249 mg, 2.05 mmol) was added Ti(OEt)₄ (0.94 g, 4.10 mmol). After stirring at 50° C. for 16 h, the mixture was poured into saturated NaHCO₃ (20 mL) and diluted with EtOAc (10.0 mL). The resulting slurry was filtered and extracted with EtOAc (3 × 10.0 mL). The combined organic layer was washed with brine (2 × 30.0 mL), dried over anhydrous Na₂SO₄, filtered and concentrated. The residue was purified by flash column (0~30% of EtOAc in PE) to give the product (550 mg) as an oil. The product was purified by flash column (0~30% of EtOAc in PE) to give the product (350 mg, 1.09 mmol, 64% yield) as a solid. ¹H NMR (400 MHz, CDC13) δ_{H} = 8.69 (d, 1H), 8.27-8.11 (m, 2H), 2.97 (s, 3H), 2.83 (s, 3H), 1.37 (s, 9H).

### (R)methyl-N-[(1S)-1-[3-(2-methyl-4-pyridyl)-1,2,4-thiadiazol-5-yl]ethyl]propanesulfinamide (C-37)

To a solution of (R,E)-2-methyl-N-[1-[3-(2-methyl-4-pyridyl)-1,2,4-thiadiazol-5-yl]ethylidene]propane-2-sulfinamide (350 mg, 1.09 mmol) in THF (4.0 mL) was added L-Selectride (2.17 mL, 2.17 mmol) at -78° C. After stirring at -78° C. for 0.5 h, the mixture was poured into saturated NH₄C1 (20.0 mL) and extracted with EtOAc (2 × 10.0 mL). The combined organic layer was washed with brine (2 × 20.0 mL), dried over anhydrous Na₂SO₄, filtered and concentrated. The residue was purified by flash column (0~10% of MeOH in DCM) to give the product (270 mg, 0.832 mmol, 77% yield) as a solid. ¹H NMR (400 MHz, CDCl₃) δ_{H} = 8.65 (d, 1H), 8.21-7.95 (m, 2H), 5.10-4.89 (m, 1H), 2.75 (s, 3H), 1.84 (d, 3H), 1.34 (s, 9H).

### (1S)[3-(2-methyl-4-pyridyl)-1,2,4-thiadiazol-5-yl]ethanamine (C-38)

To a solution of (R)-2-methyl-N-[(1S)-1-[3-(2-methyl-4-pyridyl)-1,2,4-thiadiazol-5-yl]ethyl]propane-2-sulfinamide (270 mg, 0.83 mmol) in 1,4-Dioxane (5.0 mL) was added 4 M HCl/dioxane (3 mL) at 25° C. After stirring at 25° C. for 1 h, the mixture was concentrated to give the product as a solid. ¹H NMR (MeOD, 400 MHz) δ_{H} = 8.89 (d, 1H), 8.75 (s, 1H), 8.71-8.65 (m, 1H), 5.39-5.17 (m, 1H), 2.92 (s, 3H), 1.85 (d, 3H).

### 2-methyl-N-[(1S)-1-[3-(2-methyl-4-pyridyl)-1,2,4-thiadiazol-5-yl]ethyl]-5-(trifluoromethyl)pyrazole-3-carboxamide (40)

To a solution of 2-methyl-5-(trifluoromethyl)pyrazole-3-carboxylic acid (229 mg, 1.18 mmol) in DCM (8.0 mL) was added DIEA (937 mg, 7.26 mmol) and T₃P (2.71 g, 2.72 mmol). After stirring at 25° C. for 20 mins, (1S)-1-[3-(2-methyl-4-pyridyl)-1,2,4-thiadiazol-5-yl]ethanamine hydrochloride (200 mg, 0.91 mmol) was added and the reaction mixture was stirred at 25° C. for 16 hr. The reaction mixture was quenched with water (10.0 mL) and extracted with DCM (2 × 15.0 mL). The combined organic layer was washed with brine (20.0 mL) and dried over Na₂SO₄, filtered and concentrated. The residue was purified by flash column (0~10% of MeOH in DCM) to give the product (300 mg, 0.757 mmol, 83% yield) as as a solid. The product was purified by SFC (Column DAICEL CHIRALPAK IG (250 mm * 30 mm, 10 µm) Condition 0.1% NH₃H₂O EtOH Begin B 20% End B 20% Gradient Time (min) 100% B Hold Time (min) FlowRate (ml/min) 60 Injections 35) to give the product (81.2 mg, 0.197 mmol, 26% yield) as a solid. ¹H NMR (400 MHz, CDC13) δ_{H} = 8.65 (d, 1H), 8.00 (s, 1H), 7.95-7.88 (m, 1H), 6.90 (s, 1H), 6.78-6.66 (m, 1H), 5.79-5.65 (m, 1H), 4.24 (s, 3H), 2.66 (s, 3H), 1.83 (d, 3H). ¹⁹F NMR (376.5 MHz, CDC13) δ_{F} = -62.195. LCMS Rt = 0.895 min in 1.5 min chromatography, 5-95AB, MS ESI calcd. for C₁₆H₁₆F₃N₆OS [M+H]⁺396.9, found 396.9.

Example 41. (R)-1-methyl-N-(1-(3-(2-methylpyridin-4-yl)-1,2,4-thiadiazol-5-yl)ethyl)-3-(trifluoromethyl)-1H-pyrazole-5-carboxamide (41)

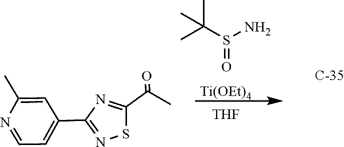

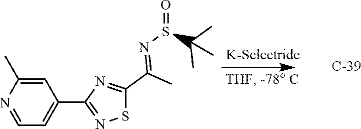

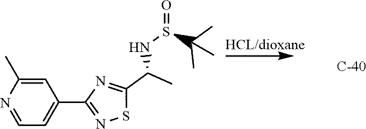

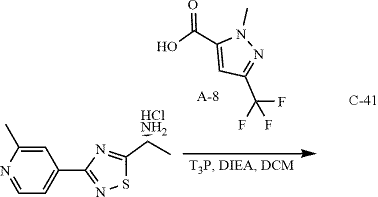

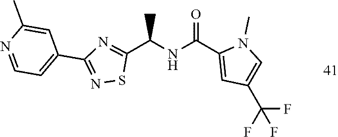 [SMILES: Cc1cc(-c2nsc([C@@H](C)NC(=O)c3cc(C(F)(F)F)cn3C)n2)ccn1]

### (S,E)methyl-N-[1-[3-(2-methyl-4-pyridyl)-1,2,4-thiadiazol-5-yl] Ethylidene] Propanesulfinamide (C-39)

To a solution of 1-[3-(2-methyl-4-pyridyl)-1,2,4-thiadiazol-5-yl]ethanone (300 mg, 1.37 mmol) in THF (5.0 mL) and (S)-2-methylpropane-2-sulfinamide (249 mg, 2.05 mmol) was added Ti(OEt)₄ (0.94 g, 4.10 mmol). After stirring at 50° C. for 16 h, the mixture was poured into saturated NaHCO₃ (20 mL) and diluted with EtOAc (10.0 mL). The resulting slurry was filtered and extracted with EtOAc (3 × 10.0 mL). The combined organic layer was washed with brine (2 × 30.0 mL), dried over anhydrous Na₂SO₄, filtered and concentrated. The residue was purified by flash column (0~30% of EtOAc in PE) to give the product (310 mg, 0.96 mmol, 70% yield) as an oil. ¹H NMR (400 MHz, CDC13) δ_{H} = 8.81-8.62 (m, 1H), 8.16-8.11 (m, 1H), 8.10-8.04 (m, 1H), 2.95 (s, 3H), 2.75 (s, 3H), 1.37 (s, 9H).

### (S)methyl-N-[(1R)-1-[3-(2-methyl-4-pyridyl)-1,2,4-thiadiazol-5-yl]ethyl] Propanesulfinamide (C-40)

To a solution of (S,E)-2-methyl-N-[1-[3-(2-methyl-4-pyridyl)-1,2,4-thiadiazol-5-yl]ethylidene]propane-2-sulfinamide (310 mg, 0.96 mmol) in THF (4.0 mL) was added K-Selectride (1.92 mL, 1.92 mmol) at -78° C. After strring at -78° C. for 0.5 h, the mixture was poured into saturated NH₄C1 (20.0 mL) and extracted with EtOAc (2 × 10.0 mL). The combined organic layer was washed with brine (2 × 20.0 mL), dried over anhydrous Na₂SO₄, filtered and concentrated. The residue was purified by flash column (0~10% of MeOH in DCM) to give the product (200 mg, 0.616 mmol, 64% yield) as a solid. ¹H NMR (400 MHz, CDC13) δ_{H} = 8.65 (d, 1H), 8.19-8.00 (m, 2H), 5.11-4.92 (m, 1H), 2.77 (s, 3H), 1.84 (d, 3H), 1.40-1.26 (m, 9H).

### (R)(3-(2-methylpyridin-4-yl)-1,2,4-thiadiazol-5-yl)ethanamine Hydrochloride (C-41)

To a solution of (S)-2-methyl-N-[(1R)-1-[3-(2-methyl-4-pyridyl)-1,2,4-thiadiazol-5-yl]ethyl]propane-2-sulfinamide (200 mg, 0.62 mmol) in 1,4-Dioxane (3.0 mL) was added 4 M HCl/dioxane (2.31 mL, 9.25 mmol) at 25° C. After stirring at 25° C. for 1 h, the mixture was concentrated to give the product (120 mg, 0.38 mmol) as as a solid. ¹H NMR (DMSO-d6, 400 MHz) δ_{H} = 9.18-9.12 (m, 2H), 8.90 (d, 1H), 8.45 (s, 1H), 8.40-8.29 (m, 1H), 5.38-5.15 (m, 1H), 2.80 (s, 3H), 1.72 (d, 3H).

### (R)methyl-N-(1-(3-(2-methylpyridin-4-yl)-1,2,4-thiadiazol-5-yl)ethyl)-3-(trifluoromethyl)-1H-pyrazole-5-carboxamide (41)

To a solution of 2-methyl-5-(trifluoromethyl)pyrazole-3-carboxylic acid (97.0 mg, 0.50 mmol) in DCM (8.0 mL) was added DIEA (409.0 mg, 3.17 mml) and T₃P (904 mg, 1.19 mmol). After stirring at 25° C. for 20 mins, (1R)-1-[3-(2-methyl-4-pyridyl)-1,2,4-thiadiazol-5-yl]ethanamine hydrochloride (100 mg, 0.45 mmol) was added and the reaction mixture was stirred at 25° C. for 16 hr. The reaction mixture was quenched with water (10.0 mL) and extracted with DCM (2 × 15.0 mL). The combined organic layer was washed with brine (20.0 mL), dried over Na₂SO₄, filtered and concentrated to give the product (140 mg, 0.32 mmol) as a solid which was purified by SFC (Column DAICEL CHIRALCEL OJ (250 mm ^{∗} 30 mm, 10 µm), Condition: 0.1%NH₃H₂O-MeOH, Begin B: 20%, End B: 20%, FlowRate (mL/min): 60, Injections: 30) to give the product (113.2 mg, 0.29 mmol, 57% yield) as a solid. ¹H NMR (400 MHz, CDC13) δ_{H}= 8.72-8.58 (m, 1H), 7.99 (s, 1H), 7.95-7.89 (m, 1H), 6.91 (s, 1H), 6.83-6.75 (m, 1H), 5.79-5.65 (m, 1H), 4.24 (s, 3H), 2.66 (s, 3H), 1.87-1.77 (m, 3H). ¹⁹F NMR (376.5 MHz, CDCl₃) δ_{F} = -62.183. LCMS Rₜ = 1.241 min in 2.0 min chromatography, 10-80AB, MS ESI calcd. for C₁₆H₁₆F₃N₆OS [M+H]⁺397.1, found 397.1.

Examples 42 and 43. 2-methyl-N-[(1S)-1-[3-[2-(methoxymethyl)-4-pyridyl]-1,2,4-thiadiazol-5-yl]ethyl]-5-(trifluoromethyl)pyrazole-3-carboxamide & 2-methyl-N-[(1R)-1-[3-[2-(methoxymethyl)-4-pyridyl]-1,2,4-thiadiazol-5-yl]ethyl]-5-(trifluoromethyl)pyrazole-3-carboxamide.

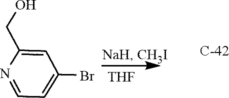

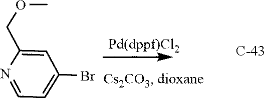

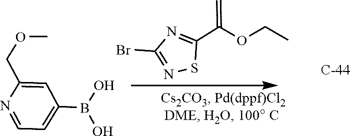

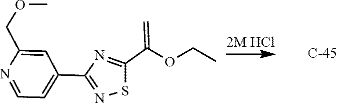

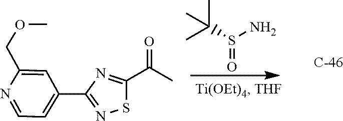

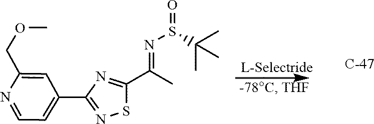

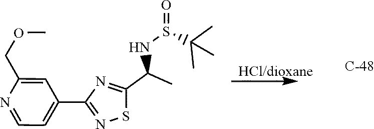

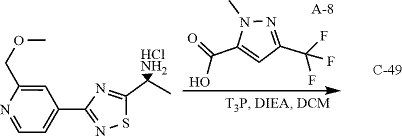

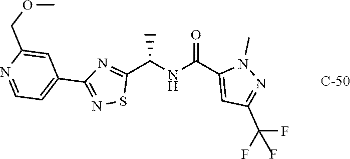

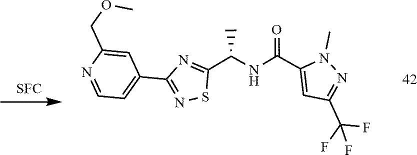 [SMILES: "SFC" ; COCc1cc(-c2nsc([C@H](C)NC(=O)c3cc(C(F)(F)F)nn3C)n2)ccn1]

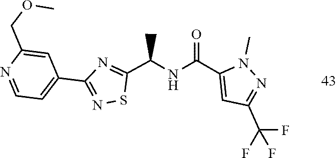 [SMILES: COCc1cc(-c2nsc([C@@H](C)NC(=O)c3cc(C(F)(F)F)nn3C)n2)ccn1]

### 4-Bromo(methoxymethyl)pyridine (C-43)

To a mixture of (4-bromo-2-pyridyl)methanol (9.0 g, 47.9 mmol) in DMF (15.0 mL) was added NaH (2.30 g, 57.4 mmol, 60%) at 0° C. under N₂. After stirring for 30 mins, the mixture of methyl iodide (3.29 mL, 52.6 mmol) in DMF (5.0 mL) was added and the mixture was stirred at 15° C. for 16 hours. The mixture poured into ice-water (30.0 mL) and the aqueous phase was extracted with EtOAc (3 × 30.0 mL). The combined organic phase was washed with brine (2 × 20 mL), dried over anhydrous Na₂SO₄, filtered and concentrated. The residue was purified by column chromatography (PE/EtOAc= 3/1 to 1/1) to afford the product (9.0 g, 44.5 mmol, 93% yield) as an oil. ¹H NMR (CDC1₃, 400 MHz) δ_{H} = 8.36 (d, 1H), 7.63 (d, 1H), 7.37 (dd, 1H), 4.57 (s, 2H), 3.51-3.46 (m, 3H).

### 2-(Methoxymethyl)-4-(4,4,5,5-tetramethyl-1,3,2-dioxaborolan-2-yl)pyridine (C-44)

To a mixture of 4-bromo-2-(methoxymethyl)pyridine (5.0 g, 24.8 mmol), 4,4,5,5-tetramethyl-2-(4,4,5,5-tetramethyl-1,3,2-dioxaborolan-2-yl)-1,3,2-dioxaborolane (6.91 g, 27.2 mmol), Pd(dppf)Cl₂ (1.81 g, 2.47 mmol) and KOAc (4.86 g, 49.5 mmol) in 1,4-Dioxane (50 mL) was stirred at 100° C. for 3 hours under N₂. The mixture was cooled to 25° C., filtered and concentrated to give the product (9.0 g, 36.1 mmol) as an oil.

### 5-Ethoxyvinyl)-3-[2-(methoxymethyl)-4-pyridyl]-1,2,4-thiadiazole (C-45)

To amixture of 3-bromo-5-(1-ethoxyvinyl)-1,2,4-thiadiazole (2.0 g, 8.51 mmol) [2-(methoxymethyl)-4-pyridyl]boronic acid (2.84 g, 17.0 mmol) and Cs₂CO₃ (5.54 g, 17.0 mmol) in DME (20.0 mL) and water (4.0 mL) was added Pd(dppf)Cl₂ (622 mg, 0.85 mmol) and heated with a microwave reactor at 90° C. for 1.5 hours. After cooling to 25° C., the reaction mixture was quenched with water (40.0 mL) and extracted with EtOAc (2 × 40.0 mL). The combined organic layer was concentrated under reduced pressure. The residue was purified by chromatography on silica gel with PE/EtOAc= 1/1 to give the product (2.10 g, 7.57 mmol, 89% yield) as an oil. ¹H NMR (CDC1₃, 400 MHz) δ_{H} = 8.71 (d, 1H), 8.34-8.23 (m, 1H), 8.07 (d, 1H), 5.63 (d, 1H), 4.71-4.63 (m, 2H), 4.59 (d, 1H), 4.09-4.02 (m, 2H), 3.53 (s, 3H), 1.46 (t, 3H).

### 1-[2-(Methoxymethyl)-4-pyridyl]-1,2,4-thiadiazol-5-yl]ethenone (C-46)

To a mixture of 5-(1-ethoxyvinyl)-3-[2-(methoxymethyl)-4-pyridyl]-1,2,4-thiadiazole (2.19 g, 7.90 mmol) in acetone (20.0 mL) was added 2 M HCl (7.90 mL, 15.8 mmol). After stirring at 50° C. for 16 h, the mixture was diluted with water (5.0 mL) and extracted with EtOAc (3 × 5.0 mL). The combined organic phase was washed with brine (20.0 mL), dried over anhydrous Na₂SO₄, filtered and concentrated to afford the product (1.60 g, 5.78 mmol, 73% yield) as an oil. LCMS Rₜ= 0.861 min in 1.5 min chromatography, 5-95AB, MS ESI calcd. for C₁₁H₁₂N₃O₂S [M+H]⁺250.1, found 249.9.

### (R,E)-N-[1-[3-[2-(methoxymethyl)-4-pyridyl]-1,2,4-thiadiazol-5-yl]ethylidene]-2-methyl-propane-2-sulfinamide (C-47)

To a solution of 1-[3-[2-(methoxymethyl)-4-pyridyl]-1,2,4-thiadiazol-5-yl]ethanone (1.0 g, 4.0 mmol) in THF (10.0 mL) and (R)-2-methylpropane-2-sulfinamide (729 mg, 6.10 mmol) was added Ti(OEt)₄ (2.75 g, 12.0 mmol). After stirring at 50° C. for 16 h, the mixture was poured into saturated NaHCO₃ (20.0 mL) and diluted with EtOAc (10.0 mL). The resulting slurry was filtered and the mother liquor was extracted with EtOAc (3 × 10.0 mL). The combined organic layer was washed with brine (2 × 30.0 mL), dried over anhydrous Na₂SO₄, filtered and concentrated. The residue was purified by flash column to the product (190 mg, 0.54 mmol, 22% yield) as an oil. ¹H NMR (CDC1₃, 400 MHz) δ_{H} = 8.74 (d, 1H), 8.46-8.41 (m, 1H), 8.25-8.18 (m, 1H), 4.84-4.78 (m, 2H), 3.57 (s, 3H), 2.97 (s, 3H), 1.37 (s, 9H).

### R)-N-[(1S)-1-[3-[2-(methoxymethyl)-4-pyridyl]-1,2,4-thiadiazol-5-yl]ethyl]-2-methyl-propane-2-sulfinamide (C-48)

To a solution of (R,E)-N-[1-[3-[2-(methoxymethyl)-4-pyridyl]-l,2,4-thiadiazol-5-yl]ethylidene]-2-methyl-propane-2-sulfinamide (190 mg, 0.54 mmol) in THF (4.0 mL) was added K-Selectride (1.08 mL, 1.08 mmol) at -78° C. After stirring at -78° C. for 0.5 h, the mixture was poured into saturated NH₄C1 (20.0 mL) and extracted with EtOAc (2 × 10.0 mL). The combined organic layer was washed with brine (2 × 20.0 mL), dried over anhydrous Na₂SO₄, filtered and concentrated. The residue was purified by flash column to give the product (130 mg, 0.37 mmol, 68% yield) as as a solid. LCMS Rₜ = 0.803 min in 1.5 min chromatography, 5-95AB, MS ESI calcd. for C15H23N₄O₂S₂ [M+H]⁺355.1, found 355.1.

### (1S)[3-[2-(methoxymethyl)-4-pyridyl]-1,2,4-thiadiazol-5-yl]ethanamine hydrochloride (C-49)

To a solution of (R)-2-methyl-N-[(1S)-1-[3-[2-(methoxymethyl)-4-pyridyl]-1,2,4-thiadiazol-5-yl]ethyl]propane-2-sulfinamide (130 mg, 0.37 mmol) in 1,4-Dioxane (5.0 mL) was added 4 M HCl/dioxane (6.0 mL, 1.83 mmol) at 25° C. After stirring at 25° C. for 1 h, the residue was filtered and concentrated to give the product (130 mg, 0.52 mmol) as as a solid. ¹H NMR (MeOD, 400 MHz) δ_{H}= 8.94 (d, 1H), 8.82 (s, 1H), 8.78-8.74 (m, 1H), 5.32-5.24 (m, 1H), 4.99 (s, 2H), 4.88-4.87 (m, 2H), 3.64 (s, 3H), 1.85 (d, 3H).

### 2-methyl-N-[(lS)-1-[3-[2-(methoxymethyl)-4-pyridyl]-l,2,4-thiadiazol-5-yl]ethyl]-5-(trifluoromethyl)pyrazole-3-carboxamide (C-50)

To a solution of 2-methyl-5-(trifluoromethyl)pyrazole-3-carboxylic acid (111 mg, 0.57 mmol) in DCM (2.0 mL) was added DIEA (0.91 mL, 5.19 mmol), T₃P (1.18 g, 1.56 mmol) at 25° C. After stirring for 10 mins, (1S)-1-[3-[2-(methoxymethyl)-4-pyridyl]-1,2,4-thiadiazol-5-yl]ethanamine hydrochloride (130 mg, 0.52 mmol) was added and the reaction mixture was stirred at 25° C. for 3 h. The reaction mixture was quenched with water (20.0 mL) and extracted with DCM (2 × 20.0 mL). The combined organic layer was washed with brine (60.0 mL) and dried over Na₂SO₄, filtered and concentrated to give the product which was purified by prep-HPLC (Column: Phenomenex Gemini-NX 80 × 30 mm × 3 µm; Condition: water(10 mM NH4HCO₃)-ACN; Begin B: 42 to 72% B over 10 minutes) to give the product (75.0 mg, 0.18 mmol, 34% yield) as a solid.

### 2-methyl-N-[(1S)-1-[3-[2-(methoxymethyl)-4-pyridyl]-1,2,4-thiadiazol-5-yl]ethyl]-5-(trifluoromethyl)pyrazole-3-carboxamide & 2-methyl-N-[(1R)-1-[3-[2-(methoxymethyl)-4-pyridyl]-1,2,4-thiadiazol-5-yl]ethyl]-5-(trifluoromethyl)pyrazole-3-carboxamide. Note that the stereochemistry is randomly assigned

2-methyl-N-[(1S)-1-[3-[2-(methoxymethyl)-4-pyridyl]-1,2,4-thiadiazol-5-yl]ethyl]-5-(trifluoromethyl)pyrazole-3-carboxamide (75.0 mg, 0.18 mmol) was purified by SFC (DAICEL CHIRALCEL AY-H (250 mm ^{∗} 30 mm, 5 µm); Condition: 0.1% NH₃H₂O-EtOH; Begin B: 15 to 15) to give 2-methyl-N-[(1S)-1-[3-[2-(methoxymethyl)-4-pyridyl]-1,2,4-thiadiazol-5-yl]ethyl]-5-(trifluoromethyl)pyrazole-3-carboxamide (61.5 mg, 0.14 mmol, 82% yield) as a solid and 2-methyl-N-[(1R)-1-[3-[2-(methoxymethyl)-4-pyridyl]-1,2,4-thiadiazol-5-yl]ethyl]-5-(trifluoromethyl)pyrazole-3-carboxamide (2.46 mg, 0.01 mmol, 3% yield) as a solid.

42: ¹H NMR (CDCl₃, 400 MHz) δ_{H} = 8.68 (d, 1H), 8.24 (s, 1H), 8.04-7.98 (m, 1H), 6.98-6.87 (m, 2H), 5.75-5.66 (m, 1H), 4.66 (s, 2H), 4.23 (s, 3H), 3.52 (s, 3H), 1.82 (d, 3H). ¹⁹F NMR (376.5 MHz, CDCl₃) δ_{F} = -62.160. LCMS Rₜ = 0.951 min in 2.0 min chromatography, 10-80 AB, MS ESI calcd. for C₁₇H₁₈F₃N₆O₂S [M+H]⁺427.1, found 427.1.

43: ¹H NMR (CDCl₃, 400 MHz) δ_{H} = 8.71 (d, 1H), 8.26 (s, 1H), 8.03 (d, 1H), 6.92 (s, 1H), 6.79 (d, 1H), 5.77-5.66 (m, 1H), 4.68 (s, 2H), 4.24 (s, 3H), 3.53 (s, 3H), 1.83 (d, 3H). ¹⁹F NMR (376.5 MHz, CDCl₃) δ_{F} = -62.169. LCMS Rₜ = 0.957 min in 2.0 min chromatography, 10-80 AB, MS ESI calcd. for C₁₇H₁₈F₃N₆O₂S [M+H]⁺427.1, found 427.1.

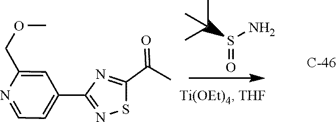

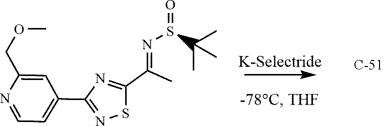

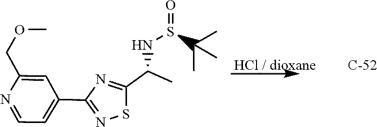

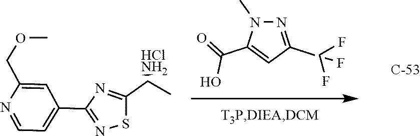

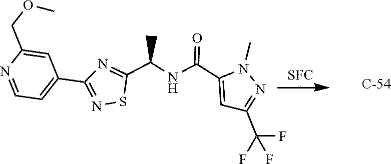

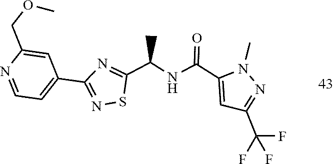 [SMILES: COCc1cc(-c2nsc([C@@H](C)NC(=O)c3cc(C(F)(F)F)nn3C)n2)ccn1]

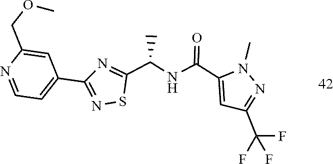 [SMILES: COCc1cc(-c2nsc([C@H](C)NC(=O)c3cc(C(F)(F)F)nn3C)n2)ccn1]

### (R,E)-N-[1-[3-[2-(methoxymethyl)-4-pyridyl]-1,2,4-thiadiazol-5-yl]ethylidene]-2-methyl-propane-2-sulfinamide (C-51)

To a solution of 1-[3-[2-(methoxymethyl)-4-pyridyl]-1,2,4-thiadiazol-5-yl]ethanone (300 mg, 1.20 mmol) in THF (5.0 mL) and (S)-2-methylpropane-2-sulfinamide (219 mg, 1.81 mmol) was added Ti(OEt)₄ (823 mg, 3.61 mmol). After stirring at 50° C. for 16 h, the mixture poured into saturated NaHCO₃ (20.0 mL) and diluted with EtOAc (10.0 mL). The resulting slurry was filtered and the mother liquor was extracted with EtOAc (3 × 10.0 mL). The combined organic layer was washed with brine (2 × 30.0 mL), dried over anhydrous Na₂SO₄, filtered and concentrated. The residue was purified by flash column (0~30% of EtOAc in PE) to give the product (90.0 mg, 0.26 mmol, 21% yield) as an oil. ¹H NMR (CDCl₃, 400 MHz) δ_{H} = 8.73 (d, 1H), 8.32-8.24 (m, 1H), 8.07 (dd, 1H), 4.69 (s, 2H), 3.54 (s, 3H), 2.97 (s, 3H), 1.37 (s, 9H).

### (R)-N-[(1S)-1-[3-[2-(methoxymethyl)-4-pyridyl]-1,2,4-thiadiazol-5-yl]ethyl]-2-methyl-propane-2-sulfinamide (C-52)

To a solution of (R,E)-N-[1-[3-[2-(methoxymethyl)-4-pyridyl]-1,2,4-thiadiazol-5-yl]ethylidene]-2-methyl-propane-2-sulfinamide (150 mg, 0.43 mmol) in THF (4.0 mL) was added K-Selectride (0.85 mL, 0.85 mmol) at -78° C. After stirring at -78° C. for 0.5 h, the mixture was poured into saturated NH₄Cl (20.0 mL) and extracted with EtOAc (2 × 10.0 mL). The combined organic layer was washed with brine (2 × 20.0 mL), dried over anhydrous Na₂SO₄, filtered and concentrated. The residue was purified by flash column (0~10% of MeOH in DCM) to give the product (120 mg, 0.34 mmol, 80% yield) as as a solid. ¹H NMR (CDCl₃, 400 MHz) δ_{H} = 8.70 (d, 1H), 8.25 (s, 1H), 8.03 (dd, 1H), 5.07-4.98 (m, 1H), 4.67 (s, 2H), 3.66 (d, 1H), 3.53 (s, 3H), 1.84 (d, 3H), 1.33 (s, 9H).

### (1R)[3-[2-(methoxymethyl)-4-pyridyl]-1,2,4-thiadiazol-5-yl]ethanamine hydrochloride (C-53)

To a solution of (S)-N-[(1R)-1-[3-[2-(methoxymethyl)-4-pyridyl]-1,2,4-thiadiazol-5-yl]ethyl]-2-methyl-propane-2-sulfinamide (120 mg, 0.34 mmol) in 1,4-dioxane (5.0 mL) was added 4 M HCl/dioxane (6.0 mL, 1.69 mmol) at 25° C. After stirring at 25° C. for 1 h, the residue was filtered and concentrated to give the product (84.0 mg, 0.29 mmol, 87% yield) as as a solid which was used directly for the next step.

### N-[(1R)[3-[2-(methoxymethyl)-4-pyridyl]-1,2,4-thiadiazol-5-yl]ethyl]-2-methyl-5-(trifluoromethyl)pyrazole-3-carboxamide (C-54)

A mixture of 2-methyl-5-(trifluoromethyl)pyrazole-3-carboxylic acid (84.7 mg, 0.44 mmol), T₃P (766 mg, 1.01 mmol) and DIEA (0.47 mL, 2.68 mmol) in DCM (8.0 mL) was stirred at 25° C. for 20 mins. (1R)-1-[3-[2-(methoxymethyl)-4-pyridyl]-1,2,4-thiadiazol-5-yl]ethanamine hydrochloride (84.0 mg, 0.29 mmol) was added. After stirring at 25° C. for 1 hour, the reaction mixture was quenched with water (10.0 mL) and extracted with DCM (2 × 15.0 mL). The combined organic layer was washed with brine (20.0 mL) and dried over Na₂SO₄, filtered and concentrated to give the product (100 mg, 0.23 mmol, 70% yield) as an oil. ¹H NMR (CDC1₃, 400 MHz) δ_{H} = 8.63 (d, 1H), 8.57-8.50 (m, 1H), 8.38-8.27 (m, 1H), 7.47-7.33 (m, 1H), 7.14-7.09 (m, 1H), 5.79-5.64 (m, 1H), 4.99-4.84 (m, 2H), 4.26 (s, 3H), 3.58 (s, 3H), 1.90 (d, 3H).

### N-[(1R)[3-[2-(methoxymethyl)-4-pyridyl]-1,2,4-thiadiazol-5-yl]ethyl]-2-methyl-5-(trifluoromethyl)pyrazole-3-carboxamide & N-[(1S)[3-[2-(methoxymethyl)-4-pyridyl]-1,2,4-thiadiazol-5-yl]ethyl]-2-methyl-5-(trifluoromethyl)pyrazole-3-carboxamide

The mixture of N-[(1R)-1-[3-[2-(methoxymethyl)-4-pyridyl]-1,2,4-thiadiazol-5-yl]ethyl]-2-methyl-5-(trifluoromethyl)pyrazole-3-carboxamide (100 mg, 0.23 mmol) was purified by SFC (Column DAICEL CHIRALCEL OJ (250 mm * 30 mm, 10 µm), Condition 0.1% NH₃H₂O-EtOH, Begin B 15%, End B 15%, FlowRate (mL/min) 60) to give N-[(1R)-1-[3-[2-(methoxymethyl)-4-pyridyl]-1,2,4-thiadiazol-5-yl]ethyl]-2-methyl-5-(trifluoromethyl)pyrazole-3-carboxamide (38.9 mg, 0.09 mmol, 39% yield) as a solid and (R)-N-(1-(3-(2-(methoxymethyl)pyridin-4-yl)-1,2,4-thiadiazol-5-yl)ethyl)-1-methyl-3-(trifluoromethyl)-1H-pyrazole-5-carboxamide (10.0 mg) as a solid. N-[(1S)-1-[3-[2-(methoxymethyl)-4-pyridyl]-1,2,4-thiadiazol-5-yl]ethyl]-2-methyl-5-(trifluoromethyl)pyrazole-3-carboxamide (10.0 mg) was purified by SFC (Column DAICEL CHIRALCEL OJ (250 mm * 30 mm, 10 µm), Condition 0.1% NH₃H₂O-EtOH, Begin B 15%, End B 15%, FlowRate (mL/min) 60) to give N-[(1S)-1-[3-[2-(methoxymethyl)-4-pyridyl]-1,2,4-thiadiazol-5-yl]ethyl]-2-methyl-5-(trifluoromethyl)pyrazole-3-carboxamide (3.41 mg, 0.008 mmol, 34% yield) as a solid.

43: ¹H NMR (CDC1₃, 400 MHz) δ_{H} = 8.70 (d, 1H), 8.31-8.18 (m, 1H), 8.02 (dd, 1H), 6.96-6.87 (m, 1H), 6.80 (d, 1H), 5.76-5.65 (m, 1H), 4.67 (s, 2H), 4.24 (s, 3H), 3.53 (s, 3H), 1.83 (d, 3H). ¹⁹F NMR (376.5 MHz, DMSO-d₆) δ_{F} -62.174. LCMS Rₜ = 0.948 min in 2.0 min chromatography, 10-80AB, MS ESI calcd. for C₁₇H₁₈F₃N₆O₂S [M+H]⁺427.1, found 427.0.

42: ¹H NMR (CDC1₃, 400 MHz) δ_{H} = 8.71 (d, 1H), 8.31-8.23 (m, 1H), 8.03 (d, 1H), 6.94-6.89 (m, 1H), 6.77 (d, 1H), 5.81-5.61 (m, 1H), 4.68 (s, 2H), 4.24 (s, 3H), 3.53 (s, 3H), 1.83 (d, 3H). ¹⁹F NMR (376.5 MHz, CDC1₃) δ_{F} -62.174. LCMS Rₜ = 0.956 min in 2.0 min chromatography, 10-80 AB, MS ESI calcd. for C₁₇H₁₈F₃N₆O₂S [M+H]⁺427.1, found 427.1.

Example 44. Synthesis of 2-methyl-N-[(1S)-1-[3-(2-methoxy-4-pyridyl)-1,2,4-thiadiazol-5-yl]ethyl]-5-(trifluoromethyl)pyrazole-3-carboxamide (44)

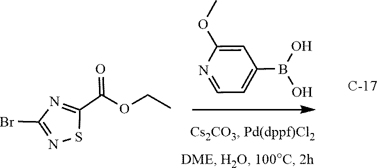

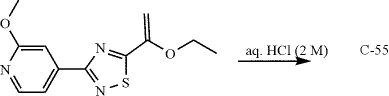

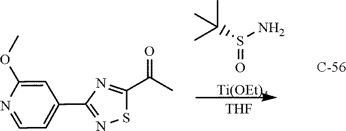

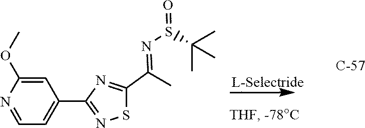

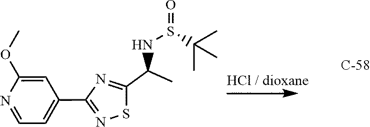

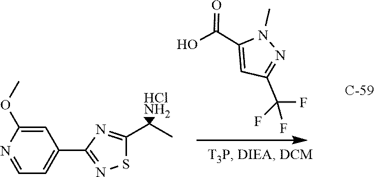

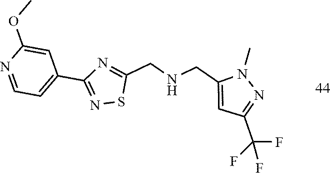 [SMILES: COc1cc(-c2nsc(CNCc3cc(C(F)(F)F)nn3C)n2)ccn1]

### 5-Ethoxyvinyl)-3-(2-methoxypyridin-4-yl)-1,2,4-thiadiazole (C-55)

To a mixture of (2-methoxy-4-pyridyl)boronic acid (1.27 g, 8.29 mmol) and 3-bromo-5-(1-ethoxyvinyl)-1,2,4-thiadiazole (1.50 g, 6.38 mmol) and C_{S2}CO₃ (4.16 g, 12.7 mmol) in Water (1.0 mL) and DME (10.0 mL, 6.38 mmol) was added Pd(dppf)C1₂ (0.7 g, 0.96 mmol) under N₂. After stirring at 100° C. for 1 h, the mixture was filtered and the filtrated was concentrated to remove dioxane. The aqueous layer was extracted with EtOAc (3 × 20.0 mL). The combined organic layers were washed with brine (30.0 mL), dried over anhydrous Na₂SO₄, filtered and concentrated. The residue was purified by flash column (10-40% of EtOAc in PE) to give the product (1.30 g, 4.44 mmol, 70% yield) as an oil. ¹H NMR (CDC1₃, 400 MHz) δ_{H} = 8.35-8.26 (m, 1H), 7.82-7.69 (m, 1H), 7.63 (s, 1H), 5.58 (d, 1H), 4.56 (d, 1H), 4.06-3.97 (m, 5H), 1.50-1.39 (m, 3H).

### 1-(2-Methoxypyridin-4-yl)-1,2,4-thiadiazol-5-yl)ethenone (C-56)

To a mixture of 5-(1-ethoxyvinyl)-3-(2-methoxy-4-pyridyl)-1,2,4-thiadiazole (1.30 g, 4.94 mmol) in acetone (15.0 mL) was added 12 HC1 (2.0 mL, 4.94 mmol). After stirring at 50° C. for 16 h, the mixture was diluted with water (10 mL) and extracted with EtOAc (15 mL × 3). The combined organic phase was washed with brine (20 mL), dried over anhydrous Na₂SO₄, filtered and concentrated to afford the product (1.1 g, 4.21 mmol, 85% yield) as an oil. ¹H NMR (CDC1₃, 400 MHz) δ_{H} = 8.36 (d, 1H), 7.80 (d, 1H), 7.69 (s, 1H), 4.06 (s, 3H), 2.82 (s, 3H).

### (R,E)-N-(1-(3-(2-methoxypyridin-4-yl)-1,2,4-thiadiazol-5-yl)ethylidene)-2-methylpropane-2-sulfinamide (C-57)

To a solution of 1-[3-(2-methoxy-4-pyridyl)-1,2,4-thiadiazol-5-yl]ethanone (300 mg, 1.28 mmol) in THF (5.0 mL) and (R)-2-methylpropane-2-sulfinamide (232 mg, 1.91 mmol) was added Ti(OEt)₄ (0.87 g, 3.83 mmol). The mixture was stirred at 50° C. for 16 h, then cooled to 25° C. before it was poured into a rapidly stirred solution of NaHCO₃ (10 mL). After the solution was stirred for 5 min, celite was stirred into the slurry and the suspension was filtered through a pad of celite. The solids were washed with EtOAc (3 × 10 mL) and the combined filtrates were transferred to a separatory funnel. The aqueous portion was separated and extracted with EtOAc (2 × 10 mL), and the combined organic portions were dried over Na₂SO₄, filtered, and evaporated under reduced pressure. The product was purified by column chromatography (increasing polarity from 5% to 20% EtOAc in pentane as eluant) to give the product (300 mg, 0.80 mmol, 63% yield) as an oil. ¹H NMR (CDC1₃, 400 MHz) δ_{H} = 8.32 (d, 1H), 7.74 (d, 1H), 7.64 (s, 1H), 4.02 (s, 3H), 2.95 (s, 3H), 1.36 (s, 9H).

### (R)-N-((S)-1-(3-(2-methoxypyridin-4-yl)-1,2,4-thiadiazol-5-yl)ethyl)-2-methylpropane-2-sulfinamide (C-58)

To a solution of (R,E)-N-[1-[3-(2-methoxy-4-pyridyl)-1,2,4-thiadiazol-5-yl]ethylidene]-2-methyl-propane-2-sulfinamide (300 mg, 0.89 mmol) in THF (5 mL) was added K-Selectride (1.77 mL, 1.77 mmol) at -78° C. After strring at -78° C. for 0.5 h, the mixture was poured into saturated NH₄C1 (20 mL) and extracted with EtOAc (2 × 10 mL). The combined organic layer was washed with brine (2 × 20 mL), dried over anhydrous Na₂SO₄, filtered and concentrated. The residue was purified by flash column (0~50% of EtOAc in PE) to give the product (150 mg, 0.40 mmol, 45% yield) as as a solid. ¹H NMR (CDC1₃, 400 MHz) δ_{H} = 8.36-8.27 (m, 1H), 7.78-7.72 (m, 1H), 7.64 (s, 1H), 5.06-4.95 (m, 1H), 4.04 (s, 3H), 1.85-1.80 (m, 3H), 1.33 (s, 9H).

### (1S)[3-(2-methoxy-4-pyridyl)-1,2,4-thiadiazol-5-yl]ethanamine (C-59)

To a solution of (R)-N-[(1S)-1-[3-(2-methoxy-4-pyridyl)-1,2,4-thiadiazol-5-yl]ethyl]-2-methyl-propane-2-sulfinamide (140 mg, 0.41 mmol) in 1,4-Dioxane (5.0 mL) was added 4 M HCl/dioxane (6.0 mL, 2.06 mmol) at 25° C. After stirring at 25° C. for 1 h, the residue was filtered and concentrated to give (1S)-1-[3-(2-methoxy-4-pyridyl)-1,2,4-thiadiazol-5-yl]ethanamine hydrochloride (120 mg, 0.508 mmol) as as a solid. ¹H NMR (MeOD, 400 MHz) δ_{H} = 8.54-8.38 (m, 1H), 8.12-8.05 (m, 1H), 8.00 (s, 1H), 5.33-5.18 (m, 1H), 4.18 (s, 3H), 1.83 (d, 3H).

### 2-methyl-N-[(1S)-1-[3-(2-methoxy-4-pyridyl)-1,2,4-thiadiazol-5-yl]ethyl]-5-(trifluoromethyl)pyrazole-3-carboxamide (44)

To a solution of 2-methyl-5-(trifluoromethyl)pyrazole-3-carboxylic acid (128 mg, 0.66 mmol) in DCM (8.0 mL) was added DIEA (524 mg, 4.06 mmol) and T₃P (1.16 g, 1.52 mmol). After stirring at 25° C. for 20 mins, (1S)-1-[3-(2-methoxy-4-pyridyl)-1,2,4-thiadiazol-5-yl]ethanamine hydrochloride (120 mg, 0.51 mmol) was added and the reaction mixture was stirred at 25° C. for 16 hr. The reaction mixture was quenched with water (20.0 mL) and extracted with DCM (2 × 15.0 mL). The combined organic layer was washed with brine (20.0 mL) and dried over Na₂SO₄, filtered and concentrated. The residue was purified by flash column (0~60% of EtOAc in PE) to give the product (210 mg, 0.509 mmol) as as a solid. The product was purified by SFC (Column DAICEL CHIRALCEL OJ (250 mm * 30 mm, 10 µm) Condition 0.1%NH₃H₂0 MeOH Begin B 30% End B 30% Gradient Time (min) 100% B Hold Time (min) FlowRate (mL/min) 60 Injections 30) to give the product (38.0 mg, 0.092 mmol, 18% yield) as as a solid. ¹H NMR (CDC1₃, 400 MHz) δ_{H} = 8.30 (d, 1H), 7.70 (d, 1H), 7.60 (s, 1H), 6.88 (s, 1H), 6.75-6.60 (m, 1H), 5.81-5.55 (m, 1H), 4.24 (s, 3H), 4.00 (s, 3H), 1.82 (d, 3H). ¹⁹F NMR (376.5 MHz, CDC1₃) δ_{F} = -62.186. LCMS Rₜ = 1.066 min in 1.5 min chromatography, 5-95AB, MS ESI calcd. for C₁₆H₁₆F₃N₆O₂S [M+H]⁺412.9, found 412.9.

Examples 44 and 45. Synthesis of (R)-N-(1-(3-(2-methoxypyridin-4-yl)-1,2,4-thiadiazol-5-yl)ethyl)-1-methyl-3-(trifluoromethyl)-1H-pyrazole-5-carboxamide & (S)-N-(1-(3-(2-methoxypyridin-4-yl)-1,2,4-thiadiazol-5-yl)ethyl)-1-methyl-3-(trifluoromethyl)-1H-pyrazole-5-carboxamide

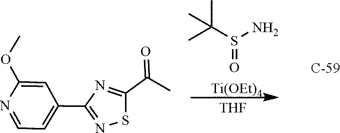

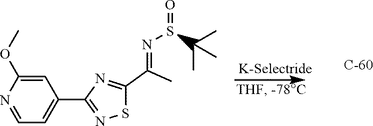

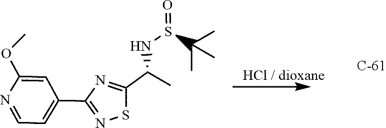

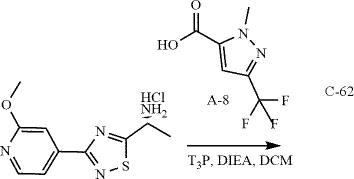

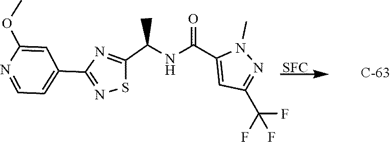

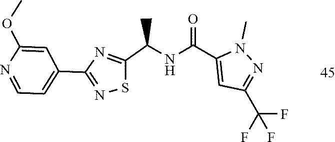 [SMILES: COc1cc(-c2nsc([C@@H](C)NC(=O)c3cc(C(F)(F)F)nn3C)n2)ccn1]

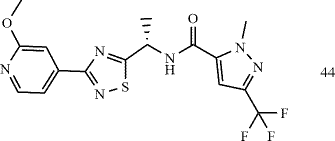 [SMILES: COc1cc(-c2nsc([C@H](C)NC(=O)c3cc(C(F)(F)F)nn3C)n2)ccn1]

### (S,E)-N-(1-(3-(2-methoxypyridin-4-yl)-1,2,4-thiadiazol-5-yl)ethylidene)-2-methylpropane-2-sulfinamide (C-60)

To a solution of 1-[3-(2-methoxy-4-pyridyl)-1,2,4-thiadiazol-5-yl]ethanone (300 mg, 1.28 mmol) in THF (5.0 mL) was added and(S)-2-methylpropane-2-sulfinamide (232 mg, 1.91 mmol) and Ti(OEt)₄ (0.87 g, 3.83 mmol). After stirring at 50° C. for 16 h, the mixture was cooled to 25° C. and poured into sat. NaHCO₃ (10.0 mL). After stirring for 5 min, celite was stirred into the slurry and the suspension was filtered through a pad of celite. The solids were washed with EtOAc (3 × 10.0 mL) and the combined filtrates were extracted with EtOAc (2 × 10.0 mL). The combined organic layer was dried over Na₂SO₄, filtered, and concentrated under reduced pressure to give the product which was purified by column chromatography (EtOAc in PE, 5%~20%) to give the product (230 mg, 0.612 mmol, 48% yield) as an oil. ¹H NMR (CDC1₃, 400 MHz) δ_{H} = 8.34 (d, 1H), 7.83-7.77 (m, 1H), 7.68 (s, 1H), 4.06 (s, 3H), 2.95 (s, 3H), 1.36 (s, 9H).

### (S)-N-((R)-1-(3-(2-methoxypyridin-4-yl)-1,2,4-thiadiazol-5-yl)ethyl)-2-methylpropane-2-sulfinamide (C-61)

To a solution of (S,E)-N-[1-[3-(2-methoxy-4-pyridyl)-1,2,4-thiadiazol-5-yl]ethylidene]-2-methyl-propane-2-sulfinamide (200 mg, 0.59 mmol) in THF (3.0 mL) was added K-Selectride (1.18 mL, 1.18 mmol) at -78° C. After strring at -78° C. for 0.5 h, the mixture was poured into saturated NH₄C1 (20.0 mL) and extracted with EtOAc (2 × 10.0 mL). The combined organic layer was washed with brine (2 × 20.0 mL), dried over anhydrous Na₂SO₄, filtered and concentrated under reduced pressure. The residue was purified by flash column (0-50% of EtOAc in PE) to give the product (100 mg, 0.27 mmol, 45% yield) as as a solid. LCMS Rₜ = 0.921 min in 1.5 min chromatography, 5-95AB, MS ESI calcd. for C₁₄H₂₁N₄O₂S₂ [M+H]⁺341.1, found 341.1.

### (R)(3-(2-methoxypyridin-4-yl)-1,2,4-thiadiazol-5-yl)ethanamine Hydrochloride (C-62)

To a solution of (S)-2-methyl-N-[(1R)-1-[3-(2-methoxy-4-pyridyl)-1,2,4-thiadiazol-5-yl]ethyl]propane-2-sulfinamide (100 mg, 0.29 mmol) in dioxane (0.50 mL) was added 4 M HCl/dioxane (1.10 mL, 4.41 mmol) at 25° C. After stirring at 25° C. for 1 hour, the reaction mixture was filtered and the residue was washed with dioxane (5.0 mL) to give the product (80.0 mg, 0.24 mmol) as as a solid. LCMS Rt = 0.679 min in 1.5 min chromatography, 5-95AB, MS ESI calcd. for C₁₀H₁₃ N₄OS [M+H]⁺237.1, found 237.1.

### (R)-N-(3-(2-methoxypyridin-4-yl)-1,2,4-thiadiazol-5-yl)ethyl)-1-methyl-3-(trifluoromethyl)-1H-pyrazole-5-carboxamide (C-63)

To a solution of 2-methyl-5-(trifluoromethyl)pyrazole-3-carboxylic acid (74.8 mg, 0.39 mmol) in DCM (8.0 mL) was added DIEA (306 mg, 2.37 mmol), T₃P (676 mg, 0.89 mmol). After stirring at 25° C. for 20 mins, (1R)-1-[3-(2-methoxy-4-pyridyl)-1,2,4-thiadiazol-5-yl]ethanamine (70.0 mg, 0.30 mmol) was added and the reaction mixture was stirred at 25° C. for 16 hr. The reaction mixture was quenched with water (10.0 mL) and extracted with DCM (2 × 15.0 mL). The combined organic layer was washed with brine (20.0 mL), dried over Na₂SO₄, filtered and concentrated to give the product (120 mg, 0.26 mmol) as a solid which was purified by prep-HPLC (Column: Welch Xtimate C18 150 * 25 mm * 5 µm; Condition: water (10 mM NH₄HCO₃)- ACN; Begin B: 46, End B: 76) to give the product (60.0 mg, 0.131 mmol) as a solid. LCMS Rₜ = 0.755 min in 1.0 min chromatography, 5-95AB, MS ESI calcd. for C₁₆H₁₆F₃N₆O₂S [M+H]⁺413.1, found 413.1.

### (R)-N-(3-(2-methoxypyridin-4-yl)-1,2,4-thiadiazol-5-yl)ethyl)-1-methyl-3-(trifluoromethyl)-1H-pyrazole-5-carboxamide & (S)-N-(3-(2-methoxypyridin-4-yl)-1,2,4-thiadiazol-5-yl)ethyl)-1-methyl-3-(trifluoromethyl)-1H-pyrazole-5-carboxamide

2-methyl-N-[(1R)-1-[3-(2-methoxy-4-pyridyl)-1,2,4-thiadiazol-5-yl] ethyl]-5-(trifluoromethyl)pyrazole-3-carboxamide (60.0 mg, 0.131 mmol) was purified by SFC (Column DAICEL CHIRALCEL OJ (250 mm * 30 mm, 10 µm), Condition: 0.1%NH₃H₂O-MeOH, Begin B: 30%, End B: 30%, FlowRate (mL/min): 60, Injections: 30) to 2-methyl-N-[(1S)-1-[3-(2-methoxy-4-pyridyl)-1,2,4-thiadiazol-5-yl]ethyl]-5-(trifluoromethyl)pyrazole-3-carboxamide (2.14 mg, 4% yield) and 2-methyl-N-[(1R)-1-[3-(2-methoxy-4-pyridyl)-1,2,4-thiadiazol-5-yl]ethyl]-5-(trifluoromethyl)pyrazole-3-carboxamide (35.4 mg, 59% yield) as a solid.

44: ¹H NMR (CDC1₃, 400 MHz) δ_{H} = 8.36-8.25 (m, 1H), 7.75-7.67 (m, 1H), 7.60 (s, 1H), 6.88 (s, 1H), 6.70-6.60 (m, 1H), 5.78-5.62 (m, 1H), 4.24 (s, 3H), 4.00 (s, 3H), 1.91-1.73 (m, 3H). ¹⁹F NMR (376.5 MHz, CDC1₃) δ_{F} = -62.175. LCMS Rₜ= 0.271 min in 2.0 min chromatography, 50-100 AB, MS ESI calcd. for C₁₆H₁₆F₃N₆O₂S [M+H]⁺413.1, found 413.1.

45: ¹H NMR (CDC1₃, 400 MHz) δ_{H} = 8.41-8.21 (m, 1H), 7.77-7.65 (m, 1H), 7.60 (s, 1H), 6.89 (s, 1H), 6.77-6.55 (m, 1H), 5.70 (t, 1H), 4.24 (s, 3H), 4.00 (s, 3H), 1.92-1.73 (m, 3H). ¹⁹F NMR (376.5 MHz, CDC1₃) δ_{F} = -62.177. LCMS Rₜ = 0.905 min in 2.0 min chromatography, 50-100 AB, MS ESI calcd. for C₁₆H₁₆F₃N₆O₂S [M+H]⁺413.1, found 413.1.

Examples 46 and 47: Synthesis of 2-methyl-N-[rac-(1S)-1-[3-(1-piperidyl)-1,2,4-thiadiazol-5-yl]ethyl]-5-(trifluoromethyl)pyrazole-3-carboxamide & 2-methyl-N-[rac-(1R)-1-[3-(1-piperidyl)-1,2,4-thiadiazol-5-yl]ethyl]-5-(trifluoromethyl)pyrazole-3-carboxamide

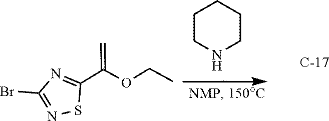

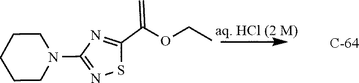

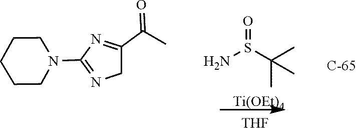

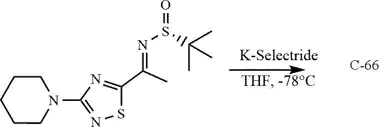

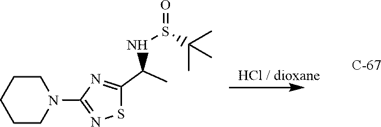

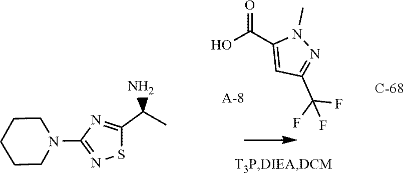

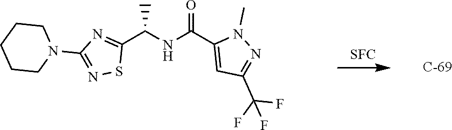 [SMILES: C[C@H](NC(=O)c1cc(C(F)(F)F)nn1C)c1nc(N2CCCCC2)ns1 ; "SFC"]

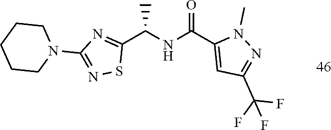 [SMILES: C[C@H](NC(=O)c1cc(C(F)(F)F)nn1C)c1nc(N2CCCCC2)ns1]

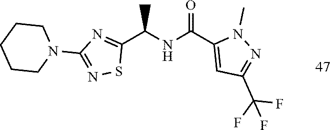 [SMILES: C[C@@H](NC(=O)c1cc(C(F)(F)F)nn1C)c1nc(N2CCCCC2)ns1]

### 5-Ethoxyvinyl)-3-(piperidin-1-yl)-1,2,4-thiadiazole (C-64)

A mixture of 3-bromo-5-(1-ethoxyvinyl)-1, 2, 4-thiadiazole (1.0 g, 4.25 mmol) and piperidine (1.81 g, 21.3 mmol) in DMF (10.0 mL) was stirred at 150° C. for 10 mins. After cooling to 20° C., the mixture was diluted with water (5.0 mL) and extracted with DCM (3 × 5.0 mL). The combined organic phase was washed with brine (20 mL), dried over anhydrous Na₂SO₄, filtered and concentrated. The residue was purified by flash column (8-10% of EtOAc in PE) to afford the product (700 mg, 2.78 mmol, 65% yield) as an oil. ¹H NMR (CDC1₃, 400 MHz) δ_{H} = 5.36 (d, 1H), 4.41 (d, 1H), 3.96 (q, 2H), 3.75-3.59 (m, 4H), 1.63 (s, 6H), 1.40 (t, 3H).

### 1-(Piperidin-1-yl)-1, 2, 4-thiadiazol-5-yl)ethenone (C-65)

To a mixture of 5-(1-ethoxyvinyl)-3-(1-piperidyl)-1, 2, 4-thiadiazole (700 mg, 2.92 mmol) in acetone (8.0 mL) was added HC1 (2 M) (10.0 mL, 2.92 mmol). After stirring at 45° C. for 2 days, the mixture was diluted with water (10.0 mL) and extracted with EtOAc (3 × 10.0 mL). The combined organic phase was washed with brine (20.0 mL), dried over anhydrous Na₂SO₄, filtered and concentrated to afford the product as an oil (600 mg, 2.78 mmol, 95% yield). ¹H NMR (CDC1₃, 400 MHz) δ_{H} = 3.71 (s, 4H), 2.68 (s, 3H), 1.66 (s, 6H).

### (R,E)methyl-N-[1-[3-(1-piperidyl)-1,2,4-thiadiazol-5-yl] Ethylidene] Propanesulfinamide (C-66)

To a solution of 1-[3-(1-piperidyl)-1,2,4-thiadiazol-5-yl]ethanone (300 mg, 1.42 mmol) in THF (5.0 mL) and rac-(R)-2-methylpropane-2-sulfinamide (258 mg, 2.13 mmol) was added Ti(OEt)₄ (0.97 g, 4.26 mmol). After stirring at 50° C. for 16 h, the residue was poured into NaHCO₃ (5.0 mL) and stirred for 20 min. The mixture was filtered with diatomite and the filtrate was extracted with EtOAc (3 × 5.0 mL). The combined organic phase was washed with brine (2 × 5.0 mL), dried over anhydrous Na₂SO₄, filtered and concentrated. The residue was purified by silica gel chromatography (PE/EtOAc= 5/1) to afford the product as a solid (230 mg, 0.73 mmol, 52 yield). ¹H NMR (CDC1₃, 400 MHz) δ_{H} = 3.71-3.67 (m, 4H), 2.85-2.79 (m, 3H), 1.65 (s, 6H), 1.31 (s, 9H).

### (R)methyl-N-[(1S)-1-[3-(1-piperidyl)-1,2,4-thiadiazol-5-yl]ethyl]propanesulfinamide (C-67)

K-Selectride (1.46 mL, 1.46 mmol) was added to a solution of (R,E)-2-methyl-N-[1-[3-(1-piperidyl)-1,2,4-thiadiazol-5-yl]ethylidene]propane-2-sulfinamide (230 mg, 0.73 mmol) in THF (3 mL) at -78° C. for 0.5 h. The mixture was poured into saturated NH₄C1 (2.0 mL) and extracted with EtOAc (2 × 2.0 mL). The combined organic layer was washed with brine (2 × 2.0 mL), dried over anhydrous Na₂SO₄, filtered and concentrated. The residue was purified by flash column (0-10% of MeOH in DCM) to give the product (200 mg, 0.63 mmol, 86% yield) as a solid. 1H NMR (CDC1₃, 400 MHz) δ_{H} = 4.87-4.76 (m, 1H), 3.72-3.65 (m, 4H), 1.75-1.70 (m, 3H), 1.67-1.62 (m, 6H), 1.41 (s, 1H), 1.29 (s, 9H).

### (1S)[3-(1-piperidyl)-1,2,4-thiadiazol-5-yl]ethanamine (C-68)

To a solution of (R)-2-methyl-N-[(1S)-1-[3-(1-piperidyl)-1,2,4-thiadiazol-5-yl]ethyl]propane-2-sulfinamide (200 mg, 0.63 mmol) in 1,4-Dioxane (1.0 mL) was added 4 M HCl/dioxane (1.0 mL, 19.9 mmol) at 25° C. After stirring at 25° C. for 3 hour, the reaction mixture was concentrated in vacuum to give the product (100 mg, 0.47 mmol, 75% yield) as a solid. 1H NMR (DMSO-d₆, 400 MHz) δ_{H} = 8.79-8.74 (m, 2H), 4.97-4.83 (m, 1H), 3.66-3.59 (m, 4H), 1.63-1.52 (m, 9H).

### 2-methyl-N-[(1S)-1-[3-(1-piperidyl)-1,2,4-thiadiazol-5-yl]ethyl]-5-(trifluoromethyl)pyrazole-3-carboxamide (C-69)

To a solution of 2-methyl-5-(trifluoromethyl)pyrazole-3-carboxylic acid (90.6 mg, 0.47 mmol) in DCM (0.50 mL) was added DIEA (0.74 mL, 4.24 mmol), T₃P (484 mg, 1.27 mmol) at 25° C. After stirring for 20 mins, (1S)-1-[3-(1-piperidyl)-1,2,4-thiadiazol-5-yl]ethanamine hydrochloride (90.0 mg, 0.42 mmol) was added and the reaction was stirred at 25° C. for 16 hour. The reaction was quenched by water (1.0 mL) and extracted with DCM (2 × 1.0 mL). The combined organic layer was washed with brine (1.0 mL) and dried over Na₂SO₄, filtered and concentrated. The residue was purified by flash column (0-30% of EtOAc in PE) to give the product (140 mg, 0.36 mmol, 85% yield) as a solid. ¹H NMR (CDC1₃, 400 MHz) δ_{H} = 6.83 (s, 1H), 6.67-6.61 (m, 1H), 5.56-5.48 (m, 1H), 4.23 (s, 3H), 3.69-3.64 (m, 4H), 1.69 (d, 3H), 1.65 (s, 6H).

### 2-methyl-N-[(1S)-1-[3-(1-piperidyl)-1,2,4-thiadiazol-5-yl]ethyl]-5-(trifluoromethyl)pyrazole-3-carboxamide & 2-methyl-N-[(1R)-1-[3-(1-piperidyl)-1,2,4-thiadiazol-5-yl]ethyl]-5-(trifluoromethyl)pyrazole-3-carboxamide

The residue of 2-methyl-N-[(1S)-1-[3-(1-piperidyl)-1,2,4-thiadiazol-5-yl]ethyl]-5-(trifluoromethyl)pyrazole-3-carboxamide (140 mg, 0.36 mmol) was purified by SFC (Column DAICEL CHIRALCEL OJ (250 mm * 30 mm, 10 µm), Condition 0.1%NH₃H₂O ETOH, Begin B 25%, End B 25%, Flowrate(mL/min) 60) to afford 2-methyl-N-[(1S)-1-[3-(1-piperidyl)-1,2,4-thiadiazol-5-yl]ethyl]-5-(trifluoromethyl)pyrazole-3-carboxamide (75.3 mg, 0.19 mmol) as a solid and 2-methyl-N-[(1R)-1-[3-(1-piperidyl)-1,2,4-thiadiazol-5-yl]ethyl]-5-(trifluoromethyl)pyrazole-3-carboxamide (14.3 mg, 0.04 mmol) as a solid.

46: ¹H NMR (CDC1₃, 400 MHz) δ_{H} = 6.82 (s, 1H), 6.67-6.61 (m, 1H), 5.60-5.42 (m, 1H), 4.22 (s, 3H), 3.69-3.64 (m, 4H), 1.69 (d, 3H), 1.65 (s, 6H). LCMS Rₜ = 1.623 min in 2.0 min

chromatography, 10-80 AB, MS ESI calcd. for C₁₅H₂₀F₃N₆OS [M+H]⁺389.2, found 389.2. 100%ee.

47: ¹H NMR (CDC1₃, 400 MHz) δ_{H} = 6.82 (s, 1H), 6.67-6.61 (m, 1H), 5.59-5.45 (m, 1H), 4.22 (s, 3H), 3.69-3.64 (m, 4H), 1.69 (d, 3H), 1.65 (s, 6H). LCMS Rₜ = 1.622 min in 2.0 min chromatography, 10-80 AB, MS ESI calcd. for C₁₅H₂₀F₃N₆OS [M+H]⁺389.1, found 389.1. 98.6%ee.

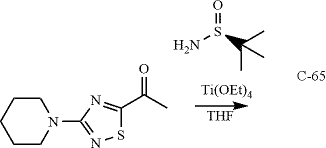

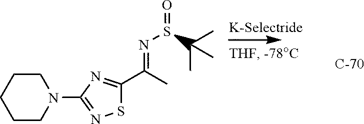

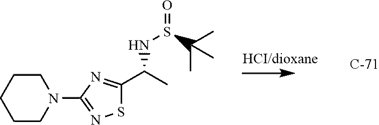

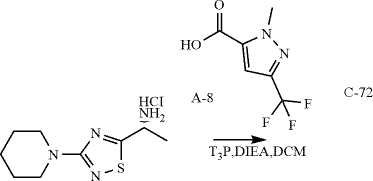

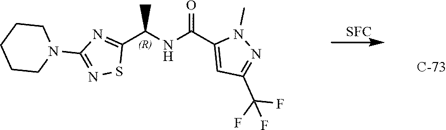 [SMILES: C[C@@H](NC(=O)c1cc(C(F)(F)F)nn1C)c1nc(N2CCCCC2)ns1 ; "SFC"]

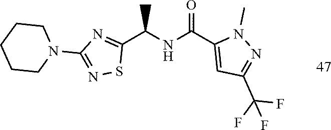 [SMILES: C[C@@H](NC(=O)c1cc(C(F)(F)F)nn1C)c1nc(N2CCCCC2)ns1]

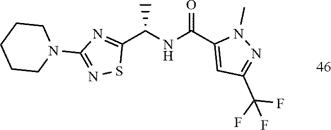 [SMILES: C[C@H](NC(=O)c1cc(C(F)(F)F)nn1C)c1nc(N2CCCCC2)ns1]

### (S, E)methyl-N-(1-(3-(piperidin-1-yl)-1, 2, 4-thiadiazol-5-yl) Ethylidene) Propanesulfinamide (C-70)

To a solution of 1-[3-(1-piperidyl)-1,2,4-thiadiazol-5-yl]ethanone (300 mg, 1.42 mmol) in THF (5.0 mL) was added rac-(S)-2-methylpropane-2-sulfinamide (258 mg, 2.13 mmol) and Ti(OEt)₄ (0.97 g, 4.26 mmol). After stirring at 50° C. for 16 h, the reaction was poured into NaHCO₃ (5.0 mL) and stirred for 20 min. The mixture was filtered with diatomite and the filtrate was extracted with EtOAc (3 × 5 mL). The combined organic phase was washed with brine (2 × 5.0 mL), dried over anhydrous Na₂SO₄, filtered and concentrated. The residue was purified by flash column (0-20% of EtOAc in PE) to afford the product (200 mg, 0.64 mmol, 45% yield) as a solid. ¹H NMR (CDC1₃, 400 MHz) δ_{H} = 3.72-3.64 (m, 4H), 2.82 (s, 3H), 1.65 (s, 6H), 1.31 (s, 9H).

### (S)methyl-N-(1-(3-(piperidin-1-yl)-1,2,4-thiadiazol-5-yl)ethyl)propanesulfinamide (C-71)

K-Selectride (1.27 mL, 1.27 mmol) was added to a solution of (S,E)-2-methyl-N-[1-[3-(1-piperidyl)-1, 2, 4-thiadiazol-5-yl]ethylidene]propane-2-sulfinamide (200 mg, 0.64 mmol) in THF (3.0 mL) at -78° C. After stirring at -78° C. for 30 mins, the mixture was poured into saturated NH₄C1 (2.0 mL) and extracted with EtOAc (2 × 2 mL). The combined organic layer was washed with brine (2 × 2.0 mL), dried over anhydrous Na₂SO₄, filtered and concentrated in vacuum. The residue was purified by flash column (0-10% of EtOAc in PE) to give the product (150 mg, 0.43 mmol, 68% yield) as an oil. 1H NMR (CDC1₃, 400 MHz) δ_{H} = 4.83-4.78 (m, 1H), 3.74-3.68 (m, 4H), 1.75-1.71 (m, 4H), 1.69-1.61 (m, 6H), 1.29 (s, 9H).

### (R)(3-(piperidinyl)-1, 2, 4-thiadiazol-5-yl)ethanamine Hydrochloride (C-72)

To a solution of (S)-2-methyl-N-[1-[3-(1-piperidyl)-1, 2, 4-thiadiazol-5-yl]ethyl]propane-2-sulfinamide (150 mg, 0.47 mmol) in 1,4-Dioxane (1.0 mL) was added 4 M HCl/dioxane (346 mg, 9.48 mmol) at 25 C. After stirring at 25 C for 1 hour, the reaction mixture was filtered and the residue was washed with dioxane (5.0 mL) to give the product (100 mg, 0.42 mmol, 89% yield) as a solid. 1H NMR (DMSO-d₆, 400 MHz) δ_{H} = 8.80 (s, 3H), 4.90 (br d, 1H), 3.63 (br d, 3H), 1.58 (br d, 9H).

### (R)methyl-N-(1-(3-(piperidinyl)-1, 2, 4-thiadiazol-5-yl)ethyl)-3-(trifluoromethyl)-1H-pyrazole-5-carboxamide (C-73)

To a solution of 2-methyl-5-(trifluoromethyl) pyrazole-3-carboxylic acid (100.6 mg, 0.52 mmol) in DCM (2.0 mL) was added DIEA (608 mg, 4.71 mmol) and T₃P (107 g, 1.41 mmol). After stirring at 25° C. for 30 mins, (R)-1-(3-(piperidin-1-yl)-1, 2, 4-thiadiazol-5-yl) ethanamine hydrochloride (100 mg, 0.47 mmol) was added and the reaction was stirred at 25° C. for 1 h. The reaction was quenched by water (20.0 mL) and extracted with DCM (2 × 20.0 mL). The combined organic layer was washed with brine (60.0 mL), dried over Na₂SO₄, filtered and concentrated in vacuum to give the product (200 mg, 0.46 mmol, 98% yield) as an oil. ¹H NMR (CDC1₃, 400 MHz) δ_{H}= 6.83 (s, 1H), 6.75-6.62 (m, 1H), 5.54-5.47 (m, 1H), 4.23 (s, 3H), 3.69-3.64 (m, 4H), 1.69 (d, 3H), 1.68-1.61 (m, 6H).

### (R)methyl-N-(1-(3-(piperidinyl)-1,2,4-thiadiazol-5-yl)ethyl)-3-(trifluoromethyl)-1H-pyrazole-5-carboxamide & (S)methyl-N-(1-(3-(piperidinyl)-1,2,4-thiadiazol-5-yl)ethyl)-3-(trifluoromethyl)-1H-pyrazole-5-carboxamide

2-methyl-N-[1-[3-(1-piperidyl)-1,2,4-thiadiazol-5-yl]ethyl]-5-(trifluoromethyl)pyrazole-3-carboxamide (200 mg, 0.51 mmol) was purified by SFC (Column: DAICEL CHIRALCEL OJ (250 mm * 30 mm, 10 µm); Condition: 0.1%NH₃H₂O-EtOH; Begin B: 25; End B: 25) to give 2-methyl-N-[(1S)-1-[3-(1-piperidyl)-1,2,4-thiadiazol-5-yl]ethyl]-5-(trifluoromethyl)pyrazole-3-carboxamide (10.4 mg, 0.03 mmol, 5% yield) as a solid and 2-methyl-N-[(1R)-1-[3-(1-piperidyl)-1,2,4-thiadiazol-5-yl]ethyl]-5-(trifluoromethyl)pyrazole-3-carboxamide (38.5 mg, 0.10 mmol, 19% yield) as a solid.

46: ¹H NMR (CDC1₃, 400 MHz) δ_{H} = 6.83 (s, 1H), 6.65 (br d, 1H), 5.56-5.47 (m, 1H), 4.22 (s, 3H), 3.69-3.62 (m, 4H), 1.69 (d, 3H), 1.68-1.62 (m, 6H).¹⁹F NMR (376.5 MHz, CDC1₃) δ_{F} -62.168.LCMS Rt = 1.070 min in 2.0 min chromatography, 30-90 AB, MS ESI calcd. for C₁₅H₂₀F₃N₆OS [M+H]⁺389.1, found 389.1. 99.2%ee.

47: ¹H NMR (CDC1₃, 400 MHz) δ_{H}= 6.83 (s, 1H), 6.65 (br d, 1H), 5.56-5.47 (m, 1H), 4.22 (s, 3H), 3.69-3.62 (m, 4H), 1.69 (d, 3H), 1.68-1.62 (m, 6H). ¹⁹F NMR (376.5 MHz, CDC1₃) δ_{F} -62.168. LCMS Rₜ = 1.073 min in 2.0 min chromatography, 30-90AB, MS ESI calcd. for C₁₅H₂₀F₃N₆OS [M+H]⁺389.1, found 389.1. 99.9%ee.

### Example 48. Efficacy of Exemplary Compounds in the Inhibition of KCNT1

### KCNT1-WT-Basal - Patch Clamp Assay

Inhibition of KCNT1 (KNa1.1, Slack) was evaluated using a tetracycline inducible cell line (HEK-TREX). Currents were recorded using the SyncroPatch 384PE automated, patch clamp system. Pulse generation and data collection were performed with PatchController384 V1.3.0 and DataController384 V1.2.1 (Nanion Technologies). The access resistance and apparent membrane capacitance were estimated using built-in protocols. Current were recorded in perforated patch mode (10 µM escin) from a population of cells. The cells were lifted, triturated, and resuspended at 800,000 cells/ml. The cells were allowed to recover in the cell hotel prior to experimentation. Currents were recorded at room temperature. The external solution contained the following (in mM): NaCl 105, NMDG 40, KC1 4, MgC1₂ 1, CaC1₂ 5 and HEPES 10 (pH = 7.4, Osmolarity ~300 mOsm). The extracellular solution was used as the wash, reference and compound delivery solution. The internal solution contained the following (in mM): NaCl 70, KF 70, KC1 10, EGTA 5, HEPES 5 and Escin 0.01 (pH = 7.2, Osmolarity ~295 mOsm). Escin is made at a 5 mM stock in water, aliquoted, and stored at -20° C. The compound plate was created at 2x concentrated in the extracellular solution. The compound was diluted to 1:2 when added to the recording well. The amount of DMSO in the extracellular solution was held constant at the level used for the highest tested concentration. A holding potential of -80 mV with a 100 ms step to 0 mV was used. Mean current was measured during the step to 0 mV. 100 µM Bepridil was used to completely inhibit KCNT1 current to allow for offline subtraction of non-KCNT1 current. The average mean current from 3 sweeps was calculated and the % inhibition of each compound was calculated. The % Inhibition as a function of the compound concentration was fit with a Hill equation to derive IC₅₀, slope, min and max parameters. If KCNT1 inhibition was less than 50% at the highest tested concentration or if an IC₅₀ could not be calculated, then a percent inhibition was reported in place of the IC₅₀.

Results from this assay are summarized in Table 1 below. In this table, “A” indicates IC₅₀ of less than or equal to 1 µM; “B” indicates inhibition of between 1 µM to 20 µM; and “C” indicates inhibition of greater than or equal to 20 µM.

TABLE 1

| Compound No. | KCNT1 WT IC₅₀ (µM) |
|---|---|
| 1 | A |
| 2 | A |
| 3 | B |
| 4 | A |
| 5 | A |
| 6 | A |
| 7 | A |
| 8 | C |
| 9 | B |
| 10 | A |
| 11 | A |
| 12 | A |
| 13 | A |
| 14 | A |
| 15 | B |
| 16 | A |
| 17 | B |
| 18 | B |
| 19 | A |
| 20 | B |
| 21 | A |
| 22 | C |
| 23 | B |
| 24 | B |
| 25 | A |
| 26 | C |
| 27 | B |
| 28 | C |
| 29 | B |
| 30 | B |
| 31 | A |
| 32 | A |
| 33 | B |
| 34 | B |
| 35 | A |
| 36 | A |
| 37 | B |
| 38 | B |
| 39 | A |
| 40 | B |
| 41 | B |
| 42 | B |
| 43 | B |
| 44 | A |
| 45 | A |
| 46 | B |
| 47 | B |

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A pharmaceutical composition comprising a compound having the Formula A: 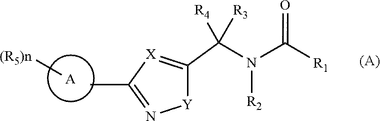
X is CR₇ or N and Y is S; or
X is CR₇ and Y is O;
ring A is selected from the group consisting of phenyl, 6-membered heteroaryl, and 5-7 membered heterocyclyl;
Rₗ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH2-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₆alkoxy, C₁₋₆haloalkoxy, and C₃₋scycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂Rₛ, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 6-membered heterocyclyl or 6-membered heteroaryl, R₁ is not thiophene;
provided that when R₃ is hydrogen and ring A is 6-membered heteroaryl or 5-membered heterocyclyl, R₁ is not phenyl; or a pharmaceutically acceptable salt thereof,
and a pharmaceutically acceptable carrier.

2. A pharmaceutical composition comprising a compound having the Formula A-1: 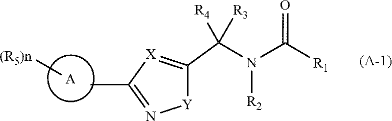
X is CR₇ or N and Y is S; or
X is CR₇ and Y is O;
ring A is 6-membered heteroaryl;
R₁ is selected from the group consisting of phenyl, 5-6 membered heteroaryl, -CH₂ phenyl, 5-8 membered carbocyclyl, and 5-10 membered heterocyclyl; wherein the phenyl, 5-6 membered heteroaryl, -CH2-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₆alkoxy, C₁₋₆haloalkoxy, and C₃₋scycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₆alkyl, C₁₋₆alkylene-O-C₁₋₆alkyl, C₁₋₆haloalkyl, C₁₋₆alkoxy, C₁₋₆haloalkoxy, —S(O)₂Rₛ, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 6-membered heteroaryl, R₁ is not thiophene or phenyl; or a pharmaceutically acceptable salt thereof,
and a pharmaceutically acceptable carrier.

3. The pharmaceutical composition of claim 1 or 2, wherein ring A is pyridyl.

4. The pharmaceutical composition of any one of claims 1-3, wherein the compound is a compound of Formula A-1A or Formula A-1B: 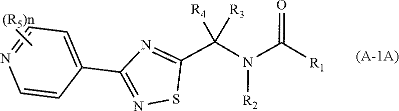 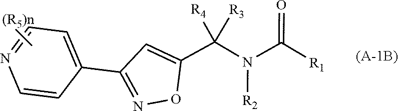
or a pharmaceutically acceptable salt thereof.

5. A pharmaceutical composition comprising a compound having the Formula A-2: 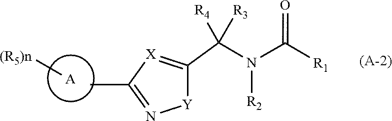
X is CR₇ or N and Y is S; or
X is CR₇ and Y is O;
ring A is 5-7 membered heterocyclyl;
R₁ is selected from the group consisting of phenyl, 5-6 membered heteroaryl, -CH₂ phenyl, 5-8 membered carbocyclyl, and 5-10 membered heterocyclyl; wherein the phenyl, 5-6 membered heteroaryl, -CH2-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of hydrogen, C₁₋₆alkyl, C₁₋₆haloalkyl, C₁₆alkoxy, C₁₋₆haloalkoxy, and C₃₋scycloalkyl, wherein the C₁₋₆alkyl is optionally substituted with C₁₋₆alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂Rₛ, —S(O)₂—N(R^{g})₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3;
provided that when R₃ is hydrogen and ring A is 5-6-membered heterocyclyl, R₁ is not thiophene or phenyl; or a pharmaceutically acceptable salt thereof,
and a pharmaceutically acceptable carrier.

6. The pharmaceutical composition of claim 1 or 5, wherein the compound is a compound of Formula A-2A: 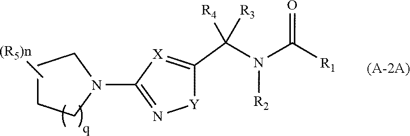
wherein q is 1 or 2;
or a pharmaceutically acceptable salt thereof.

7. The pharmaceutical composition of any one of claims 1-6, wherein X is N and Y is S.

8. The pharmaceutical composition of any one of claims 1-6, wherein X is CH and Y is O.

9. The pharmaceutical composition of any one of claims 1-8, wherein R₃ is C₁₋₆alkyl.

10. The pharmaceutical composition of any one of claims 1-8, wherein R₃ is hydrogen.

11. The pharmaceutical composition of any one of claims 1-10, wherein R₂ is hydrogen.

12. The pharmaceutical composition of any one of claims 1-11, wherein R₅ is C₁₋₆alkyl, C₁-₆alkylene-O-C₁₋₆alkyl, C₁₋₆haloalkyl, C₁₋₆alkoxy, or C₃₋₈cycloalkyl.

13. The pharmaceutical composition of any one of claims 1-12, wherein R₁ is 5-6 membered heteroaryl optionally substituted with one or more R₆.

14. The The pharmaceutical composition of claim 13, wherein the heteroaryl is pyrazolyl.

15. The pharmaceutical composition of any one of claims 1-12, wherein R₁ is phenyl optionally substituted with one or more R₆.

16. The pharmaceutical composition of any one of claims 1-12, wherein R₁ is -CH₂-phenyl optionally substituted with one or more R₆.

17. The pharmaceutical composition of any one of claims 1-12, wherein R₁ is 10-membered heterocyclyl optionally substituted with one or more R₆.

18. The pharmaceutical composition of claim 17, wherein the 10-membered heterocyclyl is a bicyclic heterocyclyl.

19. The pharmaceutical composition of any one of claims 1-18, wherein R₆ is halogen, C₁₋₆alkyl, or C₁₋₆haloalkyl.

20. A compound having the Formula I: 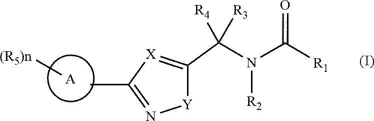
or a pharmaceutically acceptable salt thereof, wherein:
X is CR₇ or N and Y is S; or
X is CR₇ and Y is O;
ring A is selected from the group consisting of phenyl, 6-membered heteroaryl, and 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-8 membered carbocyclyl, and 5-10 membered heterocyclyl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂Rs, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

21. A compound having the Formula I-A: 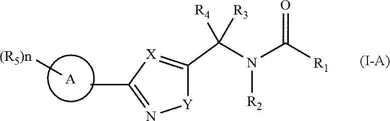
or a pharmaceutically acceptable salt thereof, wherein:
X is CR₇ or N and Y is S; or
X is CR₇ and Y is O;
ring A is 6-membered heteroaryl or 5-7 membered heterocyclyl;
R₁ is selected from the group consisting of phenyl, 5-6 membered heteroaryl, -CH₂-phenyl, 5-8 membered carbocyclyl, and 5-10 membered heterocyclyl; wherein the phenyl, 5-6 membered heteroaryl, -CH₂-phenyl, 5-10 membered carbocyclyl, and 5-10 membered heterocyclyl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆alkylene-O-C₁₋₆alkyl, C₁₋₆haloalkyl, C₁₋₆alkoxy, C₁₋₆haloalkoxy, —S(O)₂R₈, —S(O)₂—N(R₉)₂, and C₃₋₈cycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

22. A compound having the Formula I-B: 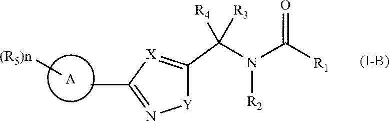
or a pharmaceutically acceptable salt thereof, wherein:
X is CR₇ or N and Y is S; or
X is CR₇ and Y is O;
ring A is phenyl or 6-membered heteroaryl;
R₁ is phenyl or 5-6 membered heteroaryl, wherein the phenyl or 5-6 membered heteroaryl is optionally substituted with one or more R₆;
R₂ is hydrogen or C₁₋₆alkyl;
R₃ is selected from the group consisting of C₁₋₆alkyl, C₁₋₆haloalkyl, C₁₋₆alkoxy, C₁₋₆haloalkoxy, and C₃₋₈cycloalkyl, wherein the C₁₋₆alkyl is optionally substituted with C₁₋₆₋ alkoxy or C₁₋₆haloalkoxy, and R₄ is hydrogen; or
R₃ and R₄ can be taken together with the carbon attached to R₃ and R₄ to form a C₃₋ scycloalkylene or 3-7 membered heterocycloalkylene;
R₅ and R₆ are each independently selected from the group consisting of halogen, C₁₋₆alkyl, C₁₋₆haloalkyl, C₁₋₆alkoxy, C₁₋₆haloalkoxy, —S(O)₂Rs, —S(O)₂—N(R₉)₂, and C₃₋ scycloalkyl;
R₇ is selected from the group consisting of hydrogen, C₁₋₆alkyl, and C₁₋₆haloalkyl;
R₈ is hydrogen or C₁₋₆alkyl;
each R₉ is independently selected from the group consisting of hydrogen, C₁₋₆alkyl, and -(C₁₋₆alkylene)-OH, or the two R₉ can be taken together with the nitrogen atom attached to the two R₉ to form a heterocycle optionally substituted with one or more substituents each independently selected from halogen and —OH; and
n is selected from the group consisting of 0, 1, 2, and 3.

23. The compound of any one of claims 20-22, wherein ring A is 6-membered heteroaryl.

24. The compound of any one of claims 20-23, wherein ring A is pyridyl.

25. The compound of any one of claims 20-23, wherein X is N and Y is S.

26. The compound of any one of claims 20-23, wherein X is CH and Y is O.

27. The compound of any one of claims 20-26, wherein R₃ is C₁₋₆alkyl.

28. The compound of any one of claims 20-27, wherein R₃ is methyl.

29. The compound of any one of claims 20-28, wherein R₂ is hydrogen.

30. The compound of any one of claims 20-21 and 23-29, wherein R₅ is C₁₋₆alkyl, C₁₋₆alkylene-O-C₁₋₆alkyl, C₁₋₆haloalkyl, C₁₋₆alkoxy, or C₃₋₈cycloalkyl.

31. The compound of any one of claims 20-21 and 23-30, wherein R₅ is cyclopropyl, -CF₃, methyl, —OCH₃, or —CH₂OCH₃.

32. The compound of any one of claims 20-30, wherein R₅ is C₃₋₈cycloalkyl or C₁₋₆haloalkyl.

33. The compound of any one of claims 20-32, wherein R₅ is cyclopropyl or -CF₃.

34. The compound of any one of claims 20-33, wherein n is 0 or 1.

35. The compound of claim 34, wherein n is 1.

36. The compound of claim 34, wherein n is 0.

37. The compound of any one of claims 20-36, wherein R₁ is 5-6 membered heteroaryl optionally substituted with one or more R₆.

38. The compound of claim 37, wherein the heteroaryl is pyrazolyl.

39. The compound of any one of claims 20-23, wherein R₁ is phenyl optionally substituted with one or more R₆.

40. The compound of any one of claims 20-21 and 23-39, wherein R₁ is -CH₂-phenyl optionally substituted with one or more R₆.

41. The compound of any one of claims 20-21 and 23-39, wherein R₁ is 10-membered heterocyclyl optionally substituted with one or more R₆.

42. The compound of claim 41, wherein the 10-membered heterocyclyl is a bicyclic heterocyclyl.

43. The compound of any one of claims 20-42, wherein R₆ is halogen, C₁₋₆alkyl, or C₁₋₆haloalkyl.

44. The compound of any one of claims 20-43, wherein R₆ is C₁₋₆alkyl or C₁₋₆haloalkyl.

45. The compound of any one of claims 20-22, wherein the compound is a compound of Formula I-IA or Formula I-IB: 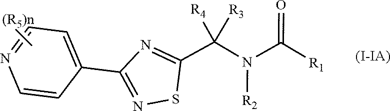 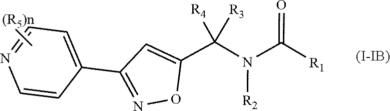
or a pharmaceutically acceptable salt thereof.

46. The compound of any one of claims 20-22 and 45, wherein the compound is a compound of Formula I-IA2 or Formula I-IB2: 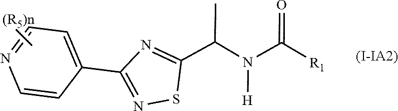 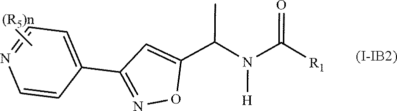
or a pharmaceutically acceptable salt thereof.

47. The compound of any one of claims 20-22 and 45-46, wherein the compound is a compound of Formula I-IA3, Formula I-IA4, Formula I-IB3, or Formula I-IB4: 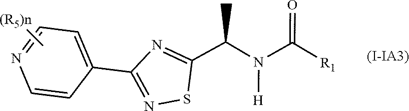 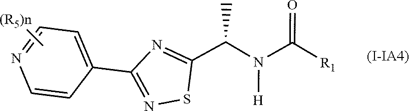 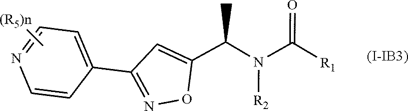 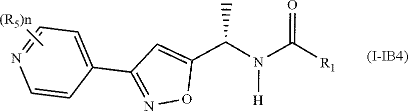
or a pharmaceutically acceptable salt thereof.

48. The compound of claim 20 or 21, wherein the compound is a compound of Formula I-IC: 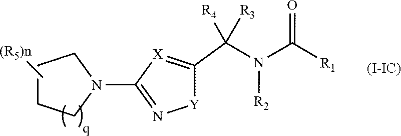
wherein q is 1 or 2;
or a pharmaceutically acceptable salt thereof.

49. The compound of any one of claims 20, 21 and 48, wherein the compound is a compound of Formula I-IC2: 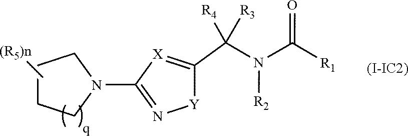
wherein q is 1 or 2;
or a pharmaceutically acceptable salt thereof.

50. The compound of claim 49, wherein the compound is a compound of Formula I-IC3 or Formula I-IC4: 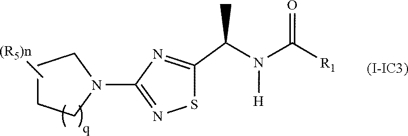 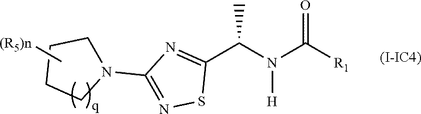
or a pharmaceutically acceptable salt thereof.

51. The compound of any one of claims 20-50, wherein R₁ is selected from the group consisting of: 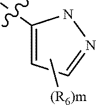 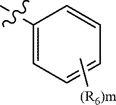 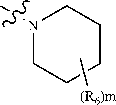 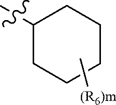 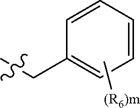 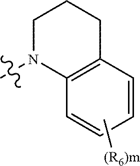 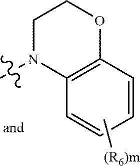
, wherein m is 0, 1, or 2.

52. The compound of claim 1, wherein the compound is selected from the group consisting of: 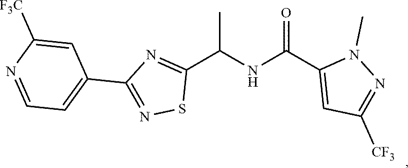 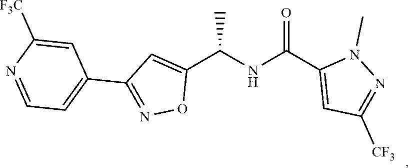 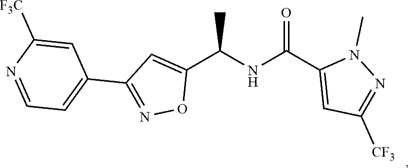 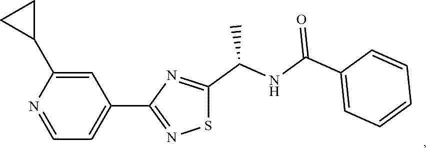 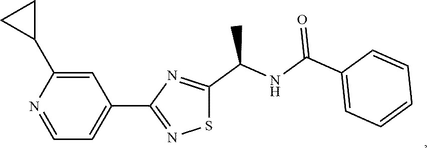 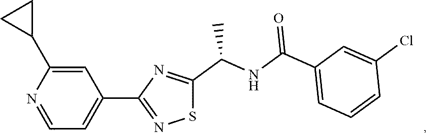 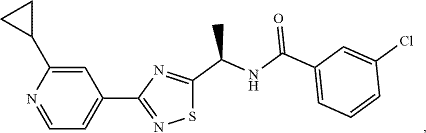 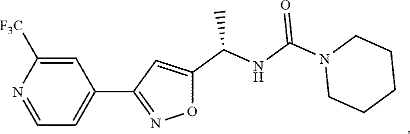 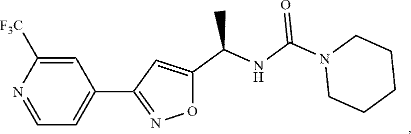 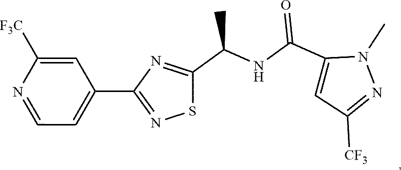 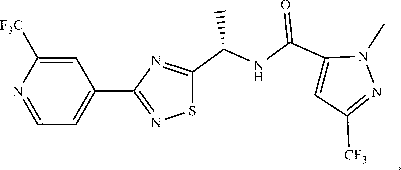 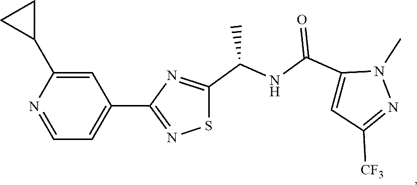 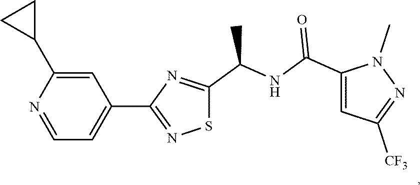 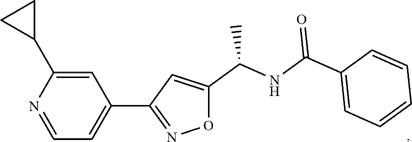 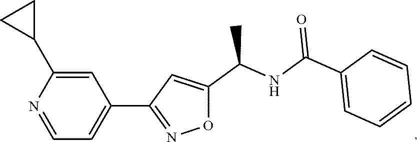 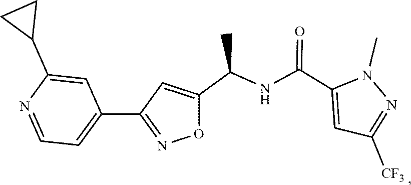 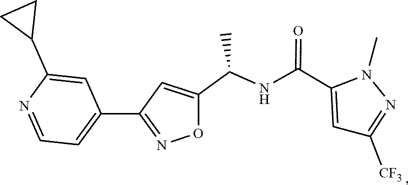 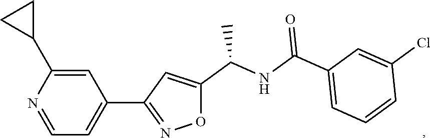 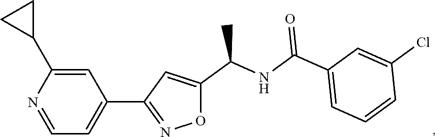 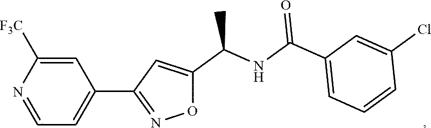 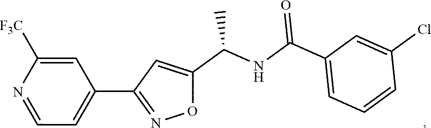 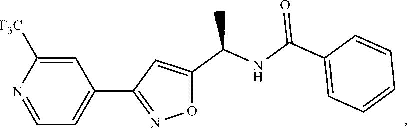 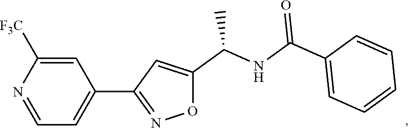 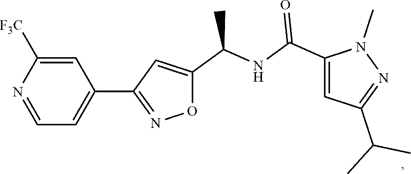 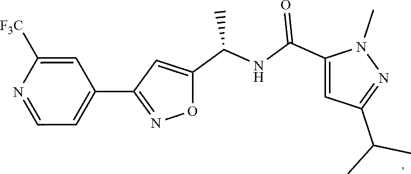 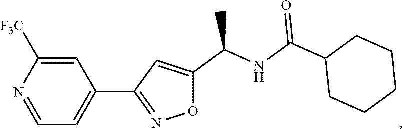 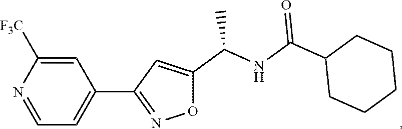 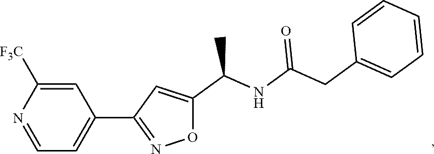 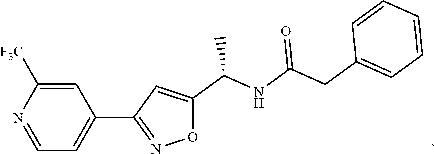 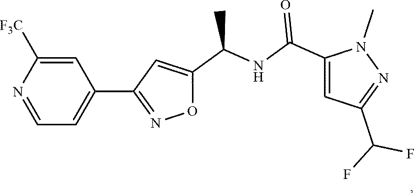 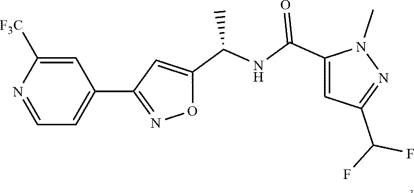 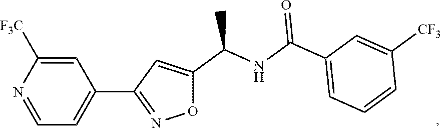 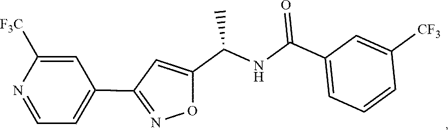 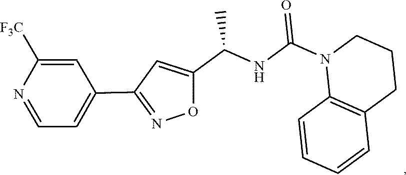 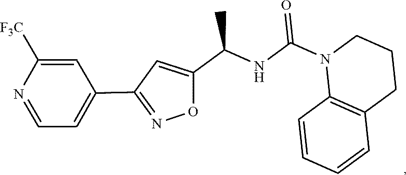 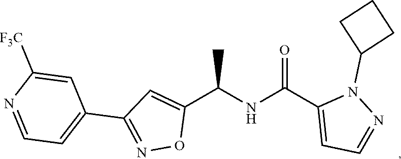 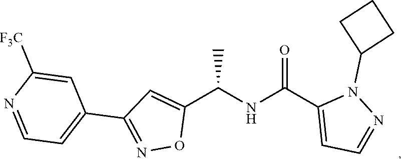 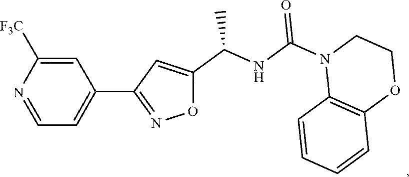 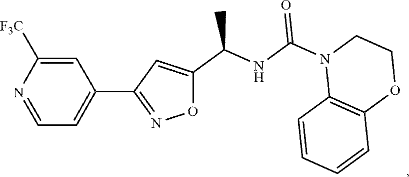 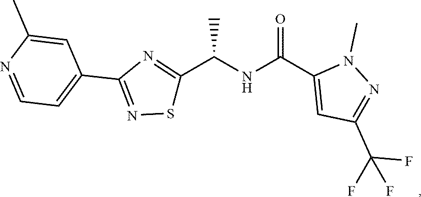 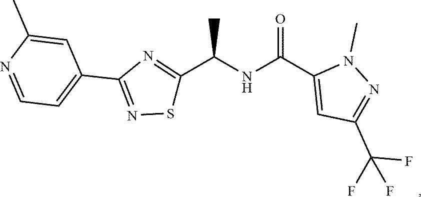 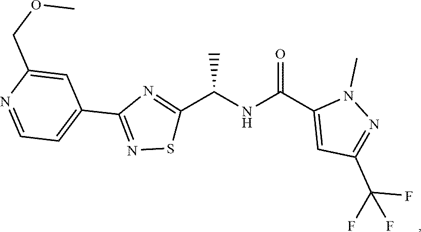 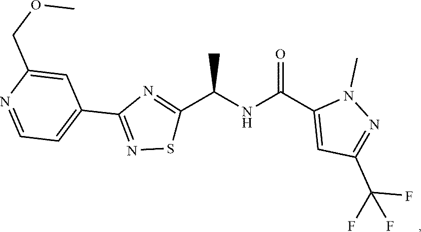 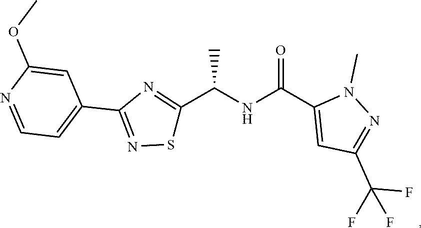 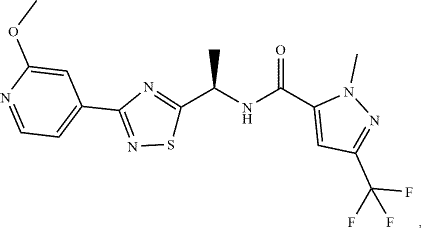 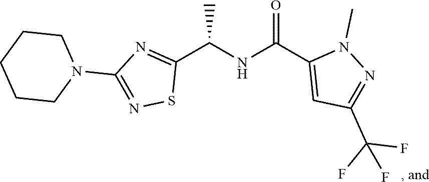 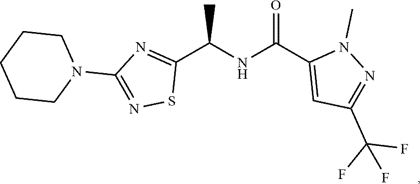
or a pharmaceutically acceptable salt thereof.

53. A pharmaceutical composition comprising a compound of any one of claims 20-52 or a pharmaceutically acceptable salt thereof and a pharmaceutically acceptable excipient.

54. A method of treating a neurological disease or disorder, wherein the method comprises administering to a subject in need thereof an effective amount of a compound of any one of claims 20-52 or a pharmaceutically acceptable salt thereof or a pharmaceutical composition of any one of claims 1-19 and 53.

55. A method of treating a disease or condition associated with excessive neuronal excitability, wherein the method comprises administering to a subject in need thereof an effective amount of a compound of any one of claims 20-52 or a pharmaceutically acceptable salt thereof or a pharmaceutical composition of any one of claims 1-19 and 53.

56. A method of treating a disease or condition associated with a gain-of-function mutation of a gene (e.g., KCNT1), wherein the method comprises administering to a subject in need thereof an effective amount of a compound of any one of claims 20-52 or a pharmaceutically acceptable salt thereof or a pharmaceutical composition of any one of claims 1-19 and 53.

57. The method of any one of claims 54-56, wherein the neurological disease or disorder, the disease or condition associated with excessive neuronal excitability, or the disease or condition associated with a gain-of-function mutation of a gene (e.g., KCNT1) is epilepsy, an epilepsy syndrome, or an encephalopathy.

58. The method of any one of claims 54-56, wherein the neurological disease or disorder, the disease or condition associated with excessive neuronal excitability, or the disease or condition associated with a gain-of-function mutation of a gene (e.g., KCNT1) is a genetic or pediatric epilepsy or a genetic or pediatric epilepsy syndrome.

59. The method of any one of claims 54-56, wherein the neurological disease or disorder, the disease or condition associated with excessive neuronal excitability, or the disease or condition associated with a gain-of-function mutation of a gene (e.g., KCNT1) is a cardiac dysfunction.

60. The method of any one of claims 54-56, wherein the neurological disease or disorder, the disease or condition associated with excessive neuronal excitability, or the disease or condition associated with a gain-of-function mutation of a gene (e.g., KCNT1) is selected from the group consisting of epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, or cerebellar ataxia).

61. The method of any one of claims 54-56, wherein the neurological disease or disorder, the disease or condition associated with excessive neuronal excitability, or the disease or condition associated with a gain-of-function mutation of a gene (e.g., KCNT1) is selected from the group consisting of cardiac arrhythmia, sudden unexpected death in epilepsy, Brugada syndrome, and myocardial infarction.

62. The method of any one of claims 54-56, wherein the neurological disease or disorder, the disease or condition associated with excessive neuronal excitability, or the disease or condition associated with a gain-of-function mutation of a gene (e.g., KCNT1) is selected from pain and related conditions (e.g. neuropathic pain, acute/chronic pain, migraine).

63. The method of any one of claims 54-56, the neurological disease or disorder, the disease or condition associated with excessive neuronal excitability, or the disease or condition associated with a gain-of-function mutation of a gene (e.g., KCNT1) is a muscle disorder (e.g. myotonia, neuromyotonia, cramp muscle spasms, spasticity).

64. The method of any one of claims 54-56, wherein the neurological disease or disorder, the disease or condition associated with excessive neuronal excitability, or the disease or condition associated with a gain-of-function mutation of a gene (e.g., KCNT1) is selected from itch and pruritis, ataxia and cerebellar ataxias.

65. The method of any one of claims 54-56, wherein the neurological disease or disorder, the disease or condition associated with excessive neuronal excitability, or the disease or condition associated with a gain-of-function mutation of a gene (e.g., KCNT1) is selected from psychiatric disorders (e.g. major depression, anxiety, bipolar disorder, schizophrenia).

66. The method of any one of claims 54-56, wherein the neurological disease or disorder or the disease or condition associated with excessive neuronal excitability and/or a gain-of-function mutation in a gene (e.g., KCNT1) is selected from the group consisting of learning disorders, Fragile X, neuronal plasticity, and autism spectrum disorders.

67. The method of any one of claims 54-56, wherein the neurological disease or disorder, the disease or condition associated with excessive neuronal excitability, or the disease or condition associated with a gain-of-function mutation of a gene (e.g., KCNT1) is selected from the group consisting of epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.
